(19) United States
(12) Patent Application Publication
(10) Pub. No.: US 2007/0032554 A1
(43) Pub. Date: Feb. 8, 2007

(54) Treatment of stuttering and other communication disorders with norepinephrine reuptake inhibitors

(75) Inventor: Douglas Kenneth Kelsey, Zionville, IN (US)

Correspondence Address: ELI LILLY & COMPANY, PATENT DIVISION, P.O. BOX 6288, INDIANAPOLIS, IN, 46206-6288, US

(73) Assignee: ELILILLY AND COMPANY a Corporation

(21) Appl. No.: 10/568,269

(22) PCT Filed: Aug. 25, 2004
(86) PCT No.: PCT/US04/25591

§ 371 Date: Feb. 14, 2006

Related U.S. Application Data
(60) Provisional application No. 60/498,018, filed on Aug. 27, 2003.

(51) Int. Cl. A61K 31/138

(52) U.S. Cl. 514/651

(57) ABSTRACT

Provided are methods and medicaments for treating stuttering or another communication disorder, comprising administering to a patient in need of such treatment an effective amount of a selective norepinephrine reuptake inhibitor.

### BACKGROUND OF THE INVENTION

1. Field of the Invention

The present invention relates to the fields of pharmaceutical chemistry and central nervous system medicine. More specifically, the present invention relates to methods of treating communication disorders, such as stuttering, in children, adolescents, and adults by administering selective norepinephrine reuptake inhibitors to patients in need of such treatment.

2. Description of Related Art

The Diagnostic and Statistical Manual ofMental Disorders, Fourth Edition (DSM-IV) (1994), American Psychiatric Association, Washington, D.C., pp. 55-65, describes a number of communication disorders usually first diagnosed in infancy, childhood, or adolescence. These include stuttering, expressive language disorder, mixed receptive-expressive language disorder, phonological disorder, and communication disorder not otherwise specified. Stuttering is perhaps the most well known of these disorders.

Stuttering is a speech disorder in which the normal flow of speech is disrupted by frequent repetitions or prolongations of speech sounds, syllables, or words, or by an individual's inability to start a word. The speech disruptions may be accompanied by rapid eye blinks, tremors of the lips and/or jaw, or other struggle behaviors of the face or upper body that a person who stutters may use in an attempt to speak. Certain situations, such as speaking before a group of people or talking on the telephone, tend to make stuttering more severe, whereas other situations, such as singing or speaking alone, often improve fluency. Stuttering is also referred to as stammering, especially in England, and by a broader term, dysfluent speech.

Characteristics of stuttering are described in Section 307.0 of the DSM-IV at pp. 63-65. While all individuals are dysfluent at times, the person who stutters is differentiated from someone with normal speech disfluencies by the kind and amount of dysfluencies.

Characteristics of stuttering include:
Repetition of sounds, syllables, parts of words, whole words, and phrases
Prolongation, or stretching, of sounds or syllables
Tense pauses, hesitations, and/or no sound between words
Speech that occurs in spurts, as the client tries to initiate or maintain voice
Related behaviors, for example reactions that accompany stuttering such as tense muscles in the lips, jaw, and/or neck; tremor of the lips, jaw, and/or tongue during attempts to speak; foot tapping, eye blinks, head turns, etc. (to try to escape from the stuttering); etc. There are many related behaviors that can occur and vary from person to person.
Variability in stuttering behavior, depending on the speaking situation, the communication partner(s), and the speaking task. A person who stutters may experience more fluency in the speech-language pathologist's office than in a classroom or workplace. There may be no difficulty making a special dinner request at home, but extreme difficulty ordering a meal in a restaurant. Conversation with a spouse may be easier, and more fluent, than that with a boss. A person may be completely fluent when singing, but experience significant stuttering when talking on the telephone.
A feeling of loss of control. The person who stutters may experience sound and word fears, situational fears, anticipation of stuttering, embarrassment, and a sense of shame. Certain sounds or words may be avoided. One word may be substituted for another that is thought to be harder to say. Or, certain speaking situations may be avoided altogether. For example, a person who stutters may always wait for someone else to answer the phone. Or, he or she may walk around a store for an hour rather than ask sales staff where an item can be found. These reactions to stuttering occur in more advanced stages.

Repetitions and prolongations are essential features of stuttering. The presence of the other listed behaviors varies from person to person.

Developmental stuttering (DS), with or without associated psychiatric illness, is the most common form, and includes all cases with gradual onset in childhood that are not the result of acquired brain damage. Persistent developmental stuttering (PDS) is DS that has not undergone spontaneous or speech therapy-induced remission. Acquired stuttering, which is much rarer than DS, may occur in previously fluent individuals. This form may be neurogenic, resulting from brain damage associated with, for example, stroke, traumatic brain injury, Alzheimer's disease, renal dialysis, Parkinson's disease, and progressive supranuclear palsy (Heuer et al. (1996) Ear Nose Throat J. 75:161-168; Brazis et al. (1996) Localization in Clinical Neurology, Third Ed., Little, Brown and Company, Boston, Mass., p.515).

Based on neuroimaging research data and the effectiveness of dopamine receptor antagonists in DS, this form of stuttering appears to have a hyperdopaminergic origin.

It is estimated that over three million Americans stutter. Stuttering affects individuals of all ages, but occurs most frequently in young children between the ages of 2 and 6 who are developing language. The prevalence of stuttering in prepubertal children is 1%, and drops to 0.8% in adolescence. The male-to-female ratio is approximately 3: 1. Most children outgrow their stuttering, and it is estimated that less than 1 percent of adults stutter.

Family and twin studies strongly suggest a genetic factor in the etiology of stuttering. The risk of stuttering among first-degree biological relatives is more than three times that in the general population. About 10% of daughters, and 20% of sons, of men who stutter will also stutter.

There is at present no cure for stuttering. However, a variety of treatments are available that may improve stuttering to some degree. These include speech therapy to improve fluency and success in communication; parent education to restructure the child's speaking environment to reduce episodes of stuttering; and the use of interventions such as electronic devices or medications. Electronic devices which help an individual control fluency may be more of a bother than a help in most speaking situations, and are often abandoned by individuals who stutter. Medications that affect brain function often have side effects that make them difficult to use for long-term treatment.

Many medications have been studied for use in treating stuttering. Evidence suggests that persons who stutter exhibit hypometabolism of the striatum and increased dopamine activity (Wu et al. (1995) Neuroreport 6:501-5; Wu et al. (1997) Neuroreport 8:767-70; Wu et al. (1997) In: Hulstijn W, Peters HRM, van Lieshout PHHM, eds. Speech production: motor control, brain research and fluency disorders. International Congress Series 1146. Amsterdam: Excerpta Medica 339-41). Drugs that boost dopamine levels exacerbate stuttering. Ritalin has a similar effect. Tricyclic antidepressants have proved ineffective, and in fact stuttering has been reported as an adverse event with the use of these compounds.

In contrast, the dopamine antagonist haloperidol has been shown in replicated, double-blind trials to reduce the symptoms of stuttering, leading to the hypothesis that D₂ receptor antagonists may be important in the treatment of developmental stuttering (J. P. Brady (1991) Am. J. Psychiatry 148:1309-16). Unfortunately, as this drug is not well tolerated by this patient population and carries substantial risk of extrapyramidal symptoms and tardive dyskinesia, it is not recommended for the treatment of stuttering.

In a recent small study, Maguire et al. ((2000) J. Clin. Psychopharmacology 20:479-482) demonstrated that the serotonin-dopamine antagonist risperidone may be effective in the treatment of developmental stuttering, and recommended further investigations of risperidone for this purpose. While extrapyramidal symptoms and akathisia were not found with the use of risperidone, sedation was common, and some participants developed transient sexual and menstrual cycle side effects that resolved with discontinuance of the medication, or with a reduction in dose. These side effects are thought to be due to the elevation of the hormone prolactin by risperidone (haloperidol also raises prolactin levels in some patients).

Paroxetine and Sertraline, selective serotonin reuptake inhibitors, are also used for the treatment of stuttering, but cause a number of undesirable side effects.

Newer medications more narrowly target dopamine receptors. Olanzapine (Zyprexa), has been used successfully to treat developmental and acquired stuttering in children, adolescents, and adults (Lavid et al. (1999) Annals of Clinical Psychiatry 11(4): 233-236; Lavid et al. (2000) Presented at the annual meeting of the American Psychiatric Association, Chicago III., 2000). Side effects were mostly limited to slight weight gain and drowsiness.

The present invention addresses the need in the art for improved treatments for stuttering that are both safe and effective.

### SUMMARY OF THE INVENTION

Accordingly, in a first aspect, the present invention provides a method of treating stuttering or another communication disorder, comprising administering to a patient in need of such treatment an effective amount of a selective norepinephrine reuptake inhibitor. The selective norepinephrine reuptake inhibitor can be, but is not limited to, any of the compounds disclosed herein.

In another aspect, the present invention provides the use of a selective norepinephrine reuptake inhibitor, such as any of the compounds disclosed herein, or other selective norepinephrine reuptake inhibitors, for the manufacture of a medicament for the treatment of stuttering or another communication disorder.

Further scope of the applicability of the present invention will become apparent from the detailed description provided below. However, it should be understood that the detailed description and specific examples, while indicating preferred embodiments of the present invention, are given by way of illustration only since various changes and modifications within the spirit and scope of the invention will become apparent to those skilled in the art from this detailed description.

### DETAILED DESCRIPTION OF THE INVENTION

The following detailed description of the invention is provided to aid those skilled in the in practicing the present invention. Even so, the following detailed description should not be construed to unduly limit the present invention as modifications and variations in the embodiments discussed herein can be made by those of ordinary skill in the art without departing from the spirit or scope of the present inventive discovery.

The contents of each of the references cited herein are herein incorporated by reference in their entirety.

Costa and Kroll ((2000) Canadian Medical Association Journal 162(13):1849-1855) disclose definitions and features of the various types of stuttering:

Developmental Stuttering—Stuttering with gradual onset in childhood as a disturbance in the normal fluency and time patterning of speech.

Persistent Developmental Stuttering—Developmental stuttering that has not undergone spontaneous or speech therapy-induced remission.

Acquired Stuttering—Stuttering that occurs more or less abruptly in previously fluent individuals. Neurogenic acquired stuttering results from brain damage, involves repetitions, prolongations, and blocks, and is not associated with grimaces, eye-blinking, or social anxiety. Psychogenic acquired stuttering begins suddenly after emotional trauma, and involves repetition of initial or stressed syllables, indifference toward dysfluency, dysfluency that never fluctuates, and persistence of normal eye contact.

Diagnostic criteria for stuttering set forth in the DSM-IV (p. 65) are:

Diagnostic Criteria for 307.0 Stuttering

A. Disturbance in the normal fluency and time patterning of speech (inappropriate for the individual's age), characterized by frequent occurrences of one or more of the following:

(1) sound and syllable repetitions

(2) sound prolongations

(3) interjections

(4) broken words (e.g., pauses within a word)

(5) audible or silent blocking (filled or unfilled pauses in speech)

(6) circumlocutions (word substitutions to avoid problematic words)

(7) words produced with an excess of physical tension

(8) monosyllabic whole-word repetitions (e.g., “I-I-I-1 see him”)

B. The disturbance in fluency interferes with academic or occupational achievement or with social communication.

C. If a speech-motor or sensory deficit is present, the speech difficulties are in excess of those usually associated with these problems.

Any of the types of stuttering described above, whether presenting alone, or comorbidly with cluttering, phonological disorder, expressive or mixed receptive-expressive language disorder, attention-deficit hyperactivity disorder (ADHD), or mental retardation, can be treated or prevented by the methods of the present invention. Patients will receive benefit from the use of norepinephrine reuptake inhibitors in the amelioration of the symptoms of stuttering regardless of whether a comorbid condition is present. Patients suffering from both stuttering and a comorbid condition, for example attention-deficit hyperactivity disorder, will receive benefit in the amelioration of symptoms of both conditions via the methods of the present invention. Therefore, in addition to methods for treating stuttering presenting alone, the present invention encompasses methods of treating stuttering comorbid with any of the conditions listed immediately above, comprising administering to a patient in need of treatment of both stuttering and such comorbid condition an effective amount of a selective norepinephrine reuptake inhibitor.

For clarification, cluttering is characterized by unpredictable, fast, and jerky outpourings of words and phrases, including slurred or omitted syllables and improper phrasing and pauses (Kaplan and Sadock (1998) Synopsis of Psychiatry, Eighth Ed., Williams and Wilkins, Baltimore, Md., pp. 1175-7). Phonological disorder (DSM-IV section 315.39) involves, in part, failure to produce developmentally expected speech sounds appropriate for the individual's age and dialect, and can involve errors in sound production, use, representation, or organization. Expressive language disorder (DSM-IV section 315.31) involves, in part, an impairment in expressive language development as demonstrated by scores on standardized individually administered measures of expressive language development substantially below those obtained from standardized measures of both nonverbal intellectual capacity and receptive language development. Mixed receptive-expressive language disorder (DSM-IV section 315.32) involves, in part, an impairment in both receptive and expressive language development as demonstrated by scores on standardized individually administered measures of both receptive and expressive language development that are substantially below those obtained from standardized measures of nonverbal intellectual capacity. Attention-deficit hyperactivity disorder (DSM-IV sections 314.00 and 314.01) involves, in part, a persistent pattern of inattention and/or hyperactivity-impulsivity that is more frequent and severe than is typically observed in individuals at a comparable level of development.

Specific diagnostic criteria in the DSM-IV for expressive language disorder, mixed receptive-expressive language disorder, phonological disorder, communication disorder not otherwise specified, as well as for attention-deficit hyperactivity disorder, each of which can exist comorbidly with developmental stuttering, are as follows:

Diagnostic Criteria for 315.31 Expressive Language Disorder

A. The scores obtained from standardized individually administered measures of expressive language development are substantially below those obtained from standardized measures of both nonverbal intellectual capacity and receptive language development. The disturbance may be manifest clinically by symptoms that include having a markedly limited vocabulary, making errors in tense, or having difficulty recalling words or producing sentences with developmentally appropriate length or complexity.

B. The difficulties with expressive language interfere with academic or occupational achievement or with social communication.

C. Criteria are not met for Mixed Receptive-Expressive Language Disorder or a Pervasive Developmental Disorder.

D. If Mental Retardation, a speech-motor or sensory deficit, or environmental deprivation is present, the language difficulties are in excess of those usually associated with these problems.

Diagnostic Criteria for 315.32 Mixed Receptive-Expressive Language Disorder

A. The scores obtained from a battery of standardized individually administered measures of both receptive and expressive language development are substantially below those obtained from standardized measures of nonverbal intellectual capacity. Symptoms include those for Expressive Language Disorder as well as difficulty understanding words, sentences, or specific types of words, such as spatial terms.

B. The difficulties with receptive and expressive language significantly interfere with academic or occupational achievement or with social communication.

C. Criteria are not met for a Pervasive Developmental Disorder.

D. If Mental Retardation, a speech-motor or sensory deficit, or environmental deprivation is present, the language difficulties are in excess of those usually associated with these problems.

Diagnostic Criteria for 315.39 Phonological Disorder

A. Failure to use developmentally expected speech sounds that are appropriate for age and dialect (e.g., errors in sound production, use, representation, or organization such as, but not limited to, substitutions of one sound for another [use of /t/ for target /k/ sound] or omissions of sounds such as final consonants).

B. The difficulties in speech sound production interfere with academic or occupational achievement or with social communication.

C. If Mental Retardation, a speech-motor or sensory deficit, or environmental deprivation is present, the speech difficulties are in excess of those usually associated with these problems.

307.9 Communication Disorder Not Otherwise Specified

This category is for disorders in communication that do not meet criteria for any specific Communication Disorder; for example, a voice disorder (i.e., an abnormality of vocal pitch, loudness, quality, tone, or resonance).

Diagnostic Criteria for Attention-Deficit/Hyperactivity Disorder

A. Either (1) or (2):

(1) six (or more) of the following symptoms of inattention have persisted for at least 6 months to a degree that is maladaptive and inconsistent with developmental level:

Inattention

(a) often fails to give close attention to details or makes careless mistakes in schoolwork, work, or other activities

(b) often has difficulty sustaining attention in tasks or play activities

(c) often does not seem to listen when spoken to directly

(d) often does not follow through on instructions and fails to finish schoolwork, chores, or duties in the workplace (not due to oppositional behavior or failure to understand instructions)

(e) often has difficulty organizing tasks and activities

(f) often avoids, dislikes, or is reluctant to engage in tasks that require sustained mental effort (such as schoolwork or homework)

(g) often loses things necessary for tasks or activities (e.g., toys, school assignments, pencils, books, or tools)

(h) is often easily distracted by extraneous stimuli

(i) is often forgetful in daily activities

(2) six (or more) of the following symptoms of hyperactivity-impulsivity have persisted for at least 6 months to a degree that is maladaptive and inconsistent with developmental level:

Hyperactivity

(a) often fidgets with hands or feet or squirms in seat

(b) often leaves seat in classroom or in other situations in which remaining seated is expected

(c) often runs about or climbs excessively in situations in which it is inappropriate (in adolescents or adults, may be limited to subjective feelings of restlessness)

(d) often has difficulty playing or engaging in leisure activities quietly

(e) is often “on the go” or often acts as if “driven by a motor”

(f) often talks excessively

Impulsivity

(g) often blurts out answers before questions have been completed

(h) often has difficulty awaiting turn

(i) often interrupts or intrudes on others (e.g., butts into conversations or games)

B. Some hyperactive-impulsive or inattentive symptoms that caused impairment were present before age 7 years.

C. Some impairment from the symptoms is present in two or more settings (e.g., at school [or work] and at home).

D. There must be clear evidence of clinically significant impairment in social, academic, or occupational functioning.

E. The symptoms do not occur exclusively during the course of a Pervasive Developmental Disorder, Schizophrenia, or other Psychotic Disorder and are not better accounted for by another mental disorder (e.g., Mood Disorder, Anxiety Disorder, Dissociative Disorder, or a Personality Disorder).

Code based on type:

314.01 Attention-Deficit/Hyperactivity Disorder, Combined Type: if both Criteria A1 and A2 are met for the past 6 months

314.00 Attention-Deficit/Hyperactivity Disorder, Predominantly Inattentive Type: if Criterion A1 is met but Criterion A2 is not met for the past 6 months

314.01 Attention-Deficit/Hyperactivity Disorder, Predominantly Hyperactive-Impulsive Type: if Criterion A2 is met but Criterion A1 is not met for the past 6 months

The methods of the present invention are effective in the treatment of patients who are children, adolescents, or adults, and there is no significant difference in the symptoms or the details of the manner of treatment among patients of different ages. In general terms, for purposes of the present invention, a child is considered to be a patient below the age of puberty, an adolescent is considered to be a patient from the age of puberty up to about 18 years of age, and an adult is considered to be a patient of 18 years or older.

Norepinephrine Reuptake Inhibitors Useful in the Present Invention

To the best of the inventor's knowledge, norepinephrine reuptake inhibitors have not been employed to treat stuttering.

Many compounds, including those discussed at length below, are selective norepinephrine reuptake inhibitors, and no doubt many more will be identified in the future. Practice of the present invention encompasses the use of norepinephrine reuptake inhibitors that exhibit 50% effective concentrations of about 1000 nM or less in the protocol described by Wong et al. (1985) Drug Development Research, 6:397. Preferred norepinephrine reuptake inhibitors useful in the methods of the present invention are those that are selective for the inhibition of norepinephrine reuptake relative to their ability to act as direct agonists or antagonists at other receptors. Preferably, the compounds useful in the methods of the present invention are selective for the inhibition of norepinephrine reuptake relative to direct agonist or antagonist activity at other receptors by a factor of at least ten, and even more preferably by a factor of at least one hundred.

Norepinephrine reuptake inhibitors useful in the methods of the present invention include, but are not limited to:

1. Atomoxetine (formerly known as tomoxetine), (R)-(−)-N-methyl-3-(2-methyl-phenoxy)-3-phenylpropylamine, is usually administered as the hydrochloride salt. Atomoxetine was first disclosed in U.S. Pat. No. 4,314,081. The term “atomoxetine” will be used here to refer to any acid addition salt or the free base of the molecule. See, for example, Gehiert et al. (1993) Neuroscience Letters 157:203-206, for a discussion of atomoxetine's activity as a norepinephrine reuptake inhibitor;

2. Reboxetine (Edronax™; Prolift™; Vestra™; Norebox™), 2-[α-(2-ethoxy)phenoxy-benzyl]morpholine, first disclosed in U.S. Pat. No. 4,229,449 for the treatment of depression, is usually administered as the racemate. Reboxetine is a selective norepinephrine reuptake inhibitor. The term “reboxetine” as used herein refers to any acid addition salt or the free base of the molecule existing as the racemate or either enantiomer, i.e., (S,S)-reboxetine or (R,R)-reboxetine. The use of (S,S)-reboxetine as a preferred selective norepinephrine reuptake inhibitor is disclosed in PCT International Publication No. WO 01/01973.

3. Compounds of formula I:

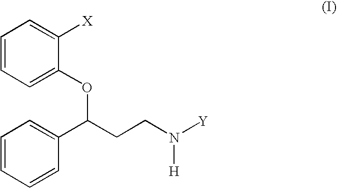

wherein X is C₁-C₄ alkylthio, and Y is C₁-C₂ alkyl or a pharmaceutically acceptable salt thereof. The compounds of formula 1 have been described in U.S. Pat. No. 5,281,624, and in Gehlert et al. (1 995) Life Sciences, 55(22):1915-1920. These compounds are disclosed as being inhibitors of norepinephrine reuptake in the brain. It should be noted that these compounds exist as stereoisomers, and accordingly include not only the racemates, but also the isolated individual isomers as well as mixtures of the individual isomers. For example, the compounds of formula 1 include the following exemplary species:

N-ethyl-3-phenyl-3-(2-methylthiophenoxy)propyl-amine benzoate;

(R)-N-methyl-3-phenyl-3-(2-propylthiophenoxy)-propylamine hydrochloride;

(S)-N-ethyl-3-phenyl-3-(2-butylthiophenoxy)propyl-amine;

N-methyl-3-phenyl-3-(2-ethylthiophenoxy)propyl-amine malonate;

(S)-N-methyl-3-phenyl-3-(2-tert-butylthiophenoxy)-propylamine naphthalene-2-sulfonate; and

(R)-N-methyl-3-(2-methylthiophenoxy)-3-phenyl-propylamine.

4. A compound of formula (IA) 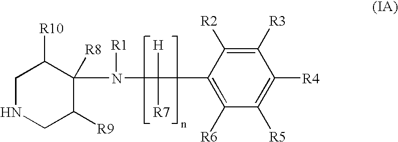
wherein n is 1, 2 or 3; R¹ is C₂-C₁₀alkyl, C2-C₁₀alkenyl, C₃-C₈cycloalkyl or C₄-C₁₀cycloalkylalkyl, wherein one C—C bond within any cycloalkyl moiety is optionally substituted by an O—C or C═C bond and wherein each group is optionally substituted with from 1 to 7 halogen substituents and/or with from 1 to 3 substituents each independently selected from hydroxy, cyano, C₁-C₄alkyl and C₁-C₄alkoxy; R2 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy); R3 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 or R4 forms a further benzene ring (optionally substituted with from 1 to 3 substituents each independently selected from halogen, C₁-C₄alkyl and C₁-C₄alkoxy); R4 is H, C₁-C₄alkyl (optionally substituted with from 1 to 7 halogen atoms), C₁-C₄alkyl-S(O)ₓ— wherein x is 0, 1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy); R5 is H, C₁-C₄alkyl (optionally substituted with from 1 to 7 halogen atoms), C₁-C₄alkoxy (optionally substituted with from 1 to 7 halogen atoms) or halogen; R6 is H, C₁-C₄alkyl (optionally substituted with from 1 to 7 halogen atoms), C₁-C₄alkoxy (optionally substituted with from 1 to 7 halogen atoms) or halogen; R7 is H or C₁-C₄alkyl; R8 is H or C₁-C₄alkyl; R9 is H, halogen, hydroxy, cyano, C₁-C₄alkyl or C₁-C₄alkoxy; and R10 is H, halogen, hydroxy, cyano, C₁-C₄alkyl or C₁-C₄alkoxy; or a pharmaceutically acceptable salt thereof, with the proviso that the compound N-ethyl-N-benzyl-4-piperidinamine is excluded.

With respect to compounds of formula (IA), the term “C₂-C₁₀alkyl” means a monovalent unsubstituted saturated straight-chain or branched-chain hydrocarbon radical having from 2 to 10 carbon atoms.

With respect to compounds of formula (IA), the term “C₂-C₁₀alkenyl” means a monovalent unsubstituted unsaturated straight-chain or branched-chain hydrocarbon radical having from 2 to 10 carbon atoms and containing at least one carbon-carbon double bond.

With respect to compounds of formula (IA), the term “C₃-C₈cycloalkyl” means a monovalent unsubstituted saturated cyclic hydrocarbon radical having from 3 to 8 carbon atoms.

With respect to compounds of formula (IA), the term “C₄-C₁₀cycloalkylalkyl” means a monovalent unsubstituted saturated cyclic hydrocarbon radical having from 3 to 9 carbon atoms linked to the point of substitution by a divalent unsubstituted saturated straight-chain or branched-chain hydrocarbon radical having at least 1 carbon atom.

With respect to compounds of formula (IA), the phrase “wherein one C—C bond within any cycloalkyl moiety is optionally substituted by an O—C or C═C bond” means that either (i) any two adjacent carbon atoms within a cycloalkyl ring may be linked by a double bond rather than a single bond (with the number of substituents on each carbon atom being reduced accordingly), or that (ii) one of any two adjacent C atoms within a cycloalkyl ring (and any substituents thereon) may be replaced by an oxygen atom. Examples of R¹ groups encompassed by this phrase include but are not limited to:

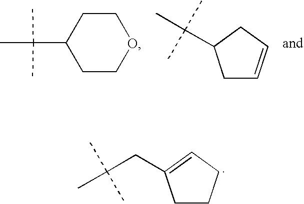

With respect to compounds of formula (IA), the term “halo” or “halogen” means F, Cl, Br or I.

With respect to compounds of formula (IA), the term “C₁-C₄alkoxy” means a monovalent unsubstituted saturated straight-chain or branched-chain hydrocarbon radical having from 1 to 4 carbon atoms linked to the point of substitution by an O atom.

With respect to compounds of formula (IA), the term “phenoxy” means a monovalent unsubstituted phenyl radical linked to the point of substitution by an O atom.

With respect to compounds of formula (IA), in the above definitions, similar terms specifying different numbers of C atoms take an analogous meaning.

Preferred compounds of formula (IA) are those wherein n is 1 or 2. More preferably, n is 1.

Preferred compounds of formula (IA) are those wherein R7 is H or methyl. More preferably R7 is H.

Preferred compounds of formula (IA) are those wherein R8 is H.

Preferred compounds of formula (IA) are those wherein R9 is H or fluoro. More preferably, R9 is H.

Preferred compounds of formula (IA) are those wherein R10 is H or fluoro. More preferably, R10 is H.

Preferred compounds of formula (IA) are those wherein R¹ is C₂-C₆alkyl, C₂-C₆alkenyl, C₃-C₆cycloalkyl or C₄-C₇cycloalkylalkyl, each of which is optionally substituted with from 1 to 3 halogen atoms or a methoxy radical. More preferably, R¹ is C₂-C₆alkyl (optionally substituted with from 1 to 3 halogen atoms or a methoxy radical), C₂-C₆alkenyl, C₃-C₆cycloalkyl or C₄-C₇cycloalkylalkyl. Suitable C₂-C6alkyl groups (optionally substituted with from 1 to 3 halogen atoms or a methoxy radical) include, for example, ethyl, n-propyl, isopropyl, n-butyl, isobutyl, n-pentyl, 3-methylbutyl, 1,2-dimethylpropyl, 1-ethylpropyl, 3,3-dimethylbutyl, 2-ethylbutyl, 3,3,3-trifluoropropyl, 4,4,4-trifluorobutyl and 2-methoxyethyl. Suitable C₂-C₆alkenyl groups include, for example, 2-metbyl-2-propenyl. Suitable C₃-C₆cycloalkyl groups include, for example, cyclopentyl. Suitable C₄-C₇cycloalkylalkyl groups include, for example, cyclohexylmethyl or cyclopropylmethyl.

Preferred compounds of formula (IA) are those wherein R¹ is a C₂-C₁₀alkyl group optionally substituted with from 1 to 7 halogen substituents and/or with from 1 to 3 substituents each independently selected from hydroxy, cyano and C₁-C₄alkoxy. More preferably, R¹ is a C₂-CIoalkyl group optionally substituted with from 1 to 3 substituents each independently selected from halogen, hydroxy and C₁-C₄alkoxy. More preferably R¹ is C₂-C₆alkyl optionally substituted with from 1 to 3 halogen atoms or a methoxy radical. Still more preferably R¹ is C₂-C₆alkyl. Still more preferably, Rl is selected from ethyl, n-propyl, isopropyl, n-butyl, isobutyl, n-pentyl, 3-methylbutyl, 1 ,2-dimethylpropyl, 1-ethylpropyl, 3,3-dimethylbutyl and 2-ethylbutyl. Most preferably R¹ is selected from n-propyl, n-butyl and isobutyl.

Preferred compounds of formula (IA) are those wherein R2 is H, C₁-C₄alkyl (optionally substituted with from 1 to 7 halogen atoms), C₁-C₄alkyl-S(O)ₓ— wherein x is 0 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy) or phenoxy (optionally substituted with from 1 to 3 substituents each independently selected from halogen, C₁-C₄alkyl and C₁-C₄alkoxy), or together with R3 forms a further benzene ring (optionally substituted with from 1 to 3 substituents each independently selected from halogen, C₁-C₄alkyl and C₁-C₄alkoxy). More preferably, R2 is H, C₁-C₂alkyl (optionally substituted with from 1 to 5 halogen atoms), C₁-C₄alkyl-S(O)ₓ— wherein x is 0 or 2 (optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy) or phenoxy (optionally substituted with from 1 to 3 substituents each independently selected from halogen, C₁-C₂alkyl and C₁-C₂alkoxy), or together with R3 forms a further benzene ring (optionally substituted with from 1 to 3 substituents each independently selected from halogen, C₁-C₂alkyl and C₁-C₂alkoxy). Still more preferably, R2 is H, methyl, trifluoromethyl, methylthio, tert-butylthio, trifluoromethylthio, methylsulfonyl, methoxy, ethoxy, difluoromethoxy, trifluoromethoxy, cyano, fluoro, chloro, bromo, phenyl or phenoxy, or together with R3 forms a further benzene ring.

Preferred compounds of formula (IA) are those wherein R2 is not H. More preferably, R2 is C₁-C₄alkyl (optionally substituted with from 1 to 7 halogen atoms), C₁-C₄alkyl-S(O)ₓ— wherein x is 0 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy) or phenoxy (optionally substituted with from 1 to 3 substituents each independently selected from halogen, C₁-C₄alkyl and C₁-C₄alkoxy), or together with R3 forms a further benzene ring (optionally substituted with from 1 to 3 substituents each independently selected from halogen, C₁-C₄alkyl and C₁-C₄alkoxy). More preferably, R2 is C₁-C₂alkyl (optionally substituted with from 1 to 5 halogen atoms), C₁-C₂alkyl-S(O)ₓ— wherein x is 0 or 2 (optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy) or phenoxy (optionally substituted with from 1 to 3 substituents each independently selected from halogen, C₁-C₂alkyl and C₁-C₂alkoxy), or together with R3 forms a further benzene ring (optionally substituted with from 1 to 3 substituents each independently selected from halogen, C₁-C₂alkyl and C₁-C₂alkoxy). Still more preferably, R2 is methyl, trifluoromethyl, methylthio, tert-butylthio, trifluoromethylthio, methylsulfonyl, methoxy, ethoxy, difluoromethoxy, trifluoromethoxy, cyano, fluoro, chloro, bromo, phenyl or phenoxy, or together with R3 forms a further benzene ring.

Preferred compounds of formula (IA) are those wherein R3 is H, C₁-C₄alkyl (optionally substituted with from 1 to 7 halogen atoms), C₁-C₄alkyl-S— (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 or R4 forms a further benzene ring (optionally substituted with from 1 to 3 substituents each independently selected from halogen, C₁-C₄alkyl and C₁-C₄alkoxy). More preferably, R3 is H, C₁-C₂alkyl (optionally substituted with from 1 to 5 halogen atoms), C₁-C₂alkyl-S— (optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy), phenoxy (optionally substituted with from 1 to 3 substituents each independently selected from halogen, C₁-C₂alkyl and C₁-C₂alkoxy) or —CO₂(C₁-C₂alkyl), or together with R2 or R4 forms a further benzene ring (optionally substituted with from 1 to 3 substituents each independently selected from halogen, C₁-C₂alkyl and C₁-C₂alkoxy). Still more preferably, R3 is H, methyl, trifluoromethyl, trifluoromethylthio, methoxy, ethoxy, difluoromethoxy, trifluoromethoxy, cyano, fluoro, chloro, bromo, phenyl, phenoxy or CO₂CH₃, or together with R2 or R4 forms a further benzene ring.

Preferred compounds of formula (IA) are those wherein R4 is H, C₁-C₄alkyl (optionally substituted with from 1 to 7 halogen atoms), C₁-C₄alkyl-S— (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), or —CO₂(C₁-C₄alkyl), or together with R3 forms a further benzene ring (optionally substituted with from 1 to 3 substituents each independently selected from halogen, C₁-C₄alkyl and C₁-C₄alkoxy). More preferably, R4 is H, C₁-C₂alkyl (optionally substituted with from 1 to 5 halogen atoms), C₁-C₂alkyl-S— (optionally substituted with from 1 to 5 halogen atoms), C₁-C₂alkoxy (optionally substituted with from 1 to 5 halogen atoms), cyano, halogen, phenyl (optionally substituted with from 1 to 3 substituents each independently selected from halogen, C₁-C₂alkyl and C₁-C₂alkoxy), or —CO₂(C₁-C₂alkyl), or together with R3 forms a further benzene ring (optionally substituted with from 1 to 3 substituents each independently selected from halogen, C₁-C₂alkyl and C₁-C₂alkoxy). Still more preferably, R4 is H, methyl, trifluoromethyl, methylthio, methoxy, trifluoromethoxy, cyano, fluoro, chloro, phenyl or CO₂CH₃, or together with R3 forms a further benzene ring.

Preferred compounds of formula (lA) are those wherein R5 is H, C₁-C₄alkyl (optionally substituted with from 1 to 5 halogen atoms), C₁-C4alkoxy (optionally substituted with from 1 to 5 halogen atoms) or halogen. More preferably, R5 is H, C₁-C₄alkyl, C₁-C₄alkoxy or halogen. Still more preferably, R5 is H, methyl, methoxy, fluoro or chloro.

Preferred compounds of formula (IA) are those wherein R6 is H, C₁-C₄alkyl (optionally substituted with from 1 to 5 halogen atoms) or halogen. More preferably, R6 is H, C₁-C₄alkyl or halogen. Still more preferably, R6 is H, methyl, fluoro or chloro.

Preferred compounds of formula (IA) are those wherein the group 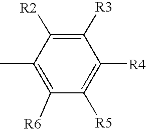
is phenyl, 2-methylphenyl, 2-(trifluoromethyl)phenyl, 2-(methylthio)phenyl, 2-(tertbutylthio)phenyl, 2-(trifluoromethylthio)phenyl, 2-(methylsulfonyl)phenyl, 2-methoxyphenyl, 2-ethoxyphenyl, 2-(difluoromethoxy)phenyl, 2-(trifluoromethoxy)phenyl, 2-cyanophenyl, 2-fluorophenyl, 2-chlorophenyl, 2-bromophenyl, 2-biphenyl, 2-phenoxyphenyl, 3-methylphenyl, 3-(trifluoromethyl)phenyl, 3-(trifluoromethylthio)phenyl, 3-methoxyphenyl, 3-ethoxyphenyl, 3-(difluoromethoxy)phenyl, 3-(trifluoromethoxy)phenyl, 3-cyanophenyl, 3-fluorophenyl, 3-chlorophenyl, 3-bromophenyl, 3-biphenyl, 3-phenoxyphenyl, 3-(methoxycarbonyl)phenyl, 4-methylphenyl, 4-(trifluoromethyl)phenyl, 4-(methylthio)phenyl, 4-methoxyphenyl, 4-(trifluoromethoxy)phenyl, 4-cyanophenyl, 4-fluorophenyl, 4-chlorophenyl, 4-biphenyl, 4-(methoxycarbonyl)phenyl, 2,3-dichlorophenyl, 2,4-dimethylphenyl, 2,4-bis(trifluoromethyl)phenyl, 2,4-dimethoxyphenyl, 2,4-difluorophenyl, 2,4-dichlorophenyl, 2,5-dimethylphenyl, 2,6-dimethylphenyl, 2,6-dichlorophenyl, 2-chloro-6-fluorophenyl, 2-fluoro-6-(trifluoromethyl)phenyl, 3,4-dichlorophenyl, 3,5-dimethylphenyl, 3,5-dimethoxyphenyl, 3,5-difluorophenyl, 3,5-dichlorophenyl, 3-fluoro-5-(trifluoromethyl)phenyl, 5-fluoro-2-(trifluoromethylphenyl), 5-fluoro-2-methoxyphenyl, 4-fluoro-2-(trifluoromethyl)phenyl, 1-naphthyl or 2-naphthyl.

A further embodiment provides a group (Group A) of compounds of formula (IA) above, wherein R2, R3, R4, R5 and R6 are all H.

urther embodiment provides a group (Group B) of compounds of formula (IA) above, wherein one of R2, R3, R4, R5 and R6 is not H and the others are H.

Compounds of Group B include those (Group B2) wherein R3, R4, R5 and R6 are all H and R2 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

Compounds of Group B also include those (Group B3) wherein R2, R4, R5 and R6 are all H and R3 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

Compounds of Group B also include those (Group B4) wherein R2, R3, R5 and R6 are all H and R4 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

A further embodiment provides a group (Group C) of compounds of formula (IA) above, wherein two of R2, R3, R4, R5 and R6 are not H and the others are H.

Compounds of Group C include those (Group C2,3) wherein R4, R5 and R6 are all H; R2 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy); and R3 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R2 forms a further benzene ring (optionally substituted with from 1 to 3 substituents each independently selected from halogen, C₁-C₄alkyl and C₁-C₄alkoxy).

Compounds of Group C also include those (Group C2,4) wherein R3, R5 and R6 are all H; R2 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and R4 is C₁-C₄alkyl (optionally substituted with from 1 to 7 halogen atoms), C₁-C₄alkyl-S(O)X— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl).

Compounds of Group C also include those (Group C2,5) wherein R3, R4 and R6 are all H; R2 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and R5 is C₁-C₄alkyl (optionally substituted with from 1 to 7 halogen atoms), C₁-C₄alkoxy (optionally substituted with from 1 to 7 halogen atoms) or halogen.

Compounds of Group C also include those (Group C2,6) wherein R3, R4 and R5 are all H; R2 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and R6 is C₁-C₄alkyl (optionally substituted with from 1 to 7 halogen atoms), C₁-C₄alkoxy (optionally substituted with from 1 to 7 halogen atoms) or halogen.

Compounds of Group C also include those (Group C3,4) wherein R2, R5 and R6 are all H; R3 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R4 forms a further benzene ring (optionally substituted with from 1 to 3 substituents each independently selected from halogen, C₁-C₄alkyl and C₁-C₄alkoxy); and R4 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl), or together with R3 forms a further benzene ring (optionally substituted with from 1 to 3 substituents each independently selected from halogen, C₁-C₄alkyl and C₁-C₄alkoxy).

Compounds of Group C also include those (Group C3,5) wherein R2, R4 and R6 are all H; R3 is C₁-C₄alkyl (optionally substituted with from 1 to 7 halogen atoms), C₁-C₄alkyl-S(O)ₓ— wherein x is 0,1 or 2 (optionally substituted with from 1 to 7 halogen atoms), C₁-C₄alkoxy (optionally substituted with from 1 to 7 halogen atoms), cyano, halogen, phenyl (optionally substituted with from 1 to 3 substituents each independently selected from halogen, C₁-C₄alkyl and C₁-C₄alkoxy), phenoxy (optionally substituted with from 1 to 3 substituents each independently selected from halogen, C₁-C₄alkyl and C₁-C₄alkoxy) or —CO₂(C₁-C₄alkyl); and R5 is C₁-C₄alkyl (optionally substituted with from 1 to 7 halogen atoms), C₁-C₄alkoxy (optionally substituted with from 1 to 7 halogen atoms) or halogen.

For compounds of Formula (IA) falling within any one of groups A, B, B2, B3, B4, C, C2,3, C2,4, C2,5, C2,6, C3,4 and C3,5 described above, n is preferably 1 or 2, more preferably 1.

For compounds of Formula (IA) falling within any one of groups A, B, B2, B3, B4, C, C2,3, C2,4, C2,5, C2,6, C3,4 and C3,5 described above, R7 is preferably H or methyl, more preferably H.

For compounds of Formula (IA) falling within any one of groups A, B, B2, B3, B4, C, C2,3, C2,4, C2,5, C2,6, C3,4 and C3,5 described above, R8 is preferably H.

For compounds of Formula (IA) falling within any one of groups A, B, B2, B3, B4, C, C2,3, C2,4, C2,5, C2,6, C3,4 and C3,5 described above, R9 is preferably H or fluoro, more preferably H.

For compounds of Formula (IA) falling within any one of groups A, B, B2, B3, B4, C, C2,3, C2,4, C2,5, C2,6, C3,4 and C3,5 described above, R10 is preferably H or fluoro, more preferably H.

For compounds of Formula (IA) falling within any one of groups A, B, B2, B3, B4, C, C2,3, C2,4, C2,5, C2,6, C3,4 and C3,5 described above, R¹ is preferably a C₂-C₁₀alkyl group optionally substituted with from 1 to 7 halogen substituents and/or with from 1 to 3 substituents each independently selected from hydroxy, cyano and C₁-C₄alkoxy.

For compounds of Formula (IA) falling within any one of groups A, B, B2, B3, B4, C, C2,3, C2,4, C2,5, C2,6, C3,4 and C3,5 described above, n is preferably 1, R7, R8, R9 and R10 are preferably H and R¹ is preferably a C₂-C₁₀alkyl group optionally substituted with from 1 to 7 halogen substituents and/or with from 1 to 3 substituents each independently selected from hydroxy, cyano and C₁-C₄alkoxy.

5. A compound of formula (IB) 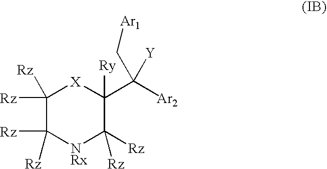
wherein Rx is H; Ry is H or C₁-C₄ alkyl; each Rz is independently H or C₁-C₄ alkyl; X represents O; Y represents OH or OR; R is C₁-C₄ alkyl; Ar₁ is a phenyl ring or a 5- or 6-membered heteroaryl ring each of which may be substituted with 1, 2, 3, 4 or 5 substituents (depending upon the number of available substitution positions) each independently selected from C₁-C₄ alkyl, O(C₁-C₄ alkyl), S(C₁-C₄ alkyl), halo, hydroxy, pyridyl, thiophenyl and phenyl optionally substituted with 1, 2, 3, 4 or 5 substituents each independently selected from halo, C₁-C₄ alkyl, or O(C₁-C₄ alkyl); and Ar₂ is a phenyl ring or a 5- or 6-membered heteroaryl ring each of which may be substituted with 1, 2, 3, 4 or 5 substituents (depending upon the number of available substitution positions) each independently selected from C₁-C₄ alkyl, O(C₁-C₄ alkyl) and halo; wherein each above-mentioned C₁-C₄ alkyl group is optionally substituted with one or more halo atoms; or a pharmaceutically acceptable salt thereof.

Preferred compounds of formula (IB) above are those wherein Ar₁ is phenyl, pyridyl, pyrimidyl, thiazolyl, isothiazolyl, oxazolyl, isoxazolyl, thiophenyl, furanyl, imidazolyl, triazolyl, oxadiazolyl or thiadiazolyl, each of which may be substituted with 1, 2, 3, 4 or 5 substituents (depending upon the number of available substitution positions) each independently selected from C₁-C₄ alkyl, O(C₁-C₄ alkyl), S(C₁-C₄ alkyl), halo, hydroxy, pyridyl, thiophenyl and phenyl optionally substituted with 1, 2, 3, 4 or 5 substituents each independently selected from halo, C₁-C₄ alkyl, or O(C₁-C₄ alkyl); and Ar₂ is phenyl, pyridyl, pyrimidyl, thiazolyl, isothiazolyl, oxazolyl, isoxazolyl, thiophenyl, furanyl, imidazolyl or triazolyl each of which may be substituted with 1, 2, 3, 4 or 5 substituents (depending upon the number of available substitution positions) each independently selected from C₁-C₄ alkyl, O(C₁-C₄ alkyl) and halo; wherein each above-mentioned C₁-C₄ alkyl group is optionally substituted with one or more halo atoms.

For the compounds of formula (IB) above, it is preferred that Ar₁ is a phenyl ring or a 5- or 6-membered heteroaryl ring substituted with 1, 2, 3, 4 or 5 substituents, more preferably with 1 or 2 substituents.

For the compounds of formula (IB) above, when Ar₁ is a substituted phenyl ring or a substituted 5- or 6-membered heteroaryl ring, it is preferred that not more than one of those substituents is a pyridyl, thiophenyl or optionally substituted phenyl group.

Preferred compounds of formula (IB) above are those wherein Ar₁ includes a substituent attached at the 2-position. That is, the substituent is attached to the atom adjacent to that which forms the point of attachment of Ar₁ to the methylene group connecting Ar₁ to the rest of the molecule. For example, when Ar₁ is phenyl, it is preferably ortho-substituted.

Further preferred compounds of formula (IB) above are those wherein Rx is H; Ry is H or C₁-C₄ alkyl; each Rz is independently H or C₁-C₄ alkyl; X represents O; Y represents OH or OR; R is C₁-C₄ alkyl; and Ar₁ and Ar₂ are each independently selected from the group consisting of phenyl, and substituted phenyl; and pharmaceutically acceptable salts thereof. In this further preferred embodiment, the group Ar₁ may be substituted or unsubstituted phenyl. For example, Ar₁ may be unsubstituted phenyl or, preferably phenyl substituted with 1, 2, 3, 4 or 5 substituents, preferably with 1 or 2, for example 1, substituent. When disubstituted, the substituted phenyl group is preferably substituted at the 2- and 5- positions. When monosubstituted, the substituted phenyl group is preferably substituted in the 2- position. Suitable substituents include C₁-C₄ alkyl, O(C₁-C₄ alkyl), S(C₁-C₄ alkyl), halo, and phenyl, optionally substituted with, for example, halo, C₁-C₄ alkyl, or O(C₁-C₄ alkyl). In this further preferred embodiment, the group Ar₂ may be substituted or unsubstituted phenyl. For example, Ar₂ may be phenyl substituted with 1, 2, 3, 4 or 5 substituents, preferably with 1 substituent. Suitable substituents include C₁-C₄ alkyl, O(C₁-C₄ alkyl), and especially, halo.

“C₁-C₄ alkyl” as used in respect of compounds of formula (IB) includes straight and branched chain alkyl groups of 1, 2, 3 or 4 carbon atoms, and may be unsubstituted or substituted. C₁-C₂ alkyl groups are preferred. Suitable substituents include halo, especially Cl and/or F. Thus the term “C₁-C₄ alkyl” includes haloalkyl. A particularly preferred substituted C₁-C₄ alkyl group is trifluoromethyl. Similar terms defining different numbers of C atoms (e.g. “C₁-C₃ alkyl”) take an analogous meaning. When Ry is C₁-C₄ alkyl it is preferably unsubstituted. When Rz is C₁-C₄ alkyl it is preferably unsubstituted. When R is C₁-C₄ alkyl it is preferably unsubstituted.

“5-membered heteroaryl ring” as used in respect of compounds of formula (IB) means a 5-membered aromatic ring including at least one heteroatom independently selected from N, O and S. Preferably there are not more than three heteroatoms in total in the ring. More preferably there are not more than two heteroatoms in total in the ring. More preferably there is not more than one heteroatom in total in the ring. The term includes, for example, the groups thiazolyl, isothiazolyl, oxazolyl, isoxazolyl, thiophenyl, furanyl, pyrrolyl, imidazolyl, triazolyl, oxadiazolyl and thiadiazolyl.

“6-membered heteroaryl ring” as used in respect of compounds of formula (IB) means a 6-membered aromatic ring including at least one heteroatom independently selected from N, O and S. Preferably there are not more than three heteroatoms in total in the ring. More preferably there are not more than two heteroatoms in total in the ring. More preferably there is not more than one heteroatom in total in the ring. The term includes, for example, the groups pyridyl, pyrimidyl, pyrazinyl, pyridazinyl and triazinyl.

“Halo” as used in respect of compounds of formula (lB) includes F, Cl, Br and I, and is preferably F or Cl.

“Pyridyl” as used in respect of compounds of formula (IB) includes 2-pyridyl, 3-pyridyl and 4-pyridyl.

“Pyrimidyl” as used in respect of compounds of formula (IB) includes 2-pyrimidyl, 4-pyrimidyl and 5-pyrimidyl.

“Pyridazinyl” as used in respect of compounds of formula (IB) includes 3-pyridazinyl and 4-pyridazinyl.

“Pyrazinyl” as used in respect of compounds of formula (IB) includes 2-pyrazinyl and 3-pyrazinyl.

“Triazinyl” as used in respect of compounds of formula (IB) includes 2-(1,3,5-triazinyl), 3-, 5- and 6-(1,2,4-triazinyl) and 4- and 5-(1,2,3-triazinyl).

“Thiazolyl” as used in respect of compounds of formula (IB) includes 2-thiazolyl, 4-thiazolyl and 5-thiazolyl.

“Isothiazolyl” as used in respect of compounds of formula (IB) includes 3-isothiazolyl, 4-isothiazolyl, and 5-isothiazolyl.

“Oxazolyl” as used in respect of compounds of formula (IB) includes 2-oxazolyl, 4-oxazolyl and 5-oxazolyl.

“Isoxazolyl” as used in respect of compounds of formula (IB) includes 3-isoxazolyl, 4-isoxazolyl, and 5-isoxazolyl.

“Thiophenyl” as used in respect of compounds of formula (IB) includes 2-thiophenyl and 3-thiophenyl.

“Furanyl” as used in respect of compounds of formula (IB) includes 2-furanyl and 3-furanyl.

“Pyrrolyl” as used in respect of compounds of formula (IB) includes 2-pyrrolyl and 3-pyrrolyl.

“Imidazolyl” as used in respect of compounds of formula (IB) includes 2-imidazolyl and 4-imidazolyl.

“Triazolyl” as used in respect of compounds of formula (IB) includes 1-triazolyl, 4-triazolyl and 5-triazolyl.

“Oxadiazolyl” as used in respect of compounds of formula (IB) includes 4- and 5-(1,2,3-oxadiazolyl), 3- and 5—(1,2,4-oxadiazolyl), 3-(1,2,5-oxadiazolyl), 2-(1,3,4-oxadiazolyl).

“Thiadiazolyl” as used in respect of compounds of formula (1B) includes 4- and 5-(1,2,3-thiadiazolyl), 3- and 5-(1,2,4-thiadiazolyl), 3-(1,2,5-thiadiazolyl), 2-(1,3,4-thiadiazolyl).

For the compounds of formula (IB) above, Ry is preferably H or Me. More preferably Ry is H.

For the compounds of formula (IB) above, each Rz is preferably H or Me with 0, 1, 2 or 3 of Rz being Me. More preferably only 1 Rz is Me. Most preferably all Rz are H.

For the compounds of formula (IB) above, Y is preferably OH or OMe. More preferably, Y is OH.

For the compounds of formula (IB) above, it is preferred that Ry and all Rz are H and Y is OH.

For the compounds of formula (IB) above, the preferred stereochemistry is shown below:

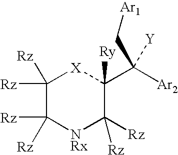

A preferred group of compounds of formula (IB) is represented by the formula (IIB) 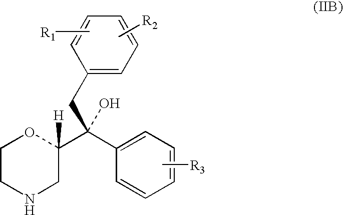
wherein R₁ and R₂ are each independently selected from H, C₁-C₄ alkyl, O(C₁-C₄ alkyl), S(C₁-C₄ alkyl), halo and phenyl; and R₃ is selected from H, C₁-C₄ alkyl and halo; and pharmaceutically acceptable salts thereof.

For the compounds of formula (IB) or (IIB) above, R₁ is preferably C₁-C₃ alkyl (especially trifluoromethyl), O(C₁-C₃ alkyl) (especially methoxy or trifluoromethoxy), F or phenyl (Ph). R₂ is preferably H. R₂ is also preferably F. R₃ is preferably H.

Especially preferred compounds of formula (IB) are 1-morpholin-2-yl-1-phenyl-2-(2-trifluoromethoxy-phenyl)-ethanol and 2-(5-fluoro-2-methoxy-phenyl)-1-morpholin-2-yl-1-phenyl-ethanol. For both of these compounds the (S,R) stereoisomer is preferred. For both of these compounds the preferred salt form is the hydrochloride salt.

6. A compound of formula (IC) 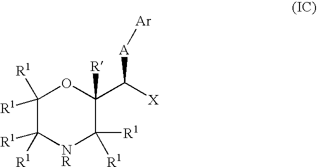
wherein: A is S or O; R is H; Ar is a phenyl group optionally substituted with 1, 2, 3, 4 or 5 substituents each independently selected from C₁-C₄ alkyl, O(C₁-C₄ alkyl), S(C₁-C₄ alkyl), halo, hydroxy, CO₂(C₁-C₄ alkyl), pyridyl, thiophenyl and phenyl optionally substituted with 1, 2, 3, 4 or 5 substituents each independently selected from halo, C₁-C₄ alkyl, or O(C₁-C₄ alkyl); X is a phenyl group optionally substituted with 1, 2, 3, 4 or 5 substituents each independently selected from halo, C₁-C₄ alkyl, or O(C₁-C₄ alkyl); a C₁-C₄ alkyl group; a C₃-C₆ cycloalkyl group or a CH₂(C₃-C₆ cycloalkyl) group; R¹ is H or C₁-C₄ alkyl; each R¹ is independently H or C₁-C₄ alkyl; wherein each above-mentioned C₁-C₄ alkyl group is optionally substituted with one or more halo atoms; or a pharmaceutically acceptable salt thereof; with the proviso that, when A is O, X is a C₁-C₄ alkyl group, a C₃-C₆ cycloalkyl group or a CH₂(C₃-C₆ cycloalkyl) group.

For the compounds of formula (IC) above, it is preferred that A is S.

For the compounds of formula (IC) above, it is preferred that Ar is phenyl substituted with 1, 2, 3, 4 or 5 substituents, more preferably with 1 or 2 substituents. When Ar is a substituted phenyl, it is preferred that not more than one of those substituents is a pyridyl, thiophenyl or optionally substituted phenyl group.

Preferred compounds of formula (IC) above are those wherein Ar is ortho-substituted.

Further preferred compounds of formula (IC) above are those of formula (ICa) 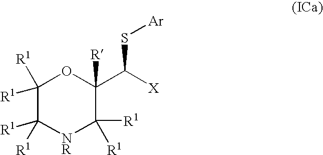
wherein: R is H; Ar is a phenyl group; X is a phenyl SSgroup; R¹ is H or C₁-C₄ alkyl; each R¹ is independently H or C₁-C₄ alkyl; and pharmaceutically acceptable salts thereof. For these further preferred compounds, the group Ar may be substituted or unsubstituted phenyl. For example, Ar may be unsubstituted phenyl or, preferably phenyl substituted with 1, 2, 3, 4 or 5 substituents, preferably with 1 or 2, for example 1, substituent. When disubstituted, the substituted phenyl group is preferably substituted at the 2- and 5-positions When monosubstituted, the substituted phenyl group is preferably substituted in the 2- position. Suitable substituents include C₁-C₄ alkyl, O(C₁-C₄ alkyl), S(C₁-C₄alkyl), halo, and phenyl optionally substituted with, for example, halo, C₁-C₄ alkyl, or O(C₁-C₄ alkyl). For these further preferred compounds, the group X may be substituted or unsubstituted phenyl. For example, X may be phenyl substituted with 1, 2, 3, 4 or 5 substituents, preferably with 1 substituent. Suitable substituents include C₁-C₄ alkyl, O(C₁-C₄ alkyl), and halo.

“C₁-C₄ alkyl” as used in respect of compounds of formula (IC) includes straight and branched chain alkyl groups of 1, 2, 3 or 4 carbon atoms, and may be unsubstituted or substituted. C₁-C₂ alkyl groups are preferred. Suitable substituents include halo. Thus the term “C₁-C₄ alkyl” includes haloalkyl. Similar termns defining different numbers of C atoms (e.g. “C₁-C₃ alkyl”) take an analogous meaning. When R¹ is C₁-C₄ alkyl it is preferably unsubstituted. When R¹ is C₁-C₄ alkyl it is preferably unsubstituted.

“C₃-C₆ cycloalkyl” as used in respect of compounds of formula (IC) includes cyclopropyl, cyclobutyl, cyclopentyl and cyclohexyl.

“Halo” as used in respect of compounds of formula (IC) includes F, Cl, Br and I, and is preferably F or Cl.

“Pyridyl” as used in respect of compounds of formula (IC) includes 2-pyridyl, 3-pyridyl and 4-pyridyl.

“Thiophenyl” as used in respect of compounds of formula (IC) includes 2-thiophenyl and 3-thiophenyl.

For the compounds of formula (IC) above, R¹ is preferably H or Me. More preferably R¹ is H.

For the compounds of formula (IC) above, each R¹ is preferably H or Me with 0, 1, 2 or 3 of R¹ being Me. More preferably only 1 R¹ is Me. Most preferably all R¹ are H.

For the compounds of formula (IC) above, it is preferred that R¹ and all R¹ are H.

A particularly preferred substituted C₁-C₄ alkyl group for the group Ar is trifluoromethyl.

A preferred group of compounds of formula (IC) is represented by the formula (IIC); 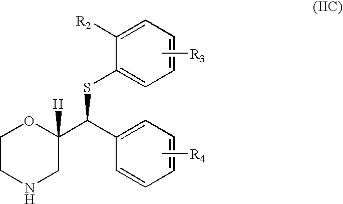
wherein R₂ and R₃ are each independently selected from H, C₁-C₄ alkyl, O(C₁-C₄ alkyl), S(C₁-C₄ alkyl), halo and phenyl; and R₄ is selected from H and C₁-C₄ alkyl; and pharmaceutically acceptable salts thereof. R₂ is preferably C₁-C₃ alkyl (especially trifluoromethyl), O(C₁-C₃ alkyl) (especially methoxy or trifluoromethoxy), F or Ph. R₃ is preferably H. R₃ is also preferably F. R₄ is preferably H.

7. A compound of formula (ID) 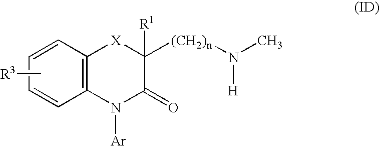
wherein —X— is —C(R⁴R⁵)—, —O— or —S—; n is 2 or 3; R¹ is H or C₁-C₄ alkyl; R³ is H, halo, C₁-C₄ alkyl, O(C₁-C₄ alkyl), nitrile, phenyl or substituted phenyl; R⁴ and R⁵ are each independently selected from H or C₁-C₄ alkyl; Ar— is selected from the group consisting of 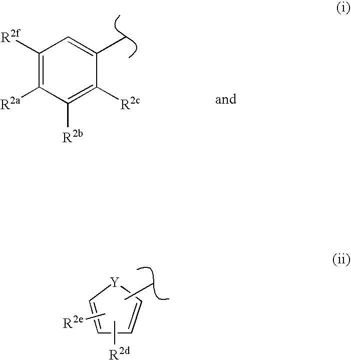
in which R^{2a} is H, halo, methyl or ethyl; R^{2b} is H, halo or methyl; R^{2c} is H, halo, methyl, trifluoromethyl, nitrile, or methoxy; R^{2d} is H, halo, methyl or ethyl; R^{2e} is H, halo, methyl, trifluoromethyl, nitrile, or methoxy; R^{2f} is H, or fluoro; —Y— is —O—, —S— or —N(R⁶)—; and R⁶ is H or methyl and pharmaceutically acceptable salts thereof.

The term “C₁-C₄ alkyl” as used in respect of compounds of formula (ID) includes straight and branched chain alkyl groups of 1, 2, 3 or 4 carbon atoms. Thus the term “C₁-C₄ alkyl” includes methyl, ethyl, n-propyl, isopropyl, n-butyl, isobutyl, sec-butyl and tert-butyl. C₁-C₂ alkyl groups are preferred. A particularly preferred C₁-C₄ alkyl group is methyl or ethyl.

The term “halo” as used in respect of compounds of formula (ID) includes F, Cl, Br and I, and is preferably F or Cl.

The term “substituted phenyl” as used in respect of compounds of formula (ID) means phenyl substituted with 1, 2, 3, 4 or 5 substituents, preferably with 1 or 2, for example 1, substituent. Suitable substituents include C₁-C₄ alkyl, O(C₁-C₄ alkyl), S(C₁-C₄alkyl), halo, and phenyl optionally substituted with, for example, C₁-C₄ alkyl, O(C₁-C₄alkyl), S(C₁-C₄ alkyl), or halo.

The terms “O(C₁-C₄ alkyl)” or “S(C₁-C₄ alkyl)” as used in respect of compounds of formula (ID) mean a C₁-C₄ alkyl group as defined above linked to the point of substitution via an oxygen or a sulphur atom. An O(C₁-C₄ alkyl) or S(C₁-C₄ alkyl) group includes for example methoxy, ethoxy, thiomethyl or thioethyl.

Preferred compounds of formula (ID) are represented by the formula (IDa) 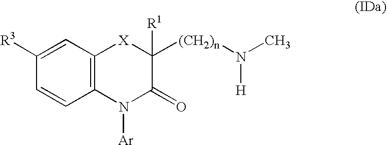
wherein —X—, n, R¹, R³ and Ar have the values as defined for formula (ID) above.

Compounds of formula (ID) or (IDa) wherein —X— is —C(R⁴R⁵)— are preferred. Even more preferred are compounds of formula (ID) or (IDa) wherein —X— is —C(R⁴R⁵)— and R⁴ and R⁵ are both H or R⁴ and R⁵ are both the same C₁-C₄ alkyl.

Compounds of formula (ID) or (IDa) wherein Ar is (i) are also preferred. Preferably Ar is (i) and R^{2c} is H. Even more preferred are compounds of formula (ID) or (IDa) wherein Ar is (i), R^{2c} is H, and (a) R^{2a} is H or methyl, R^{2b} is H and R^{2f} is H or (b) R^{2a} is H, R^{2b} is halo, preferably fluoro or chloro and R^{2f} is H or fluoro.

Another group of preferred compounds of formula (ID) or (IDa) are compounds wherein Ar is (ii) and —Y— is —S—. More preferably Ar is 2-thiophenyl or 3-thiophenyl.

A further preferred group of compounds of formula (ID) is represented by the formula (IID) 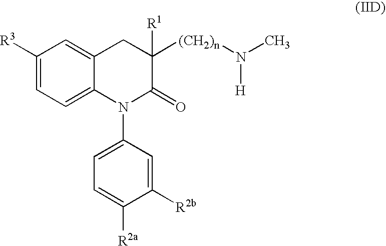
wherein n is 2 or 3; R¹ is H or C₁-C₄ alkyl; R³ is H, halo, phenyl or substituted phenyl; R^{2a} is H, halo, methyl or ethyl; R^{2b} is H, halo or methyl; and pharmaceutically acceptable salts thereof.

Preferred compounds of formulae (ID), (IDa) and (IID) are those wherein n is 3, or wherein R¹ is H, methyl, ethyl or n-propyl, or wherein R³ is H or halo.

8. A compound of formula (IE) 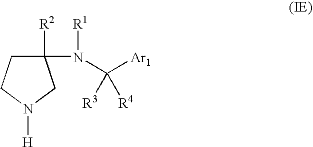
wherein R¹ is C₁-C₆ alkyl (optionally substituted with 1, 2 or 3 halo substituents and/or with 1 substituent selected from —S—(C₁-C₃ alkyl), —O—(C₁-C₃ alkyl) (optionally substituted with 1, 2 or 3 F atoms), —O—(C₃-C₆ cycloalkyl), —SO₂—(C₁-C₃ alkyl), —CN, —COO—(C₁-C₂ alkyl) and —OH); C₂-C₆ alkenyl; —(CH₂)_{q}—Ar₂; or a group of formula (i) or (ii) 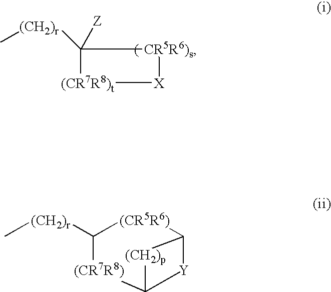
R², R³ and R⁴ are each independently selected from hydrogen or C₁-C₂ alkyl; R⁵, R⁶, R⁷ and R⁸ are at each occurrence independently selected from hydrogen or C₁-C₂ alkyl; —X— is a bond, —CH₂—, —CH═CH—, —O—, —S—, or —SO₂—; —Y— is a bond, —CH₂— or —O—; -Z is hydrogen, —OH or —O—(C₁-C₃ alkyl); p is 0, 1 or 2; q is 0, 1 or 2; r is 0 or 1; s is 0, 1, 2 or 3; t is 0, 1, 2 or 3; Ar₁ is phenyl, pyridyl, thiazolyl, benzothiophenyl or naphthyl; wherein said phenyl, pyridyl or thiazolyl group may be substituted with 1, 2 or 3 substituents each independently selected from halo, cyano, C₁-C₄ alkyl (optionally substituted with 1, 2 or 3 F atoms), —O—(C₁-C₄ alkyl) (optionally substituted with 1, 2 or 3 F atoms) and —S—(C₁-C₄ alkyl) (optionally substituted with 1, 2 or 3 F atoms) and/or with 1 substituent selected from pyridyl, pyrazole, phenyl (optionally substituted with 1, 2 or 3 halo substituents) and phenoxy (optionally substituted with 1, 2 or 3 halo substituents); and wherein said benzothiophenyl or naphthyl group may be optionally substituted with 1, 2 or 3 substituents each independently selected from halo, cyano, C₁-C₄ alkyl (optionally substituted with 1, 2 or 3 F atoms), —O—(C₁-C₄ alkyl) (optionally substituted with 1, 2 or 3 F atoms), and —S—(C₁-C₄ alkyl) (optionally substituted with 1, 2 or 3 F atoms); Ar₂ is naphthyl, pyridyl, thiazolyl, furyl, thiophenyl, benzothiophenyl, or phenyl, wherein said naphthyl, pyridyl, thiazolyl, furyl, thiophenyl, benzothiophenyl, or phenyl may be substituted with 1, 2 or 3 substituents each independently selected from halo, C₁-C₄ alkyl (optionally substituted with 1, 2 or 3 F atoms) and —O—(C₁-C₄ alkyl) (optionally substituted with 1, 2 or 3 F atoms); and pharmaceutically acceptable salts thereof; provided that (a) the cyclic portion of the group of formula (i) must contain at least three carbon atoms and not more than seven ring atoms; (b) when —X— is —CH═CH—, then the cyclic portion of the group of formula (i) must contain at least five carbon atoms; and (c) when -Z is —OH or —O—(C₁-C₃ alkyl), then —X— is —CH₂—; (d) when —Y— is —O— then p cannot be 0; and (e) the compound 3-[(phenylmethyl)-(3S)-3-pyrrolidinylamino]-propanenitrile is excluded.

With respect to formula (IE) the term “C₁-C₆ alkyl” means a monovalent unsubstituted saturated straight-chain or branched-chain hydrocarbon radical having from 1 to 6 carbon atoms.

With respect to formula (IE) the term “C₂-C₆ alkenyl” means a monovalent unsubstituted unsaturated straight-chain or branched-chain hydrocarbon radical having from 2 to 6 carbon atoms and containing at least one carbon-carbon double bond.

With respect to formula (IE) the term “C₃-C₆ cycloalkyl” means a monovalent unsubstituted saturated cyclic hydrocarbon radical having from 3 to 6 carbon atoms.

With respect to formula (IE) the term “C₁-C₆ alkylene” means a divalent unsubstituted saturated straight-chain or branched-chain hydrocarbon radical having from 1 to 6 carbon atoms.

With respect to formula (IE) the term “halo” or “halogen” means F, Cl, Br or I.

With respect to formula (IE) the term “C₁-C₄ difluoroalkyl” means a monovalent unsubstituted saturated straight-chain or branched-chain hydrocarbon radical having from 1 to 4 carbon atoms wherein two hydrogen atoms are substituted with two fluoro atoms. Preferably the two fluoro atoms are attached to the same carbon atom.

With respect to formula (IE) the term “C₁-C₄ trifluoroalkyl” means a monovalent unsubstituted saturated straight-chain or branched-chain hydrocarbon radical having from 1 to 4 carbon atoms wherein three hydrogen atoms are substituted with three fluoro atoms. Preferably the three fluoro atoms are attached to the same carbon atom.

With respect to formula (IE) the term “phenoxy” means a monovalent unsubstituted phenyl radical linked to the point of substitution by an O atom.

With respect to formula (IE) the term “pyridyl” includes 2-pyridyl, 3-pyridyl and 4-pyridyl.

With respect to formula (IE) the term “furyl” includes 2-furyl and 3-furyl. 2-furyl is preferred.

With respect to formula (IE) the term “thiophenyl” includes 2-thiophenyl and 3-thiophenyl.

With respect to formula (IE) the term “thiazolyl” includes 2-thiazolyl, 4-thiazolyl and 5-thiazolyl.

With respect to formula (IE) the term “pyrazole” includes 1-pyrazole, 3-pyrazole and 4-pyrazole. 1-pyrazole is preferred.

With respect to formula (IE) the term “benzothiophenyl” includes 2-benzo[b]thiophenyl, 3-benzo[b]thiophenyl, 4-benzo[b]thiophenyl, 5-benzo[b]thiophenyl, 6-benzo[b]thiophenyl and 7-benzo[b]thiopbenyl.

With respect to formula (IE) the term “naphthyl” includes 1-naphthyl, and 2-naphthyl. 1-naphthyl is preferred.

With respect to formula (IE), similar terms specifying different numbers of C atoms take an analogous meaning. For example the terms “C₁-C₄ alkyl” and “C₁-C₃ alkyl” mean a monovalent unsubstituted saturated straight-chain or branched-chain hydrocarbon radical having from 1 to 4 and 1 to 3 carbon atoms respectively. The term “C₁-C₄ alkyl” includes methyl, ethyl, n-propyl, iso-propyl, n-butyl, iso-butyl, sec-butyl, and tert-butyl. The term “C₁-C₃ alkyl” includes methyl, ethyl, n-propyl and iso-propyl.

With respect to formula (IE) it will be appreciated that when s is 2 or 3, then each R⁵ and/or each R⁶ can be different. In the same way when t is 2 or 3, then each R⁷ and/or each R⁸ can be different.

Preferred compounds of formula (IE) are those wherein R¹ is C₁-C₆ alkyl, C₂-C₆ alkenyl, —(CH₂)ₘ—CF₃, —(CH₂)ₙ—S—(C₁-C₃ alkyl), —CH₂—COO—(C₁-C₂ alkyl), —(C₁-C₅ alkylene)-O—(C₁-C₃ alkyl), —(C₁-C₅ alkylene)-O—(C₃-C₆ cycloalkyl), —(C₁-C₅ alkylene)-SO₂—(C₁-C₃ alkyl), —(C₁-C₅ alkylene)-OCF₃, —(C₁-C₆ alkylene)-OH, —(C₁-C₅ alkylene)-CN, —(CH₂)_{q}—Ar₂ or a group of formula (ia), (ib) or (ii) 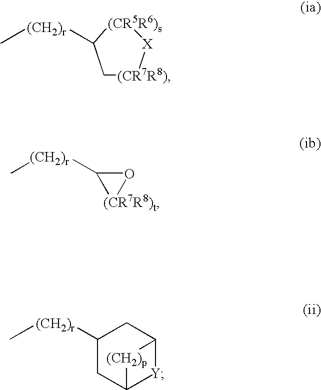
R², R³, R⁴, R⁵, R⁶, R⁷, R⁸, —X—, —Y—, p, q, r and s have the values defined above; m is 1, 2 or 3; n is 1, 2 or 3; t is 2, 3 or 4; —Ar₁ is phenyl, pyridyl, thiazolyl or naphthyl; wherein said phenyl, pyridyl or thiazolyl group may be substituted with 1, 2 or 3 substituents each independently selected from halo, trifluoromethyl, cyano, C₁-C₄ alkyl, —O—(C₁-C₄ alkyl), —O—(C₁-C₄ difluoroalkyl), —O—(C₁-C₄ trifluoroalkyl), —S—(C₁-C₄ alkyl), —S—(C₁-C₂ trifluoroalkyl) and/or with 1 substituent selected from pyridyl, pyrazole, phenyl (optionally substituted with 1, 2 or 3 halo substituents) and phenoxy (optionally substituted with 1, 2 or 3 halo substituents); and wherein said naphthyl group may be optionally substituted with 1, 2 or 3 substituents each independently selected from halo, trifluoromethyl, cyano, C₁-C₄ alkyl, —O—(C₁-C₄ alkyl), —O—(C₁-C₄ difluoroalkyl), —O—(C₁-C₄ trifluoroalkyl), —S—(C₁-C₄ alkyl), —S—(C₁-C₂ trifluoroalkyl); Ar₂ is naphthyl, pyridyl, thiazolyl, furyl, thiophenyl, benzothiophenyl, or phenyl, wherein said naphthyl, pyridyl, thiazolyl, furyl, thiophenyl, benzothiophenyl, or phenyl may be substituted with 1, 2 or 3 substituents each independently selected from halo, C₁-C₄ alkyl, trifluoromethyl and —O— (C₁-C₄ alkyl); and pharmaceutically acceptable salts thereof.

Preferred compounds of formula (IE) are those wherein R² is hydrogen. In another preferred embodiment R³ and R⁴ are hydrogen. More preferably R², R³ and R⁴ are hydrogen.

Preferred compounds of formula (IE) are those wherein each R⁵ and R⁶ is hydrogen. In another preferred embodiment each R⁷ and R⁸ is hydrogen. More preferably R⁵, R⁶, R⁷ and R⁸ are hydrogen.

Preferred compounds of formula (IE) are those wherein R¹ is C₁-C₆ alkyl. More preferably R¹ is n-propyl, 1-methylethyl, 2-methylpropyl, 3,3-dimethylpropyl.

Preferred compounds of formula (IE) are those wherein R¹ is —(C₄-C₅ alkylene)-OH. More preferably R¹ is 2,2-dimethyl-2-hydroxyethyl or 3,3-dimethyl-3-hydroxypropyl.

Preferred compounds of formula (IE) are those wherein R¹ is a group of formula (i) and each R⁵ and R⁶ is hydrogen. More preferably each R⁵, R⁶, R⁷ and R⁸ is hydrogen.

Preferred compounds of formula (IE) are those wherein R¹ is a group of formula (ii) and each R⁵ and R⁶ is hydrogen. More preferably each R⁵, R⁶, R⁷ and R⁸ is hydrogen.

Preferred compounds of formula (IE) are those wherein R¹ is a group of formnula (i), r is O, s is 2, t is 2,-Z is hydrogen and —X— is —O—, —S— or —SO₂—. More preferably R¹ is a group of formula (i), r is 0, s is 2, t is 1 or 2, -Z is hydrogen and —X— is —O—.

Preferred compounds of formula (IE) are those wherein R¹ is a group of formula (i), r is 0, s is 1, 2 or 3, t is 1, -Z is hydrogen and —X— is —CH₂—.

Preferred compounds of formula (IE) are those wherein R¹ is a group of formula (i), r is 1, s is 0, 1, 2 or 3, t is 1, -Z is hydrogen and —X— is —CH₂—.

Preferred compounds of formula (IE) are those wherein R¹ is a group of the formula (ia). More preferably R¹ is a group of the formnula (ia) and each R⁵, R⁶, R⁷and R⁸ is hydrogen.

Preferred compounds of formula (IE) are those wherein R¹ is a group of the formula (ib). More preferably R¹ is a group of the formula (ib), r is 1, t is 3, and each R⁷and R⁸ is hydrogen.

Preferred compounds of formula (IE) are those wherein R¹ is —(CH₂)ₘ—CF₃. More preferably R¹ is —(CH₂)ₘ—CF₃ and m is 1, 2, or 3.

Preferred compounds of formula (IE) are those wherein R¹ is —(CH₂)ₙ—S—(C₁-C₃ alkyl). More preferably R¹ is —(CH₂)₃—S—CH₃.

Preferred compounds of formnula (IE) are those wherein R¹ is —CH₂—COO—(C₁-C₂ alkyl). More preferably R¹ is —CH₂—COOCH₃.

Preferred compounds of formula (IE) are those wherein R¹ is —(C₁-C₅ alkylene)-O—(C₁-C₃ alkyl). More preferably R¹ is —(C₃-C₄ alkylene)-OCH₃.

Preferred compounds of formula (IE) are those wherein R¹ is —(C₁-C₅ alkylene)-O—(C₃-C₆ cycloalkyl). More preferably R¹ is —CH₂-CH₂—O—cyclobutyl.

Preferred compounds of formula (IE) are those wherein R¹ is —(C₁-C₅ alkylene)-SO₂—(C₁-C₃ alkyl).

Preferred compounds of formula (IE) are those wherein R¹ is —(C₁-C₅ alkylene)-OCF₃. More preferably R¹ is —CH₂-CH₂—OCF₃.

Preferred compounds of formula (IE) are those wherein R¹ is —(C₁-C₅ alkylene)-CN. More preferably R¹ is —(C₂-C₄ alkylene)-CN. Most preferably —CH₂—CH₂—CN or —CH₂—C(CH₃)₂—CN.

Preferred compounds of formula (IE) are those wherein R¹ is —(CH₂)_{q}—Ar₂, and q is 1. More preferably R¹ is —(CH₂)_{q}—Ar₂, q is 1 and —Ar₂ is pyridyl, phenyl or phenyl substituted with 1, 2 or 3 substituents each independently selected from halo, trifluoromethyl or C₁-C₄ alkyl.

Preferred compounds of formula (IE) are those wherein —Ar₁ is phenyl; phenyl substituted with 1, 2 or 3 substituents each independently selected from halo, trifluoromethyl and C₁-C₄ alkyl and/or with 1 substituent selected from phenyl, phenyl substituted with 1, 2 or 3 halo substituents, pyridyl, pyrazole, phenoxy and phenoxy substituted with 1, 2 or 3 halo substituents; pyridyl; or pyridyl substituted with 1, 2 or 3 substituents each independently selected from halo, trifluoromethyl and C₁-C₄ alkyl and/or with 1 substituent selected from phenyl and phenyl substituted with 1, 2 or 3 halo substituents. More preferably —Ar₁ is phenyl or phenyl substituted with 1, 2 or 3 substituents each independently selected from halo, trifluoromethyl and C₁-C₄ alkyl and/or with 1 substituent selected from phenyl, phenyl substituted with 1, 2 or 3 halo substituents, pyridyl, pyrazole, phenoxy and phenoxy substituted with 1, 2 or 3 halo substituents. Most preferably —Ar₁ is phenyl substituted with 1 or 2 substituents each independently selected from halo, trifluoromethyl and C₁-C₄ alkyl and/or with 1 substituent selected from phenyl, phenyl substituted with 1, 2 or 3 halo substituents, pyridyl, pyrazole, phenoxy and phenoxy substituted with 1, 2 or 3 halo substituents. Suitable —Ar₁ groups include, for example, 2-methylthiophenyl, 2-methylphenyl, 2-fluorophenyl, 2-chlorophenyl, 2-isopropoxyphenyl, 2-trifluoromethylphenyl, 2-difluoromethoxyphenyl, 2-methoxyphenyl, 2-ethoxyphenyl, 2-(1,1′-biphenyl), 2-phenoxyphenyl, 2-benzylphenyl, 3-trifluoromethoxyphenyl, 3-chlorophenyl, 3-trifluoromethylphenyl, 3-methylphenyl, 3-trifluorothiomethoxyphenyl, 3-methoxyphenyl, 4- trifluoromethylphenyl, 4-chlorophenyl, 4-fluorophenyl, 3,5-dichlorophenyl, 3,5-dimethylphenyl, 3-trifluoromethyl-5-fluorophenyl, 3,5-difluorophenyl, 2,3-dichlorophenyl, 2,3-dimethylphenyl, 2-chloro-3-trifluoromethylphenyl, 2-chloro-3-methylphenyl, 2-methyl-3-chlorophenyl, 2,4-dichlorophenyl, 2,4-dimethyl, 2,4-difluorophenyl, 2-chloro-4-fluorophenyl, 2-trifluoromethyl-4-fluorophenyl, 2-fluoro-4-trifluoromethylphenyl, 2-methyl4-chlorophenyl, 2-methoxy-4-fluorophenyl, 2-trifluoromethyl-5-fluorophenyl, 2,5-dimethylphenyl, 4-fluoro-[1,1′-biphenyl]-2-yl, 2-chloro-5-fluorophenyl, 2-(trifluoromethyl)-6-fluorophenyl, 2-chloro-6-fluorophenyl, 3,4-dichlorophenyl, and 3-chloro-4-fluorophenyl. In general when —Ar₁ is phenyl substituted with pyridyl, 3-pyridyl is preferred.

Preferred compounds of formula (IE) are those wherein —Ar₁ is pyridyl or pyridyl substituted with 1, 2 or 3 substituents each independently selected from halo, trifluoromethyl and C₁-C₄ alkyl and/or with 1 substituent selected from phenyl and phenyl substituted with 1, 2 or 3 halo substituents. More preferably —Ar₁ is pyridyl substituted with 1 or 2 substituents each independently selected from halo, trifluoromethyl and C₁-C₄ alkyl and/or with 1 substituent selected from phenyl and phenyl substituted with 1, 2 or 3 halo substituents. Suitable —Ar₁ groups include, for example, 3-phenyl-2-pyridyl. In general when —Ar₁ is a substituted pyridyl, substituted 2-pyridyl is preferred.

9. A compound of formula (IF) 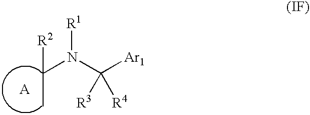
wherein 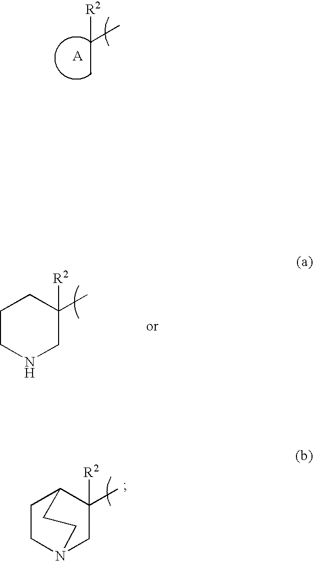
is a group of formnula (a) or (b)
R¹ is C₁-C₆ alkyl (optionally substituted with 1, 2 or 3 halo substituents and/or with 1 substituent selected from —S—(C₁-C₃ alkyl), —O—(C₁-C₃ alkyl) (optionally substituted with 1, 2 or 3 F atoms), —O—(C₃-C₆ cycloalkyl), —SO₂—(C₁-C₃ alkyl), —CN, —COO—(C₁-C₂ alkyl) and —OH); C₂-C₆ alkenyl; —(CH₂)_{q}—Ar₂; or a group of formula (i) or (ii) 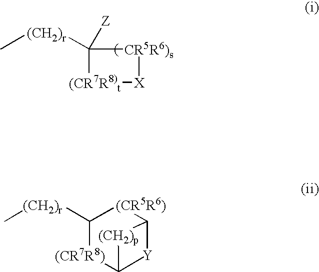
R², R³ and R⁴ are each independently selected from hydrogen or C₁-C₂ alkyl; R⁵, R⁶, R⁷ and R⁸ are at each occurrence independently selected from hydrogen or C₁-C₂ alkyl; —X— is a bond, —CH₂—, —CH═CH—, —O—, —S-, or —SO₂—; —Y— is a bond, —CH₂— or —O—; -Z is hydrogen, —OH or —O—(C₁-C₃ alkyl); p is 0, 1 or 2; q is 0, 1 or 2; r is 0 or 1; s is 0, 1, 2 or 3; t is 0, 1, 2 or 3; Ar₁ is phenyl, pyridyl, thiazolyl, benzothiophenyl or naphthyl; wherein said phenyl, pyridyl or thiazolyl group may be substituted with 1, 2 or 3 substituents each independently selected from halo, cyano, C₁-C₄ alkyl (optionally substituted with 1, 2 or 3 F atoms), —O—(C₁-C₄ alkyl) (optionally substituted with 1, 2 or 3 F atoms) and —S—(C₁-C₄ alkyl) (optionally substituted with 1, 2 or 3 F atoms) and/or with 1 substituent selected from pyridyl, pyrazole, phenyl (optionally substituted with 1, 2 or 3 halo substituents), benzyl and phenoxy (optionally substituted with 1, 2 or 3 halo substituents); and wherein said benzothiophenyl or naphthyl group may be optionally substituted with 1, 2 or 3 substituents each independently selected from halo, cyano, C₁-C₄ alkyl (optionally substituted with 1, 2 or 3 F atoms), —O—(C₁-C₄ alkyl) (optionally substituted with 1, 2 or 3 F atoms), and —S—(C₁-C₄ alkyl) (optionally substituted with 1, 2 or 3 F atoms); Ar₂ is naphthyl, pyridyl, thiazolyl, furyl, thiophenyl, benzothiophenyl, or phenyl, wherein said naphthyl, pyridyl, thiazolyl, furyl, thiophenyl, benzothiophenyl, or phenyl may be substituted with 1, 2 or 3 substituents each independently selected from halo, C₁-C₄ alkyl (optionally substituted with 1, 2 or 3 F atoms) and —O—(C₁-C₄ alkyl) (optionally substituted with 1, 2 or 3 F atoms); or a pharmaceutically acceptable salt thereof; provided that (a) the cyclic portion of the group of formula (i) must contain at least three carbon atoms and not more than seven ring atoms; (b) when —X— is —CH═CH—, then the cyclic portion of the group of formula (i) must contain at least five carbon atoms; and (c) when -Z is —OH or —O—(C₁-C₃ alkyl), then —X— is —CH₂—; and (d) when —Y— is —O— then p cannot be 0.

With respect to formula (IF) the term “C₁-C₆ alkyl” means a monovalent unsubstituted saturated straight-chain or branched-chain hydrocarbon radical having from 1 to 6 carbon atoms.

With respect to formula (IF) the term “C₂-C₆ alkenyl” means a monovalent unsubstituted unsaturated straight-chain or branched-chain hydrocarbon radical having from 2 to 6 carbon atoms and containing at least one carbon-carbon double bond.

With respect to formula (IF) the term “C₃-C₆ cycloalkyl” means a monovalent unsubstituted saturated cyclic hydrocarbon radical having from 3 to 6 carbon atoms.

With respect to formula (IF) the term “C₁-C₆ alkylene” means a divalent unsubstituted saturated straight-chain or branched-chain hydrocarbon radical having from 1 to 6 carbon atoms.

With respect to formula (IF) the term “halo” or “halogen” means F, Cl, Br or I.

With respect to formula (IF) the term “C₁-C₄ difluoroalkyl” means a monovalent unsubstituted saturated straight-chain or branched-chain hydrocarbon radical having from 1 to 4 carbon atoms wherein two hydrogen atoms are substituted with two fluoro atoms. Preferably the two fluoro atoms are attached to the same carbon atom.

With respect to formula (IF) the term “C₁-C₄ trifluoroalkyl” means a monovalent unsubstituted saturated straight-chain or branched-chain hydrocarbon radical having from 1 to 4 carbon atoms wherein three hydrogen atoms are substituted with three fluoro atoms. Preferably the three fluoro atoms are attached to the same carbon atom.

With respect to formula (IF) the term “phenoxy” means a monovalent unsubstituted phenyl radical linked to the point of substitution by an O atom.

With respect to formula (IF) the term “pyridyl” includes 2-pyridyl, 3-pyridyl and 4-pyridyl.

With respect to formula (IF) the term “furyl” includes 2-furyl and 3-furyl. 2-furyl is preferred.

With respect to formula (IF) the term “thiophenyl” includes 2-thiophenyl and 3-thiophenyl.

With respect to formula (IF) the term “thiazolyl” includes 2-thiazolyl, 4-thiazolyl and 5-thiazolyl.

With respect to formula (IF) the term “pyrazole” includes 1-pyrazole, 3-pyrazole and 4-pyrazole. 1-pyrazole is preferred.

With respect to formula (IF) the term “benzothiophenyl” includes 2-benzo[b]thiophenyl, 3-benzo[b]thiophenyl, 4-benzo[b]thiophenyl, 5-benzo[b]thiophenyl, 6-benzo[b]thiophenyl and 7-benzo[b]thiophenyl.

With respect to formula (IF) the term “naphthyl” includes 1-naphthyl, and 2-naphthyl. 1-naphthyl is preferred.

With respect to formula (IF), similar terms specifying different numbers of C atoms take an analogous meaning. For example the terms “C₁-C₄ alkyl” and “C₁-C₃ alkyl” mean a monovalent unsubstituted saturated straight-chain or branched-chain hydrocarbon radical having from 1 to 4 and 1 to 3 carbon atoms respectively. The term “C₁-C₄ alkyl” includes methyl, ethyl, n-propyl, iso-propyl, n-butyl, iso-butyl, sec-butyl, and tert-butyl. The term “C₁-C₃ alkyl” includes methyl, ethyl, n-propyl and iso-propyl.

With respect to formula (IF), it will be appreciated that when s is 2 or 3, then each R⁵ and/or each R⁶ can be different. In the same way when t is 2 or 3, then each R⁷ and/or each R⁸ can be different.

Preferred compounds of formula (IF) are those of formula (IF′) 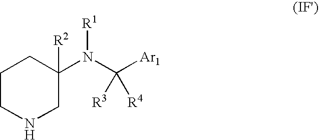
wherein R¹, R², R³, R⁴ and Ar₁ have the values defined in formula (IF) above.

Preferred compounds of formula (IF) are those of formula (IF″) 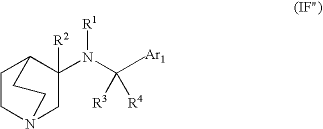
wherein R¹, R², R³, R⁴ and Ar₁ have the values defined in formula (IF) above.

Preferred compounds of formula (IF) are those wherein R¹ is C₁-C₆ alkyl, C₂-C₆ alkenyl, —(CH₂)ₘ—CF₃, —(CH₂)ₙ—S—(C₁-C₃ alkyl), —CH₂—COO—(C₁-C₂ alkyl), —(C₁-C₅ alkylene)-O—(C₁-C₃ alkyl), —(C₁-C₅ alkylene)-O—(C₃-C₆ cycloalkyl), —(C₁-C₅ alkylene)-SO₂—(C₁-C₃ alkyl), —(C₁-C₅ alkylene)-OCF₃, —(C₁-C₆ alkylene)-OH, —(C₁-C₅ alkylene)-CN, —(CH₂)_{q}—Ar₂ or a group of formula (ia), (ib) or (ii) 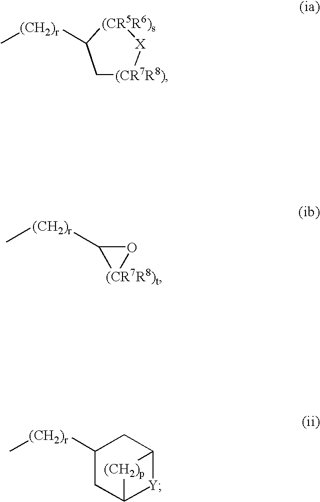
R², R³, R⁴, R⁵, R⁶, R⁷, R⁸, —X—, —Y—, p, q, r and s have the values defined above; m is 1, 2 or 3; n is 1, 2 or 3; t is 2, 3 or 4; —Ar₁ is phenyl, pyridyl, thiazolyl or naphthyl; wherein said phenyl, pyridyl or thiazolyl group may be substituted with 1, 2 or 3 substituents each independently selected from halo, trifluoromethyl, cyano, C₁-C₄ alkyl, —O—(C₁-C₄ alkyl), —O—(C₁-C₄ difluoroalkyl), —O—(C₁-C₄ trifluoroalkyl), —S—(C₁-C₄ alkyl), —S—(C₁-C₂ trifluoroalkyl) and/or with 1 substituent selected from pyridyl, pyrazole, phenyl (optionally substituted with 1, 2 or 3 halo substituents) and phenoxy (optionally substituted with 1, 2 or 3 halo substituents); and wherein said naphthyl group may be optionally substituted with 1, 2 or 3 substituents each independently selected from halo, trifluoromethyl, cyano, C₁-C₄ alkyl, —O—(C₁-C₄ alkyl), —O—(C₁-C₄ difluoroalkyl), —O—(C₁-C₄ trifluoroalkyl), —S—(C₁-C₄ alkyl), —S—(C₁-C₂ trifluoroalkyl); Ar₂ is naphthyl, pyridyl, thiazolyl, furyl, thiophenyl, benzothiophenyl, or phenyl, wherein said naphthyl, pyridyl, thiazolyl, furyl, thiophenyl, benzothiophenyl, or phenyl may be substituted with 1, 2 or 3 substituents each independently selected from halo, C₁-C₄ alkyl, trifluoromethyl and —O—(C₁-C₄ alkyl).

Preferred compounds of formula (IF) are those wherein R² is hydrogen. In another preferred embodiment R³ and R⁴ are hydrogen. More preferably R², R³ and R⁴ are hydrogen.

Preferred compounds of formula (IF) are those wherein each R⁵ and R⁶ is hydrogen. In another preferred embodiment each R⁷ and R⁸ is hydrogen. More preferably R⁵, R⁶, R⁷ and R⁸ are hydrogen.

Preferred compounds of formula (IF) are those wherein R¹ is C₁-C₆ alkyl. More preferably R¹ is n-propyl, 1-methylethyl (i-propyl), 2-methylpropyl (i-butyl), 2-methylbutyl, 2,2-dimethylbutyl.

Preferred compounds of formula (IF) are those wherein R¹ is —(C₄-C₅ alkylene)-OH. More preferably R¹ is 2,2-dimethyl-2-hydroxyethyl or 3,3-dimethyl-3-hydroxypropyl.

Preferred compounds of formula (IF) are those wherein R¹ is a group of formula (i) and each R⁵ and R⁶ is hydrogen. More preferably each R⁵, R⁶, R⁷ and R⁸ is hydrogen.

Preferred compounds of formula (IF) are those wherein R¹ is a group of formula (ii) and each R⁵ and R⁶ is hydrogen. More preferably each R⁵, R⁶, R⁷ and R⁸ is hydrogen.

Preferred compounds of formula (IF) are those wherein R¹ is a group of formula (i), r is 0 or 1, s is 2, t is 1 or 2, -Z is hydrogen and —X— is —O—, —S— or —SO₂—. More preferably R¹ is a group of formula (i), r is 0 or 1, s is 2, t is 1 or 2, -Z is hydrogen and —X— is —O—, for example tetrahydro-2H-pyran-4-yl, tetrahydrofuran-3-yl or (tetrahydrofuran-3-yl)methyl. Most preferably R¹ is a group of formula (i), r is 0, s is 2, t is 1 or 2, -Z is hydrogen and —X— is —O—, for example tetrahydro-2H-pyran-4-yl or tetrahydrofuran-3-yl.

Preferred compounds of formula (IF) are those wherein R¹ is a group of formula (i), r is 0, s is 1, 2 or 3, t is 1, -Z is hydrogen and —X— is —CH₂—, for example cyclobutyl, cyclopentyl or cyclohexyl.

Preferred compounds of formula (IF) are those wherein R¹ is a group of formula (i), r is 1, s is 0, 1, 2 or 3, t is 1, -Z is hydrogen and —X— is —CH₂—.

Preferred compounds of formula (IF) are those wherein R¹ is a group of the formula (ia). More preferably R¹ is a group of the formula (ia) and each R⁵, R⁶, R⁷and R⁸ is hydrogen.

Preferred compounds of formula (IF) are those wherein R¹ is a group of the formula (ib). More preferably R¹ is a group of the formula (ib), r is 1, t is 3, and each R⁷ and R⁸ is hydrogen.

Preferred compounds of formula (IF) are those wherein R¹ is —(CH₂)ₘ—CF₃. More preferably R¹ is —(CH₂)ₘ—CF₃ and m is 1, 2, or 3.

Preferred compounds of formula (IF) are those wherein R¹ is —(CH₂)ₙ—S—(C₁-C₃ alkyl). More preferably R¹ is —(CH₂)₃—S—CH₃.

Preferred compounds of formula (IF) are those wherein R¹ is —CH₂—COO—(C₁-C₂ alkyl). More preferably R¹ is —CH₂—COOCH₃.

Preferred compounds of formula (IF) are those wherein R¹ is —(C₁-C₅ alkylene)-O—(C₁-C₃ alkyl). More preferably R¹ is —(C₃-C₄ alkylene)-OCH₃.

Preferred compounds of formula (IF) are those wherein R¹ is —(C₁-C₅ alkylene)-O—(C₃-C₆ cycloalkyl). More preferably R¹ is —CH₂-CH₂—O-cyclobutyl.

Preferred compounds of formula (IF) are those wherein R¹ is —(C₁-C₅ alkylene)-SO₂—(C₁-C₃ alkyl).

Preferred compounds of formula (IF) are those wherein R¹ is —(C₁-C₅ alkylene)-OCF₃. More preferably R¹ is —CH₂—CH₂—OCF₃.

Preferred compounds of formula (IF) are those wherein R¹ is —(C₁-C₅ alkylene)-CN. More preferably R¹ is —(C₂-C₄ alkylene)-CN. Most preferably —CH₂—CH₂—CN or —CH₂—C(CH₃)₂—CN.

Preferred compounds of formula (IF) are those wherein R¹ is —(CH₂)_{q}—Ar₂, and q is 1. More preferably R¹ is —(CH₂)_{q}—Ar₂, q is 1 and —Ar₂ is pyridyl, phenyl or phenyl substituted with 1, 2 or 3 substituents each independently selected from halo, trifluoromethyl, C₁-C₄ alkyl or O—(C₁-C₄ alkyl).

Preferred compounds of formula (IF) are those wherein —Ar₁ is phenyl; phenyl substituted with 1, 2 or 3 substituents each independently selected from halo, trifluoromethyl and C₁-C₄ alkyl and/or with 1 substituent selected from phenyl, phenyl substituted with 1, 2 or 3 halo substituents, pyridyl, pyrazole, phenoxy and phenoxy substituted with 1, 2 or 3 halo substituents; pyridyl; or pyridyl substituted with 1, 2 or 3 substituents each independently selected from halo, trifluoromethyl and C₁-C₄ alkyl and/or with 1 substituent selected from phenyl and phenyl substituted with 1, 2 or 3 halo substituents. More preferably —Ar₁ is phenyl or phenyl substituted with 1, 2 or 3 substituents each independently selected from halo, trifluoromethyl and C₁-C₄ alkyl and/or with 1 substituent selected from phenyl, phenyl substituted with 1, 2 or 3 halo substituents, pyridyl, pyrazole, phenoxy and phenoxy substituted with 1, 2 or 3 halo substituents. Most preferably —Ar₁ is phenyl substituted with 1 or 2 substituents each independently selected from halo, trifluoromethyl and C₁-C₄ alkyl and/or with 1 substituent selected from phenyl, phenyl substituted with 1, 2 or 3 halo substituents, pyridyl, pyrazole, phenoxy and phenoxy substituted with 1, 2 or 3 halo substituents. Suitable —Ar₁ groups include, for example, 2-methylthiophenyl, 2-methylphenyl, 2-fluorophenyl, 2-chlorophenyl, 2-isopropoxyphenyl, 2-trifluoromethylphenyl, 2-difluoromethoxyphenyl, 2-methoxyphenyl, 2-ethoxyphenyl, 2-(1,1′-biphenyl), 2-phenoxyphenyl, 2-benzylphenyl, 3-trifluoromethoxyphenyl, 3-chlorophenyl, 3-trifluoromethylphenyl, 3-methylphenyl, 3-trifluorothiomethoxyphenyl, 3-methoxyphenyl, 5 4- trifluoromethylphenyl, 4-chlorophenyl, 4-fluorophenyl, 3,5-dichlorophenyl, 3,5-dimethylphenyl, 3-trifluoromethyl-5-fluorophenyl, 3,5-difluorophenyl, 2,3-dichlorophenyl, 2,3-dimethylphenyl, 2-chloro-3-trifluoromethylphenyl, 2-chloro-3-methylphenyl, 2-methyl-3-chlorophenyl, 2,4-dichlorophenyl, 2,4-dimethyl, 2,4-difluorophenyl, 2-chloro-4-fluorophenyl, 2-trifluoromethyl-4-fluorophenyl, 2-fluoro-4-trifluoromethylphenyl, 2-methyl-4-chlorophenyl, 2-methoxy-4-fluorophenyl, 2-trifluoromethyl-5-fluorophenyl, 2,5-dimethylphenyl, 4-fluoro-[1,1′-biphenyl]-2-yl, 2-chloro-5-fluorophenyl, 2-(trifluoromethyl)-6-fluorophenyl, 2-chloro-6-fluorophenyl, 3,4-dichlorophenyl, and 3-chloro-4-fluorophenyl. In general when —Ar₁ is phenyl substituted with pyridyl, 3-pyridyl is preferred.

Preferred compounds of formula (IF) are those wherein —Ar₁ is pyridyl or pyridyl substituted with 1, 2 or 3 substituents each independently selected from halo, trifluoromethyl and C₁-C₄ alkyl and/or with 1 substituent selected from phenyl and phenyl substituted with 1, 2 or 3 halo substituents. More preferably —Ar₁ is pyridyl substituted with 1 or 2 substituents each independently selected from halo, trifluoromethyl and C₁-C₄ alkyl and/or with 1 substituent selected from phenyl and phenyl substituted with 1, 2 or 3 halo substituents. Suitable —Ar₁ groups include, for example, 3-phenyl-2-pyridyl. In general when —Ar₁ is a substituted pyridyl, substituted 2-pyridyl is preferred.

10. A compound of formula (IG) 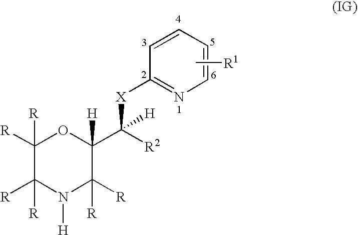
wherein —X— is —S— or —O—; each R is independently selected from H or C₁-C₄ alkyl; R¹ is H, C₁-C₄ alkyl, C₁-C₄ alkoxy, halo, cyano, trifluoromethyl, trifluoromethoxy, —NR³R⁴, —CONR³R⁴, —COOR³ or a group of the formula (i) 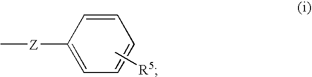
R²is C₁-C₄ alkyl, phenyl or phenyl substituted with 1, 2 or 3 substituents each independently selected from C₁-C₄ alkyl, C₁-C₄ alkoxy, nitro, hydroxy, cyano, halo, trifluoromethyl, trifluoromethoxy, benzyl, benzyloxy, —NR⁶R⁷, —CONR⁶R⁷, COOR⁶, —SO₂NR⁶R⁷ and —SO₂R⁶; R⁵ is selected from C₁-C₄ alkyl, C₁-C₄ alkoxy, carboxy, nitro, hydroxy, cyano, halo, trifluoromethyl, trifluoromethoxy, benzyl, benzyloxy, —NR⁸R⁹, —CONR⁸R⁹, —SO₂NR⁸R⁹ and —SO₂R⁸; R³, R⁴, R⁶, R⁷, R⁸ and R⁹ are each independently selected from H or C₁-C₄ alkyl; and -Z- is a bond, —CH₂—, or —O—; or a pharmaceutically acceptable salt thereof.

With respect to formula (IG) the term “C₁-C₄ alkyl” means a monovalent unsubstituted saturated straight-chain or branched-chain hydrocarbon radical having from 1 to 4 carbon atoms. Thus the term “C₁-C₄ alkyl” includes methyl, ethyl, n-propyl, isopropyl, n-butyl, isobutyl, sec-butyl and tert-butyl.

With respect to formula (IG) the term “C₁-C₄ alkoxy” means a monovalent unsubstituted saturated straight-chain or branched-chain hydrocarbon radical having from 1 to 4 carbon atoms linked to the point of substitution by an 0 atom. Thus the term “C₁-C₄ alkoxy” includes methoxy, ethoxy, n-propoxy, isopropoxy, n-butoxy, isobutoxy, sec-butoxy.

With respect to formula (IG) the term “halo” or “halogen” means F, Cl, Br or I.

Preferred compounds of formula (IG) are those wherein —X— is —S—.

Preferred compounds of formula (IG) are those wherein —X— is —O—.

Preferred compounds of formula (IG) are those wherein R² is phenyl.

Preferred compounds of formula (IG) are those wherein all R groups are hydrogen.

Preferred compounds of formula (IG) are those represented by the formula (IIG) 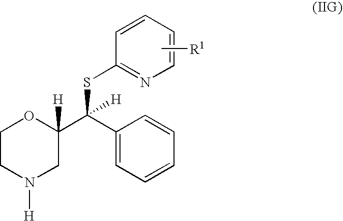
wherein R¹ is H, C₁-C₄ alkyl, C₁-C₄ alkoxy, halo, cyano, trifluoromethyl, trifluoromethoxy, —NR³R⁴, —CONR³R⁴, —COOR³ or a group of the formula (i) 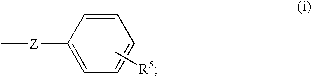
R⁵ is selected from C₁-C₄ alkyl, C₁-C₄ alkoxy, carboxy, nitro, hydroxy, cyano, halo, trifluoromethyl, trifluoromethoxy, benzyl, benzyloxy, —NR⁸R⁹, —CONR⁸R⁹, —SO₂NR⁸R⁹ and —SO₂R⁸; R³, R⁴, R⁸ and R⁹ are each independently selected from H or C₁-C₄ alkyl; -Z- is a bond, —CH₂—, or —O—; or a pharmaceutically acceptable salt thereof.

Preferred compounds of formula (IG) or (IIG) are those wherein the substituent R¹ is in the three position of the pyridine ring as numbered in formula (IG) above. More preferably said substituent R¹ is H, C₁-C₄ alkyl, halo, cyano, —CONR³R⁴, trifluoromethyl or a group of the formula (i). When R¹ is —CONR³R⁴, then R³ and R⁴ are both preferably H. When R¹ is C₁-C₄ alkyl, then it is preferably methyl.

Preferred compounds of formula (IG) or (IIG) are those wherein the substituent R¹ is a group of the formula (i).

Preferred compounds of formula (IG) or (IIG) are those wherein R¹ is a group of the formula (i), -Z- is a bond, and R⁵ is H or halo.

Preferred compounds of formula (IG) or (IIG) are those wherein R¹ is a group of the formula (i), -Z- is —CH₂— or —O—, and R⁵ is H.

Preferred compounds of formula (IG) or (IIG) are those wherein the substituent R¹ is in the five position of the pyridine ring as numbered in formula (IG) above. More preferably said substituent R¹ is selected from bromo, chloro or iodo.

Compounds within the scope of Formulae (IA), (IB), (IC), (ID), (IE), (IF) and (IG) above are inhibitors of norepinephrine reuptake. Certain compounds within the scope of Formulae (IA), (IB), (IC), (ID), (IE), (IF) and (IG) above are selective inhibitors of norepinephrine reuptake.

Biogenic amine transporters control the amount of biogenic amine neurotransmitters in the synaptic cleft. Inhibition of the respective transporter leads to a rise in the concentration of that neurotransmitter within the synaptic cleft. Compounds of Formulae (IA), (IB), (IC), (ID), (IE), (IF) and (IG) above and their pharmaceutically acceptable salts preferably exhibit a Kᵢ value less than 500 nM at the norepinephrine transporter as determined using the scintillation proximity assay as described below. More preferred compounds of Formulae (IA), (IB), (IC), (ID), (IE), (IF) and (IG) above and their pharmaceutically acceptable salts exhibit a Kᵢ value less than 100 nM at the norepinephrine transporter. More preferred compounds of Formulae (IA), (IB), (IC), (ID), (IE), (IF) and (IG) above and their pharmaceutically acceptable salts exhibit a Kᵢ value less than 50 nM at the norepinephrine transporter. Especially preferred compounds of Formulae (IA), (IB), (IC), (ID), (IE), (IF) and (IG) above and their pharmaceutically acceptable salts exhibit a Kᵢ value less than 20 nM at the norepinephrine transporter. Preferably, these compounds selectively inhibit the norepinephrine transporter relative to the serotonin and dopamine transporters by a factor of at least five, more preferably by a factor of at least ten.

In addition, the compounds of Formulae (IA), (IB), (IC), (ID), (IE), (IF) and (IG) above of the present invention are preferably acid stable. Advantageously, they have a reduced interaction (both as substrate and inhibitor) with the liver enzyme Cytochrome P450 (CYP2D6). That is to say, they preferably exhibit less than 75% metabolism via the CYP2D6 pathway according to the CYP2D6 substrate assay described below and they preferably exhibit an IC50 of >6 μM according to the CYP2D6 inhibitor assay described below.

While all compounds exhibiting norepinephrine reuptake inhibition are useful for the methods of the present invention, certain are preferred. It is preferred that the norepinephrine reuptake inhibitor is selective for the reuptake of norepinephrine over the reuptake of other neurotransmitters. It is also preferred that the norepinephrine reuptake inhibitor does not exhibit signigicant direct agonist or antagonist activity at other receptors. It is especially preferred that the norepinephrine reuptake inhibitor be selected from atomoxetine, reboxetine, (S,S)-reboxetine, (R)-N-methyl-3-(2-methyl-thiophenoxy)-3-phenylpropylamine, and compounds of Formulae (I), (IA), (IB), (IC), (ID), (IE), (IF) and (IG) above.

The present invention encompasses pharmaceutical compositions comprising the compounds disclosed herein, or pharmaceutically acceptable salts thereof, together with a pharmaceutically acceptable carrier, diluent, or excipient.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

Many of the compounds used in this invention are amines, and accordingly react with any of a number of inorganic and organic acids to form pharmaceutically acceptable acid addition salts. Since some of the free amines of the compounds of this invention are typically oils at room temperature, it is preferable to convert the free amines to their pharmaceutically acceptable acid addition salts for ease of handling and administration, since the latter are routinely solid at room temperature. Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, b-hydroxybutyrate, glycollate, tartrate, methanesulfonate, prop anesul fonate, naphthalene-1-sulfonate, naphthalene-2-sulfonate, mandel ate and the like. Preferred pharmaceutically acceptable salts are those formed with hydrochloric acid.

Pharmaceutically acceptable salts of the compounds of Formulae (IA), (IB), (IC), (ID) (IE), (IF) and (IG) above include acid addition salts, including salts formed with inorganic acids, for example hydrochloric, hydrobromic, nitric, sulphuric or phosphoric acids, or with organic acids, such as organic carboxylic or organic sulphonic acids, for example, acetoxybenzoic, citric, glycolic, o- mandelic-l, mandelic-dl, mandelic d, maleic, mesotartaric monohydrate, hydroxymaleic, fumaric, lactobionic, malic, methanesulphonic, napsylic, naphtalenedisulfonic, naphtoic, oxalic, palmitic, phenylacetic, propionic, pyridyl hydroxy pyruvic, salicylic, stearic, succinic, sulphanilic, tartaric, 2-hydroxyethane sulphonic, toluene-p-sulphonic, and xinafoic acids.

In addition to the pharmaceutically acceptable salts, other salts can serve as intermediates in the purification of compounds, or in the preparation of other, for example pharmaceutically acceptable, acid addition salts, or are useful for identification, characterization, or purification.

The present invention encompasses the administration of a composition that exhibits (preferably selective) norepinephrine reuptake inhibitor activity. The composition can comprise one or more agents that, individually or together, inhibit norepinephrine reuptake preferably in a selective manner.

Dosages

The dosages of the drugs used in the methods of the present invention must, in the final analysis, be set by the physician in charge of the case using knowledge of the drugs, the properties of the drugs alone or in combination as determined in clinical trials, and the characteristics of the patient including diseases other than that for which the physician is treating the patient. General outlines of the dosages, and some preferred dosages, are as follows: intravenous, or intradermal administration), intra-pulmonary, vaginal, rectal, intranasal, ophthalmic, or intraperitoneal administration, or by an implantable extended release device. Oral administration is preferred. The route of administration can be varied in any way, limited by the physical properties of the drugs, the convenience of the patient and the caregiver, and other relevant circumstances (Remington's Pharmaceutical Sciences (1990) 18th Edition, Mack Publishing Co.).

The pharmaceutical compositions are prepared in a manner well known in the pharmaceutical art. The carrier or excipient can be a solid, semi-solid, or liquid material that can serve as a vehicle or medium for the active ingredient. Suitable carriers or excipients are well known in the art. The pharmaceutical composition can be adapted for oral, inhalation, parenteral, or topical use and can be administered to the patient in the form of tablets, capsules, aerosols, inhalants, suppositories, solutions, suspensions, or the like.

The compounds of the present invention can be administered orally, for example, with an inert diluent or capsules or compressed into tablets. For the purpose of oral therapeutic administration, the compounds can be incorporated with excipients and used in the form of tablets, troches, capsules, elixirs, suspensions, syrups, wafers, chewing gums and the like. These preparations should contain at least 4% of the compound of the present invention, the active ingredient, but can be varied depending upon the particular form and can conveniently be between 4% to about 70% of the weight of the unit. The amount of the compound present in compositions is such that a suitable dosage will be obtained. Preferred compositions and preparations according to the present invention can be determined by a person skilled in the art.

The tablets, pills, capsules, troches, and the like can also contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin can be added or a flavoring agent such as peppermint, methyl salicylate or orange flavoring. When the dosage unit form is a capsule, it can contain, in addition to materials of the above type, a liquid carrier such as polyethylene glycol or a fatty oil.

Atomoxetine:

In adults and older adolescents: from about 5 mg/day to about 200 mg/day; preferably in the range from about 60 to about 150 mg/day; more preferably from about 60 to about 130 mg/day; and still more preferably from about 50 to about 120 mg/day;

In children and younger adolescents: from about 0.2 to about 3.0 mg/kg/day; preferably in the range from about 0.5 to about 1.8 mg/kg/day;

Reboxetine: Racemic reboxetine can be administered to an individual in an amount in the range of from about 2 to about 20 mg per patient per day, more preferably from about 4 to about 10 mg/day, and even more preferably from about 6 to about 10 mg/day. Depending on the formulation, the total daily dosage can be administered in smaller amounts up to two times per day. A preferred adult daily dose of optically pure (S,S) reboxetine can be in the range of from about 0.1 mg to about 10 mg, more preferably from about 0.5 mg to about 8 to 10 mg, per patient per day. The effective daily dose of reboxetine for a child is smaller, typically in the range of from about 0.1 mg to about 4 to about 5 mg/day. Treatments using compositions containing optically pure (S,S)-reboxetine are about 5 to about 8.5 times more effective in inhibiting the reuptake of norepinephrine than compositions containing a racemic mixture of (R,R)- and (S,S)-reboxetine, and therefore lower doses can be employed. PCT International Publication No. WO 01/01973 contains additional details concerning the dosing of (S,S) reboxetine.

Compounds of formula I: from about 0.01 mg/kg to about 20 mg/kg; preferred daily doses are from about 0.05 mg/kg to 10 mg/kg; more preferably from about 0.1 mg/kg to about 5 mg/kg;

Compounds of formulae (IA), (lB), (IC), (ID), (IE), (IF) and (IG) above: from about 5 to about 500 mg, more preferably from about 25 to about 300 mg, of the active ingredient per patient per day.

Administration

The compounds disclosed herein can be administered by various routes, for example systemically via oral (including buccal or sublingual), topical (including buccal, sublingual, or transdermal), parenteral (including subcutaneous, intramuscular, Other dosage unit forms can contain other various materials that modify the physical form of the dosage unit, for example, as coatings. Thus, tablets or pills can be coated with sugar, shellac, or other coating agents. A syrup can contain, in addition to the present compounds, sucrose as a sweetening agent and certain preservatives, dyes and colorings and flavors. Materials used in preparing these various compositions should be pharmaceutically pure and non-toxic in the amounts used.

A formulation useful for the administration of R-(−)-N-methyl 3-((2-methylphenyl)oxy)-3-phenyl-1-aminopropane hydrochloride (atomoxetine) comprises a dry mixture of R-(−)-N-methyl 3-((2-methylphenyl)oxy)-3-phenyl-1-aminopropane hydrochloride with a diluent and lubricant. A starch, such as pregelatinized corn starch, is a suitable diluent and a silicone oil, such as dimethicone, a suitable lubricant for use in hard gelatin capsules. Suitable formulations are prepared containing about 0.4 to 26% R-(−)-N-methyl 3-((2-methylphen-yl)oxy)-3-phenyl-1-aminopropane hydrochloride, about 73 to 99% starch, and about 0.2 to 1.0% silicone oil. Tables 1 and 2 illustrate particularly preferred formulations:

TABLE 1

| Ingredient (%) | 2.5 mg | 5 mg | 10 mg | 18 mg | 20 mg | 25 mg | 40 mg | 60 mg |
|---|---|---|---|---|---|---|---|---|
| R-(−)-N-methyl 3- | 1.24 | 2.48 | 4.97 | 8.94 | 9.93 | 12.42 | 19.87 | 22.12 |
| ((2-methylphenyl) | | | | | | | | |
| oxy)-3- | | | | | | | | |
| phenyl-1- | | | | | | | | |
| aminopropane | | | | | | | | |
| hydrochloride | | | | | | | | |
| Dimethicone | 0.5 | 0.5 | 0.5 | 0.5 | 0.5 | 0.5 | 0.5 | 0.5 |
| Pregelatinized | 98.26 | 97.02 | 94.53 | 90.56 | 89.57 | 87.08 | 79.63 | 77.38 |
| Starch | | | | | | | | |

TABLE 2

| Ingredient | | | | | | | | |
|---|---|---|---|---|---|---|---|---|
| (mg/capsule) | 2.5 mg | 5 mg | 10 mg | 18 mg | 20 mg | 25 mg | 40 mg | 60 mg |
| R-(−)-N-methyl 3- | 2.86 | 5.71 | 11.43 | 20.57 | 22.85 | 28.57 | 45.71 | 68.56 |
| ((2-methylphenyl) | | | | | | | | |
| oxy)-3- | | | | | | | | |
| phenyl-1- | | | | | | | | |
| aminopropane | | | | | | | | |
| hydrochloride | | | | | | | | |
| Dimethicone | 1.15 | 1.15 | 1.15 | 1.15 | 1.15 | 1.15 | 1.15 | 1.55 |
| Pregelatinized | 225.99 | 223.14 | 217.42 | 208.28 | 206.00 | 200.28 | 183.14 | 239.89 |
| Starch | | | | | | | | |
| Capsule Fill Weight | 230 | 230 | 230 | 230 | 230 | 230 | 230 | 310 |
| (mg) | | | | | | | | |
| Capsule Size | 3 | 3 | 3 | 3 | 3 | 3 | 3 | 2 |

For the purpose of parenteral therapeutic administration, the compounds of the present invention can be incorporated into a solution or suspension. These preparations typically contain at least 0.1% of a compound of the invention, but can be varied to be between 0.1 and about 90% of the weight thereof. The amount of the compound of formula I present in such compositions is such that a suitable dosage will be obtained. The solutions or suspensions can also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Preferred compositions and preparations are able to be determined by one skilled in the art.

The compounds of the present invention can also be administered topically, and when done so the carrier can suitably comprise a solution, ointment, or gel base. The base, for example, can comprise one or more of the following: petrolatum, lanolin, polyethylene glycols, bees wax, mineral oil, diluents such as water and alcohol, and emulsifiers, and stabilizers. Topical formulations can contain a concentration of the compound, or its pharmaceutical salt, from about 0.1 to about 10% w/v (weight per unit volume).

The compositions are preferably formulated in a dosage unit form, i.e., physically discrete units suitable as unitary doses for human subjects and other mammals, each unit containing a predetermined quantity of active material calculated to produce the desired therapeutic effect, in association with a suitable pharmaceutical carrier, diluent, or excipient.

The following examples are provided to illustrate various aspects of the present invention, and should not be construed to be limiting thereof in any way.

### Preparation of Compounds of Formula (1A)

Compounds of formula (IA) may be prepared by conventional organic chemistry techniques and also by solid phase synthesis. In the present specification the abbreviation “boc” refers to the N-protecting group t-butyloxycarbonyl. In the present specification the abbreviation “TFA” refers to trifluoroacetic acid. In the present specification the abbreviation “DMF” refers to dimethylformamide. In the present specification the abbreviation “SPE” refers to solid phase extraction. In the present specification the abbreviation “ACE-Cl” refers to ct-chloroethyl chloroformate.

When R8 is H, a suitable three-step conventional synthesis is outlined in Scheme 1A shown below.

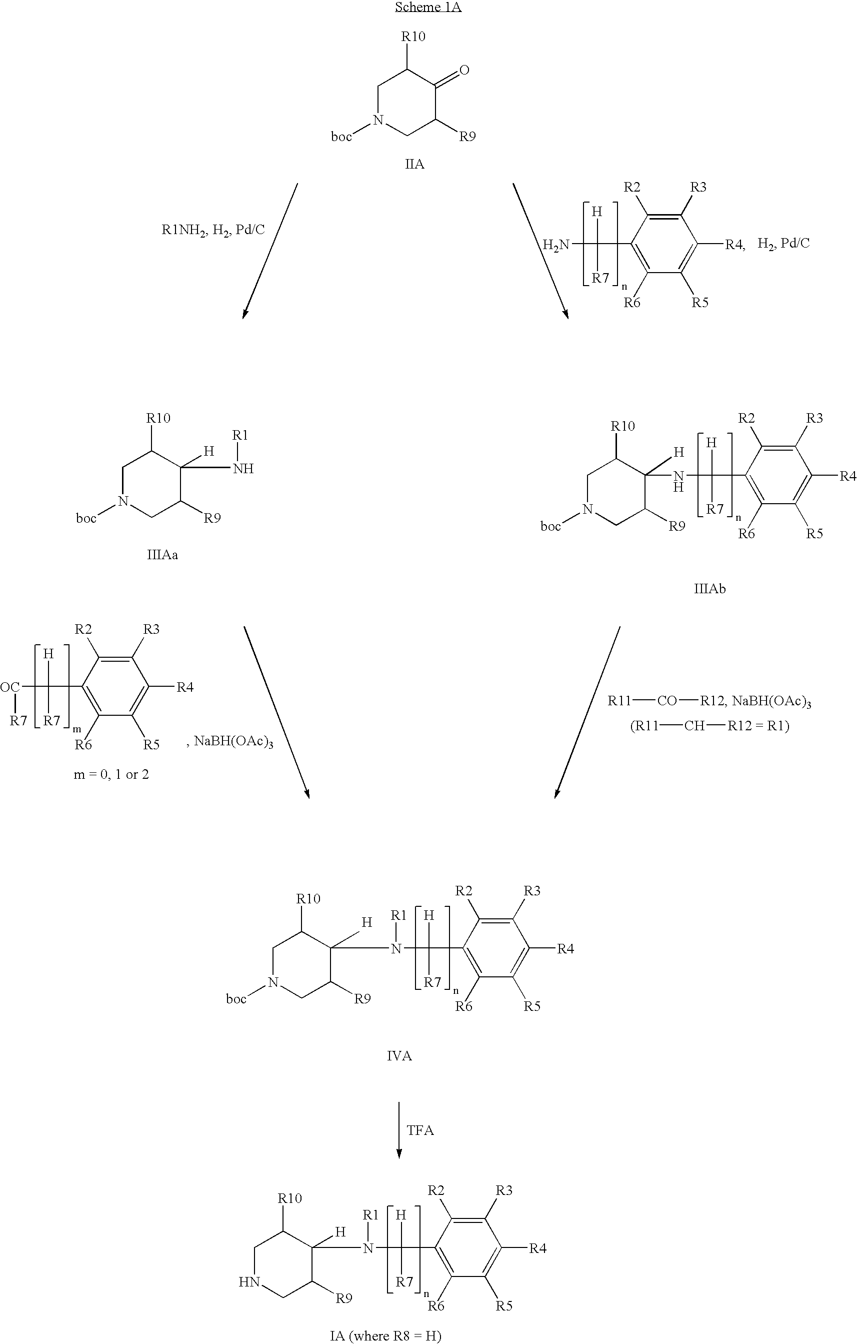

A boc-protected 4-piperidone (IIA) is reductively aminated with an amine to provide a 4-amino-piperidine (IIIAa or IIIAb). A second reductive amination with an aldehyde or ketone provides a boc-protected compound of formula (IA) (IVA). The boc group is removed under acidic conditions to provide a compound of formula (IA) (where R8 is H). If desired, the compound of formula (IA) (where R8 is H) may be converted to a suitable salt by addition of a suitable quantity of a suitable acid. In the schemes above (and below) R1 to R7, R9, R10 and n are as previously defined, m is 0, 1 or 2 and R11 and R12 are chosen such that R11-CH—R12=R1.

Although the boc N-protecting group is used in the above illustration, it will be appreciated that other N-protecting groups (for example acetyl, benzyl or benzoxycarbonyl) could also be used together with a deprotection step appropriate for the N-protecting group used. Similarly, other reducing agents (for example NaBH₄ or LiAIH₄) may be used in the reductive amination steps and other acids (for example HCl) may be used in the deprotection step.

As an alternative to the second reductive amination step, compound IIIAa or IIIAb may be subjected to an alkylation step as shown in Scheme 1B below (L represents a suitable leaving group—for example Br or tosyl).

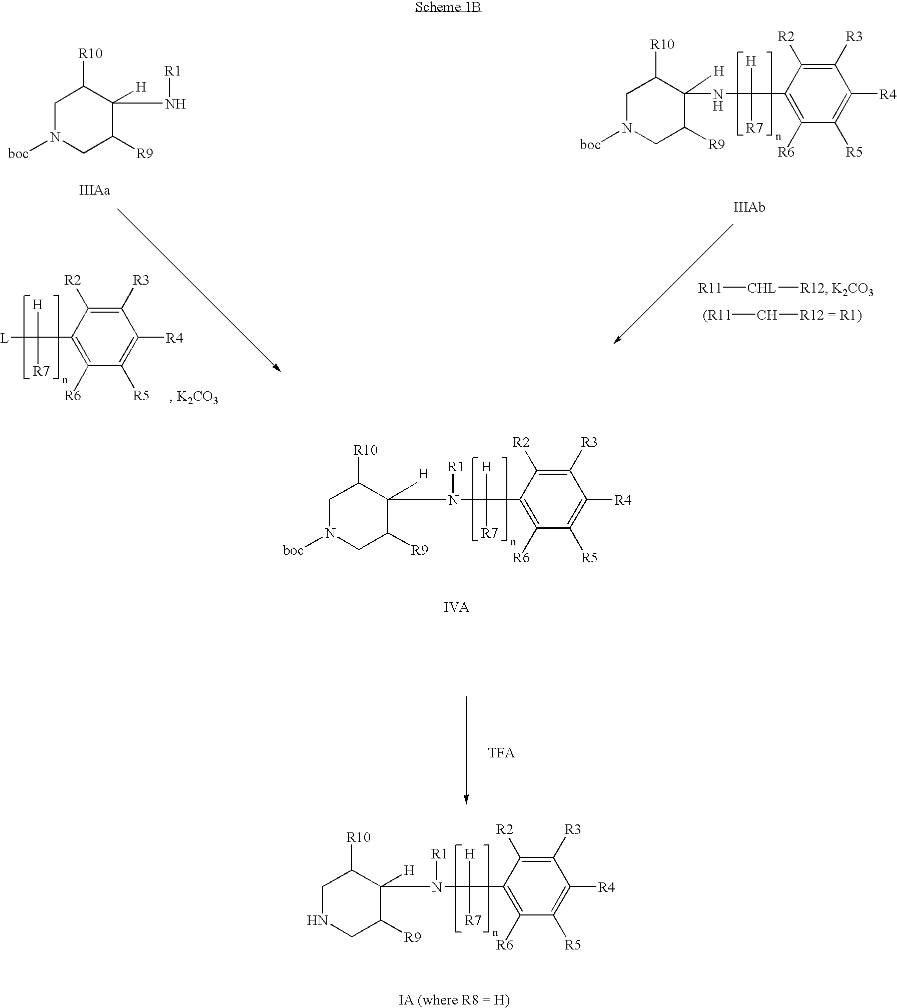

Once again, N-protection other than boc may also be used together with a suitable deprotection step. Similarly, bases other than potassium carbonate (e.g NaH) may be used for the alkylation step

Using essentially the same chemical reactions as in the first scheme above, the compounds of formula (IA) (where R8 is H) may also be prepared by a solid phase parallel synthesis technique as outlined in Scheme 1C shown below. 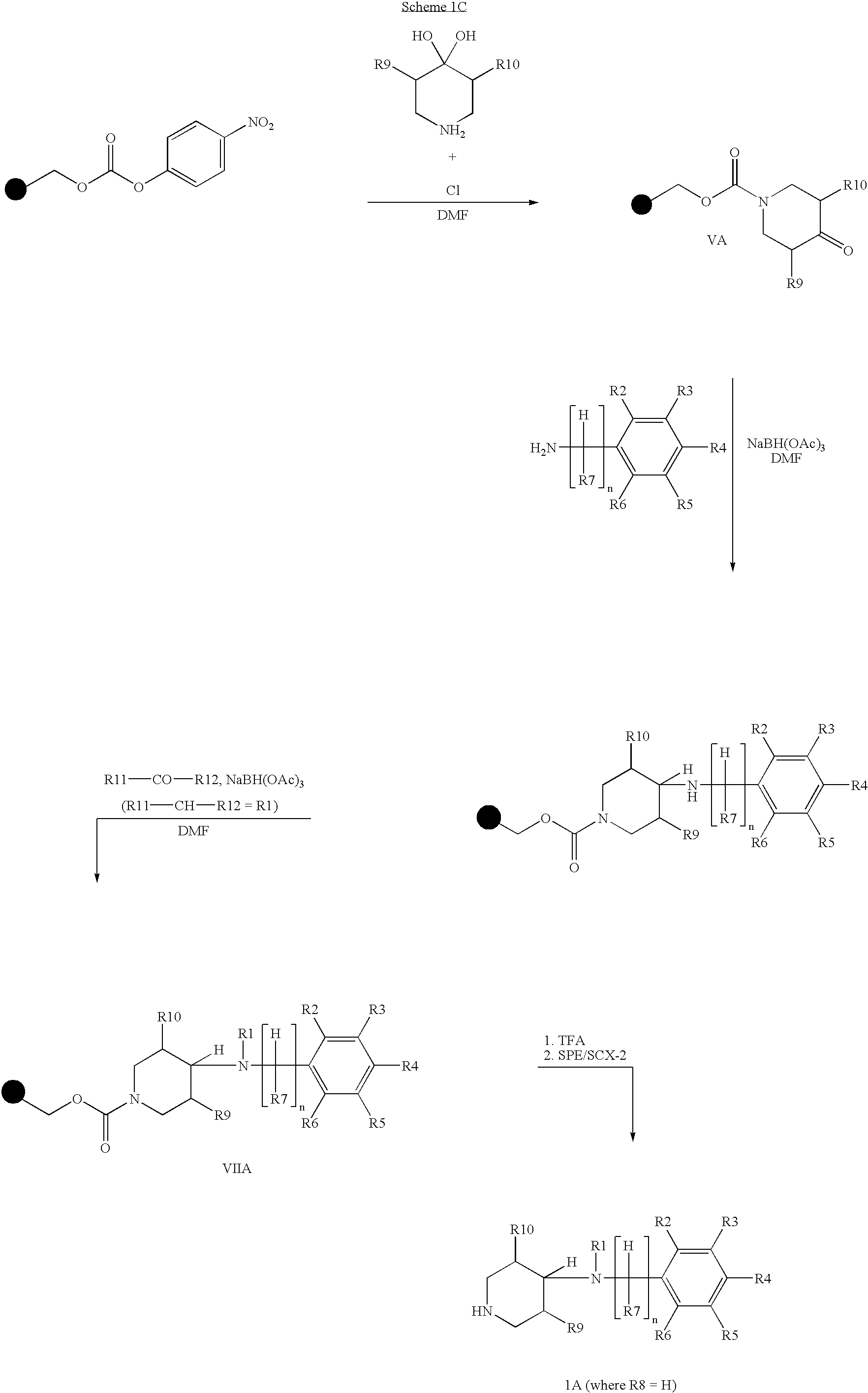
A piperidone hydrate is attached to a polystyrene resin to provide a resin bound piperidone (VA). Aliquots are reductively aminated to provide a resin bound secondary amine (VIA) that can undergo a further reductive amination with an aldehyde or ketone to give the tertiary amine (VIIA). Acidic cleavage from the resin and SPE provides compounds of formula (IA) (where R8 is H) which may be purified by ion exchange methods using, for example, the SCX-2 ion exchange resin.

Although NaBH(OAc)₃ is used in the above illustration, it will be appreciated that other reducing agents (for example NaBH₄ or LiAIH₄) may be used in the reductive amination steps and other acids (for example HCl) may be used in the deprotection step. Solid phase resins other than the p-nitrophenylcarbonate-polystyrene resin illustrated above may also be employed.

When R8 is C₁-C₄alkyl, a conventional synthetic route is outlined in Scheme 1D shown below.

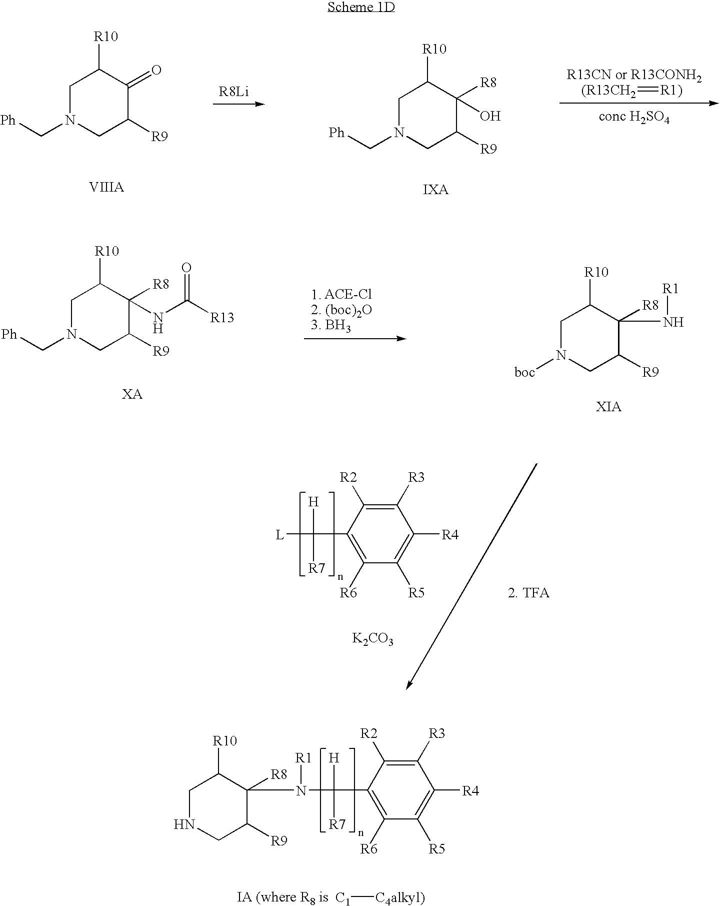

A benzyl-protected 4-piperidone (VIIIA) is alkylated with an alkyllithium reagent to provide a 4-amino-piperidinot (IXA). Treatment with an alkylnitrile or alkylamide under strongly acidic conditions provides a secondary amide (XA) which may be deprotected, boc-protected and reduced to provide a secondary amine (XIA). Alkylation of the secondary amine (XIA) followed by removal of the boc group provides a compound of formnula (IA) (where R8 is C₁-C₄alkyl). In the scheme above L is a leaving group as previously defined and R13 is chosen such that R13—CH₂═R1.

Although the benzyl and boc N-protecting groups are used in the above illustration, it will be appreciated that other N-protecting groups could also be used in their place together with deprotection steps appropriate for those N-protecting groups. Similarly, other reducing agents may be used in the amidecarbonyl reduction step and other organometallics or bases may be used in the respective alkylation steps.

### Preparation of Compounds of Formula (IB)

A general scheme outlining the synthetic routes to compounds of Formulae (IB) wherein Y is OH is shown below (Scheme 1B). For clarity, Ar₂ is shown as phenyl and Ry and Rz are shown as H. It will be appreciated that analogous methods could be applied for other possible identities of Ar₂, Ry and Rz.

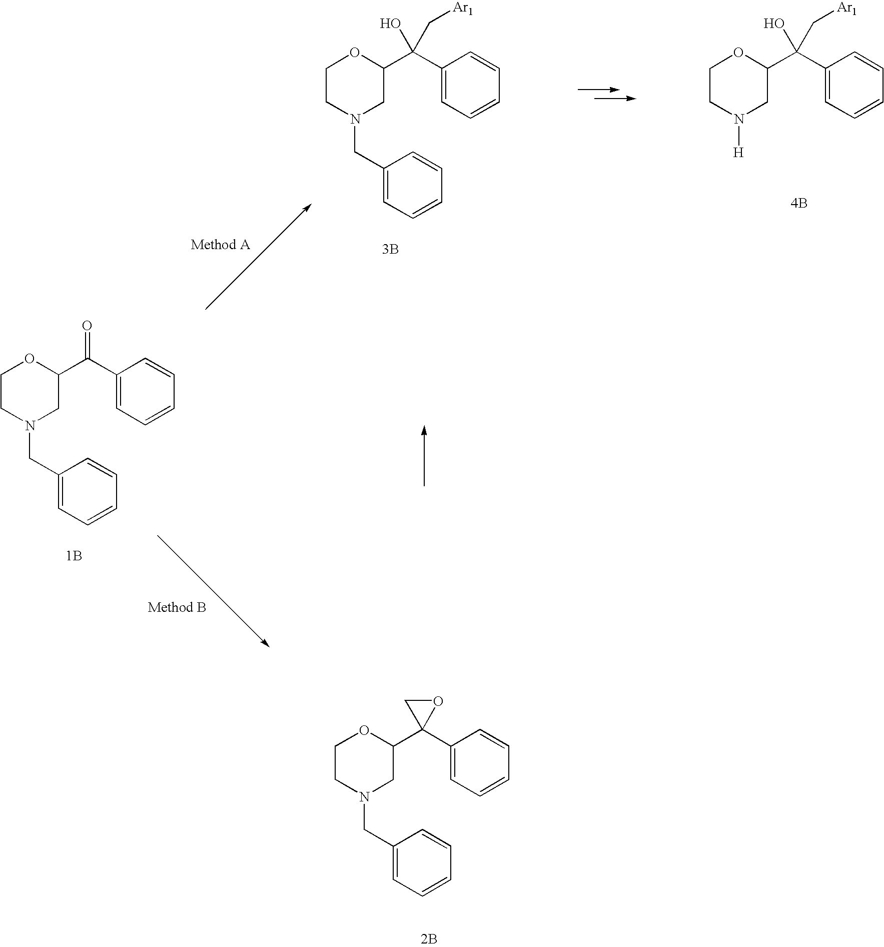

Compounds of Formulae (IB) can be prepared by conventional organic chemistry techniques from an N-benzyl-ketomorpholine of type 1 B by addition of a suitable organometallic derivative (method A), or via the addition of a suitable organometallic reagent to an epoxide of type 2B (method B), as outlined in Scheme 1B.

The racemic intermediates of type 1B can be obtained as outlined in Scheme 2B by condensation of an N-benzyl cyanomorpholine 5B (J. Med. Chem. 1993, 36, pp 683-689) with a suitable aryl organometallic reagent followed by acid hydrolysis. Chiral HPLC separations of the racemic N-benzyl-aryl-ketomorpholine of type 1B gives the required single enantiomer, i.e., the (2S)-N-benzyl-aryl-ketomorpholine of type 6B (Scheme 2B).

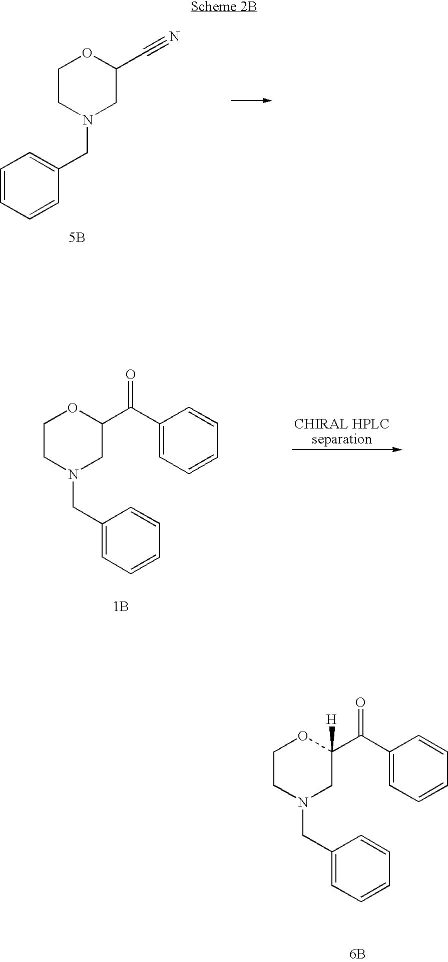

Condensation of a chiral (2S)-N-benzyl-aryl-ketomorpholine of type 6B with a commercially available benzylic magnesium halide or a benzylic magnesium halide prepared using standard Grignard techniques from the corresponding halo-benzylic derivative gives a tertiary alcohol of type 3B without any observed epimerisation of the existing asymmetric center (ee's/de's determinations can be carried out using chiral HPLC) and with very high overall diastereoisomeric excesses (see Scheme 3B). The final compounds of type 4B can be obtained after cleavage of the N-benzyl protecting group on a compound of type 3B. The deprotection can be done using catalytic palladium hydrogenolysis, or carbamate exchange with ACE-Cl (1-Chloroethyl chloroformate), giving intermediates of type 7B, followed by methanolysis as shown in Scheme 3B.

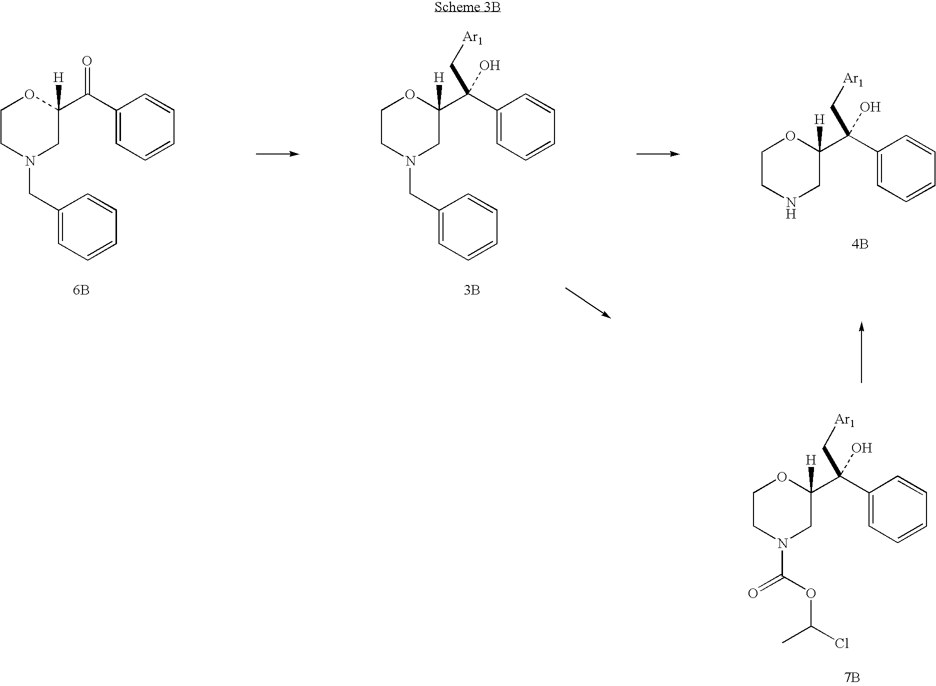

The intermediates 3B can be further elaborated using for example organometallic type couplings between an ortho bromide derivative of type 8B and an arylboronic acid as shown in Scheme 4B. For clarity, Ar₁ and its substituent (R₁) are shown as phenyl and substitution occurs at the 2-position. It will be appreciated that analogous methods could be applied for other possible identities of Ar₁ and R₁ and other possible substitution positions. This approach can also be carried out by solid phase synthetic methods as described in more detail in the specific examples below.

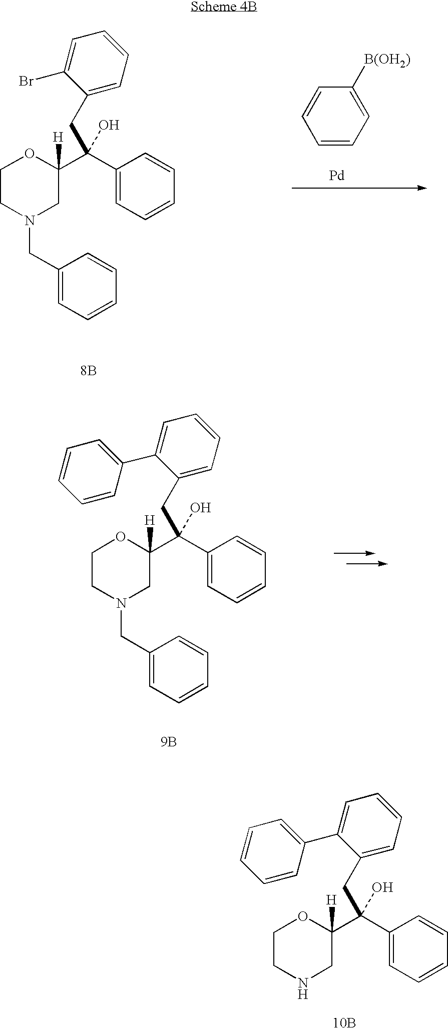

An alternative route for the preparation of the compounds of Formulae (IB) is method B (see Scheme 1B). Formation of the intermediate epoxides of type 2B from racemic N-benzyl-ketomorpholines of type 1B, can be done using for example trimethyl sulfoxonium iodide and a suitable base, for example sodium hydride. Condensation of 2B with a commercially available aryl organometallic, or an aryl organometallic prepared from the corresponding halo aryl derivative, gives the intermediates of type 3B, as mixtures of diastereoisomers. Final deprotections can be done as described above (see Scheme 3B). Final compounds made using method B can be purified using chiral HPLC.

Compounds of Formula (IB) of the present invention wherein Y is OR and R is C₁-C₄ alkyl, can be synthesized by standard alkylation of intermediates of type 3B prior to deprotection of the morpholine N-atom as shown in Scheme 5B. Suitable strong bases will be known to the person skilled in the art and include, for example, sodium hydride. Similarly, suitable alkylating agents will be known to the person skilled in the art and include, for example, C₁-C₄ alkyl halides such as methyl iodide.

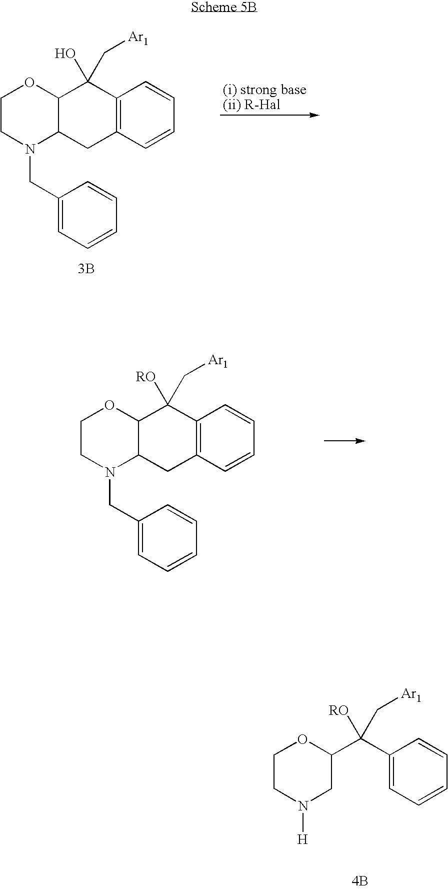

### Preparation of Compounds of Formula (IC)

Compounds of formula (IC) may be prepared by conventional organic chemistry techniques from N-benzyl-cyanomorpholine 1C (Route A) or N-benzyl-morpholinone 2C (Route B) as outlined in Scheme IC below: For clarity, X is shown as phenyl and R′ and R¹ are shown as H. It will be appreciated that analogous methods could be applied for other possible identities of X, R′ and R¹.

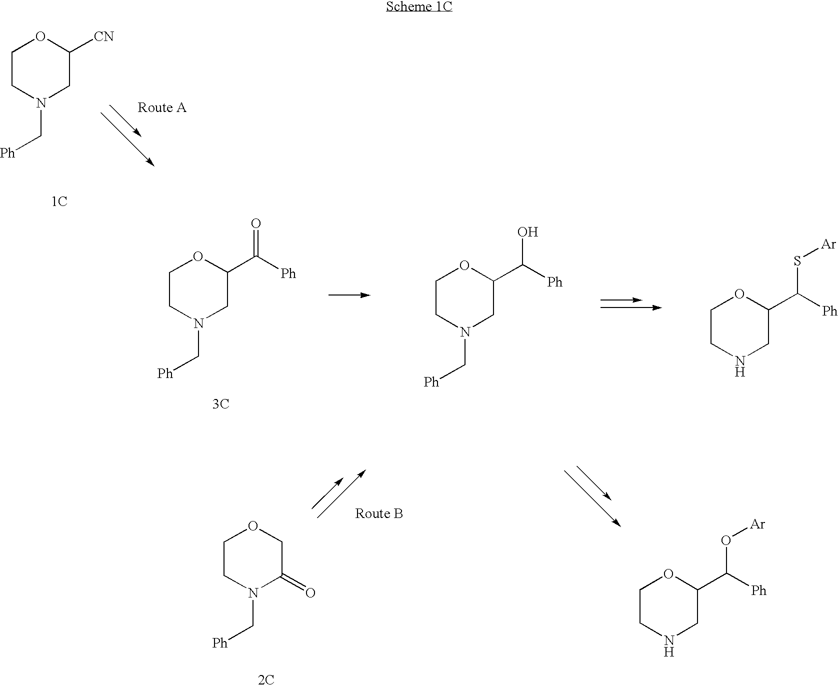

More detail of Route A is given in Scheme 2C:

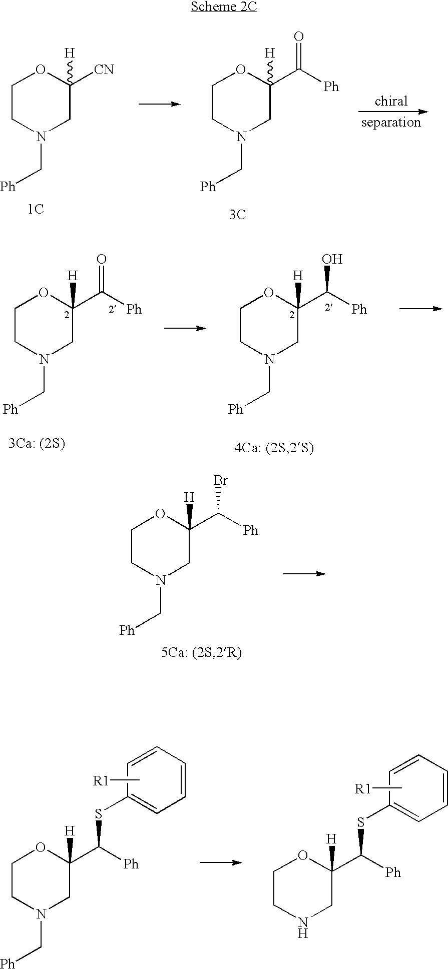

The amino alcohol 4Ca can be obtained by reaction of N-benzyl-cyanomorpholine 1C with a Grignard reagent, followed by acid hydrolysis to give racemic phenyl ketone 3C which may be separated on chiral HPLC. (2S)-Phenyl ketone 3Ca may then be reduced with DIP-Cl to give 4Ca in high diastereomeric excess. The amino alcohol 4Ca is converted into benzyl bromide 5Ca, to give the desired N-substituted aryl thio morpholines after displacement with the requisite aryl thiol. N-substituted aryloxy morpholines may be obtained in an analogous manner by displacement with the requisite hydroxyaryl compound. Alternatively, N-substituted aryloxy morpholines may be obtained by addition of a strong base, such as sodium hydride, to the amino alcohol 4Ca to form a nucleophilic alkoxide followed by an SNAr reaction with an Ar group substituted with a suitable leaving group (e.g. F). Deprotection of the tertiary amine gives the final products.

Detail of route B is given in Scheme 3C:

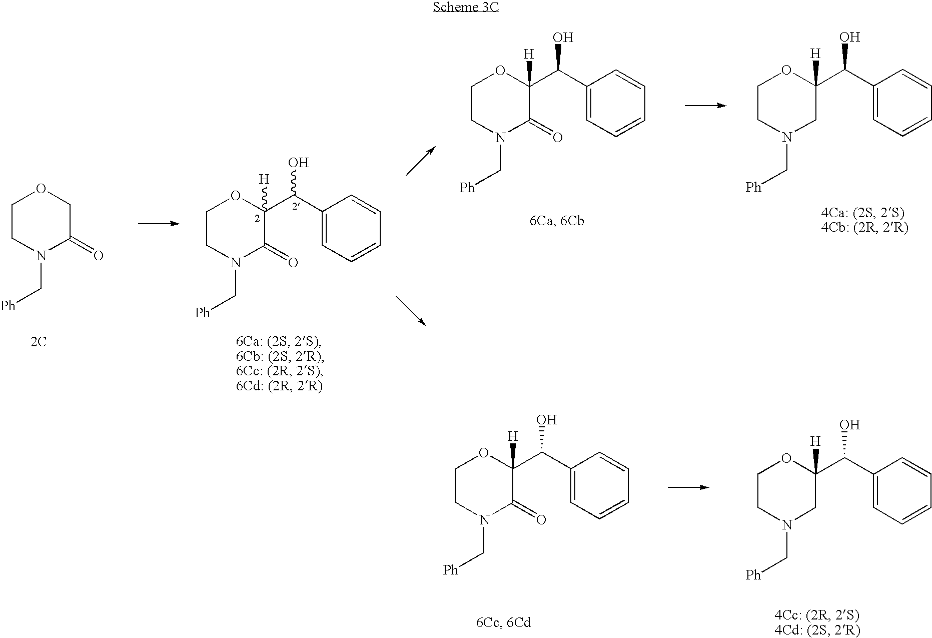

Treatment of N-benzyl morpholinone 2C with a strong base such as lithium diisopropylamide at low temperature followed by addition of benzaldehyde gives aldol adducts 6Ca-6Cd as a 2:1 mixture of diastereomer pairs 6Ca,6Cb and 6Cc,6Cd, which may be separated using conventional chromatographic techniques. Reduction with a borane reagent at elevated temperatures gives diasteremeric amino alcohol pairs 4Ca,4Cb and 4Cc,4Cd respectively.

Amino alcohol pair 4Ca,4Cb may be converted to bromide 5Ca,5Cb and further to racemic aryl thio morpholines as outlined in Scheme 4C. Amino alcohol pair 4Cc,4Cd may be converted into the corresponding mesylate. Displacement with the requisite thiol, followed by removal of the nitrogen protecting group furnishes aryl thiol morpholines as racemic mixtures of two diastereomers. The racemic aryl thiol morpholines may be separated into enantiomerically pure products using chiral HPLC technology. N-substituted aryloxy morpholines may be obtained in an analogous manner by displacement with the requisite hydroxyaryl compound.

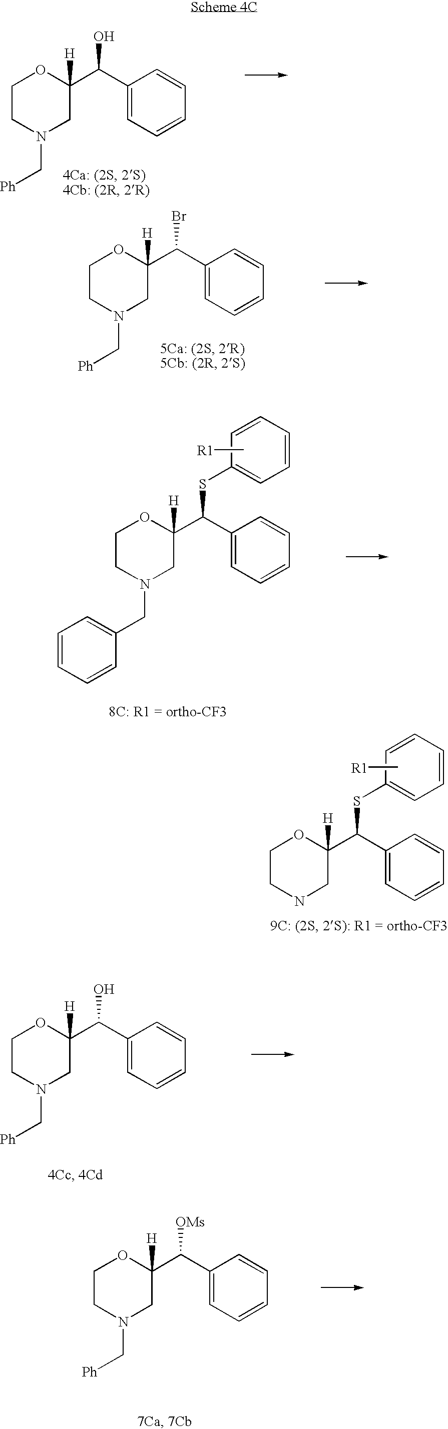

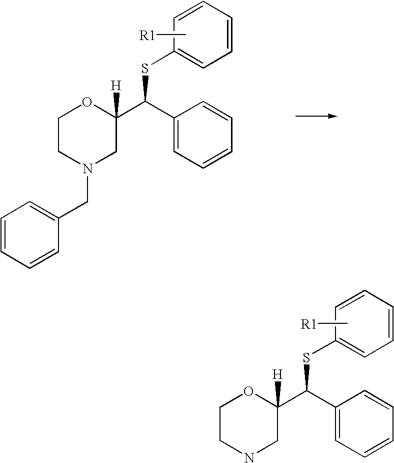

Aryl-substituted morpholines 33C, 35C, 37C may be obtained from morpholinone 2C as outlined in Scheme 5C:

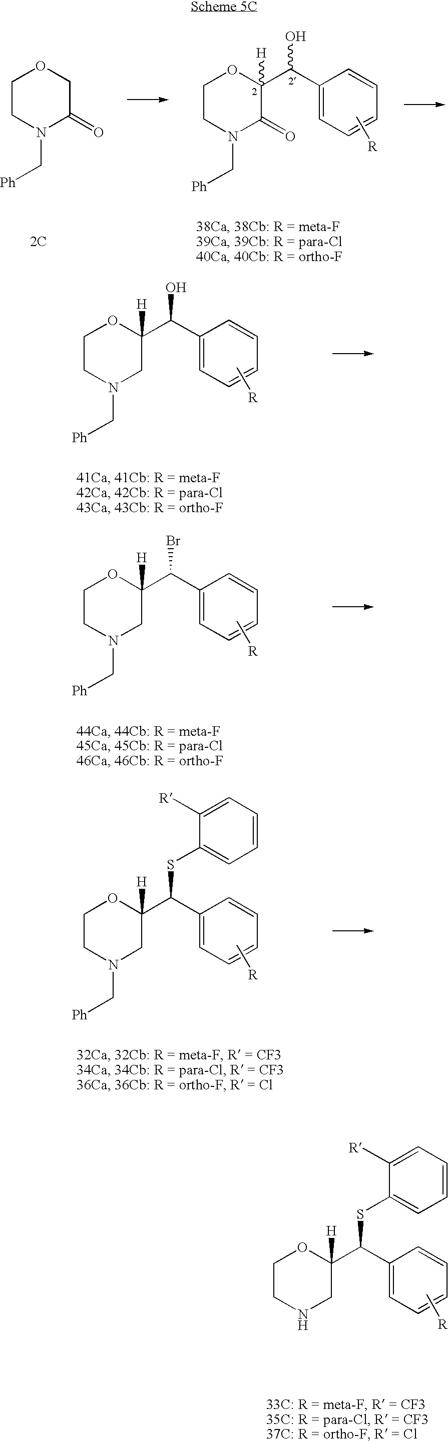

An alternative route to 9C is outlined in Scheme 6C. This route makes use of a chiral auxiliary and gives 9C in enantiomerically pure form.

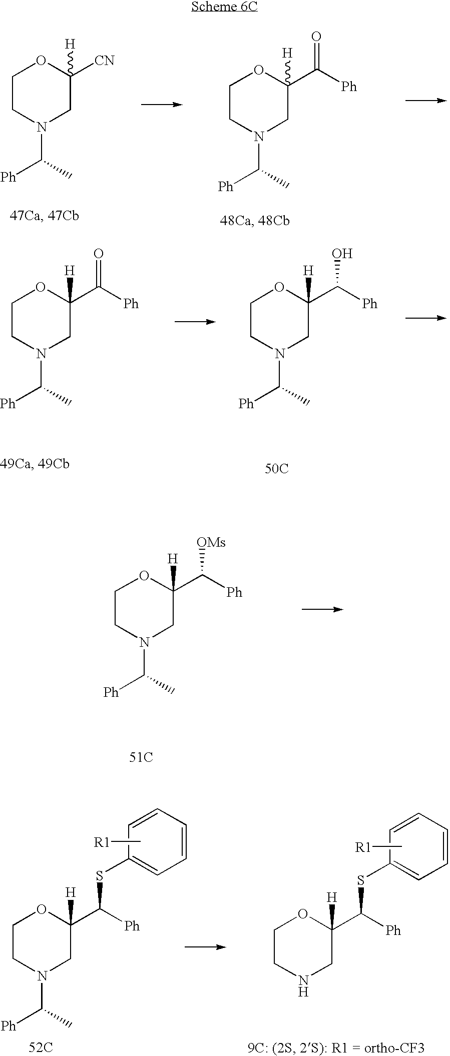

### Pretaration of Compounds of Formula (ID)

Compounds of formula (ID) may be prepared using the following methods. General schemes outlining the synthetic routes used to prepare racemic products are given below. All active racemates may be separated into single enantiomers using chiral HPLC and may be readily converted into suitable salts.

Compounds of formula (ID) wherein Ar is (i) and R^{2c} is H may be prepared as shown in Scheme 1D below:

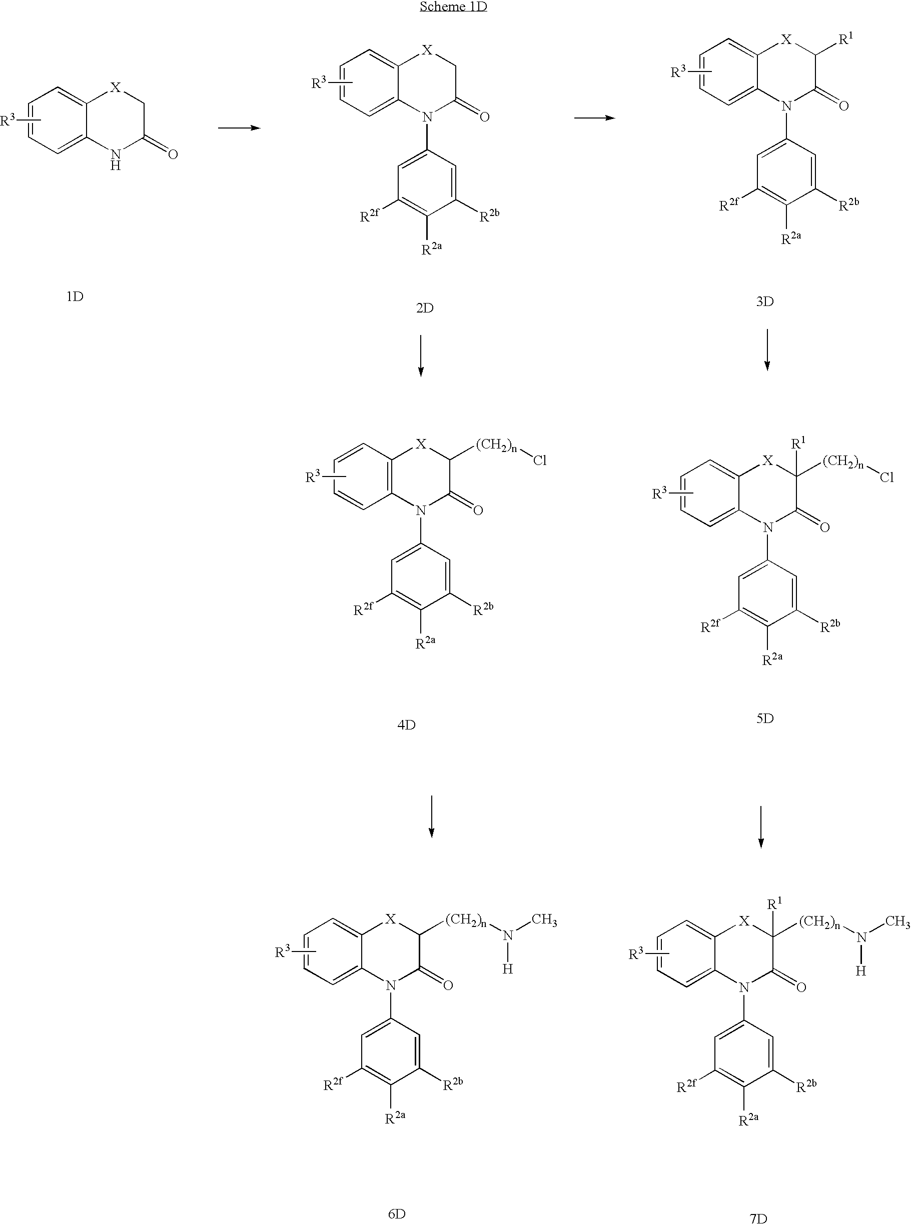

Quinolin-2-one 1D or its corresponding 4-oxo and 4-thio derivatives can be N-arylated using modified conditions to those reported by Buchwald, (J. Am. Chem. Soc., 123, 2001, p. 7727). For example the quinolin-2-one 1D is reacted with 3 equivalents of Ar—Br wherein Ar is (i) and R^{2c} is H, 0.2 equivalents of trans-cyclohexanediamine, 0.2 equivalent of copper iodide (CuI), 2.1 equivalents of potassium carbonate (K₂CO₃), in an organic solvent such as 1,4-dioxane at a temperature of 125° C. overnight. The resulting N-arylated quinolin-2-one 2D can be alkylated by treatment with a strong base such as lithium hexamethyldisilazide (LiHMDS) at temperatures of −78° C. in a suitable organic solvent such as tetrahydrofuran (THF), followed by the addition of an alkyl halide such as alkyl iodide to give the corresponding 3-alkylated-N-arylated quinolin-2-one derivative 3D. Using the same alkylating conditions above with a 1,2-dihaloethane, such as 1-bromo-2-chloroethane, or a 1,3-dihalopropane, such as 1-bromo-3-chloropropane, as alkylating agents provides 4D or 5D wherein n is 2 or 3 respectively. These halo analogues were chosen as ideal precursors to the desired amine products. For instance, treatment of 4D or 5D with aqueous methylamine, in the presence of a catalytic amount of a suitable iodide, such as potassium iodide (KI), in ethanol at 100° C. provided the racemic amine products 6D and 7D respectively, in moderate yields.

Compounds of formula (ID) wherein Ar is (i), R^{2c} is H and n is 3 may be prepared using alternative chemistry as shown in Scheme 2D.

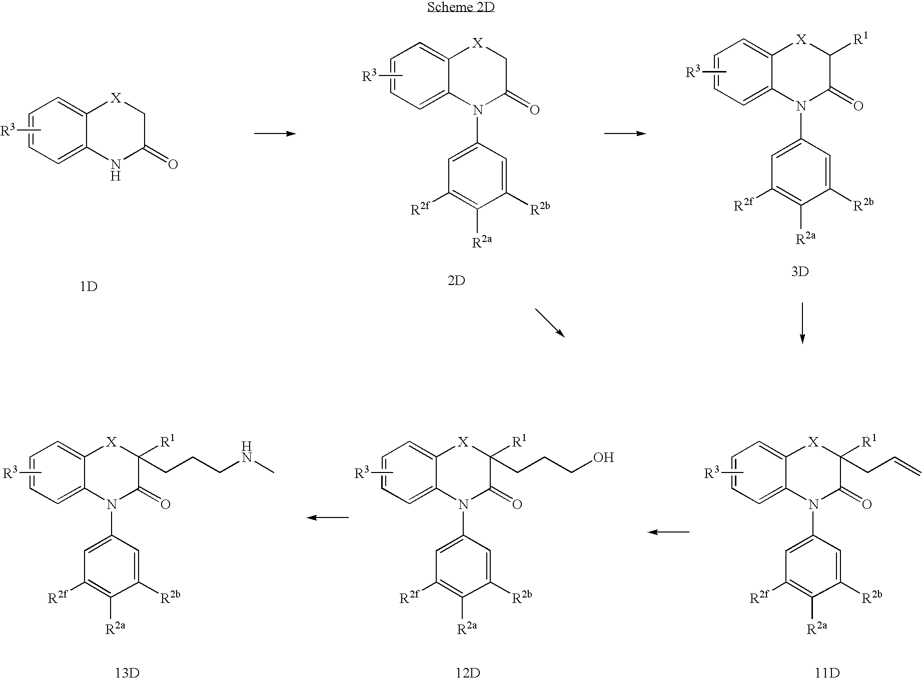

Quinolin-2-ones 2D and 3D can be alkylated using the aforementioned alkylating procedure using an allyl halide e.g. allyl bromide as the alkylating agent to give the corresponding 3-allyl-N-arylated-quinolin-2-ones 11D. Said allyl analogues could then be converted to the corresponding primary alcohols 12D by a hydroboration procedure involving a suitable borane, such as 9-BBN in a suitable solvent such as THF. Oxidative work up using for example reaction conditions such as aqueous hydrogen peroxide in a solvent such as ethanol, in the presence of a suitable base, such as sodium hydroxide, gave moderate to good yields of alcohol products after column chromatography purification. The alcohols were cleanly converted into their mesylates, by reaction of a mesyl halide such as mesyl chloride in the presence of a suitable base such as triethylamine in a suitable solvent such as THF at a suitable temperature such as 0° C. to room temperature. The resulting mesylates are used directly in the amination step described above in Scheme 1D to provide good yields of the final racemic targets 13D.

In order to prepare a range of N-arylated analogues advanced intermediates were prepared that could undergo N-arylations with a range of substituted aryl halides, such as aryl bromides or iodides, 2 and 3-halothiophenes, 2 and 3-halofurans or 2 and 3-halopyrroles. The synthetic route used to prepare intermediates 19D is shown below in Scheme 3D.

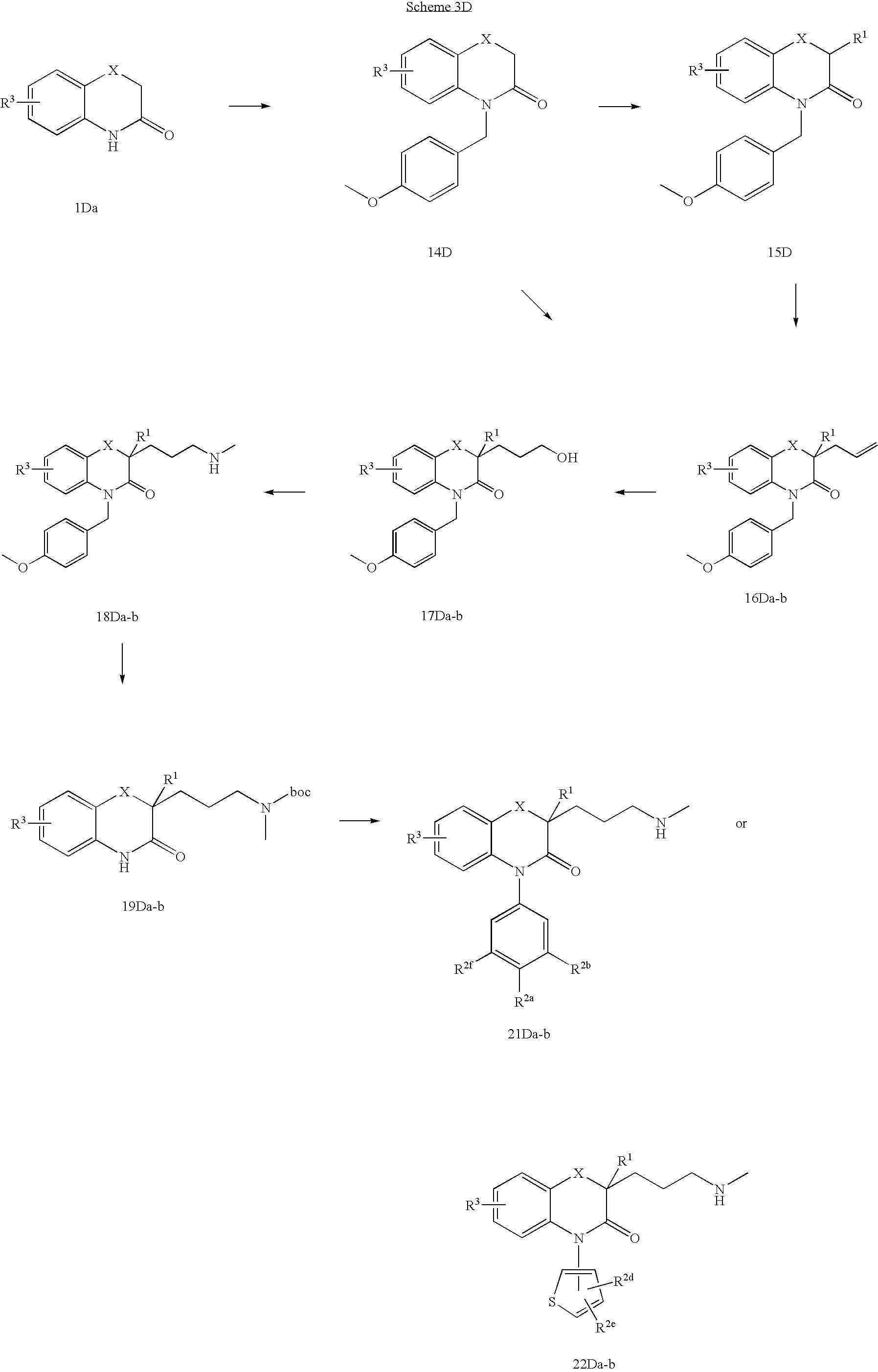

Compounds of formula (ID) wherein n is 3 may be prepared as shown in Scheme 3D. This method is particularly suitable for compounds wherein Ar is (i) and R^{2c} is H or Ar is (ii), wherein —Y— is —S—.

Quinolin-2-one 1D can be protected using a suitable amide-protecting group such as those described in T. W. Greene, “Protective Groups in Organic Synthesis”, John Wiley and Sons, New York, N.Y., 1991, hereafter referred to as “Greene”. For example quinolin-2-one 1D can be protected with a 4-methoxybenzyl group. The protection reaction can be carried out for example using a suitable base, such as sodium hydride in a suitable solvent, such as dimethylfornamide, followed by reaction with a 4-methoxybenzyl halide, such as 4-methoxybenzyl chloride, to give the corresponding N-protected derivative 14D in good yield. This intermediate can be converted directly to the allyl analogue 16Da, wherein R¹═H, in a manner described earlier or converted into the alkyl analogue 15D which can be subsequently alkylated with a allyl halide to give the allyl analogue 16Db, wherein R¹ is C₁-C₄ alkyl. Using the same hydroboration, mesylation and amination sequence described in Scheme 2D provided both amines 18Da-b. Deprotection of protected quinolin-2-one could be achieved using any suitable deprotection conditions as those shown in Greene. For example, the 4-methoxybenzyl group could be cleaved cleanly using trifluoroacetic acid and anisole at 65° C. The resultant product could be selectively protected on the secondary amine with a suitable nitrogen protecting group as those described in Greene. For example, the secondary amine can be protected with a Boc group. The reaction can be carried out with Boc anhydride in a suitable solvent such as THF to provide multi gram quantities of 19Da-b. Reaction of 19Da-b with various aryl bromides using the previously described N- arylation conditions, deprotection using suitable deprotecting conditions such as those described in Greene gave a range of final racemic targets 21Da-b or 22Da-b. For example, for compounds protected with a Boc group they can be deprotected in the presence of trifluoroacetic acid (TFA) in a suitable organic solvent such as dichoromethane (DCM).

Intermediates 19Da-b wherein R³ is a halo group, for example chloro or bromo, can be used to provide compounds of formula (ID) wherein R³ is a phenyl group, such as compound 24D, via a Suzuki coupling, see Scheme 4D below.

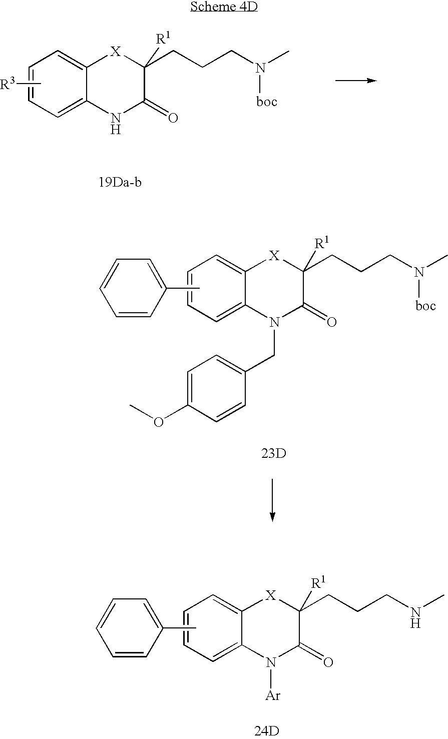

Intermediates 19Da-b, wherein R³ is for example bromo can be N-protected with a suitable amide protecting group for example 4-methoxybenzyl as described in Scheme 3D above and then coupled with phenylboronic acid under Suzuki conditions to provide the phenyl analogues 23D. Deprotection of the 4-methoxybenzyl group with TFA, followed by protection of the resulting secondary amine with a suitable nitrogen protecting group such as Boc followed by subsequent N-arylation and Boc deprotection using the previously described methodology gave the final target 24D.

It will be appreciated that compounds of formula (IDa) wherein R³ is bromo or chloro can be prepared as shown in Schemes 1D to 4D above starting from the corresponding haloquinolin-2-ones. Alternatively, they can be prepared from the corresponding quinolin-2-one 1D wherein R³ is hydrogen as mentioned above including an extra step comprising the halogenation of a suitable intermediate at some stage of the synthesis. For example quinolin-2-one 1D in Scheme 2D can be halogenated using N-chlorosuccinimide in a suitable solvent such as DMF at a suitable temperature such as room temperature to give the corresponding 6-chloro-quinolin-2-one ID wherein R³ is Cl.

Alternatively intermediates (19Da-b) wherein R³ is H in Scheme 3D can be halogenated in the presence of N-chloro and N-bromosuccinimide in a suitable solvent such as DMF to give the corresponding 6-chloro and 6-bromoquinolin-2-ones (20Da-c).

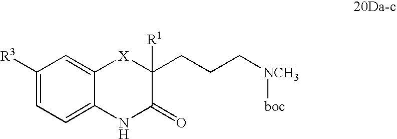

It will be appreciated that Schemes ID to 4D above relate to methods for the preparation of compounds of formula (ID) wherein Ar is (i) and R^{2c} is hydrogen. Compounds of formula (ID) wherein Ar is (i) and R^{2c} can be other than hydrogen, can be prepared using any of the general methods mentioned above, starting from the corresponding N-arylated quinolin-2-one 27D. A general method for preparing said intermediates is illustrated in Scheme 5D. Commercially available 3-(2-Bromo-phenyl)-propionic acids 25D can be converted to amide 26D using standard amide coupling conditions and converted to the N-arylated quinolin-2-ones 27D by an intramolecular, palladium catalysed cyclisation according to the method of Buchwald et al (Tetrahedron, 1996, 52, p.7525).

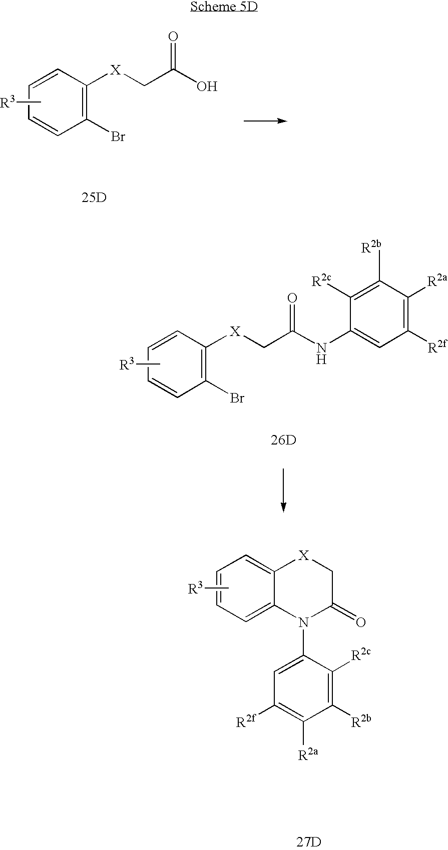

### Preparation of Compounds of Formula (IE)

Compounds of formula (IE) may be prepared by conventional organic chemistry techniques and also by solid phase synthesis. Compounds of formula (IE) can be prepared via the 3-aminopyrrolidine intermediate of formula (IVE) as illustrated in the Scheme 1E below:

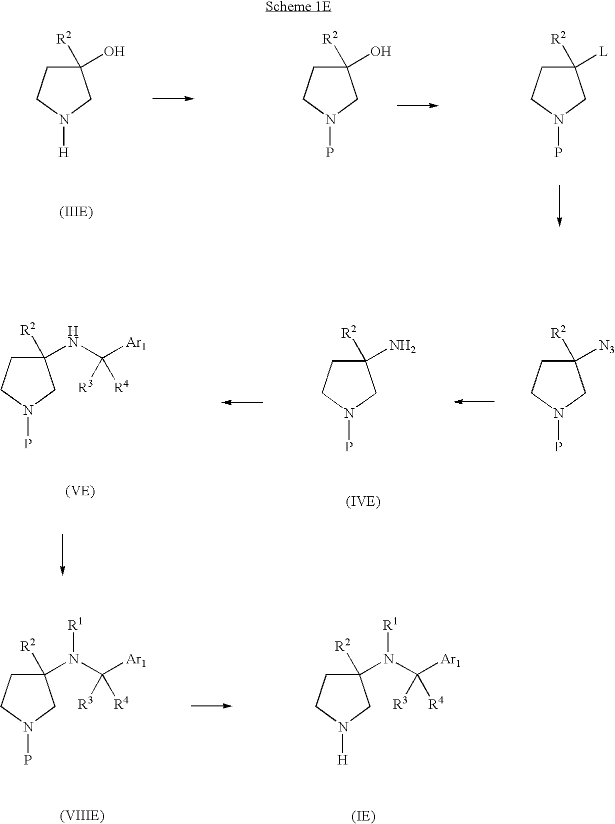

Commercially available 3-hydroxypyrrolidine of formula (IIIE) wherein R² is hydrogen, can be protected using a suitable nitrogen-protecting group such as those described in T. W. Greene, “Protective Groups in Organic Synthesis”, John Wiley and Sons, New York, N.Y., 1991, hereafter referred to as “Greene”. For example 3-R-hydroxypyrrolidine (IIIE) can be protected with a tert-butoxycarbonyl group, (boc). The protection reaction can be carried out for example using Boc anhydride in a suitable solvent such as for example tetrahydrofuran (THF) or dichloromethane (DCM) in the presence of a base such as tryethylamine (TEA) or 4—(dimethylamino)pyridine (DMAP). It will be appreciated that for compounds of formula (IE) wherein R² is C₁-C₂ alkyl, the 3-hydroxypyrrolidine of formula (IIIE) can be prepared from the readily available 3-pyrrolidinone via addition of the appropriate C₁-C₂ alkyl organometallic. The hydroxy group of the N-protected-3-hydroxypyrrolidine can be converted into a suitable leaving group (L) such as for example chloride, bromide, iodide or mesylate. For example the N-protected-hydroxypyrrolidine can be converted to the mesylate in the presence of mesyl chloride and a suitable base such as triethylamine in a solvent such as DCM. Said mesylate is subsequently displaced with the corresponding azide in a suitable solvent such as dimethylformnamide (DMF) or dimethylsulphoxide (DMSO). This azide intermediate can be converted to the corresponding N-protected-aminopyrrolidine of formula (IVE) via hydrogenation in the presence of a suitable catalyst such as Palladium on charcoal and in a suitable solvent such as methanol or ethanol.

For compounds of formula (IE) wherein R⁴ is H, intermediate (IVE) can be alkylated via reductive alkylation with a ketone of formula R³—CO—Ar₁ wherein R³ and Ar, have the values for formula (IE) above. The reductive alkylation can be carried out for example as a hydrogenation reaction in the presence of a suitable catalyst such as Palladium on charcoal and a suitable solvent such as for example ethanol. Alternatively, said reductive alkylation can be carried out in the presence of a suitable borane such as sodium triacetoxyborohydride, NaBH(OAc)₃ and optionally in the presence of a suitable acid such as acetic acid, in a suitable solvent such as for example dichoroethane (DCE).

Alternatively, intermediate of formula (VE) wherein R⁴ is H can be prepared as shown in Scheme 2E below by reductive alkylation of readily available 3-aminopyrrolidine of formula (VIE) wherein R² has the values defined for formula (IE) above, followed by the protection of the nitrogen in the pyrrolidine ring using a suitable protecting group such as those defined in Greene.

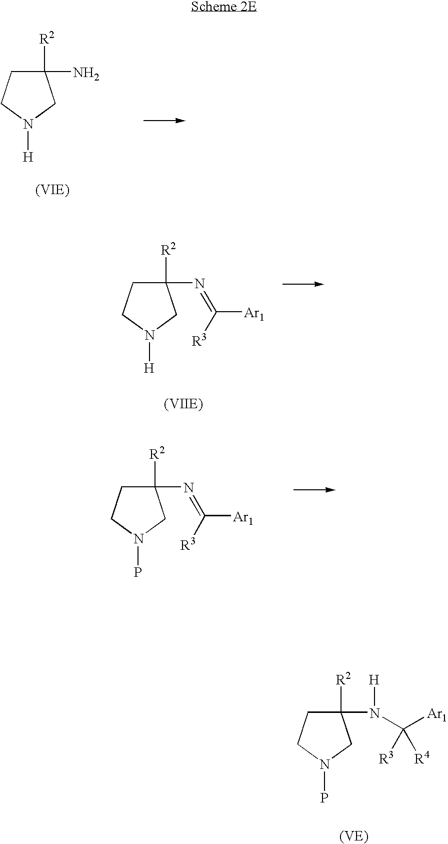

For example the reductive alkylation can be carried out in the presence of a ketone of formula Ar₁—CO—R³ wherein Ar₁ and R³ have the values defined for formula (IE) above. Initial condensation of the amino pyrrolidine with the ketone is undertaken in the presence of a suitable acid such as p-toluenesulphonic acid, in a suitable solvent such as toluene. The resultant imino pyrrolidine intermediate can then be protected with for example a boc group. The reaction can be carried out in the presence of boc anhydride and a suitable base such as DMAP, in a suitable solvent such as DCM. Said imine is reduced via hydrogenation in the presence of a suitable catalyst such as palladium on charcoal, in a suitable solvent such as ethanol to give the corresponding amine of formula (VE).

Intermediate of formula (VE) can be converted to compounds of formula (VIIIE) via reductive alkylation with an aldehyde of formula R⁹—CHO, wherein R⁹ is chosen such that R⁹—CH₂═R¹ and R¹ has the values defined for formula (IE) above. The reductive alkylation can be carried out using standard methods, for instance as those mentioned above with the ketone Ar₁—CO—R³.

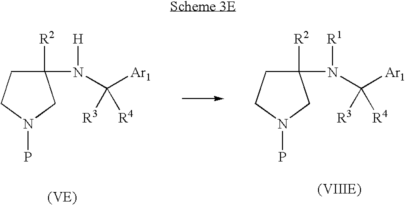

For example a compound of formula (VE) can be alkylated with R⁹—CHO in the presence of a suitable borane, such as NaBH(OAc)₃, optionally in the presence of an acid such as acetic acid, in the presence of a suitable solvent such as dichloroethane (DCE).

For compounds of formula (IE) wherein R³ and R⁴ are hydrogen the alkylation of intermediate (VE) can be carried out with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIE)ₐ. It will be appreciated that the same reaction can be carried out using Ar₁—CR³R⁴-L₁ wherein R³ and R⁴ are C₁-C₂ alkyl.

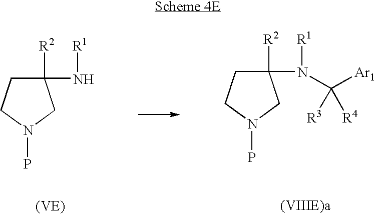

Compounds of formula (IE) wherein R¹ is —CH₂—COO—(C₁-C₂ alkyl) can be prepared by reacting intermediate (VE) with a compound of formula L₂-CH₂—COO—(C₁-C₂ alkyl) wherein L₂ is a suitable leaving group such as for example bromo, chloro or iodo. Said reaction can be carried out in the presence of a suitable base such as sodium hydride, in a suitable solvent such as dimethylformamide.

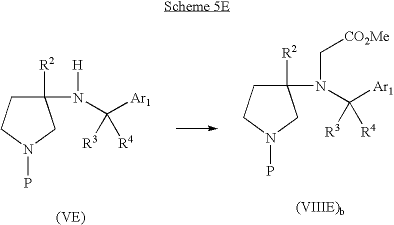

Compounds of formula (IE) wherein R¹ is —(CH₂)ₘ—CF₃ can be prepared by reacting intermediate (VE) with a compound of formula HOOC—(CH₂)ₘ_{₁}—CF₃, wherein m₁ is 0, 1 or 2. The acid may be activated as its anhydride or acyl chloride, and is reacted in the presence of a suitable base such as triethylamine and a catalytic amount of DMAP, in a suitable solvent such as DCM. The resulting amide can be reduced to the amine of formula (VIIIE), in the presence of a suitable borane. For example, for compounds wherein m is 1, the reduction can be carried out in the presence of BH₃-Me₂S borane-dimethyl sulphide complex, in a suitable solvent such as THF.

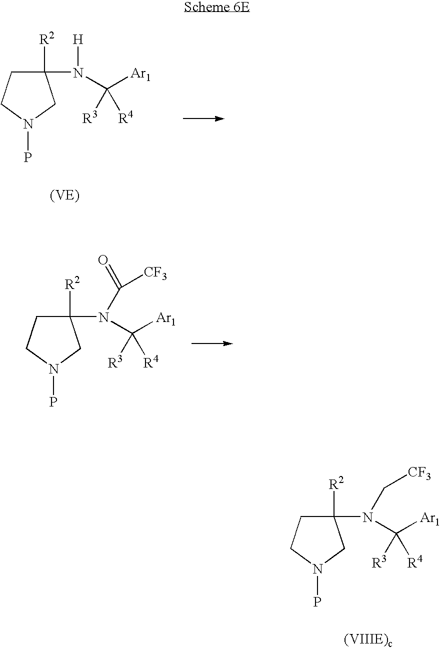

Compounds of formula (IE) wherein R¹ is —C₁-C₆ alkylene)-OH can be prepared by reacting intermediate (VE) with an epoxide. For example for compounds wherein R¹ is —CH₂—C(CH₃)₂—OH, the intermediate of formula (VE) is reacted with 2,2-dimethyloxirane, in a suitable solvent such as aqueous ethanol.

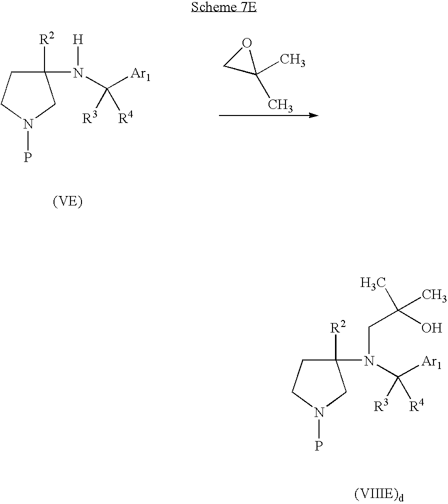

Alternatively compounds of formula (IE) wherein R¹ is —(C₁-C₆alkylene)-OH can be prepared by reacting intermediate (VE) with an w-haloalkanoate, such as methylbromoacetate, in the presence of a base such a sodium hydrogen carbonate in a solvent such as acetonitrile. The intermediate ester is then reacted with 2 equivalents of methyl magnesium bromide in THF to yield the tertiary alcohol(VIIIE)_{d}:

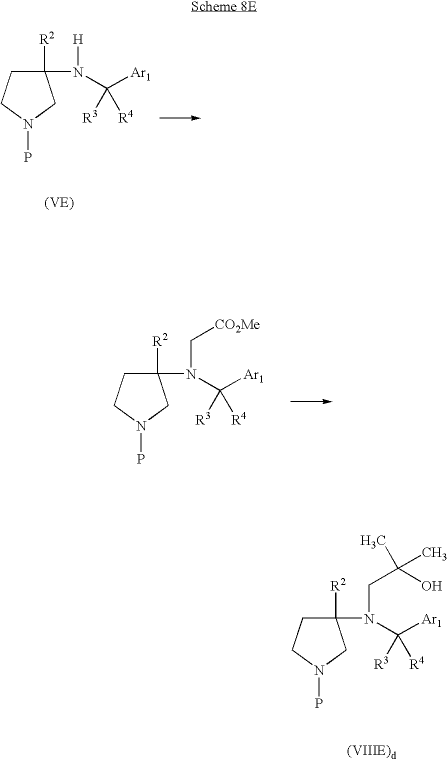

It will be appreciated that the Scheme 8E above applies to alkylene chains longer than —CH₂—.

Compounds of formula (IE) wherein R¹ is —C₂-C₆ alkenyl, —(CH₂)ₙ—S—(C₁-C₃ alkyl), —(C₁-C₅ alkylene)-O—(C₁-C₃ alkyl), —(C₁-C₅ alkylene)-O—(C₃-C₆ cycloalkyl), —(C₁-C₅ alkylene)-SO₂—(C₁-C₃ alkyl), —(C —C₅ alkylene)-OCF₃, or —(C₁-C₅ alkylene)-CN, can be prepared via alkylation of intermediate (VE) with a compound of formula L₂-C₂-C₆ alkenyl, L₂-(CH₂)ₙ—S—(C₁-C₃ alkyl), L₂-(C₁-C₅ alkylene)-O—(C₁-C₃ alkyl), L₂-(C₁-C₅ alkylene)-O—(C₃-C₆ cycloalkyl), L₂-(C₁-C₅ alkylene)-SO₂—(C₁-C₃ alkyl), L₂-(C₁-C₅ alkylene)-OCF₃, or L₂-(C₁-C₅ alkylene)-CN respectively, wherein L₂ is a suitable leaving group such as chloro, bromo, iodo or mesylate, in the presence of a suitable base such as potassium carbonate and a suitable solvent such as acetonitrile, to give the corresponding intermediate of formula (VIIIE)ₑ.

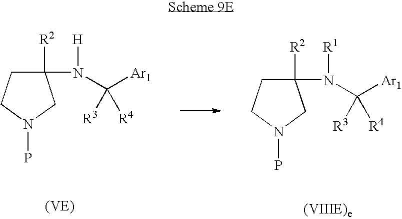

Compounds of formula (IE) wherein R is a group of formula (i) can be prepared using the synthesis illustrated in Scheme 10E for compounds wherein R¹ is 4-tetrahydropyranyl. The compound of formula (IVE) can be alkylated via reductive alkylation using standard methods, as those mentioned above with the ketone Ar₁—CO—R³. For example compound of formula (IVE) can be alkylated with 4-tetrahydropyranone in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIE)_{f}. It will be appreciated that as mentioned above the same reaction can be carried out using Ar₁—CR³R⁴-L, wherein R³ and R⁴are C₁-C₂ alkyl.

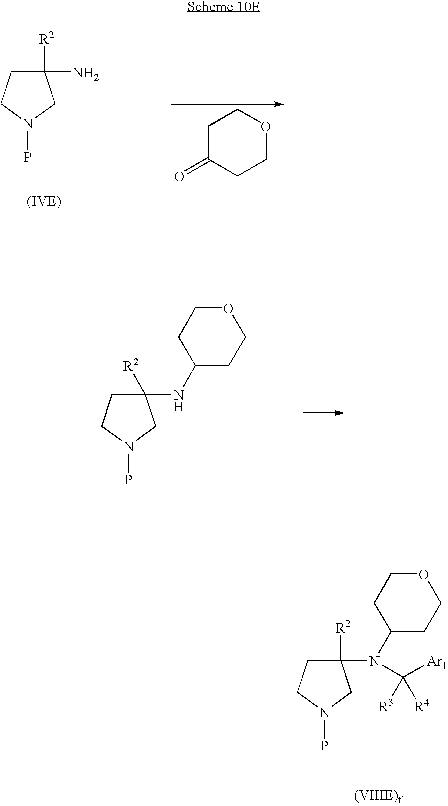

It will be appreciated that for compounds of formula (IE) wherein R¹ is a group of formula (i) and r is 1 then the reductive amination can be carried out using the same reaction conditions but using the corresponding homologous aldehyde of formula 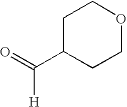
instead of the corresponding 4-tetrahydropyranone. Alternatively, compounds of formula (IE) wherein R¹ is a group of formula (i) and r is 1 can be prepared via formation of an amide, followed by reduction of this amide bond to the corresponding amine as shown in Scheme 11E below:

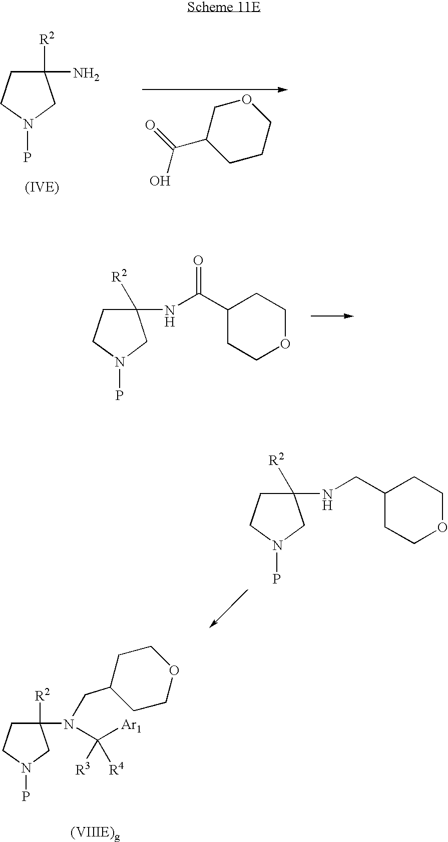

The coupling reaction can be carried out using standard methods known in the art. The reduction of the amide bond can also be carried by general methods known in the art for example using the same reduction conditions as those used in Scheme 6, such as in the presence of BH₃-Me₂S (borane-dimethyl sulphide complex), in a suitable solvent such as THF.

Alternatively, compounds of formula (IE) wherein R¹ is a group of formula (i) wherein r is 0 can be prepared by a process illustrated in Scheme 12E for compounds wherein -Z is hydrogen, s is 1, t is 2, each R⁵, R⁶, R⁷ and R⁸ are hydrogen and —X— is —O—, (i.e. R¹ is 2-tetrahydrofuranyl). The compound of formula (IVE) can be alkylated with a compound of formula: 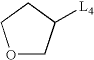
wherein L₄ is a suitable leaving group such as chloro, bromo, iodo, mesylate or tosylate, in the presence of a suitable base such as potassium carbonate and a suitable solvent such as acetonitrile, to give the corresponding secondary amine which can be subsequently alkylated with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIE)_{f}. It will be appreciated that as mentioned above the same reaction can be carried out using Ar₁—CR³R⁴-L₁ wherein R³ and R⁴are C₁-C₂ alkyl.

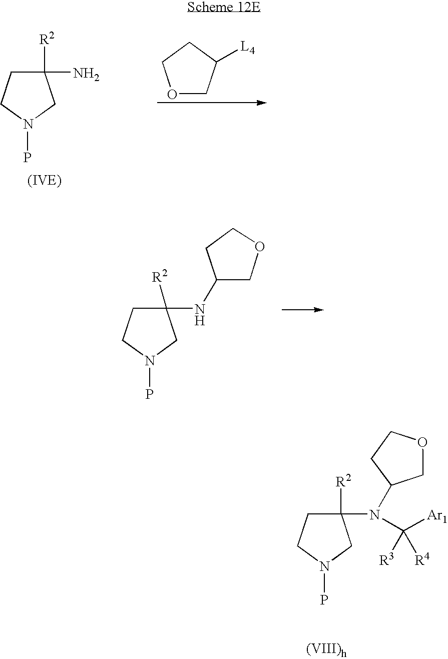

The tetrahydrofuranyl intermediates can be prepared from the corresponding 3-hydroxytetrahydrofuran, wherein the hydroxy group is converted into the leaving group using standard methods.

Compounds of formula (IE) wherein R¹ is a group of formula (i) and —X— is —SO₂— can be prepared from the corresponding intermediates (VIIIE)_{f}, wherein the thioether is oxidized to the corresponding sulphoxide as shown in Scheme 13E below:

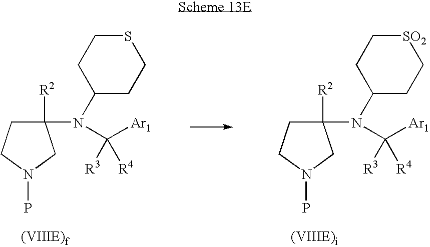

Compounds of formula (IE) wherein R¹ is a group of formula (ii) can be prepared using the synthesis illustrated in Scheme 14E for compounds wherein R¹ is oxabicyclo[3,2,1]octan-3-yl. The compound of formula (IVE) can be alkylated via reductive alkylation using standard methods, as those mentioned above with the ketone Ar₁—CO—R³. For example compound of formula (IVE) can be alkylated with oxabicyclo[3,2,1]octan-3-one in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIE)₁. It will be appreciated that as mentioned above the same reaction can be carried out using Ar₁—CR³R⁴-L₁ wherein R³ and R⁴are C₁-C₂ alkyl.

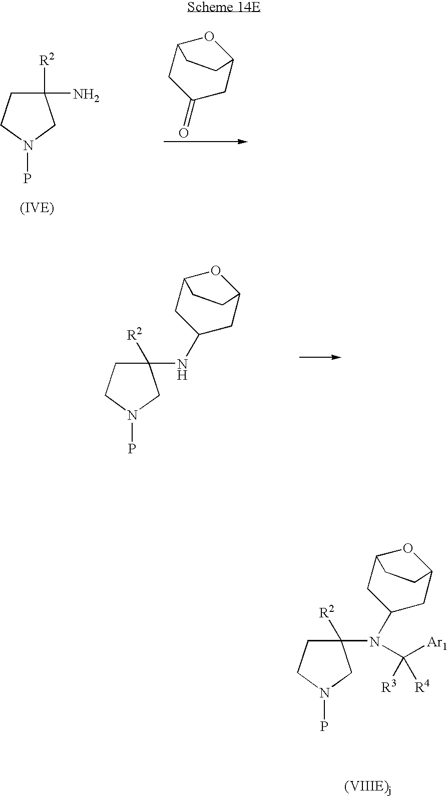

The oxabicyclo[3,2,1]octan-3-one intermediate is prepared according to the method described in A E Hill, G Greenwood and H M R Hoffmann JACS 1973, 95, 1338. It will be appreciated that for compounds of formula (IE) wherein R is a group of formula (i) and r is 1 then the reductive amination can be carried out using the same reaction conditions but using the corresponding homologous aldehyde of formula 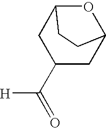
instead of the corresponding oxabicyclo[3,2,1]octan-3-one.

Compounds of formula (IE) wherein Ar₁ is a substituted or unsubstituted pyridyl group can be prepared by a process illustrated in Scheme 15E for compounds wherein R³ and R⁴ are hydrogen and Ar₁ is 3-phenylpyrid-2-yl.

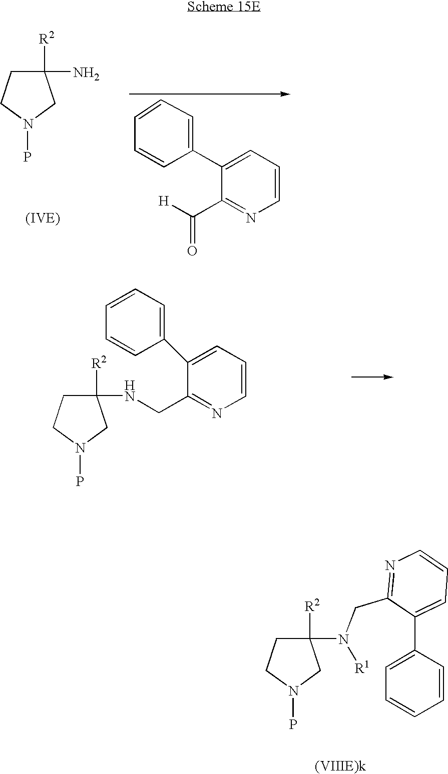

The compound of formula (IVE) can be alkylated via reductive alkylation using standard methods, as those mentioned above with the ketone Ar₁—CO—R³. For example compound of formula (IVE) can be alkylated with an aldehyde of formula: 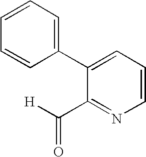
in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated using the geheral methods described above for the incorporation of R¹. The intermediate aldehyde can be prepared via reduction of readily available methyl 3-phenyl picolinate to the corresponding alcohol and subsequent oxidation to the aldehyde as shown in Scheme 16E below.

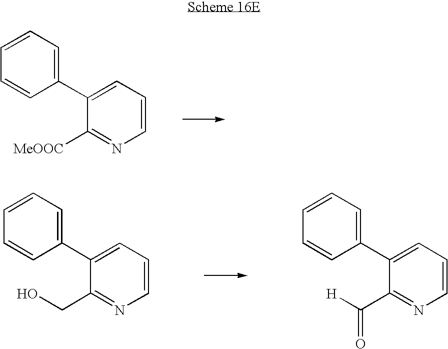

The reduction step can be carried out in the presence of a suitable reducing agent such as lithium borohydride in a suitable solvent such as tetrahydrofuran. The oxidation to the aldehyde can be carried out under Swern conditions such as oxalyl chloride and DMSO in DCM.

Compounds of formula (IE) wherein Ar₁ is a substituted or unsubstituted phenyl group can be prepared by a process illustrated in Scheme 17E for compounds wherein R³ and R⁴ are hydrogen and Ar₁ is 2-(3-pyridyl)phenyl.

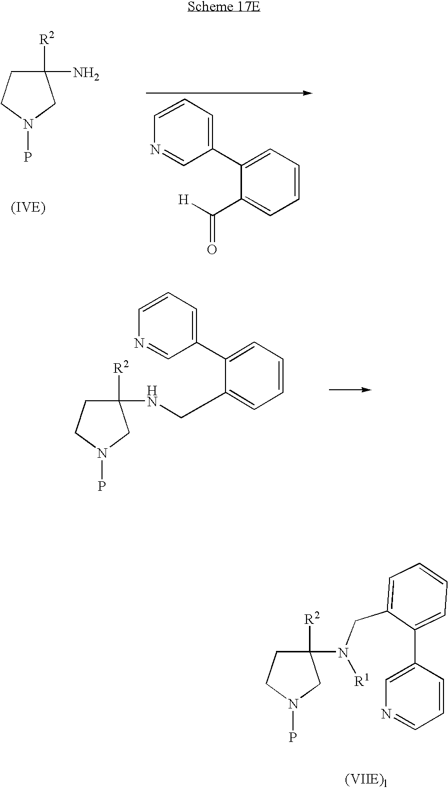

The compound of formula (IVE) can be alkylated via reductive alkylation using standard methods, as those mentioned above with the ketone Ar₁—CO—R³. For example compound of formula (IVE) can be alkylated with an aldehyde of formula: 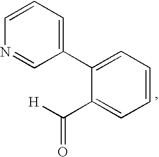
in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated using the general methods described above for the incorporation of R¹. The intermediate aldehyde can be prepared from the commercially available 2-formyl phenyl boronic acid via palladium coupling in the presence of 3-bromopyridine, a suitable palladium catalyst such as Pd(PPh₃)₄ and a suitable base such as potassium carbonate in a suitable solvent such as acetonitrile, as shown in Scheme 18E below.

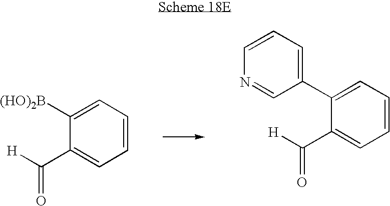

Compounds of formula (IE) wherein Ar₁ is a phenyl group substituted with a 1-pyrazole group can be prepared by a process illustrated in Scheme 19E.

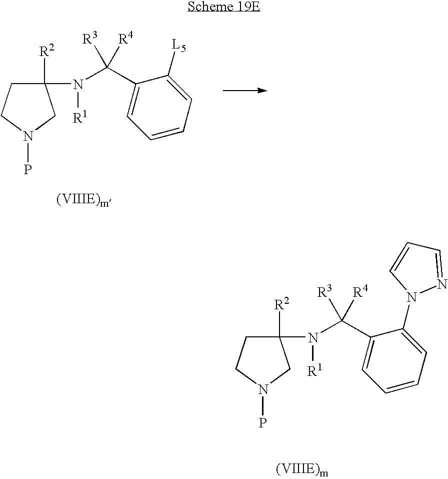

The pyrazole group can be incorporated by reacting a compound of formula (VIIIE)_{m′}, wherein L₅ is a suitable leaving group such as bromo, chloro or iodo, with pyrazole in the presence of a suitable base such as potassium carbonate and a catalytic amount of copper iodide in a suitable solvent such as for example DMF. The compound of formula (VIIIE)_{m′} can be prepared by any of the methods mentioned above for compounds wherein Ar1 is a phenyl group substituted with a halogen atom such as chloro, bromo or iodo.

It will be appreciated that any of the intermediates (VIIIE), (VIIIE)ₐ₋ₘ are then deprotected using suitable deprotecting conditions such as those discussed in Greene, to give the corresponding compounds of formula (IE). For example if the protecting group is a boc group, the deprotection reaction can be carried out in trifluoroacetic acid in a suitable solvent such as DCM. Alternatively the reaction can be carried out in ethanolic hydrochloric acid.

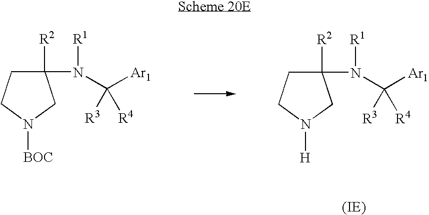

Compounds of formula (IE) wherein R³ and R⁴ are both hydrogen may also be prepared by solid phase synthesis by the route shown below in Scheme 21E below.

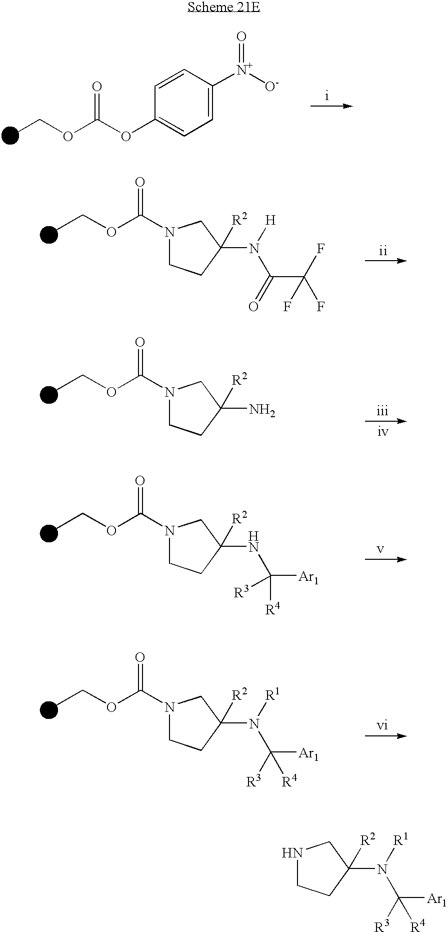

The sequence is preferably performed on a polystyrene resin. The process may be run in a combinatorial fashion such that all possible compounds from sets of precursors Ar₁CHO and R⁹CHO may be prepared, wherein R⁹ is chosen such that R⁹—CH₂═R¹, and R¹ and Ar₁ have the values defined above for formula (IE). The sequence is performed without characterisation of the resin-bound intermediates. In step (i) 3-trifluoroacetamido-pyrrolidine is bound to a solid support by reaction with 4-nitrophenyl carbonate activated polystyrene resin in the presence of a base, such as N,N-diisopropylethylamine, in a solvent such as DMF. In step (ii), the trifluoroacetamido protecting group is cleaved by hydrolysis with a base such as aqueous lithium hydroxide. In step (iii) the primary amine is then condensed with a substituted benzaldehyde in the presence of a dehydrating agent, such as trimethylorthoformate, to form the intermediate imine. In step (iv) the imine is reduced with a borane reducing agent, such as sodium cyanoborohydride, in a solvent such as DMF, containing acetic acid. In step (v) the resultant secondary amine is then reductively alkylated with an aldehyde in the presence of a reducing agent such as sodium triacetoxyborohydride in a solvent, such as DMF. In step (vi) the desired product is finally cleaved from the resin with acid, such as aqueous trifluoroacetic acid.

### Preparation of Compounds of Formula (IF)

Compounds of formula (IF) may be prepared by conventional organic chemistry techniques and also by solid phase synthesis.

Compounds of formula (IF′) can be prepared by the general methods illustrated below. It will be appreciated that the same methods can be used for compounds of formula (IF″) with the only difference that the nitrogen atom of the quinuclidines does not need to be protected as it is already a tertiary amine as it is explained in more detail below with reference to Scheme 1F.

Compounds of formula (IF′) can be prepared via the 3-aminopiperidine intermediate of formula (IVF) as illustrated in Scheme 1F below:

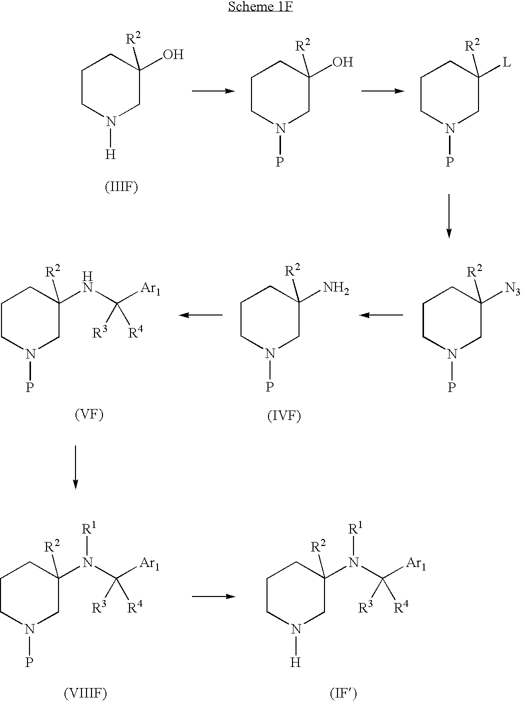

Commercially available 3-hydroxypiperidine of formula (IIIF) wherein R² is hydrogen, can be protected using a suitable nitrogen-protecting group such as those described in T. W. Greene, “Protective Groups in Organic Synthesis”, John Wiley and Sons, New York, N.Y., 1991, hereafter referred to as “Greene”. For example 3-R-hydroxypiperidine (IIIF) can be protected with a tert-butoxycarbonyl group, (boc). The protection reaction can be carried out for example using Boc anhydride in a suitable solvent such as for example tetrahydrofuran (THF) or dichloromethane (DCM) in the presence of a base such as triethylamine (TEA) or 4-(dimethylamino)pyridine (DMAP). It will be appreciated that for compounds of formula (IF) wherein R² is C₁-C₂ alkyl, the 3-hydroxypiperidine of formula (IIIF) can be prepared from the readily available 3-pyrrolidinone via addition of the appropriate C₁-C₂ alkyl organometallic. The hydroxy group of the N-protected-3-hydroxypiperidine can be converted into a suitable leaving group (L) such as for example chloride, bromide, iodide or mesylate. For example the N-protected-hydroxypiperidine can be converted to the mesylate in the presence of mesyl chloride and a suitable base such as triethylamine in a solvent such as DCM. Said mesylate is subsequently displaced with the corresponding azide in a suitable solvent such as dimethylformamide (DMF) or dimethylsulphoxide (DMSO). This azide intermediate can be converted to the corresponding N-protected-aminopiperidine of formula (IV) via hydrogenation in the presence of a suitable catalyst such as Palladium on charcoal and in a suitable solvent such as methanol or ethanol.

For compounds of formula (IF) wherein R⁴ is H, intermediate (IVF) can be alkylated via reductive alkylation with a ketone of formula R³—CO—Ar₁ wherein R³ and Ar₁ have the values for formula (IF) above. The reductive alkylation can be carried out for example as a hydrogenation reaction in the presence of a suitable catalyst such as Palladium on charcoal and a suitable solvent such as for example ethanol. Alternatively, said reductive alkylation can be carried out in the presence of a suitable borane such as sodium triacetoxyborohydride, NaBH(OAc)₃ and optionally in the presence of a suitable acid such as acetic acid, in a suitable solvent such as for example dichoroethane (DCE).

Alternatively, intermediate of formula (VF) wherein R⁴ is H can be prepared as shown in Scheme 2F below by reductive alkylation of readily available 3-aminopiperidine of formula (VIF) wherein R² has the values defined for formula (IF) above, followed by the protection of the nitrogen in the piperidine ring using a suitable protecting group such as those defined in Greene.

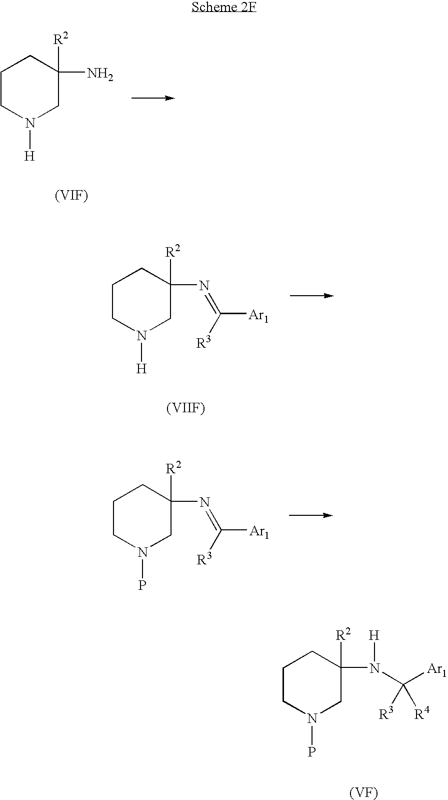

For example the reductive alkylation can be carried out in the presence of a ketone of formula Ar₁—CO—R³ wherein Ar₁ and R³ have the values defined for formula (IF) above. Initial condensation of the amino piperidine with the ketone is undertaken in the presence of a suitable acid such as p-toluenesulphonic acid, in a suitable solvent such as toluene. The resultant imino piperidine intermediate can then be protected with for example a boc group. The reaction can be carried out in the presence of boc anhydride and a suitable base such as DMAP, in a suitable solvent such as DCM. Said imine is reduced via hydrogenation in the presence of a suitable catalyst such as palladium on charcoal, in a suitable solvent such as ethanol to give the corresponding amine of formula (VF).

Intermediate of formula (VF) can be converted to compounds of formula (VIIIF) via reductive alkylation with an aldehyde of formula R⁹—CHO, wherein R⁹ is chosen such that R⁹—CH₂═R¹ and R¹ has the values defined for formula (IF) above. The reductive alkylation can be carried out using standard methods, for instance as those mentioned above with the ketone Ar₁—CO—R³.

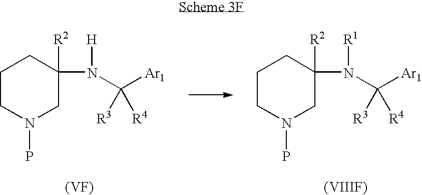

For example a compound of formula (VF) can be alkylated with R⁹—CHO in the presence of a suitable borane, such as NaBH(OAc)₃, optionally in the presence of an acid such as acetic acid, in the presence of a suitable solvent such as dichloroethane (DCE).

For compounds of formula (IF) wherein R³ and R⁴ are hydrogen the alkylation of intermediate (VF) can be carried out with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIF)ₐ. It will be appreciated that the same reaction can be carried out using Ar₁—CR³R⁴—L₁ wherein R³ and R⁴are C₁-C₂ alkyl.

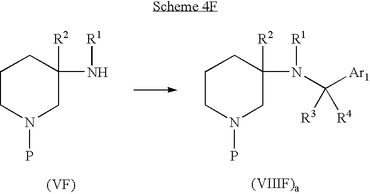

Compounds of formula (IF) wherein R¹ is —CH₂—COO—(C₁-C₂ alkyl) can be prepared by reacting intermediate (VF) with a compound of formula L₂-CH₂—COO—(C₁-C₂ alkyl) wherein L₂ is a suitable leaving group such as for example bromo, chloro or iodo. Said reaction can be carried out in the presence of a suitable base such as sodium hydride, in a suitable solvent such as dimethylformamide.

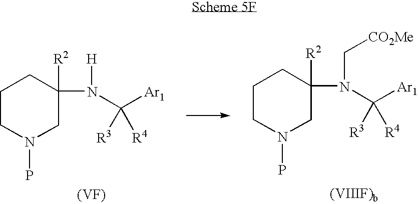

Compounds of formula (IF) wherein R¹ is —(CH₂)ₘ—CF₃ can be prepared by reacting intermediate (VF) with a compound of formula HOOC—(CH₂)₍ₘ₋₁₎—CF₃. The acid may be activated as its anhydride or acyl chloride, and is reacted in the presence of a suitable base such as triethylamine and a catalytic amount of DMAP, in a suitable solvent such as DCM. The resulting amide can be reduced to the amine of formula (VIIF)_{c} in the presence of a suitable borane. For example, for compounds wherein m is 1, the reduction can be carried out in the presence of BH₃—Me₂S borane-dimethyl sulphide complex, in a suitable solvent such as THF.

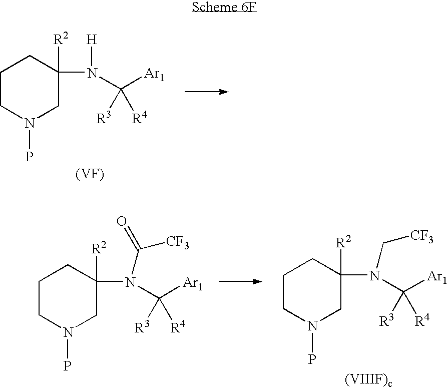

Compounds of formula (IF) wherein R¹ is —(C₁-C₆ alkylene)-OH can be prepared by reacting intermediate (VF) with an epoxide. For example for compounds wherein R¹ is —CH₂—C(CH₃)₂—OH, the intermediate of formula (VF) is reacted with 2,2-dimethyloxirane, in a suitable solvent such as aqueous ethanol.

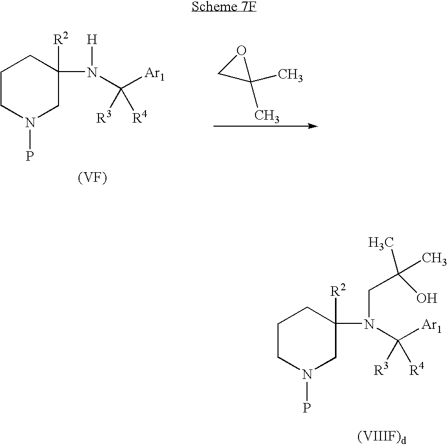

Alternatively compounds of formula (IF) wherein R¹ is —(C₁-C₆alkylene)-OH can be prepared by reacting intermediate (VF) with an ω-haloalkanoate, such as methylbromoacetate, in the presence of a base such a sodium hydrogen carbonate in a solvent such as acetonitrile. The intermediate ester is then reacted with 2 equivalents of methyl magnesium bromide in THF to yield the tertiary alcohol(VIIIF)_{d}:

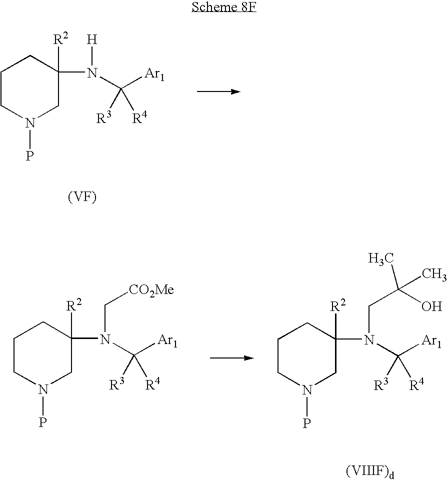

It will be appreciated that the Scheme 8F above applies to alkylene chains longer than —CH₂—.

Compounds of formula (IF) wherein R¹ is —C₂-C₆ alkenyl, —(CH₂)ₙ—S—(C₁-C₃ alkyl), —(C₁-C₅ alkylene)-O—(C₁-C₃ alkyl), —(C₁-C₅ alkylene)-O—(C₃-C₆ cycloalkyl), —(C₁-C₅ alkylene)-SO₂—(C₁-C₃ alkyl), —(C₁-C₅ alkylene)-OCF₃, or —(C₁-C₅ alkylene)-CN, can be prepared via alkylation of intermediate (VF) with a compound of formula L₂-C₂-C₆ alkenyl, L₂-(CH₂)ₙ—S—(C₁-C₃ alkyl), L₂-(C₁-C₅ alkylene)-O—(C₁ -C₃ alkyl), L₂-(C₁-C₅ alkylene)-O—(C₃-C₆ cycloalkyl), L₂-(C₁-C₅ alkylene)-SO₂—(C₁-C₃ alkyl), L₂-(C₁-C₅ alkylene)-OCF₃, or L₂-(C₁-C₅ alkylene)-CN respectively, wherein L₂ is a suitable leaving group such as chloro, bromo, iodo or mesylate, in the presence of a suitable base such as potassium carbonate and a suitable solvent such as acetonitrile, to give the corresponding intermediate of formula (VIIIF)e.

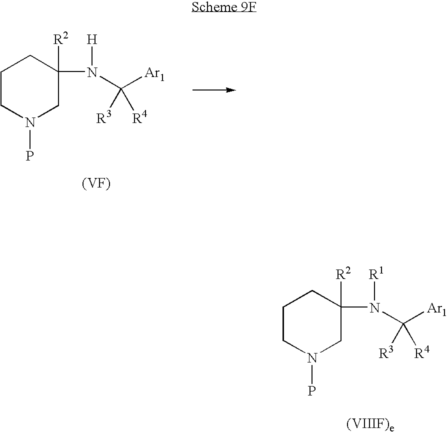

Compounds of formula (IF) wherein R¹ is a group of formula (i) can be prepared using the synthesis illustrated in Scheme 10F for compounds wherein R¹ is 4-tetrahydropyranyl. The compound of formula (IVF) can be alkylated via reductive alkylation using standard methods, as those mentioned above with the ketone Ar₁—CO—R³. For example a compound of formula (IVF) can be alkylated with 4-tetrahydropyranone in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIF)_{f}. It will be appreciated that as mentioned above the same reaction can be carried out using Ar₁—CR³R⁴-L, wherein R³ and R⁴are C₁-C₂ alkyl.

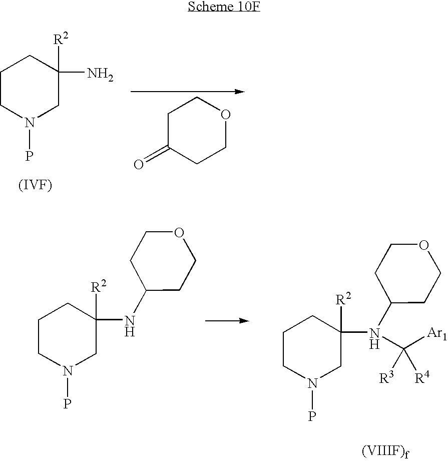

It will be appreciated that for compounds of formula (IF) wherein R¹ is a group of formula (i) and r is 1 then the reductive amination can be carried out using the same reaction conditions but using the corresponding homologous aldehyde of formula 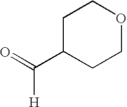
instead of the corresponding 4-tetrahydropyranone. Alternatively, compounds of formula (IF) wherein R¹ is a group of formula (i) and r is 1 can be prepared via formation of an amide, followed by reduction of this amide bond to the corresponding amine as shown in Scheme 11F below:

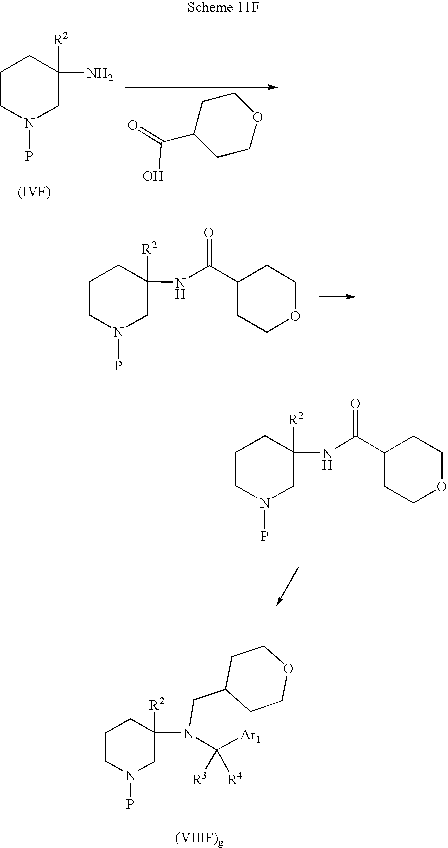

The coupling reaction can be carried out using standard methods known in the art. The reduction of the amide bond can also be carried out by general methods known in the art for example using the same reduction conditions as those used in Scheme 6F, such as in the presence of BH₃—Me₂S (borane-dimethyl sulphide complex), in a suitable solvent such as THF.

Alternatively, compounds of formula (IF) wherein R¹ is a group of formula (i) wherein r is 0 can be prepared by a process illustrated in Scheme 12F for compounds wherein -Z is hydrogen, s is 1, t is 2, each R⁵, R⁶, R⁷ and R⁸ are hydrogen and —X— is —O—, (i.e. R¹ is tetrahydrofuran-3-yl). The compound of formula (IVF) can be alkylated with a compound of formula: 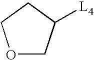
wherein L₄ is a suitable leaving group such as chloro, bromo, iodo, mesylate or tosylate, in the presence of a suitable base such as potassium carbonate and a suitable solvent such as acetonitrile, to give the corresponding secondary amine which can be subsequently alkylated with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIF)_{f}. It will be appreciated that as mentioned above the same reaction can be carried out using Ar₁—CR³R⁴-L, wherein R³ and R⁴are C₁-C₂ alkyl.

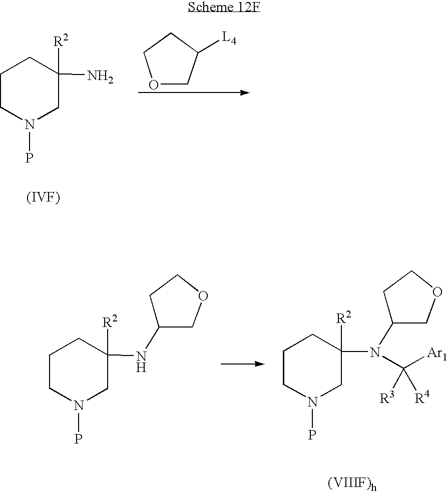

The tetrahydrofuranyl intermediates can be prepared from the corresponding 3-hydroxytetrahydrofuran, wherein the hydroxy group is converted into the leaving group using standard methods.

Compounds of formula (IF) wherein R is a group of formula (i) and —X— is —SO₂— can be prepared from the corresponding intermediates (VIIIF)_{f}, wherein the thioether is oxidized to the corresponding sulphoxide as shown in Scheme 13F below:

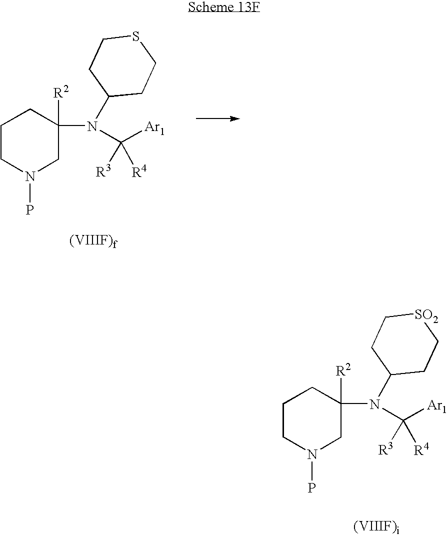

Compounds of formula (IF) wherein R¹ is a group of formula (ii) can be prepared using the synthesis illustrated in Scheme 14F for compounds wherein R¹ is oxabicyclo[3,2,1]octan-3-yl. The compound of formula (IVF) can be alkylated via reductive alkylation using standard methods, as those mentioned above with the ketone Ar¹—CO—R³. For example compound of formula (IVF) can be alkylated with oxabicyclo[3,2,1]octan-3-one in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated with a compound of formula Ar₁CH₂L₁ wherein L₁ is a suitable leaving group such as chloro, bromo, iodo or mesylate, in the presence of a suitable base such as potassium carbonate and a suitable solvent such as acetonitrile, to give the corresponding intermediate of formula (VIIIF)ⱼ. It will be appreciated that as mentioned above the same reaction can be carried out using Ar₁—CR³R⁴-L, wherein R³ and R⁴are C₁-C₂ alkyl.

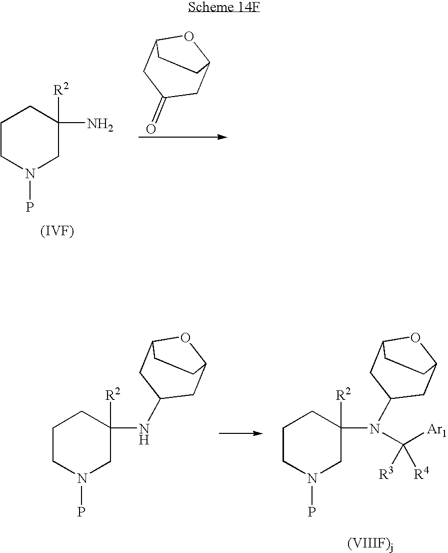

The oxabicyclo[3,2,1]octan-3-one intermediate is prepared according to the method described in A E Hill, G Greenwood and H M R Hoffmann JACS 1973, 95, 1338. It will be appreciated that for compounds of formula (IF) wherein R¹ is a group of formula (i) and r is 1 then the reductive amination can be carried out using the same reaction conditions but using the corresponding homologous aldehyde of formula 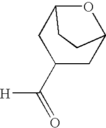
instead of the corresponding oxabicyclo[3,2,1]octan-3-one.

Compounds of formula (IF) wherein Ar₁ is a substituted or unsubstituted pyridyl group can be prepared by a process illustrated in Scheme 15F for compounds wherein R³ and R⁴ are hydrogen and Ar₁ is 3-phenylpyrid-2-yl.

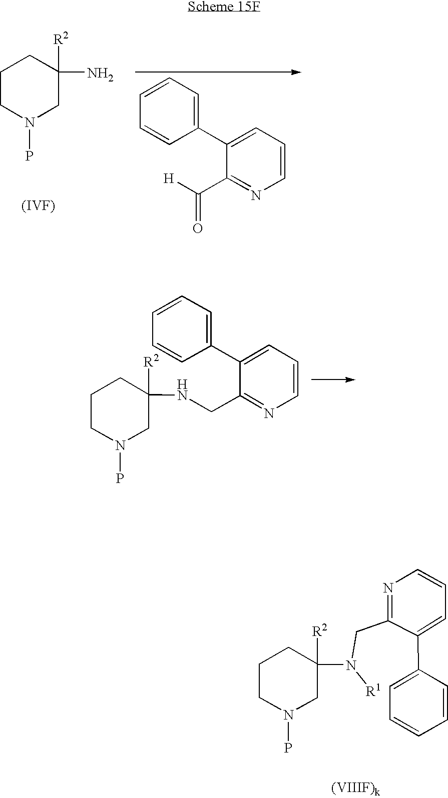

The compound of formula (IVF) can be alkylated via reductive alkylation using standard methods, as those mentioned above with the ketone Ar₁—CO—R³. For example compound of formula (IVF) can be alkylated with an aldehyde of formula: 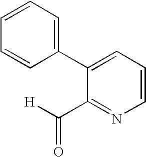
in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated using the general methods described above for the incorporation of R¹. The intermediate aldehyde can be prepared via reduction of readily available methyl 3-phenyl picolinate to the corresponding alcohol and subsequent oxidation to the aldehyde as shown in Scheme 16F below.

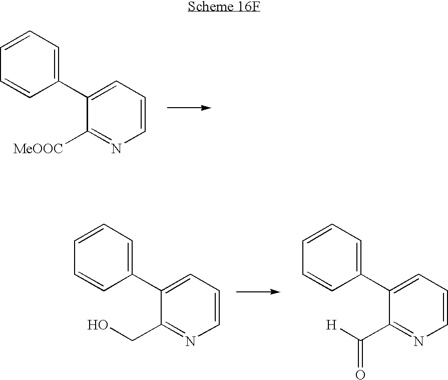

The reduction step can be carried out in the presence of a suitable reducing agent such as lithium borohydride in a suitable solvent such as tetrahydrofuran. The oxidation to the aldehyde can be carried out under Swem conditions such as oxalyl chloride and DMSO in DCM.

Compounds of formula (IF) wherein Ar₁ is a substituted or unsubstituted phenyl group can be prepared by a process illustrated in Scheme 17F for compounds wherein R³ and R⁴ are hydrogen and Ar₁ is 2-(3-pyridyl)phenyl.

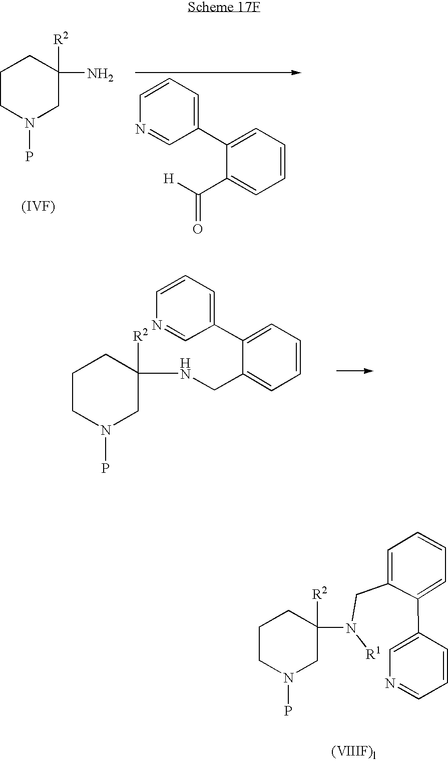

The compound of formula (IVF) can be alkylated via reductive alkylation using standard methods, as those mentioned above with the ketone Ar₁—CO—R³. For example compound of formula (IVF) can be alkylated with an aldehyde of formula: 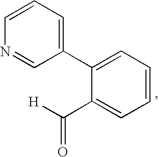
in the presence of a suitable borane, such as sodium borohydride or NaBH(OAc)₃, optionally in the presence of an acid such as acetic acid, in the presence of a suitable solvent such as dichloroethane (DCE). Then, the secondary amine can be alkylated using the general methods described above for the incorporation of R¹. The intermediate aldehyde can be prepared from the commercially available 2-formyl phenyl boronic acid via palladium coupling in the presence of 3-bromopyridine, a suitable palladium catalyst such as Pd(PPh₃)₄ and a suitable base such as potassium carbonate in a suitable solvent such as acetonitrile, as shown in Scheme 18F below.

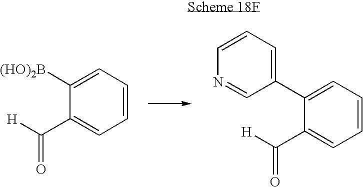

Compounds of formula (IF) wherein Ar₁ is a phenyl group substituted with a 1-pyrazole group can be prepared by a process illustrated in Scheme 19F.

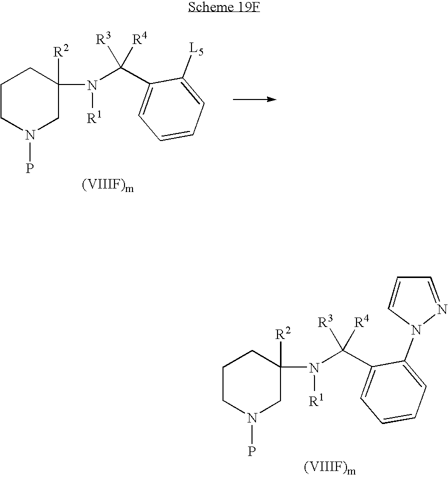

The pyrazole group can be incorporated by reacting a compound of formula (VIIIF)ₘ, wherein L₅ is a suitable leaving group such as bromo, chloro or iodo, with pyrazole in the presence of a suitable base such as potassium carbonate and a catalytic amount of copper iodide in a suitable solvent such as for example DMF. The compound of formula (VIIIF)ₘ, can be prepared by any of the methods mentioned above for compounds wherein Ar1 is a phenyl group substituted with a halogen atom such as chloro, bromo or iodo.

It will be appreciated that any of the intermediates (VIIIF), (VIIIF)ₐ₋ₘ are then deprotected using suitable deprotecting conditions such as those discussed in Greene, to give the corresponding compounds of formula (IF). For example if the protecting group is a boc group, the deprotection reaction can be carried out in trifluoroacetic acid in a suitable solvent such as DCM. Alternatively the reaction can be carried out in ethanolic hydrochloric acid.

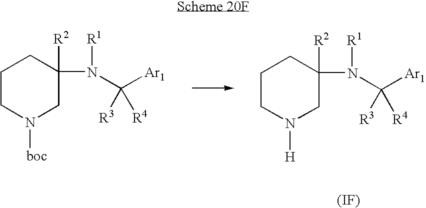

Compounds of formula (IF) wherein R³ and R⁴ are both hydrogen may also be prepared by solid phase synthesis by the route shown below as Scheme 21F.

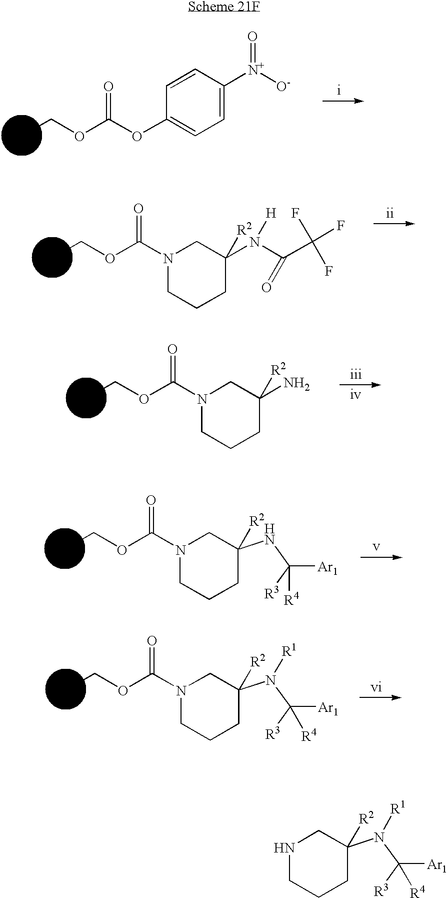

The sequence is preferably performed on a polystyrene resin. The process may be run in a combinatorial fashion such that all possible compounds from sets of precursors Ar₁CHO and R⁹CHO may be prepared, wherein R⁹ is chosen such that R⁹—CH₂═R¹, and R¹ and Ar₁ have the values defined above for formula (IF). The sequence is performed without characterisation of the resin-bound intermediates. In step (i) 3-trifluoroacetamido-piperidine is bound to a solid support by reaction with 4-nitrophenyl carbonate activated polystyrene resin in the presence of a base, such as N,N-diisopropylethylamine, in a solvent such as DMF. In step (ii), the trifluoroacetamido protecting group is cleaved by hydrolysis with a base such as aqueous lithium hydroxide. In step (iii) the primary amine is then condensed with a substituted benzaldehyde in the presence of a dehydrating agent, such as trimethylorthoformate, to form the intermediate imine. In step (iv) the imine is reduced with a borane reducing agent, such as sodium cyanoborohydride, in a solvent such as DMF, containing acetic acid. In step (v) the resultant secondary amine is then reductively alkylated with an aldehyde in the presence of a reducing agent such as sodium triacetoxyborohydride in a solvent, such as DMF. In step (vi) the desired product is finally cleaved from the resin with acid, such as aqueous trifluoroacetic acid.

### Preparation of Compounds of Formula (IG)

Compounds of formula (IG) may be prepared by conventional organic chemistry techniques from N-protected-2-cyanomorpholines as outlined in Error! Reference source not found.G below, wherein R and R² have the values defined for formula (IG) above and P is a suitable nitrogen protecting group such as those described in T. W. Greene, “Protective Groups in Organic Synthesis”, John Wiley and Sons, New York, N.Y., 1991, hereafter referred to as “Greene”. For example a suitable nitrogen protecting group is a benzyl group:

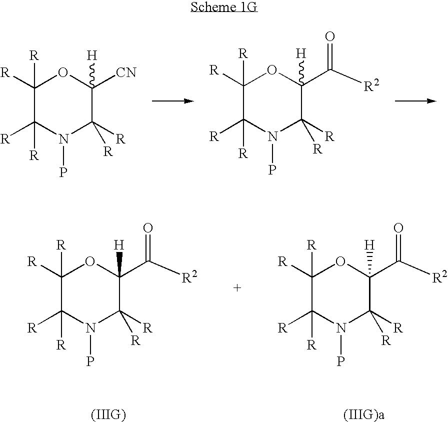

The phenyl ketone (IIIG) can be obtained by reaction of N-protected-2-cyanomorpholine with a Grignard reagent, followed by acid hydrolysis to give the racemic phenyl ketone which may be separated on chiral HPLC.

Compounds of formula (IG) can be prepared from the N-protected morpholine ketone intermediate of formula (IIIG), as illustrated in Error! Reference source not found.G below:

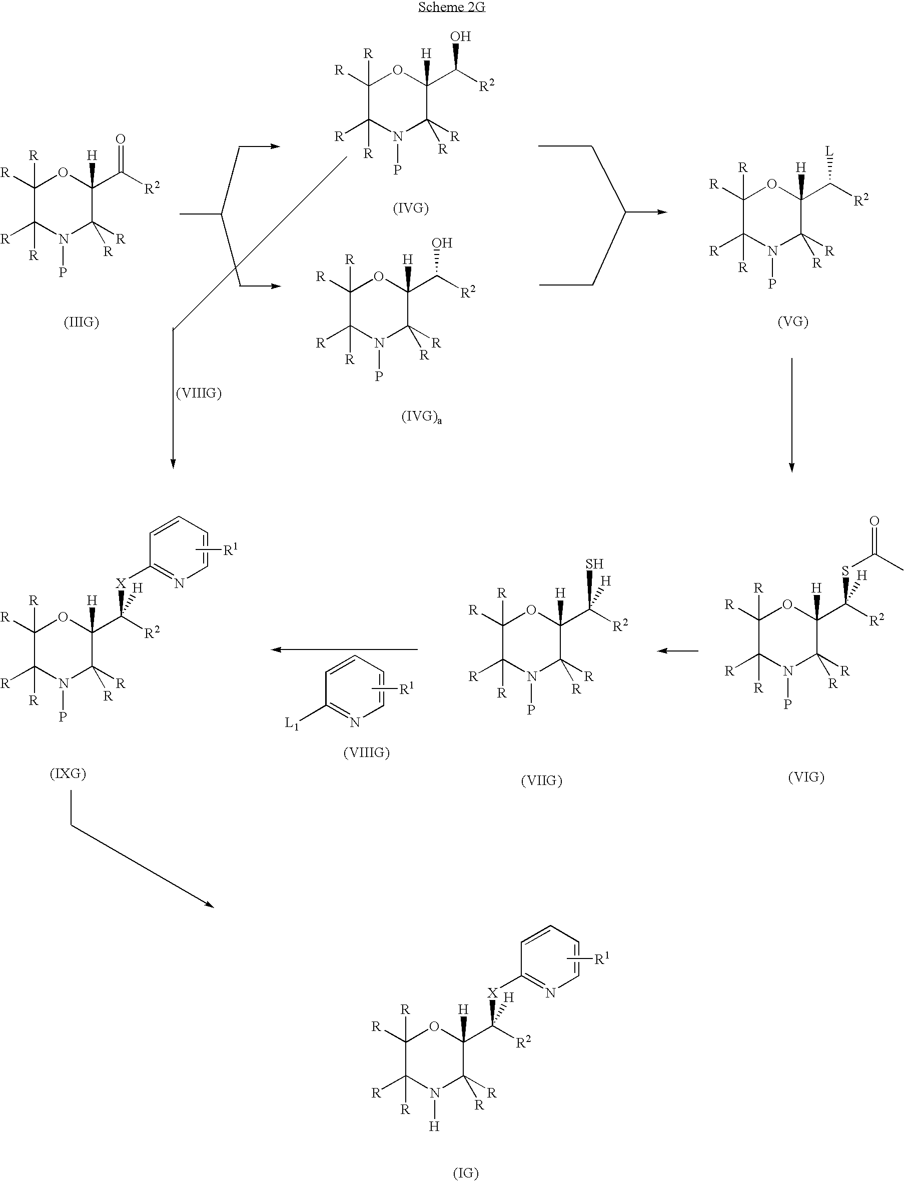

The ketone is stereoselectively reduced to the corresponding (2S) or (2R) alcohol of formula (IVG) or (IVG)ₐ using standard methods known in the art. For example it can be reduced in the presence of [(−)-B-chlorodiisopinocampheylborane] in a suitable solvent such as tetrahydrofuran (THF) to provide the (2S) alcohol.

The resulting alcohol is then transformed into a suitable leaving group L. Suitable leaving groups include halo groups, such as bromo, chloro or iodo and sulfonate groups, such as mesylate. When L is a halo group, the alcohol used will be the (2S) enantiomer (IVG) and it will be reacted with inversion of stereochemistry. For example, when L is bromo, the bromination reaction can be carried out in the presence of a brominating agent such as triphenylphosphine dibromide, in a suitable solvent such as chloroform. When L is a mesylate group, the alcohol used will be the (2R) enantiomer (IVG)ₐ and it will be reacted with retention of stereochemistry in the presence of mesylate chloride and a suitable base.

The resulting intermediate of formula (VG) can then be converted into the corresponding methylethanethioate of formula (VIG) via displacement of the leaving group with a suitable thiolacetate salt such as potassium thiolacetate in the presence of a suitable solvent such as a mixture of dimethylformamide (DMF) and tetrahydrofuran (THF).

The methanethiol intermediate of formula (VIIG) can be prepared via reaction of the methylethanethioate (VIG) with a suitable thiomethoxide such as sodium thiomethoxide in the presence of a suitable solvent such as methanol (one can use a variety of bases but thiomethoxide is preferred because it also acts as a reducing agent and prevents oxidation of thiol hence inhibiting dimerisation; Ref: O. B. Wallace & D. M. Springer, Tetrahedron Letters, 1998, 39 (18), pp2693-2694).

The pyridyl portion of the molecule is incorporated via general methods known in the art. A particularly useful method is the reaction of the methanethiol (VIIG) with a compound of the formula 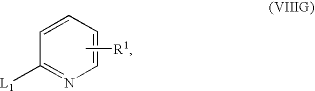
wherein R¹ has the values defined above and L₁ is a suitable leaving group such as fluoro, bromo, chloro, iodo or mesylate, in the presence of suitable base such as sodium hydride, cesium fluoride or sodium methoxide, in a suitable solvent such as DMF.

Compounds of formula (IG) wherein —X— is —O— can be prepared in an analogous fashion by reaction of the (2S) alcohol of formula (IVG) with a compound of formula (VIIIG) above.

The final step for the preparation of compounds of formula (IG) comprises deprotection of the morpholine ring. Conditions for the deprotection depend on the protecting group chosen. Suitable deprotecting conditions can be found in Greene. For example when the nitrogen protecting group is a benzyl group, the deprotection reaction can be carried out in the presence of polymer supported diisopropylamine (PS-DIEA) and 1-chloroethyl chloroformate (ACE-Cl) in a suitable solvent such as dichloromethane, followed by reaction with methanol to give compounds of formula (IG).

Compounds of formula (IG) can alternatively be prepared by the derivatisation of a suitable substituent in the pyridyl ring to give the desired substituent R¹ as shown in Scheme 3G below. For example compounds of formula (IG) wherein —R¹ is —CF₃ can be prepared via reaction of the intermediate (IXG)′ wherein L₂ is introduced into the molecule in place of R¹ in formula (VIIIG) as shown in Error! Reference source not found.G above. The group L₂ is a suitable leaving group such as for example iodo, bromo, chloro or fluoro. The leaving group is converted into a trifluoromethyl group via reaction in the presence of copper iodide, a suitable base such as for example potassium fluoride, and a suitable source of a trifluoromethyl group such as for example (trifluoromethyl)trimethylsilane, in a suitable solvent such as for example a mixture of DMF and N-methyl-pyrrolidinone (NMP). The resulting compound of formula (XG) is deprotected using the methodology described above.

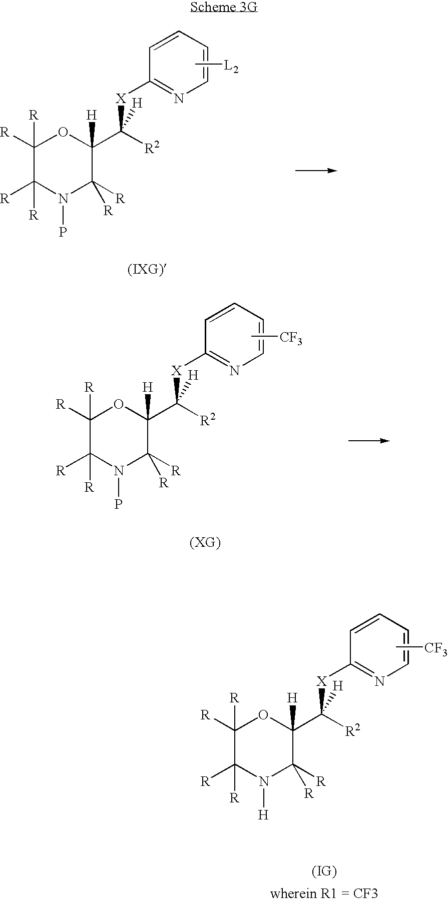

Compounds of formula (IG) wherein —X— is —S— can alternatively be prepared 20 directly from the intermediate methylethanethioate of formula (VIG) as illustrated in Error! Reference source not found.G below.

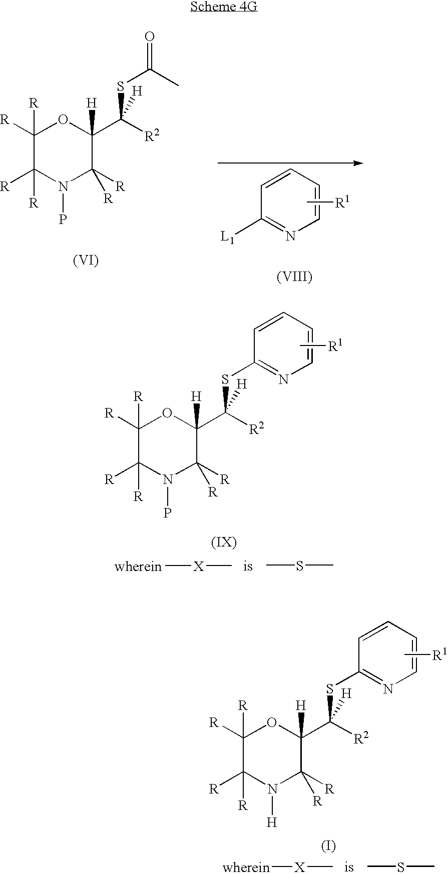

The reaction can be carried out via general methods known in the art. For example, the intermediate (VIG) can be reacted with a compound of formula (VIIIG), wherein R¹ and L₁ have the values defined above, in the presence of a suitable base such as sodium methoxide, in a suitable solvent such as for example DMF.

The resulting compound of formula (IXG) wherein —X— is —S— is then deprotected using the methods described above for Error! Reference source not found.G to give a compound of formula (IG) wherein —X— is —S—. This method is particularly useful when L₁ and R¹ are halogen groups such as for example fluoro and bromo respectively.

Alternatively, the reaction can be carried out in the presence of a suitable base such as sodium hydroxide in a suitable solvent such as a mixture of ethanol and water. This method is particularly useful when L₁ is a halogen group and —R¹ is CN or —CONR³R⁴, wherein R³ and R⁴ have the values defined for formula (IG) above.

Compounds of formula (IG) wherein —X— is —S— can also be prepared via an alternative method using the intermediate of formula (VG) as illustrated below in Error! Reference source not found.G.

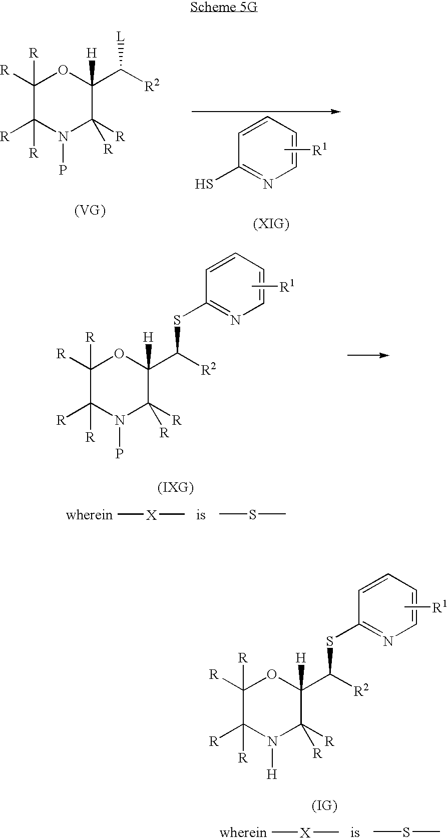

The leaving group of intermediate (VG) is displaced with a suitable thiol of formula (XIG) wherein R¹ has the values defined for formula (IG) above, in the presence of a suitable base such as potassium carbonate, in a suitable solvent such as DMF. The resulting intermediate of formula (IXG) wherein —X— is —S— is then deprotected as described in Error! Reference source not found.G above.

The intermediate of formula (VIIIG) above (including analogs wherein L₂ is introduced in place of R¹) often commercially available. This is the case for intermediates wherein L₁ is a halogen group and R¹ (or L₂) has the values selected from H, methyl, halo, cyano, trifluoromethyl, NH₂, CO₂H, CONH₂, SO₂H, SO₂NHCH₃, NCOCCl₃ and NSO₂Ph.

Intermediates of formula (VIIIG) wherein R¹ is a group of formula (i) can readily be prepared via methods known in the art. We illustrate below 3 methods for the preparation of compounds of formula (VIIIG) wherein R¹ is a group of formula (i) and -Z- has the value of a bond (Error! Reference source not found.G), —CH₂— (Error! Reference source not found.G) or —O— (Error! Reference source not found.G). It will be appreciated that these methods are only illustrative as there are many other alternative methods known in the art which can be used.

As mentioned above, intermediates of formula (VIIIG) wherein R¹ is a group of formula (i) and -Z- is a bond can be prepared via palladium coupling as illustrated in Error! Reference source not found.G below.

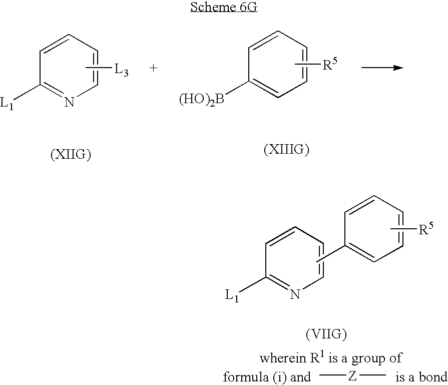

The reaction is carried out via reaction of readily available pyridines of formula (XIIG) wherein L₁ has the values mentioned above and L₃ is a suitable leaving group such as for example a halogen group such as bromo or chloro, with the corresponding phenylboronic acid of formula (XIIIG), in the presence of a suitable palladium catalyst such as for example palladium acetate, a suitable ligand such as triphenylphosphine, in a suitable solvent such as acetonitrile. Alternative palladium catalysts are known in the art, for example bis(benzonitrile)palladium(II)dichloride can be used in the presence of a suitable ligand such as for example bis(diphenylphosphine)butane and a suitable base such as sodium carbonate in a suitable solvent such as for example ethanol, to give good yields of intermediate of formula (VIIIG) wherein R¹ is a group of formula (i) and -Z- is a bond.

Intermediates of formula (VIIIG) wherein R¹ is a group of formula (i) and -Z- is —CH₂— can be prepared by the method illustrated in Error! Reference source not found.G below.

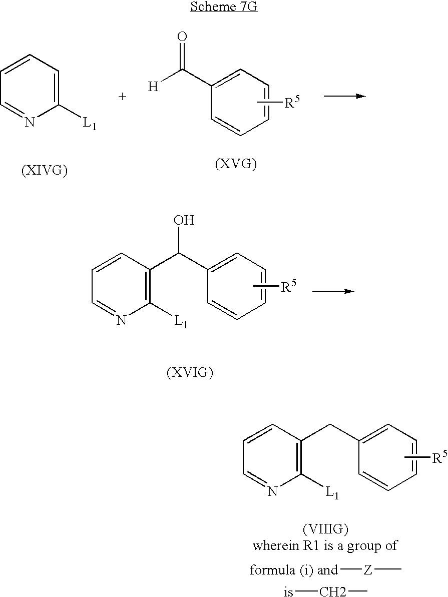

Readily available pyridine compounds of formula (XIVG) wherein L₁ has the values mentioned above (preferably fluoro) are reacted with suitable benzaldehydes of formula (XVG), wherein R⁵ has the value defined for formula (IG) above, in the presence of a suitable base such as for example n-butyllithium or lithium diisopropylamide, in a suitable solvent such as THF, to give the alcohol of formula (XVIG). Said alcohol is then reduced to give the corresponding benzyl derivative (VIIIG) wherein R¹ is a group of formula (i) and -Z- is —CH₂— via hydrogenation, in the presence of a suitable catalyst such as for example palladium on charcoal, in a suitable solvent such as for example ethanol.

Intermediates of formula (VIIIG) wherein R¹ is a group of formula (i) and -Z- is —O— can be prepared by the method illustrated below in Error! Reference source not found.G.

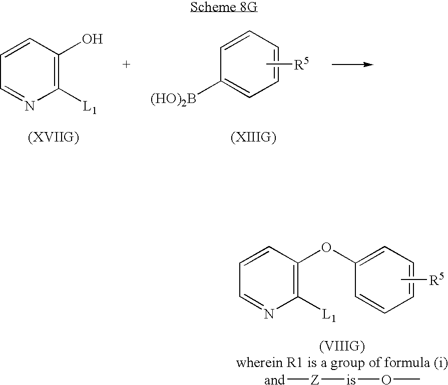

Readily available pyridinols of formula (XVIIG), wherein L₁ has the values mentioned above react with phenylboronic acids of formula (XIIIG) in the presence of copper(II)acetate, powdered 4 Å molecular sieves, and a suitable base such as triethylamine, in a suitable solvent such as for example dichloromethane to give intermediates of formula (VIIIG) wherein R¹ is a group of formula (i) and -Z- is —O—.

Compounds of formula (IG) wherein —X— is —O— may also be prepared by conventional chemistry techniques from the (2R) alcohol (IVG)ₐ using standard methods known in the art. For example as shown in Scheme 9G by reaction of said alcohol with a pyridine of the formula (XVIIIG) or the ketone tautomer of this pyridine wherein R¹ has the values defined for formula (IG) above, in the presence of a suitable phosphine such as triphenyl phosphine and diethyl azodicarboxylate, using an appropriate solvent such as THF, dimethoxyethane, (DME), or chloroform (CHCl₃), as described by D. L. Comins and G. Jianhua, in Tetrahedron Letters, 1994, 35 (18), pp 2819-2822. This reaction is usually carried out with inversion of the stereocentre to (2S)

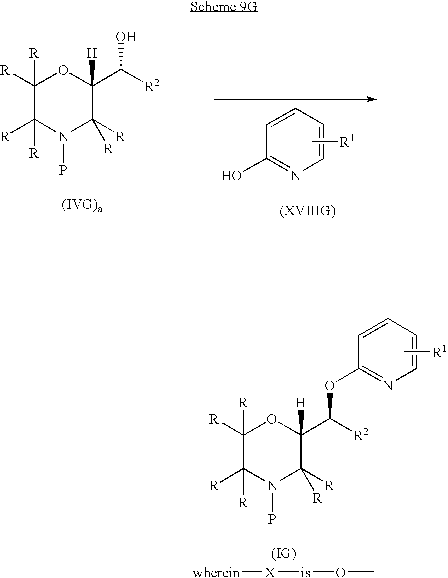

As previously mentioned, compounds of formula (IG) wherein —X— is —O— may alternatively be prepared by the reaction of the (2S) alcohol (IVG) with a pyridine of the formula (VIIIG), where L₁ is preferably chloro and R¹ has the values defined for formula (IG) above, using a suitable base such as potassium hydroxide, in a suitable solvent such as benzene or toluene, in the presence of a suitable phase transfer catalyst such as 18-Crown-6 as described by A. J. S. Duggan et al, in Synthesis, 1980, 7, p 573.

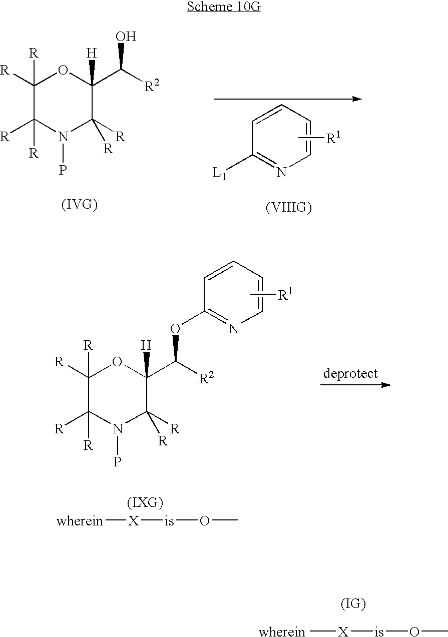

Compounds of formula (IG) wherein —X— is —O— may alternatively be prepared by the reaction of intermediate (VG) wherein L is Br with a pyridine of the formula (VIIIG) wherein -L₁ is —OAg and R¹ has the values defined for formula (IG) above, in a non-polar solvent such as benzene, as described by U. Schollkopf et al, in Liebigs Ann. Chem. 1972, 765, pp 153-170 and G. C. Hopkins et al, in J. Org. Chem. 1967, 32, pp 4040.

It will be appreciated that compounds of Formulae (IA), (IB), (IC), (ID), (IE), (IF) and (IG) above possess one or more asymmetric carbon atoms, and that in the present invention specific individual stereoisomers are preferred. In the present specification, where a structural formula does not specify the stereochemistry at one or more chiral centres, it encompasses all possible stereoisomers and all possible mixtures of stereoisomers (including, but not limited to, racemic mixtures), which can result from stereoisomerism at each of the one or more chiral centers.

The following examples illustrate compounds of of Formula (IA) above and methods for their preparation.

### EXAMPLE 1A

### N-(2-methyltropyl)-N-{(2-fluorophenyl)methyl]piperidin-4-amine fumarate

To a dry boiling tube (50 ml), under nitrogen, was added tert-butyl-4-(2-methyl-propylamino)-piperidine-1-carboxylate (0.200 g, 0.780 mmol), 2-fluorobenzaldehyde (0.087 ml, 0.102 g, 0.819 mmol), and titanium isopropoxide (0.268 ml, 0.937 mmol) to give a yellow/orange solution. This was heated to 90° C. for 2 hours. Solution cooled, and ethanol (5 ml) added. Sodium borohydride (0.030 g, 0.780 mmol) was then added and allowed to stir for 2 days. Further sodium borohydride (0.300 g, 7.80 mmol) was added, and after 6 hours, this was diluted with methanol (10 ml) with stirring for 20 hours. This was concentrated in vacuo, dissolved in dichloromethane (5 ml), and acetic anhydride (0.371 ml, 39.00 mmol) added with stirring for 30 minutes. Solution was diluted with methanol (10 ml), and passed through an SCX-2 column to give an oil (0.150 g, 0.412 mmol).

The resultant oil was dissolved in dichloromethane (5 ml), and trifluoroacetic acid (2 ml) added. Reaction was monitored by thin layer chromatography (100% ethyl acetate; reactant. r.f. 0.4, product r.f. 0.0). After 2 hours, reaction was concentrated in vacuo, azeotroped with dichloromethane (c.a. 25 ml), taken up in methanol (c.a. 5 ml), and passed through an SCX-2 column. The resultant colourless oil was purified using reverse phase chromatography, concentrated in vacuo, taken up in 5 M hydrochloric acid (10 ml), and heated to 90° C. for 3 hours. This solution was freeze dried to give an oil (0.049 g, 0.185 mmol). Resultant oil was passed through an SCX-2 column, dissolved in aqueous acetonitrile (c.a. 20 ml), and fumaric acid (0.0214 g, 0.1850 mmol) added. After 5 minutes, this was freeze dried to give a white solid (0.070 g, 0.185 mmol) as the title compound. δ_{H} (300 MHz, MeOD) 7.47 (1H, t, Ar), 7.25 (1H, m, Ar), 7.13 (1H, t, Ar), 7.02 (1H, t, Ar), 6.70 (2H, s, fumarate), 3.21 (2H, s, NCH2Ar), 3.45 (2H, d, CH), 2.95 (2H, t, CH), 2.82 (1H, t, CH), 2.29 (2H, d, NCH2), 2.00 (2H, d, CH), 1.80 (2H, t, m), 1.68 (1H, t, CH), 0.85 (6H, d, CHMe2). LCMS 12 minute gradient, Rt=1.99 mins, (M⁺+1)=265.2

### EXAMPLE 2A

### N-(3,3-dimethylbutyl)-N-[(2-biphenyl)methyl]piperidin-4-amine fumarate

To a 100 ml round bottomed flask, under nitrogen, was added the 1,1 -dimethylethyl 4-[(2-bromophenylmethyl)(3,3-dimethylbutyl)amino]piperidine-1-carboxylate (0.675 g, 1.49 mmole, 1.0 eq.), phenylboronic acid (0.363 g, 2.98 mmole, 2.0 eq.), dichlorobis(triphenylphosphine)palladium(II) (0.104 g, 0.15 mmole, 0.1 eq.), sodium carbonate (0.158 g, 2.98 mmole, 2.0 eq.) and a 1:1 mixture of tetrahydrofuran : water (50 ml). The mixture was heated at 90° C. for two hours. The reaction mixture was allowed to cool then poured into diethyl ether (100 ml). This organic mixture was washed with a solution of sodium hydroxide (2M, aqueous, 80 ml) then concentrated in vacuo to give a dark yellow oil (1.18 g). This oil was purified by automated flash chromatography using an ISCO Combiflash system (SiO₂ (120 g); 0-10% methanol (+5% 7M NH₃/MeOH) in dichloromethane gradient elution over 40 minutes) to give a yellow oil (0.683 g). This oil was further purified by automated flash chromatography using an ISCO Combiflash system (SiO₂ (120 g); ethyl acetate gradient elution over 40 minutes) to give 1,1-dimethylethyl 4-[({2-biphenyl}methyl)(3,3-dimethylbutyl)amino]piperidine-1-carboxylate as a yellow oil (0.549 g, 82%). To a solution of this oil (0.549 g, 1.22 mmole, 1.0 eq.) in dichloromethane (10 ml) was added trifluoromethanesulfonic acid (TFA) (1.36 ml, 18.27 mmole, 15 eq). The solution was stirred for one hour at room temperature. Solvent and TFA were removed in vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave a colourless oil (0.27 g). This oil was purified on the Biotage Parallel Flex Purification System (UV-guided HPLC) followed by SCX-2 treatment (to obtain the free base) to give a colourless oil (0.132 g). To a solution of this oil in methanol was added a solution of fumaric acid (0.044 g g, 0.38 mmole, 1 eq) in methanol. The mixture was left to stir for a couple of minutes, ethyl acetate and cyclohexane were then added. The resulting precipitate was collected by filtration to give the title compound as a white solid (0.121 g, 17%). OH (300 MHz, MeOD) 7.50-7.47 (1H, m, ArH), 7.35-7.18 (7H, m, ArH), 7.10-7.07 (1H, m, ArH), 6.61 (3H, s, fumarate CH), 3.58 (2H, s, CH₂Ar), 3.25-3.24 (2H, m, NCH₂), 2.74 (2H, dt, NCH₂), 2.67-2.57 (1H, m, NCH), 2.34-2.29 (2H, m, NCH₂), 1.65-1.45 (4H, m, CCH₂), 1.13-1.08 (2H, m, CH₂tBu), 0.70 (9H, s, CH₃); LCMS 12 min, Rt=4.3 min, (M⁺+1)=351.

### EXAMPLE 3A

### N-(2-ethylbutyl)-N-[(2-biphenyl)methyl]piperidin-4-amine fumarate

As method previously described for Example 2A, using 1,1-dimethylethyl 4-[(2-bromophenylmethyl)(2-ethylbutyl)amino]piperidine-1-carboxylate. Isolation of the fumarate salt from methanol, diethyl ether, cyclohexane yielded the title compound as a white solid (0.238 g, 34%). OH (300 MHz, MeOD) 7.59-7.57 (1H, m, ArH), 7.45-7.27 (7H, m, ArH), 7.19-7.16 (1H, m, ArH), 6.69 (1.5H, s, fumarate CH), 3.62 (2H, s, CH₂Ar), 3.34-3.32 (2H, m, NCH₂), 2.79 (2H, dt, NCH₂), 2.66-2.57 (1H, m, NCH), 2.21 (2H, d, NCH₂), 1.64-1.50 (4H, m, CCH₂), 1.38-1.17 (5H, m, CH(CH₂Me)₂), 0.78 (6H, t, CH₃); LCMS 12 min, Rt=5.1 min, (M⁺+1)=351.

### EXAMPLE 4A

### N-(cyclohexylmethyl)-N-[(2-biphenyl)methyl]piperidin-4-amine fumarate

(i) To a solution of cyclohexylmethylamine (0.461 g, 4.08 mmole, 1.02 eq.) in 1,2-dichloroethane (10 ml) was added 1-Boc-4-piperidone (0.797 g ml, 4.00 mmole, 1.0 eq.). To this was added a solution of sodium triacetoxyborohydride (0.865 g, 4.08 mmole, 1.02 eq.) in dimethylformamide (2 ml). This mixture was left to stir under nitrogen, at room temperature, over the weekend. To the reaction mixture was then added water (10 ml) and the mixture stirred vigorously for several minutes. The chlorinated organic layer was then run through a hydrophobic frit then diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give a pale yellow oil (1.2 g). This was purified by automated flash chromatography using an ISCO Combiflash system (SiO₂ (40 g); 0-10% methanol in ethyl acetate gradient elution over 40 minutes) to give 1,1-dimethylethyl 4-[(cyclohexylmethyl)amino]piperidine-1-carboxylate as a colourless oil (0.98 g, 83%). δ_{H} (300 MHz, CDCl₃) 4.03-4.00 (2H, m, NCH₂), 2.83-2.75 (2H, m, NCH₂), 2.60-2.49 (1H, m, NCH), 2.45 (2H, d, NCH₂), 1.18-0.83 (15H, m, CCH₂), 1.45 (9H, s, OC(CH₃)₃); LCMS 6 min, Rt=2.7 min, (M⁺+1)=297.

(ii) To a solution of 1,1-dimethylethyl 4-[(cyclohexylmethyl)amino]piperidine-1-carboxylate (0.245 g, 0.840 mmole, 1.0 eq.), 2-phenylbenzyl bromide (0.185 ml, 1.01 mmole, 1.2 eq.) in dry acetonitrile (5 ml) was added anhydrous potassium carbonate (0.19 g, 1.35 mmole, 1.6 eq.). The mixture was stirred overnight at room temperature. The reaction mixture was concentrated under vacuum to give a white solid. The white solid was taken up in dichloromethane (10 ml) and this washed with water (10 ml). The dichloromethane layer was passed through a hydrophobic frit then diluted with methanol (10 ml). This solution was loaded onto an SCX-2 (10 g) column. The column was washed with methanol (50 ml) then basic material was eluted using 2N ammonia in methanol (50 ml). Concentration of the ammonia/methanol solution under vacuum yielded a colourless oil (0.344 g, 90%). To a solution of this oil (0.344 g, 0.74 mmole, 1.0 eq.) in dichloromethane (10 ml) was added trifluoroacetic acid (TFA) (0.83 ml, 11.2 mmole, 15 eq). The solution was stirred overnight at room temperature. Solvent and TFA were removed in vacuo. The resulting oil was taken up in methanol and loaded onto an SCX-2 (10 g) column. The column was washed with methanol (50 ml). Basic material was then eluted using 2N ammonia in methanol (50 ml). Removal of solvent from the ammonia/methanol mixture under vacuum, gave a colourless oil (0.298 g, 99%). The oil was taken up in methanol. To this solution was added a solution of fumaric acid (0.095 g, 0.08 mmole, 1 eq) in methanol followed by diethyl ether and cyclohexane. The resulting precipitate was collected by filtration to give the title compound as a white solid (0.302 g, 76 %). 8H (300 MHz, MeOD) 7.58 (1H, d, ArH), 7.45-7.29 (7H, m, ArH), 7.18 (1H, d, ArH), 6.70 (2H, s, fumarate CH), 3.64 (2H, s, CH₂Ar), 3.33-3.32 (2H, m, NCH₂), 2.79 (2H, dt, NCH₂), 2.65-2.54 (1H, m, NCH), 2.17 (2H, d, NCH₂), 1.74-1.47 (9H, m, CCH₂), 1.28-1.11 (4H, m, CH, CCH₂), 0.78-0.67 (2H, m, CH₂); LCMS 12 min, Rt=5.0 min, (M⁺+1)=363.

### EXAMPLE 5A

### N-(cyclopropylmethyl)-N-[(2-biphenyl)methyl]piperidin-4-amine fumarate

As method previously described for Example 4A, using 1,1-dimethylethyl 4-[(cyclopropylmethyl)amino]piperidine-1-carboxylate and 2-phenylbenzyl bromide. Isolation of the fumarate salt from methanol and diethyl ether yielded the title compound as a white solid (0.485 g, 74%). δ_{H} (300 MHz, MeOD) 7.68 (1H, dd, ArH), 7.47-7.29 (7H, m, ArH), 7.21 (1H, d, ArH), 6.72 (2H, s, fumarate CH), 3.76 (2H, s, CH₂Ar), 3.38-3.34 (2H, m, NCH₂), 2.92-2.82 (3H, m, NCH, NCH₂), 2.32 (2H, d, NCH₂), 1.79-1.57 (4H, m, CCH₂), 0.77-0.66 (1H, m, CH), 0.46-0.40 (2H, m, CH₂), 0.03-0.02 (2H, m, CH₂); LCMS 12 min, Rt=3.5 min, (M⁺+1)=321.

### EXAMPLE 6A

### N-(3-methylbutyl)-N-[(2-phenoxyphenyl)methyl]peridin-4-amine difumarate

(i) To 10% Pd/C (1.0 g, 10% wt), under nitrogen, was added a solution of the 1-Boc-4-piperidone (10.0 g, 50.1 mmole, 1.0 eq.) and isoamylamine (4.46 g, 51.2 mmole, 1.02 eq.) in ethanol (60 ml). This was hydrogenated overnight, at 60 psi using a Parr hydrogenator. The catalyst was removed by filtration through Celite. Solvent was removed under vacuum to give 1,1-dimethylethyl 4-[(3-methylbutyl)amino]piperidine-1-carboxylate as a colourless, slightly cloudy, oil (13.59 g, 100%). δ_{H} (300 MHz, CDCl₃) 4.05-4.02 (2H, m, NCH₂), 2.82-2.75 (2H, m, NCH₂), 2.66-2.54 (3H, m, NCH, NCH₂), 1.86-1.82 (2H, m, CCH₂), 1.62 (1H, septet, CHMe₂), 1.45 (9H, s, OC(CH₃)₃), 1.41-1.17 (4H, m, CCH₂), 0.90 (6H, d, C(CH₃)₂); LCMS 6 min, Rt =2.7 min, (M⁺+1)=271.

(ii) To a solution of 1,1-dimethylethyl 4-[(3-methylbutyl)amino]piperidine-1-carboxylate in 1,2-dichloroethane (10 ml) was added 2-phenoxybenzaldehyde. To this was added a solution of sodium triacetoxyborohydride (3.0 eq.) in dimethylformamide (2 ml). This mixture was left to stir for 3 days under nitrogen, at room temperature. To the reaction mixture was added water (10 ml) and the mixture stirred vigorously for several minutes. The chlorinated organic layer was run through a hydrophobic frit to remove water, diluted with methanol (10 ml) and loaded onto an SCX-2 (10 g) column. The column was washed with methanol (50 ml) then basic material eluted with 2N ammonia in methanol. The ammonia/methanol solution was concentrated in vacuo to give 1,1-dimethylethyl 4-[(2-phenoxyphenylmethyl)(3-methylbutyl)amino]piperidine-1-carboxylate as a colourless oil. To a solution of this oil in dichloromethane (10 ml) was added trifluoroacetic acid (TFA) (15 eq). The solution was stirred overnight at room temperature. Solvent and TFA were removed in vacuo. The resulting oil was taken up in methanol and loaded onto an SCX-2 (10 g) column. The column was washed with methanol (50 ml). Basic material was then eluted using 2M ammonia in methanol (50 ml). Removal of solvent from the ammonia/methanol mixture under vacuum, gave a colourless oil. The oil was taken up in methanol. To this solution was added a solution of fumaric acid (1 eq) in methanol. The mixture was left to stir for a couple of minutes, then ethyl acetate and cyclohexane were added. The resulting precipitate was collected by filtration to give the title compound as a white solid (0.264 g, 30%). δ_{H} (300 MHz, MeOD) 7.46 (1H, dd, ArH), 7.26-7.16 (3H, m, ArH), 7.10-7.04 (1H, m, ArH), 7.00-6.95 (1H, m, ArH), 6.86-6.79 (3H, m, ArH), 6.61 (4H, s, fumarate CH), 3.68 (2H, s, CH₂Ar), 3.33-3.28 (2H, m, NCH₂), 3.04-2.96 (3H, m, NCH, NCH₂), 2.56-2.51 (2H, m, NCH₂), 1.91-1.87 (2H, m, CCH₂), 1.76-1.62 (2H, m, CCH₂), 1.52-1.41 (1H, m, CH), 1.30-1.23 (2H, m, CH₂), 0.74 (6H, d, CH₃); LCMS 12 min, Rt=4.2 min, (M⁺+1)=353.

### EXAMPLE 7A

### N-(3-methylbutyl)-N-[(2-biphenyl)methyl]piperidin-4-amine difumarate

As method previously described for Example 4A, using 1,1-dimethylethyl 4-[(3-methylbutyl)amino]piperidine-1-carboxylate and 2-phenylbenzyl bromide. Isolation of the fumarate salt from methanol and diethyl ether yielded the title compound as a white solid (0.239 g, 24%). 8H (300 MHz, MeOD) 7.49 (1H, dd, ArH), 7.35-7.18 (7H, m, ArH), 7.10 (1H, dd, ArH), 6.61 (4H, s, fumarate CH), 3.62 (2H, s, CH₂Ar), 3.25 (2H, m, NCH₂), 2.78-2.59 (3H, m, NCH, NCH₂), 2.36-2.31 (2H, m, NCH₂), 1.64-1.45 (4H, m, CCH₂), 1.42-1.31 (1H, m, CH), 1.13-1.05 (2H, m, CH₂), 0.69 (6H, d, CH₃); LCMS 12 min, Rt=4.1 min, (M⁺+1)=337.

The following examples illustrate compounds of of Formula (IB) above and methods for their preparation.

Synthesis of Intermediates.

### Preparation of (4-Benzyl-morpholin-2-yl)-phenyl-methanone

A 1600 L GL reactor under N₂ was successively loaded with 2-chloroacrylonitrile (33.2 kg, 379 moles) and toluene (114 L) at 21° C. Then, N-benzylethanolamine (57 kg, 377 moles) was added and the reaction mixture was post-agitated at room temperature for about 17 h. Then, the mixture was diluted with toluene (336 L), cooled down to −12.4° C. and potassium t-butoxide (42.3 kg, 377 moles) was added in portions (10) maintaining −13.7° C.≦Tmass≦−2.8° C. The mixture was post-agitated at about 0° C. for 2.5 h, quenched by adding ultra pure water (142.5 L) maintaining 2.1° C.≦Tmass≦8.7° C. The aqueous layer (176 kg) was separated after 35 minutes of post-stirring allowing the mixture to reach 15° C. and the toluene layer was washed with ultra pure water (142.5 L) and the aqueous layer (162 kg) was separated. The organic layer was then concentrated under reduced pressure (150 mbars) maintaining Tmass≦60° C. in order to distill 162 kg of toluene. The filtrates were then diluted with toluene (114 L) and treated with SiO₂ (Merck silica gel 60, 0.063-0.1 mm, 74.1 kg) under agitation at room temperature for 1.25 h. SiO₂ was filtered and rinsed with toluene (2×114 L). Then, the filtrates were concentrated under reduced pressure (150 mbars) maintaining Tmass≦60° C. in order to distill 351.8 kg of toluene (KF: 0.01 % w/w H₂O).

The solution of 4-Benzyl-morpholine-2-carbonitrile (169.2 kg) was diluted with toluene (157 L) and was cooled to 0° C. and phenylmagnesiumchloride (25 wt. % solution in THF, 213 kg, 389 moles, 1.36 molar equiv.) was slowly added (over 3.5 h) to the reaction mixture, maintaining the temperature at −3° C.≦Tmass≦7° C. The reaction mixture was post-stirred for 2 hours at Tmass=0° C. Then, the quench was performed by adding acetic acid (8.55 L, Tmass=5→17.2° C.), post stirring 10 minutes and cooling to 5° C. before adding an acetic acid/water mixture (229 L, 33/67 v/v). During the quench, addition was performed at such a rate that Tmass did not exceed 20° C. (typical Tmass=4.6° C. to 10.4° C.). The mixture was post-agitated overnight at RT and the aqueous layer (285.8 kg) was extracted.

The toluene layer was cooled to 0° C. and a 5 N NaOH aqueous solution (420.1 kg) was slowly added maintaining the temperature at −2.4° C.≦Tmass≦11° C. The reaction mixture was post-stirred for 1 h and the aqueous layer (494.8 kg) was extracted. The toluene layer was concentrated under reduced pressure (50 mbars) maintaining Tmass≦60° C. in order to distill 356.2 kg of toluene and isopropanol (180.4 kg) was added. The toluene was stripped off under reduced pressure (100 mbars) maintaining Tmass≦60° C. in order to distill 186.4 kg of toluene and isopropanol (135 kg) was added again to the mixture. A last distillation of toluene was performed under reduced pressure (50 mbars) maintaining Tmass≦60° C. in order to distill 131 kg of toluene and isopropanol (49.4 kg) was finally added to the mixture and the solution was stirred at RT until crystallization (17 minutes).

Ultra pure water was added (125.4 L) and the mixture was stirred overnight at RT and cooled down to about 0° C. for 1 hour. The precipitate was filtered and rinsed with a cooled water/isopropanol 50/50 v/v solution (76.6 kg). The wet precipitate was dried under vacuum at Tjack=35° C. for 96 hours to obtain the title compound as an off-white powder with 59% overall yield. The title compound can be resolved by the fractional crystallisation process described above.

### Preparation of (4-Benzyl-morpholin-2-yl)-(3-fluoro-phenyl)-methanone

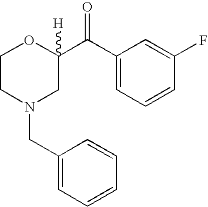

To a solution of 4-Benzyl-morpholine-2-carbonitrile (10 g, 50 mmol) in dry diethyl ether (100 ml) at −10° C. under an atmosphere of nitrogen was added (time of addition 30 minutes) a solution of 3-fluorophenylmagnesium bromide (0.5N solution in tetrahydrofuran, 120 ml, 60 mmol, 1.2 equivalents, available from Aldrich Chemical Company or Rieke Metals) and the reaction mixture was further stirred at −10° C. for 30 minutes. Then the reaction was allowed to warm to room temperature and stirred for one hour. The reaction was then cooled to 0° C. and quenched by addition of hydrochloric acid (2N aqueous solution, 50 ml) and the resulting mixture was stirred for 30 minutes at 0° C. Then the solution was concentrated in vacuo and the residue was taken-up by sodium hydroxide (2N aqueous solution, 60 ml). The aqueous solution was extracted with diethyl ether, the organics fractions were collected and dried (MgSO₄) and the solvent removed under reduced pressure to give the title compound as a brown oil (15 g, 100%). FIA [M+H]+=300.1.

### Preparation of 2-Chloromethyl-4-fluoro-1-methoxy-benzene

### a) (5-Fluoro-2-methoxy-phenyl)-methanol

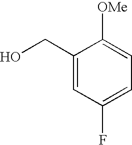

To a solution of 2-Methoxy-5-fluorobenzaldehyde (11.093g, 1 equiv.—available from Aldrich Chemical Company) in methanol at −10° C. under nitrogen atmosphere was added NaBH₄ (7.515 g, 2.7 equiv.) portionwise. The solution was allowed to warm to room temperature and after 30 minutes the reaction solvent was removed under reduced pressure and replaced with dichloromethane. This solution was poured onto ice water and further extracted with dichloromethane. The organic fractions were collected and dried (MgSO₄) and the solvent removed under reduced pressure to give the title compound as an oil (9.794 g, 87%).¹H NMR (300 MHz, CDCl₃): δ 2.58 (m, 1H), 3.81 (s, 3H), 4.63 (d, 2H, J=6.3 Hz), 6.78 (dd, 1H, J=8.9 and 4.3 Hz), 6.94 (td, 1H, J=8.5 and 3.1 Hz), 7.04 (dd, 1H, J =8.7 and 3.1 Hz).

### b) 2-Chloromethyl-4-fluoro-1-methoxy-benzene

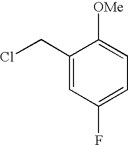

Neat (5-Fluoro-2-methoxy-phenyl)-methanol (19.587 g, 1 equiv.) was added to neat SOCl₂ (42.2 mL, 4.6 equiv.) at −78° C. under a nitrogen atmosphere and the solution was then allowed to warm to room temperature and stirred until evolution of gas had ceased. An equivalent volume of anhydrous toluene was added to the flask and the solution heated to 60° C. On cooling the reaction solution was poured onto ice water. The toluene layer was separated and dried (MgSO₄) and the solvent removed under reduced pressure. The crude material was sublimed (60-80° C./0.05 mBarr) to give the title compound as a white solid (13.40 g, 61%). ¹H NMR (300 MHz, CDCl₃): δ 3.87 (s, 3H), 4.60 (s, 2H), 6.79-7.20 (m, 3H).

### Preparation of 1-Chloromethyl-2-isopropoxy-benzene

### a) (2-Isopropoxy-phenyl)-methanol

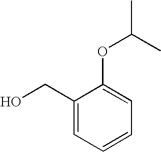

A mixture of 2-hydroxybenzyl alcohol (21.04 g, 1 equiv., available from Aldrich Chemical Company), 2-isopropyl iodide (32.3 mL, 1.9 equiv., available from Aldrich Chemical Company) and K₂CO₃ (71.42 g, 3 equiv.) in ethanol was refluxed for 3 hours. On cooling the reaction mixture was filtered and the solvent removed under reduced pressure and replaced with dichloromethane, and then filtered and the solvent removed to give the title compound as an oil (27.751 g, 99%). ¹H NMR (300MHz, CDCl₃): δ 1.37 (d, 6H, J=6.0Hz), 3.55 (bs, 1H), 4.50-4.70 (m, 3H), 6.78-6.90 (m, 2H), 7.15-7.25 (m, 2H).

### b) 1-Chloromethyl-2-isopropoxy-benzene

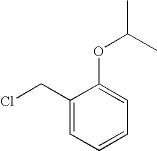

The title compound was prepared using the general procedure outlined above for the preparation of 2-Chloromethyl-4-fluoro-1-methoxy-benzene followed by the following treatment:

The crude reaction material was chromatographed on silica gel and eluted 1:9 ethyl acetate/heptane prior to distillation (40-60° C./0.05 mBar). 1H NMR (300 MHz, CDCl₃): δ 1.37 (d, 6H, J=6.0 Hz), 4.50-4.70 (m, 3H), 6.80-7.00 (m, 2H), 7.23-7.30 (m, 2H).

Synthesis of Compounds of Formula (IB).

### EXAMPLE 1B

### (S,R)-2-(2-Methoxy-phenyl)-1-morpholin-2-yl-1-phenyl-ethanol hydrochloride

### a) 1-(4-Benzyl-morpholin-2-yl)-2-(2-methoxy-phenyl)-1-phenyl-ethanol

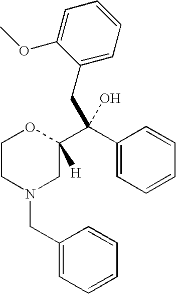

Solid magnesium turnings (9.5 g, 28 equiv.) under nitrogen atmosphere at room temperature were stirred vigorously with a magnetic stirring bar overnight. The magnesium was then covered with dry diethyl ether and to the suspension was added 1,2-dibromoethane (50 μL). A cold bath was then applied followed by dropwise addition of 1-chloromethyl-2-methoxy-benzene (18.18 g, 5 equiv. available from Aldrich Chemical Company) in diethyl ether (71 mL) which maintained the temperature at up to 15° C. The resulting black suspension was stirred at room temperature for 30 minutes and cooled down at −20° C. A solution of (4-Benzyl-morpholin-2-yl)-phenyl-methanone (4 g, 1 equiv.) in diethyl ether (50 mL) was then added dropwise via canula. The reaction mixture was left to warm to room temperature over two hours and then quenched by addition of aqueous saturated solution of NaHCO₃ (50 mL). The aqueous solution was extracted with diethyl ether, the organic phase dried with MgSO₄, evaporated in vacuo to give 7 g of a yellow amorphous solid. The compound was taken without further purification in the next step. FIA [M+H]⁺=404.

### b) 2-(2-Methoxy-phenyl)-1-morpholin-2-yl-1-phenyl-ethanol hydrochloride

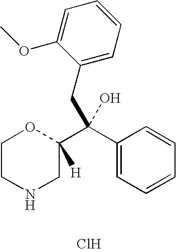

To a solution of 1-(4-Benzyl-morpholin-2-yl)-2-(2-methoxy-phenyl)-1-phenyl-ethanol (1 g, 1 equiv.) in ethyl acetate (100 mL) at room temperature under nitrogen atmosphere was added ammonium formate (3.9 g, 25 equiv.) followed by addition of palladium on charcoal (10%, 1 g.). The reaction mixture was heated to reflux for 1 hour, cooled to room temperature and then filtered through Celite. All volatiles were evaporated under vacuum, and the resulting solid was purified via preparative HPLC. The isolated white solid was taken up in ethanol. Hydrogen chloride was added (large excess of 2M solution in diethyl ether) and the mixture was stirred until it became a clear solution. Then all the volatiles were evaporated in vacuo, to give 650 mg of the title compound as white solid (75%). ¹H NMR (300 MHz, DMSO D6) δ: 2.43-2.51 (m, 2H), 2.77-2.92 (m, 2H), 3.15-3.23 (m, 3H), 3.41 (s, 3H), 4.10-4.19 (m, 2H), 6.66-6.72 (m, 2H), 6.98-7.07 (m, 2H), 7.13-7.20 (m, 5H), 9.32 (bs, 2H). LCMS (12 minute method) [M+H]⁺=314@Rt 3.96 min. single major peak.

### EXAMPLE 2B

### (S,R) 2-(2-Ethoxy-phenyl)-1-morpholin-2-yl-1-phenyl-ethanol hydrochloride

### a) 1-(4-Benzyl-morpholin-2-yl)-2-(2-ethoxy-phenyl)-1-phenyl-ethanol

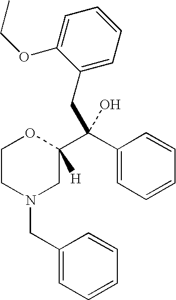

The procedure for the synthesis of example 1Ba, 1-(4-Benzyl-morpholin-2-yl)-2-(2-methoxy-phenyl)-1-phenyl-ethanol, was followed using commercially available 2-ethoxybenzylmagnesium bromide (available from Rieke-Metals) as starting material and making non-critical variations, to yield the title compound. FIA [M+H]⁺=418.

### b) 2-(2-Ethoxy-phenyl)-1-morpholin-2-yl-1-phenyl-ethanol hydrochloride

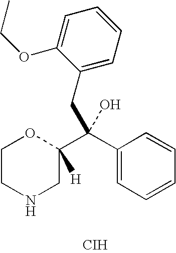

The procedure for the synthesis of example 1Bb, 2-(2-Methoxy-phenyl)-1-morpholin-2-yl-1-phenyl-ethanol hydrochloride was followed making non-critical variations, to yield the title compound. ¹H NMR (300 MHz, DMSO D6) δ: 1.11 (t, 3H, J=6.97 Hz), 2.43-2.56 (m, 1H), 2.81-2.96 (m, 2H), 3.17-3.27 (m, 3H), 3.55-3.67 (m, 2H), 3.84-3.92 (m, 1H), 4.05-4.20 (m, 2H), 6.68-6.74 (m, 2H), 7.01-7.18 (m, 8H), 8.92 (bs, 2H) ppm. LCMS (12 minute method) [M+H]⁺=328@Rt 4.57 min. single major peak.

### EXAMPLE 3B

### S,R) 2-(2-Isopropoxy-phenyl)-1-morpholin-2-yl-1-phenyl-ethanol hydrochloride

### a) 1-(4-Benzyl-morpholin-2-yl)-2-(2-isopropoxy-phenyl)-1-phenyl-ethanol

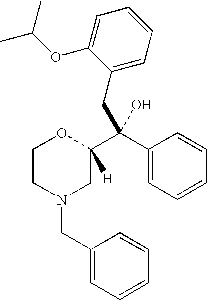

Solid magnesium turnings (4.6 g, 48 equiv.) under nitrogen atmosphere at room temperature were stirred vigorously with a magnetic stirring bar overnight. The magnesium was then covered with dry tetrahydrofuran. A cold bath was then applied followed by dropwise addition of 1-chloromethyl-2-isopropoxy-benzene (3.0 g, 4 equiv. prepared as described above) in tetrahydrofuran (40 mL). During slow addition of the electrophile no exotherm was observed so on completion of addition 3 crystals of Iodine were added to promote initiation of the reaction. After this addition the reaction temperature was allowed to spike to 50° C. then cooled rapidly to 8° C. before being left to warm to room temperature for one hour. The resulting black suspension was cooled down to −10° C. and a solution of (4-Benzyl-morpholin-2-yl)-phenyl-methanone (1.2 g, 1 equiv.) in tetrahydrofuran (10 mL) was then added dropwise. The reaction mixture was left to warm to room temperature over thirty minutes and then quenched by addition of aqueous saturated solution of NaHCO₃ (50 mL) prior to filtration through Celite. The aqueous solution was extracted with diethyl ether, the organic phase dried with MgSO₄, evaporated in vacuo to give 3 g of a yellow amorphous solid. The compound was taken without further purification in the next step. LCMS (6 minutes method) [M+H]⁺=432@Rt 3.25 min. major peak.

### b) 2-(2-Isopropoxy-phenyl)-1-morpholin-2-yl-1-phenyl-ethanol hydrochloride

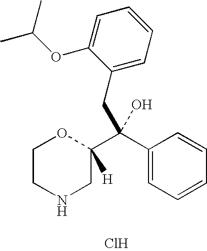

The procedure for the synthesis of example 1Bb, 2-(2-Methoxy-phenyl)-1-morpholin-2-yl-1-phenyl-ethanol hydrochloride was followed making non-critical variations, to yield the title compound. ¹H NMR (300 MHz, MeOH D3) δ: 1.12-1.16 (m, 6H), 2.51-2.55 (m, 1H), 2.89-3.14 (m, 4H), 3.56-3.60 (m, 1H), 3.82-3.92 (m, 1H), 3.99-4.03 (m, 1H), 4.17-4.22 (m, 1H), 4.36-4.44 (m, 1H), 6.50-6.55 (m, 1H), 6.66-6.73 (m, 2H), 6.92-6.98 (m, 1H), 7.07-7.20 (m, 5H) ppm. LCMS (12 minutes method) [M+H]⁺=342@Rt 4.90 min. major peak.

### EXAMPLE 4B

### (S,R) 1-(3-Fluoro-phenyl)-2-(2-methoxy-phenyl)-1-morpholin-2-yl-ethanol hydrochloride

### a) 1-(4-Benzyl-morpholin-2-yl)-1-(3-fluoro-phenyl)-2-(2-methoxy-phenyl)-ethanol

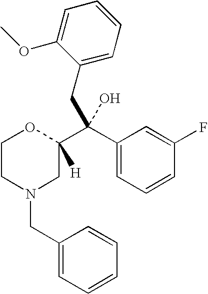

A magnetically stirred 0.25M tetrahydrofuran solution of commercially available 2-methoxybenzylmagnesium bromide (available from Rieke-Metals) (80 ml, 3equiv.) under nitrogen atmosphere was cooled to −10° C. and to this was added neat (4-Benzyl-morpholin-2-yl)-1-(3-fluoro-phenyl)-methanone (2.1 g, 1 equiv.). The solution was allowed to warm to room temperature and reaction progress followed using mass spectrometry. After 1.5 hours 2-methoxybenzylmagnesium bromide solution (14 ml, 0.5 equiv.) was again added to the reaction and after a further 0.5 hours an aqueous saturated solution of NaHCO₃ (50 mL) was added to halt the reaction. The aqueous solution was extracted with diethyl ether, the organic phase dried with MgSO₄, evaporated in vacuo to give 2.8 g of a yellow amorphous solid. The compound was taken without further purification in the next step. LCMS (6 minutes method) [M+H]⁺=422@Rt 3.03 and 2.86 min. major peaks.

### b) (S,R)-1-(3-Fluoro-phenyl)-2-(2-methoxy-phenyl)-1-morpholin-2-yl-ethanol hydrochloride

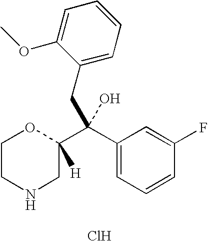

To a solution of 1-(4-Benzyl-morpholin-2-yl)-1-(3-fluoro-phenyl)-2-(2-methoxy-phenyl)-ethanol (2.8 g, 1 equiv.) in ethyl acetate (100 mL) at room temperature under nitrogen atmosphere was added ammonium formate (4.3 g, 10 equiv.) followed by addition of palladium on charcoal (10%, 2.7 g.). The reaction mixture was heated to reflux for 1 hour, cooled to room temperature and then filtered through Celite. All volatiles were evaporated under vacuum, and the resulting solid was purified via preparative HPLC to give the desired diastereoisomers. The active enantiomer was obtained after a further preparative chiral HPLC separation. The active enantiomer, a white solid, was next taken up in ethanol and hydrogen chloride was added (large excess of 2M solution in diethyl ether) and the mixture was stirred until it became a clear solution. Then all the volatiles were evaporated in vacuo, to give 447 mg of the title compound as white solid. ¹H NMR (300 MHz, DMSO D6) δ: 2.49-2.53 (m, 1H), 2.80-2.93 (m, 2H), 3.12-3.33 (m, 4H), 3.41 (s, 3H), 3.85-3.92 (m, 1H), 4.07-4.20 (m, 2H), 6.70-6.75 (m, 2H), 6.92-7.10 (m, 5H), 7.20-7.27 (m, 1H), 9.08 (bs, 2H). LCMS (12 minutes method) [M+H]⁺=332. Rt 4.11 min.

### EXAMPLE 5B

### (S,R) 1-Morpholin-2-yl-1-phenyl-2-(2-trifluoromethoxy-phenyl)-ethanol hydrochloride

### a) 1-(4-Benzyl-morpholin-2-yl)-1-phenyl-2-(2-trifluoromethoxy-phenyl)-ethanol

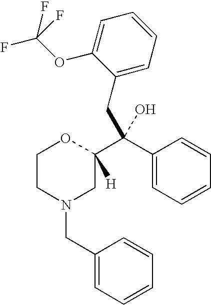

Magnesium turnings (24.2 g, 0.935 mole, 2 eq.) and diethyl ether (300 ml) were loaded in a reactor under N₂. A solution of 2-trifluoromethoxybenzyl bromide (165 g, 0.647 mole, 1.3 eq.) in diethyl ether (300 ml) was loaded in an addition funnel. Iodine crystals and a small amount of the 2-trifluoromethoxybenzyl bromide solution were added and the reaction mixture was stirred to initiate the reaction. The remainder of the 2-trifluoromethoxybenzyl bromide solution was then added drop-wise maintaining the temperature of the reaction mixture below 35° C. The mixture was stirred for another 5 minutes at 23° C. after completion of the addition. A solution of (4-Benzyl-morpholin-2-yl)-phenyl-methanone (140 g, 0.498 mole) in diethyl ether (2.1 L) was added drop-wise, maintaining the temperature of the reaction mixture below 25° C. The solution obtained was stirred for 1 hour at 20° C. The reaction mixture was quenched through the addition of a saturated aqueous NaHCO₃ solution (700 ml) and water (700 ml). The solids were filtered and washed with diethyl ether (200 ml). The filtrates were loaded into a separation funnel and the layers were separated. The aqueous layer was extracted with diethyl ether (1 L). The organic layers were combined and the filtrates were concentrated under vacuum to about 2 liters. The solution was dried over MgSO₄, filtered and the filter cake was washed with diethyl ether (200 ml). The filtrate was concentrated under vacuum to orange oil. The residue was twice dissolved in toluene (500 ml) and concentrated to a solid product. The yield of crude title compound was 235 g (103%). ¹H-NMR (CDCI₃): 6.80-7.07 ppm, 11 H, mp; 7.04-7.01 ppm, 1H, mp; 7.01-6.86 ppm, 1H, dt; 6.84-6.80 ppm, 1H, d; 3.98-4.03 ppm, 1H, dt; 3.86-3.89 ppm, 1H, dd; 3.70-3.60 ppm, 1H, dt; 3.52-3.58 ppm, 1H, d; 3.37-3.42 ppm, 1H, d; 3.13-3.37 ppm, 1H, d; 3.05-3.08 ppm, 1H, d; 2.44-2.45 ppm, 1H, d; 2.30-2.00 ppm, 3H, mp.

### b) (S,R) 1-Morpholin-2-yl-1-phenyl-2-(2-trifluoromethoxy-phenyl)-ethanol hydrochloride

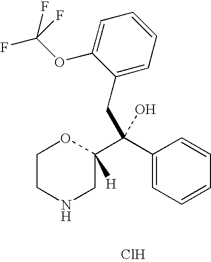

A stainless steel Buchi hydrogenation reactor was loaded with 1-(4-Benzyl-morpholin-2-yl)-1-phenyl-2-(2-trifluoromethoxy-phenyl)-ethanol (230 g, 0.503 mole), methanol (1 L), a suspension of Pd/C (10%, 46 g, 20% loading) in methanol (500 ml), and methanol (500 ml) from equipment rinses. A solution of HCl in ethanol (1.6N, 460 ml, 0.736 mole, 1.5 eq.) was added and the reactor was pressurized with H₂ (3 Bar). The reaction mixture was heated to 40° C. and stirred for 3 hours. The reaction mixture was cooled to 20° C. and flushed with N₂. The catalyst was filtered off and washed with methanol (0.5 L). The filtrates were concentrated under vacuum to a yellow solid. The yield of crude title compound was 198 g (97.5%). A reactor was loaded with crude title compound (190 g, 0.47 mole) and toluene (6.65 L) under N₂. The suspension was heated under reflux and toluene (150 ml) was added until all solid dissolved. The solution was stirred for 15 minutes more under reflux and then cooled slowly to 20° C. The suspension was stirred for 1 hour at 20° C. The solid was filtered, washed with toluene (680 ml), and dried at 40° C. under vacuum. The yield of pure anhydrous title compound was 158.5 g (83.4%).

Alternatively, the following method can be used. In a glass-lined nitrogen purged hydrogenator are charged 1-(4-Benzyl-morpholin-2-yl)-1-phenyl-2-(2-trifluoromethoxy-phenyl)-ethanol hydrochloride (150 g, 303.7 mmol), demineralized water (352 mL), i-PrOH (375 mL) and 5% Pd/C (30 g, 50% water, Johnson & Matthey type 440). The heterogeneous reaction mixture was then purged 5 times with 25 psi nitrogen then purged 5 times with 50 psi hydrogen, and the hydrogenation was performed at RT. The initial Tmass was 22° C. and the maximum Tmass during the hydrogenation was 23° C. The reactor was stirred vigorously. In-process analysis after 2 hours indicated complete hydrogenolysis. The hydrogenation was stopped after 3 hours. The nitrogen purged reaction mixture was then filtered at RT through an hyflo filter (56 g), impregnated beforehand with 75 mL of a 50/50 v/v isopropanol/water mixture and washed with 300 mL of a 50/50 v/v isopropanol/water mixture. The filtrates were stored overnight at RT. The filtrates were concentrated at 40-50° C. under reduced pressure (typical 622 g distilled). The reaction mixture was cooled to RT and post-agitated. After 3 hours, 1 mL of the solution was taken and cooled to 0° C. to initiate crystallization. These seeds were added to the reaction mixture and precipitation was observed within a few minutes. The mixture was post-agitated at RT for 2 hours. The crystals were filtered and rinsed with H₂O (30 mL). Then, the precipitate was dried under reduced pressure (400 mmHg) with a nitrogen flow (0.1 bar) for 4 hours affording the title compound as the hydrate polymorph (103.5 g, 81% yield).

### EXAMPLE 6B

### (S,R) 2-Biphenyl-2-yl-1-morpholin-2-yl-1-phen-1-ethanol hydrochloride

### a) 1-(4-Benzyl-morpholin-2-yl)-2-biphenyl-2-yl-1-phenyl-ethanol

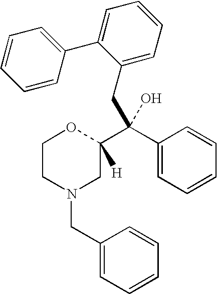

1-(4-Benzyl-morpholin-2-yl)-2-(2-bromo-phenyl)-1-phenyl-ethanol (0.50 g, 1.0 equiv. prepared according to Example 15Ba below) and phenylboronic acid (0.402 g, 3.0 equiv., available from Aldrich Chemical Company) were suspended in a mixture ethanol/water (2/1, 7.5 mL) and Pd(Ph₃)₄ (0.022 g, 0.04 equiv.), then K₂CO₃ (0.654 g, 4.30 equiv.) were added. The mixture was heated to 80° C. under nitrogen atmosphere. After 16 hours, the reaction was cooled down to room temperature and filtered through Celite, then extracted with ethyl acetate. The organic layers were combined, dried with MgSO₄, filtered and concentrated in vacuo yielding a yellow oil, which was purified by column chromatography on silica gel (10% EtOAc:Hexane) to give 0.491 g (98%) of the title compound as a white solid.

### b) (S,R) 2-Biphenyl-2-yl-1-morpholin-2-yl-1-phenyl-ethanol hydrochloride

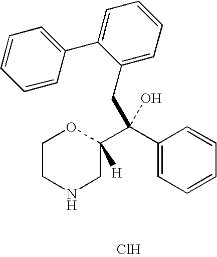

The procedure for the synthesis of example 1Bb, 2-(2-methoxy-phenyl)-1-morpholin-2-yl-1-phenyl-ethanol hydrochloride, was followed making non-critical variations, to yield the title compound.¹H NMR (300 MHz, DMSO D6) δ: 2.16-2.20 (m, 1H), 2.54-2.62 (m, 1H), 2.67-2.76 (m, 1H), 2.85-2.89 (m, 1H), 3.24 (s, 2H), 3.61-3.69 (m, 2H), 3.93-3.98 (m, 1H), 5.14 (bs, 1H), 6.80-6.92 (m, 5H), 7.04-7.17 (m, 5H), 7.27-7.30 (m, 3H), 7.36-7.39 (m, 1H). LCMS (12 minutes method) [M+H]⁺=360@Rt 5.15 min. single major peak.

### EXAMPLE 7B

### (S,R) 2-(2-Chloro-phenyl)-1-mortholin-2-yl-1-phenyl-ethanol hydrochloride

### a) 1-(4-Benzyl-morpholin-2-yl)-2-(2-chloro-phenyl)-1-phenyl-ethanol

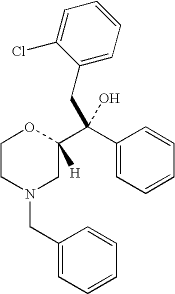

The procedure for the synthesis of example 1Ba, 1-(4-Benzyl-morpholin-2-yl)-2-(2-methoxy-phenyl)-1-phenyl-ethanol, was followed using 2-chlorobenzyl chloride (available from Aldrich Chemical Company) as starting material and making non-critical variations, to yield the title compound. FIA [M+H]⁺=408 and 410.

### b) (S,R) 2-(2-Chloro-phenyl)-1-morpholin-2-yl-1-phenyl-ethanol hydrochloride

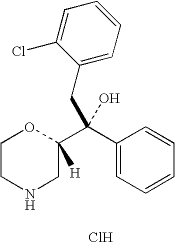

The procedure for the synthesis of example 5Bb, (S, R) 1-Morpholin-2-yl-1-phenyl-2-(2-trifluoromethoxy-phenyl)-ethanol hydrochloride, was followed making non-critical variations, to yield the title compound.¹H NMR (300 MHz, DMSO D6) δ: 2.45-2.54 (m, 1H), 2.84-2.93 (m, 2H), 3.17-3.22 (m, 1H), 3.33-3.38 (m, 3H), 3.89-3.97 (m, 1H), 4.14-4.18 (m, 2H), 7.06-7.11 (m, 2H), 7.15-7.26 (m, 7H), 9.24 (bs, 2H) ppm. LCMS (12 minutes method) [M+H]⁺=318-320@Rt 4.36 min. single peak.

### EXAMPLE 8B

### (S,R) 2-(5-Fluoro-2-methoxy-phenyl)-1-morpholin-2-yl-1-phenyl-ethanol hydrochloride

### a) 1-(4-Benzyl-morpholin-2-yl)-2-(5-fluoro-2-methoxy-phenyl)-1-phenyl-ethanol

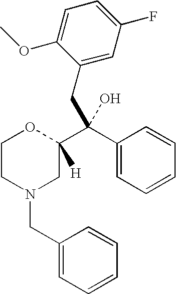

Magnesium turnings (21.6 g, 0.888 mole, 2 eq.) and diethyl ether (300 ml) were loaded in a reactor under N₂. A solution of 5-fluoro-2-methoxybenzyl chloride (116 g, 0.664 mole, 1.5 eq.) in diethyl ether (200 ml) was loaded in an addition funnel. Iodine crystals and a small amount of the 5-fluoro-2-methoxybenzyl chloride solution were added and the reaction mixture was stirred to initiate the reaction. The remainder of the 5-fluoro-2 methoxybenzyl chloride solution was then added drop-wise maintaining the temperature of the reaction mixture below 28° C. The mixture was stirred for another 5 minutes at 19° C. after completion of the addition and a white suspension was formed. A solution of (4-Benzyl-morpholin-2-yl)-phenyl-methanone (125 g, 0.444 mole) in diethyl ether (1.8 L) was added drop-wise, maintaining the temperature of the reaction mixture below 25° C. The suspension obtained was stirred for 2 hours. The reaction mixture was quenched through the addition of a saturated aqueous NaHCO₃ solution (625 ml) and water (500 ml), maintaining the temperature below 20° C. The mixture was stirred for 30 minutes and the solids were filtered, washed with water (125 ml) and diethyl ether (200 ml). The filtrates were loaded into a separation tunnel and the layers were separated. The aqueous layer was extracted with diethyl ether (1 L). The organic layers were combined and dried over MgSO₄, filtered and the filter cake was washed with diethyl ether (100 ml). The filtrates were concentrated under vacuum. The yield of title compound was 201 g as a yellow solid (107%). Title compound (200 g, 0.474 mole) was then suspended in isopropanol (400 ml) under N₂. The suspension was heated under reflux until all solids were dissolved. The solution is allowed to cool to 20° C. over 4 hours under stirring. The solid is filtered, washed with isopropanol (100 ml) and dried at 40° C. under vacuum. The yield of pure title compound is 158 g (79%). ¹H-NMR (CDCl₃): 6.99-7.26 ppm, 10H, mp; 6.60-6.71 ppm, 1H, dt; 6.49-6.60 ppm, 1H, dd; 6.31-6.44 ppm, 1H, dd; 3.92-4.01 ppm, 1H, dt; 3.80-3.90 ppm, 1H, dd; 3.64-3.73 ppm, 1H, dd; 3.59-3.64 ppm, 1H, d; 3.52-3.59 ppm, 3+1 H, 2s; 3.37-3.45 ppm, 1H, d; 3.07-3.17 ppm, 1H, d; 2.84-2.92 ppm, 1H, d; 2.43-2.53 ppm, 1H, d; 2.20-2.28 ppm, 1H, d; 1.98-2.11 ppm, 2H, mp.

### b) (S,R) 2-(5-Fluoro-2-methoxy-phenyl)-1-morpholin-2-yl-1-phenyl-ethanol hydrochloride

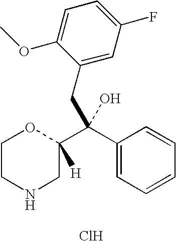

A glass hydrogenation flask was loaded with methanol (1.55 L), Pd/C (10%, 31 g, 20% loading), 1-(4-benzyl-morpholin-2-yl)-2-(5-fluoro-2-methoxy-phenyl)-1-phenyl-ethanol (155 g, 0.368 mole) and a solution of HCl in ethanol (2.5N, 233 ml, 0.582 mole, 1.6 eq.). The reactor was mounted on a Parr instrument and pressurized with H₂ (49 Psi). The reaction mixture was shaken overnight between 20° C. and 15° C. The catalyst was filtered off and washed with methanol (0.5 L). The filtrates were concentrated under vacuum. The yield of crude title compound was 109.5 g (81%). The catalyst was washed again with methanol (2×500 ml). The filtrates were combined and concentrated under vacuum. The yield of the second crop of crude title compound was 21.7 g (16%). A reactor was loaded with crude title compound (131 g, 0.356 mole) and isopropanol (1,3 L) under N₂. The suspension was heated under reflux for 4 hours. The mixture was cooled to 20° C. and the solid was filtered, washed with isopropanol (130 ml), and dried at 50° C. under vacuum. The yield of pure title compound was 115.9 g (88.5% yield).

### EXAMPLE 9B

### (S,R) 1-Morpholin-2-yl-1-phenyl-2-(2-trifluoromethylsulfanyl-phenyl)-ethanol acetate

### a) 1-(4-Benzyl-morpholin-2-yl)-1-phenyl-2-(2-trifluoromethylsulfanyl-phenyl)-ethanol

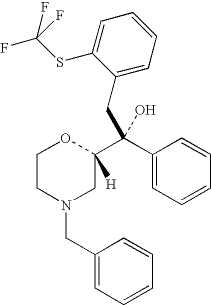

The procedure for the synthesis of example 1Ba, 1-(4-benzyl-morpholin-2-yl)-2-(2-methoxy-phenyl)-1-phenyl-ethanol, was followed using 1-bromomethyl-2-trifluoromethylsulfanyl-benzene (available from Fluorochem Ltd.) as starting material and making non-critical variations, to yield the title compound. ¹H NMR (300 MHz, CDCl₃) δ: 2.05-2.33 (m, 3H), 2.49-2.65 (m, 1H), 3.10-3.35 (m, 2H), 3.43-3.55 (m, 1H), 3.67-3.89 (m, 2H), 3.91-4.08 (m, 2H), 4.09-4.22 (m, 1H), 6.91-7.05 (m, 1H), 7.10-7.42 (m, 12H), 7.50-7.63 (m, 1H) ppm.

### b) (S, R) 1-Morpholin-2-yl-1-phenyl-2-(2-trifluoromethylsulfanyl-phenyl)-ethanol acetate

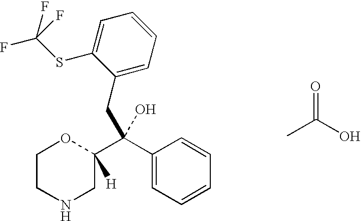

To a solution of 1-(4-benzyl-morpholin-2-yl)-1-phenyl-2-(2-trifluoromethylsulfanyl-phenyl)-ethanol (218 mg g, 1 equiv.) and solid supported Hunig's base (available from Argonaut, 1 g, 5 equiv.) in dry tetrahydrofuran (4 mL) at 0° C. under nitrogen atmosphere was added ACE-Cl (502 μL, 10 equiv.). The reaction mixture was left to warm to room temperature for 48 hours. All volatiles were evaporated under vacuum, and the resulting solid was taken-up with methanol (50 mL) and stirred at room temperature overnight. The solution was filtered through acid ion exchange column and the required fractions evaporated to dryness. The resulting solid was purified via preparative HPLC to give 62 mg of the title compound as a colourless oil. ¹H NMR (300MHz, CDCl₃) δ: 2.01 (s, 3H), 2.43-2.47 (m, 1H), 2.63-2.70 (m, 1H), 2.81-2.94 (m, 2H), 3.24 (d, 1H, J=13.57 Hz), 3.85-3.96 (m, 2H), 4.01-4.05 (m, 1H), 4.09-4.13 (m, 1H), 4.45 (bs, 4H), 6.90-6.93 (m, 1H), 7.13-7.26 (m, 7H), 7.55-7.58 (m, 1H), ppm. LCMS (12 minute method) [M+H]⁺=384@Rt 5.13 min. single peak.

### EXAMPLE 10B

### (S,R) 1-Morpholin-2-yl-1-phenyl-2-(2-trifluoromethyl-phenyl)-ethanol

### a) 4-Benzyl-2-(2-phenyl-oxiranyl)-morpholine

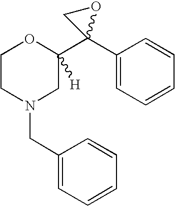

To a mixture of trimethylsulfoxonium iodide (783 mg, 1 equiv.) and sodium hydride (142 mg, 1 equiv.) in dimethylformamide (17 mL) at 0° C. under nitrogen atmosphere was added dimethylsulfoxide (251 μL, 1 equiv.) and the resulting suspension was stirred for 30 minutes. A solution of (4-Benzyl-morpholin-2-yl)-phenyl-methanone (1 g, 1 equiv.) in dimethylformamide (10 mL) was then added dropwise. Stirring was continued for 30 minutes and the reaction was stopped by addition of water (50 mL). The aqueous solution was extracted with diethyl ether, the organic phase dried with MgSO₄, and evaporated in vacuo. The crude material was purified using a column chromatography on silica gel eluting with a mixture of ethyl acetate/heptane (20/80) to give 825 mg of the title compound as a colourless oil (78%), mixture of two diastereoisomers. LCMS (6 minute method) [M+H]⁺=296@Rt 1.88 min. single peak.

### b) 1-(4-Benzyl-morpholin-2-yl)-1-phenyl-2-(2-trifluoromethyl-phenyl)-ethanol

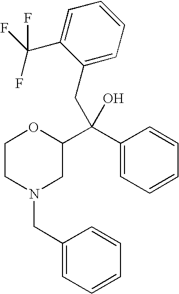

To a suspension of magnesium turnings in tetrahydrofuran (2 mL) at room temperature under nitrogen atmosphere was added a solution of 1-bromo-2-trifluoromethyl-benzene (7.6 g, 5 equiv., available from Acros) in tetrahydrofuran (32 mL) and the mixture was stirred for an hour. The solution was cooled to −78° C. and copper iodide (646 mg) was added followed by dropwise addition of a solution of 4-Benzyl-2-(2-phenyl-oxiranyl)-morpholine (2g, 1 equiv.) in tetrahydrofuran (10 mL). The resulting mixture was warmed to room temperature over 2 hours and then treated with water (10 mL). The solution was extracted with diethyl ether, the organic phase dried with MgSO₄, and evaporated in vacuo. The crude material was purified using a column chromatography on silica gel eluting with a mixture of ethyl acetate/heptane (10/90) to give 352 mg of the title compound as a colourless oil (12%). LCMS (6 minutes method) [M+H]⁺=442@Rt 3.05 min. major peak.

### c) (S,R) 1-Morpholin-2-yl-1-phenyl-2-(2-trifluoromethyl-phenyl)-ethanol

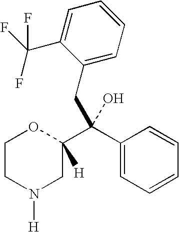

To a solution of 1-(4-Benzyl-morpholin-2-yl)-1-phenyl-2-(2-trifluoromethyl-phenyl)-ethanol (352 mg, 1 equiv.) in ethanol (15 mL) at room temperature under nitrogen atmosphere was added ammonium formate (507 mg g, 10 equiv.) followed by addition of palladium on charcoal (10%, 355 mg.). The reaction mixture was heated to reflux for 1 hour, cooled to room temperature and then filtered through Celite. All volatiles were evaporated under vacuum to give 265 mg of the title compound as white solid (94%). The enantiomeric mixture was resolved using chiral HPLC, to give the title compound as a single enantiomer. ¹H NMR (300 MHz, CDCl₃) δ: 2.25-2.30 (m, 1H), 2.56-2.64 (m, 1H), 2.75-2.87 (m, 2H), 3.18 (d, 1H, J=14.88 Hz), 3.71-3.81 (m, 2H), 3.89 (d, 1H, J=14.88 Hz), 4.02-4.05 (m, 1H), 6.83-6.86 (m, 1H), 7.09-7.34 (m, 7H), 7.53-7.55 (m, 1H) ppm. LCMS (12 minute method) [M+H]⁺=352@Rt 4.73 min. single peak.

### EXAMPLE 11B

### (S,R) 2-(2-Chloro-phenyl)-1-(3-fluoro-phenyl)-1-morpholin-2-yl-ethanol hydrochloride

### a) 1-(4-Benzyl-morpholin-2-yl)-2-(2-chloro-phenyl)-1-(3-fluoro-phenyl)-ethanol

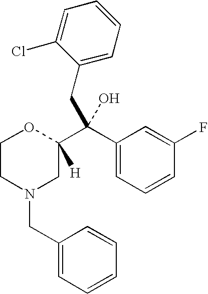

The procedure for the synthesis of 4Ba, 1-(4-Benzyl-morpholin-2-yl)-1-(3-fluoro-phenyl)-2-(2-methoxy-phenyl)-ethanol was followed using 2-chorobenzyl chloride (available from Aldrich Chemical Company) as starting material, and making non-critical variations, to yield the title compound which was taken without further purification in the next step. LCMS (6 minutes method) [M+H]⁺=426@Rt 2.85 min. major peak.

### b) (S,R) 2-(2-Chloro-phenyl)-1-(3-fluoro-phenyl)-1-morpholin-2-yl-ethanol hydrochloride

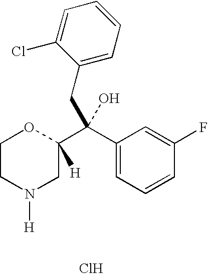

To a solution of 1-(4-Benzyl-morpholine-2-yl)-2-(2-chloro-phenyl)-1-(3-fluoro-phenyl)-ethanol. (3.2 g, 1 equiv.) in dry 1,2-dichloroethane (40 mL) under nitrogen atmosphere was added ACE-Cl (20.33 g, 5 equiv.). The reaction mixture was stirred at room temperature overnight then refluxed until completion. All volatiles were evaporated under vacuum, and the resulting residue redissolved in acetonitrile. This solution was filtered through an ion exchange column and the filtrate taken-up with methanol (50 mL) and refluxed for 3h. The solution was again filtered through acid ion exchange column and the required fractions evaporated to dryness. The resulting solid was next purified via preparative HPLC followed by chiral HPLC. The purified active enantiomer was taken up in ethanol and hydrogen chloride was added (large excess of 2M solution in diethyl ether) and the mixture stirred. Then all the volatiles were evaporated in vacuo, to give 519 mg of the title compound as a white solid (18%). ¹H NMR (300 MHz, DMSO D6) δ: 2.43-2.54 (m, 1H), 2.81-2.95 (m, 2H), 3.16-3.23 (m, 1H), 3.30-3.44 (m, 2H), 3.54 (bs, 1H), 3.92-4.00 (m, 1H), 4.15-4.23 (m, 2H), 6.96-7.29 (m, 8H), 9.32-9.45 (m, 2H). LCMS (12 minute method) [M+H]⁺=336.

### EXAMPLE 12B

### (S,R) 1-Morpholin-2-yl-1-phenyl-2-o-tolyl-ethanol hydrochloride

### a) 1-(4-Benzyl-morpholin-2-yl)-1-phenyl-2-o-tolyl-ethanol

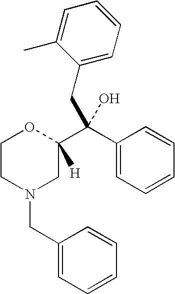

The procedure for the synthesis of example 1Ba, 1-(4-benzyl-morpholin-2-yl)-2-(2-methoxy-phenyl)-1-phenyl-ethanol, was followed using commercially available 2-methylbenzylmagnesium bromide (available from Rieke-Metals) as starting material and making non-critical variations, to yield the title compound. FIA [M+H]⁺=388.

### b) (S,R) 1-Morpholin-2-yl-1-phenyl-2-o-tolyl-ethanol hydrochloride

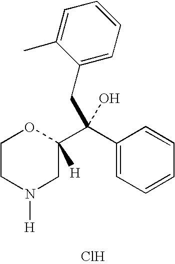

The procedure for the synthesis of example 1Bb, 2-(2-methoxy-phenyl)-1-morpholin-2-yl-1-phenyl-ethanol hydrochloride was followed making non-critical variations, to yield the title compound. ¹H NMR (300 MHz, DMSO D6) δ: 1.62 (s, 3H), 2.40-2.58 (m, 1H), 2.78-3.01 (m, 2H), 3.03-3.09 (m, 1H), 3.15-3.31 (m, 2H), 3.90-4.05 (m, 1H), 4.15-4.25 (m, 2H), 6.89-7.28 (m, 9H), 9.21-9.55 (m, 2H). LCMS (12 minute method) [M+H]⁺=298 single peak.

### EXAMPLE 13B

### (S,R) 1-Morpholin-2-yl-1,2-diphenyl-ethanol hydrochloride

### a) 1-(4-Benzyl-morpholin-2-yl)-1,2-diphenyl-ethanol

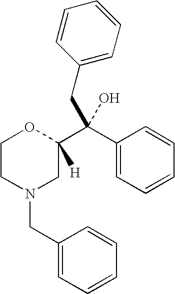

The procedure for the synthesis of example 1Ba, 1-(4-benzyl-morpholin-2-yl)-2-(2-methoxy-phenyl)-1-phenyl-ethanol, was followed using commercially available benzylmagnesium bromide (available from TCI America) as starting material and making non-critical variations, to yield the title compound. LCMS [M+H]⁺=374.1 major single peak@3.82 min.

### b) (S,R) 1-Morpholin-2-yl-1,2-diphenyl-ethanol hydrochloride

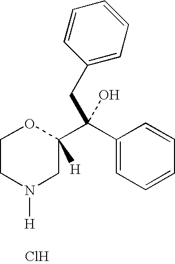

The procedure for the synthesis of example 1Bb, 2-(2-methoxy-phenyl)-1-morpholin-2-yl-1-phenyl-ethanol hydrochloride was followed making non-critical variations, to yield the title compound. ¹H NMR (300 MHz, CDDl₃) δ: 2.36-2.41 (m, 1H), 2.64-2.71 (m, 1H), 2.78-2.91 (m, 3H), 3.16-3.32 (m, 2H), 3.73-3.82 (m, 2H), 4.08-4.11 (m, 1H), 6.80-6.83 (m, 2H), 7.07-7.12 (m, 3H), 7.16-7.27 (m, 6H). LCMS [M+H]⁺=284.1 single peak@3.82 minutes.

### EXAMPLE 14B

### (S,R) 2-(2-Fluoro-phenyl)-1-morpholin-2-yl-1-phenyl-ethanol hydrochloride

### a) 1-(4-Benzyl-morpholin-2-yl)-2-(2-fluoro-phenyl)-1-phenyl-ethanol

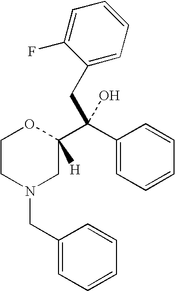

The procedure for the synthesis of example 1Ba, 1-(4-benzyl-morpholin-2-yl)-2-(2-methoxy-phenyl)-1-phenyl-ethanol, was followed using commercially available 2-fluoro-benzylmagnesium chloride (available from Rieke Metals) as starting material and making non-critical variations, to yield the title compound. FIA [M+H]⁺=392.1.

### b) (S,R) 2-(2-Fluoro-phenyl)-1-morpholin-2-yl-1-phenyl-ethanol hydrochloride

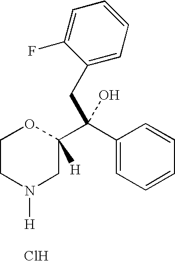

The procedure for the synthesis of example 1Bb, 2-(2-methoxy-phenyl)-1-morpholin-2-yl-1-phenyl-ethanol hydrochloride was followed making non-critical variations, to yield the title compound. ¹H NMR (300 MHz, DMSO D6) δ: 2.40-2.56 (m, 1H), 2.78-2.97 (m, 2H), 3.17-3.29 (m, 3H), 3.89-3.96 (m, 1H), 4.14-4.19 (m, 2H), 5.47 (bs, 1H), 6.82-6.94 (m, 2H), 7.01-7.25 (m, 7H), 9.28-9.38 (m, 2H). LCMS [M+H]⁺=302.1 single major peak (3.82 minutes.

### EXAMPLE 15B

### (S,R) 2-(2-bromo-phenyl)-1-phenyl-1-morpholin-2-yl-ethanol

### a) 1-(4-Benzyl-morpholin-2-yl)-2-(2-bromo-phenyl)-1-phenyl-ethanol

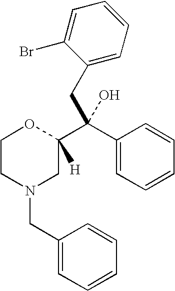

The procedure for the synthesis of example 1Ba, 1-(4-Benzyl-morpholin-2-yl)-2-(2-methoxy-phenyl)-1-phenyl-ethanol, was followed using commercially available 2-bromobenzylmagnesium bromide (available from Rieke-Metals) as starting material and making non-critical variations, to yield the title compound. FIA [M+H]⁺=452/454.

### b) (S,R) 1-Morpholin-2-yl-2-(2-bromo-phenyl)-1-phenyl-ethanol

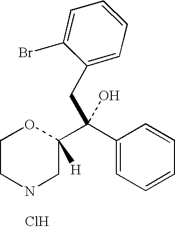

The procedure for the synthesis of example 5Bb, (S,R) 1-Morpholin-2-yl-1-phenyl-2-(2-trifluoromethoxy-phenyl)-ethanol, was followed making non-critical variations, to yield the title compound.¹H NMR (300 MHz, CDCl₃) δ: 2.64-2.68 (m, 1H), 3.02-3.21 (m, 2H), 3.27-3.33 (m, 3H), 3.45-3.50 (m, 1H), 3.63-3.68 (m, 1H), 3.99-4.09 (m, 1H), 4.20-4.24 (m, 1H), 4.29-4.34 (m, 1H), 4.87 (s, 1H), 6.98-7.21 (m, 2H), 7.24-7.59 (m, 7H) ppm. LCMS (6 minutes method) [M+H]⁺=362.3@Rt 2.85 min. single peak.

### EXAMPLE 16B

### (S,R) 2-(2′-chlor[1-1′biphenyl]-2-yl)-1-morpholin-2-yl-1-phenyl-ethanol hydrochloride

### a) 2-(2′-chloro[1-1′biphenyl]-2-yl)-1-phenyl-1-[4-(phenylmethyl)morpholin-2-yl]ethanol

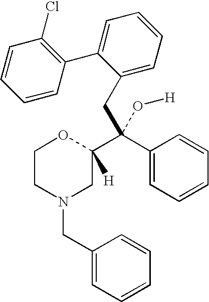

The procedure for the synthesis of example 6Ba, was followed using 2-chloro phenyl boronic acid (available from Aldrich Chemical Company) as starting material and making non-critical variations, to yield the title compound. FIA [M+H]⁺=485

### b) (S,R) 2-(2′-chloro[1-1′biphenyl]-2-yl)-1-morpholin-2-yl-1-phenyl-ethanol hydrochloride

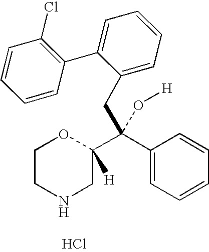

The procedure for the synthesis of example 6Bb, was followed making non-critical variations, to yield the title compound. ¹H NMR (300 MHz, CDCl₃) δ: 2.10-2.21 (m, 1H), 2.57-2.65 (m, 1H), 2.62-2.75 (m, 1H), 2.83-2.87 (m, 1H), 3.20 (s, 2H), 3.63-3.70 (m, 2H), 3.95-3.97 (m, 1H), 5.12 (bs, 1H), 6.80-6.92 (m, 5H), 7.04-7.17 (m, 5H), 7.27-7.37 (m, 3H). LCMS (12 minutes method) [M+H]⁺=393@Rt 4.75 min. single major peak.

### EXAMPLE 17B

### 4-Fluoro-2-(2-morpholin-2-yl-2-phenylpropyl)phenol hydrochloride

### a) 4-Fluoro-2-(2-morpholin-2-yl-2-phenylpropyl)phenol hydrochloride

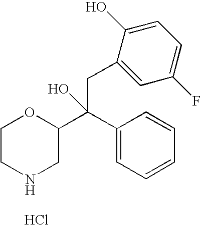

Sodium thiomethoxide (13 eq, 186 mg) was added at once to a solution of 2-{2-[5-fluoro-2-(methyloxy)phenyl]-1-methyl-1-phenylethyl}morpholine hydrochloride (75.2 mg, 0.204 mmol, synthesized as described in Example 8 above) in anydrous DMF (3 ml) in a microwave vessel. Upon addition, the reaction vessel was sealed and heated up in a CEM-Discovery microwave at 150 Watts, reaching 110° C. in 5 minutes and maintaining this temperature 6 minutes. The reaction vessel was cooled to room temperature and the reaction mixture taken into methanol (5 ml) and purified by SCX-2 chromatography to obtain the free base as clear oil (50 mg). The hydrochloride salt was obtained following general procedures as a white solid (52 mg, 72% after salt formation.). MW 353.83; C₁₈H₂₂NO₃FCl; ¹H NMR (CD₃OD): 7.29-7.26 (2H, m), 7.20-7.08 (2H, m), 6.53-6.50 (2H, m), 6.30-6.26 (1H, m), 4.18 (1H, dd, 12.6 Hz, 2.6 Hz), 4.02 (1H, dd, 10.9 Hz, 2.3 Hz), 3.86 (1H, td, 12.6 Hz, 2.6 Hz), 3.60 (1H, 1/2 AB), 3.16 (1H, d, 12.6 Hz), 3.08-2.90 (3H, m), 2.58 (1H, m); ¹⁹F NMR (CD₃OD) −128.4; LCMS: (12 min method) m/z 318.1 [M−HCl+H]⁺@Rt 3.954 min.

### EXAMPLE 18B

### 2-(2-Fluoro-6-chloro-phenyl)-1-morpholin-2-yl-1-phenyl-ethanol hydrochloride

### a) 1-(4-Benzyl-morpholin-2-yl)-2-(2-chloro-6-fluoro-phenyl)-1-phenyl-ethanol

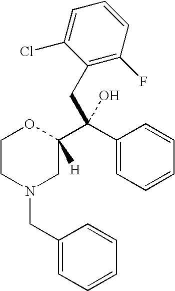

To a stirred solution of 2-chloro-6-fluorobenzyl magnesium chloride (12.8 mL, 3.20 mmol, 3 equiv., available from Rieke Metals) in anhydrous tetrahydrofuran (15 ml) at 0° C. under nitrogen was added a solution of (4-Benzyl-morpholin-2-yl)-phenyl-methanone (300 mg, 1.07 mmol, 1 equiv.) in tetrahydrofuran (5 ml) dropwise over 15 minutes. The reaction was then stirred at 0° C. for one hour. The reaction mixture was allowed to warm to room temperature over two hours and stirred for a further 18 h. The solvent was then evaporated “in vacuo” and the residue redissolved in dichloromethane (30 mL). The organic solution was washed with aqueous saturated solution of NaHCO₃ (50 mL). The aqueous solution was extracted with dichloromethane using a hydrophobic phase separator. The dichloromethane was evaporated “in vacuo” and redissolved in methanol (2 mL). The sample was bound to SCX-2 (5 g) and washed with methanol (30 mL). The sample was eluted using 2M ammonia in methanol (30 mL). The solvent was then evaporated using a reacti-therm blow down station to give 450 mg of a yellow amorphous solid. This material was used in step b) without further purification. LCMS (6 minutes method) [M+H]⁺=426@Rt 3.27 min. major peak.

### b) 2-(2-Fluoro-6-chloro-phenyl)-1-morpholin-2-yl-1-phenyl-ethanol hydrochloride

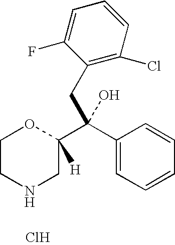

To a solution of 1-(4-Benzyl-morpholin-2-yl)-2-(2-chloro-6-fluoro-phenyl)-1-phenyl-ethanol (450 mg, 1 equiv.) in ethyl acetate (15 mL) at room temperature under nitrogen atmosphere was added ammonium formate (1.69 g, 25 equiv.) followed by addition of palladium on charcoal (10%, 450 g.). The reaction mixture was heated to reflux for 1.5 hours, cooled to room temperature and then filtered through Celite. All volatiles were evaporated under vacuum, and the resulting solid was purified via preparative HPLC. The isolated white solid was taken up in ethanol. Hydrogen chloride was added (large excess of 2M solution in diethyl ether) and the mixture was stirred until it became a clear solution. Then all the volatiles were evaporated “in vacuo”, to give 147 mg of the title compound as white solid. ¹H NMR (300 MHz, CD₃OD D4) δ: 2.51-2.61 (d, 1H), 2.79-2.91 (t, 1H), 2.96-3.09 (m, 1H), 3.09-3.16 (m, 1H), 3.32-3.54 (q, 2H), 3.82-3.97 (t, 1H), 4.09-4.24 (t, 2H), 6.73-6.84 (t, 1H), 6.93-7.08 (m, 2H), 7.08-7.21 (m, 5H). LCMS (12 minutes method) [M+H]⁺=336@Rt 4.44 min. single major peak.

### EXAMPLE 19B

### 2-(2,5-Dimethoxy-phenyl)-1-morpholin-2-yl-1-phenyl-ethanol hydrochloride

### a) 1-(4-Benzyl-morpholin-2-yl)-2-(2,5-dimethoxy-phenyl)-1-phenyl-ethanol

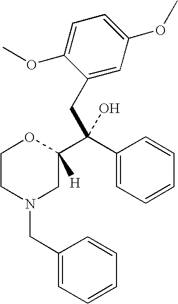

The procedure for the synthesis of example 18Ba, 1-(4-Benzyl-morpholin-2-yl)-2-(2-chloro-6-fluoro -phenyl)-1-phenyl-ethanol, using 2,5-dimethoxybenzyl magnesium chloride as starting material (available from Rieke Metals) was followed making non-critical variations, to yield the title compound. This material was used in step b) without further purification. LCMS (6 minutes method) [M+H]⁺=434@Rt 3.10 min. major peak.

### b) 2-(2,5-Dimethoxy-phenyl)-1-morpholin-2-yl-1-phenyl-ethanol hydrochloride

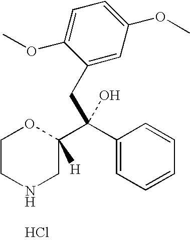

The procedure for the synthesis of example 18Bb, 2-(2-Fluoro-6-chloro-phenyl)-1-morpholin-2-yl-1-phenyl-ethanol hydrochloride, was followed making non-critical variations, to yield the title compound.¹H NMR (300 MHz, CD₃OD D4) δ: 2.53-2.62 (d, 1H), 2.86-3.10 (m, 3H), 3.13-3.27 (m, 2H), 3.36-3.51 (m, 6H), 3.81-3.93 (t, 1H), 4.02-4.08 (d, 1H), 4.15-4.25 (d, 1H), 6.28-6.33 (s, 1H), 6.49-6.64 (m, 2H), 7.06-7.22 (m, 5H). LCMS (12 minutes method) [M+H]⁺=344@Rt 4.15 min. single major peak.

### EXAMPLE 20B

### 2-(2,4-Difluoro-phenyl)-1-morpholin-2-yl-1-phenyl-ethanol hydrochloride

### a) 1-(4-Benzyl-morpholin-2-yl)-2-(2,4-difluoro-phenyl)-1-phenyl-ethanol

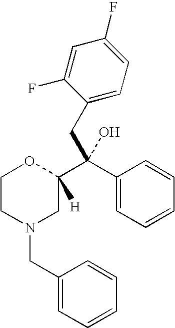

The procedure for the synthesis of example 18Ba, 1-(4-Benzyl-morpholin-2-yl)-2-(2-chloro-6-fluoro-phenyl)-1-phenyl-ethanol, using 2,4-difluorobenzyl magnesium bromide as starting material (available from Rieke Metals) was followed making non-critical variations, to yield the title compound. This material was used in step b) without further purification. LCMS (6 minutes method) [M+H]⁺=410@Rt 3.19 min. major peak.

### b) 2-(2,4-Difluoro-phenyl)-1-morpholin-2-yl-1-phenyl-ethanol hydrochloride

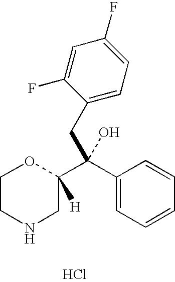

The procedure for the synthesis of example 18Bb, 2-(2-Fluoro-6-chloro-phenyl)-1-morpholin-2-yl-1-phenyl-ethanol hydrochloride, was followed making non-critical variations to yield the title compound. ¹H NMR (300 MHz, CD₃OD D4) δ: 2.48-2.59 (d, 1H), 2.87-3.09 (m, 2H), 3.11-3.17 (m, 2H), 3.26-3.38 (m, 1H), 3.81-3.95 (t, 1H), 4.02-4.11 (d, 1H), 4.13-4.25 (d, 1H), 6.48-6.60 (m, 2H), 7.70-6.98 (m, 1H) 7.08-7.28 (m, 5H). LCMS (12 minutes method) [M+H]⁺=320@Rt 4.20 min. major peak.

### EXAMPLE 21B

### Preparation of 2-(2,6-Dichloro-phenyl)-1-morpholin-2-yl-1-phenyl-ethanol hydrochloride

### a) 1-(4-Benzyl-morpholin-2-yl)-2-(2,6-dichloro-phenyl)-1-phenyl-ethanol

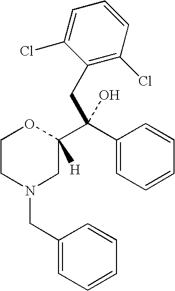

The procedure for the synthesis of example 18Ba, 1-(4-Benzyl-morpholin-2-yl)-2-(2-chloro-6-fluoro-phenyl)-1-phenyl-ethanol, using 2,6-dichlorobenzyl magnesium chloride as starting material (available from Rieke Metals) was followed making non-critical variations, to yield the title compound. This material was used in step b) without further purification. LCMS (6 minutes method) [M+H]⁺=442@Rt 3.49 min. major peak.

### b) 2-(2,6-Dichloro-phenyl)-1-morpholin-2-yl-1-phenyl-ethanol hydrochloride

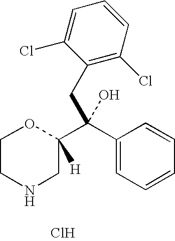

To a solution of 1-(4-Benzyl-morpholin-2-yl)-2-(2,6-dichloro-phenyl)-1-phenyl-ethanol (450 mg, 1 equiv.) in ethyl acetate (15 mL) at room temperature under nitrogen atmosphere was added ammonium formate (1.69 g, 25 equiv.) followed by addition of palladium on charcoal (10%, 45mg.). The reaction mixture was heated to reflux for 3 hour, cooled to room temperature and then filtered through Celite. All volatiles were evaporated under vacuum, and the resulting solid was purified via preparative HPLC. The isolated white solid was taken up in ethanol. Hydrogen chloride was added (large excess of 2M solution in diethyl ether) and the mixture was stirred until it became a clear solution. Then all the volatiles were evaporated “in vacuo”, to give 60 mg of the title compound as white solid.¹H NMR (300 MHz, CD₃OD D4) δ: 2.52-2.61 (d, 1H), 2.79-2.96 (t, 1H), 2.98-3.13 (t, 1H), 3.15-3.19 (s, 1H), 3.56-3.71 (q, 2H), 3.88-4.02 (t, 1H), 4.10-4.21 (d, 1H), 4.29-4.39 (d, 1H), 6.97-7.08 (m, 1H), 7.10-7.21 (m, 7H). LCMS (12 minutes method) [M+H]⁺=352@Rt 4.63 min. single major peak.

### EXAMPLE 22B

### Preparation of 2-(2,5-Dichloro-phenyl)-1-morpholin-2-yl-1-phenyl-ethanol hydrochloride

### a) 1-(4-Benzyl-morpholin-2-yl)-2-(2,5-dichloro -phenyl)-1-phenyl-ethanol

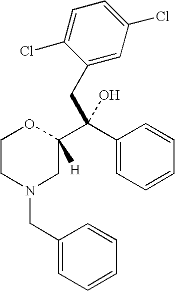

The procedure for the synthesis of example 18Ba, 1-(4-Benzyl-morpholin-2-yl)-2-(2-chloro-6-fluoro-phenyl)-1-phenyl-ethanol, using 2,5-dichlorobenzyl magnesium chloride as starting material (available from Rieke Metals) was followed making non-critical variations, to yield the title compound. This material was used in step b) without further purification. LCMS (6 minutes method) [M+H]⁺=442@Rt 3.48 min. major peak.

### b) 2-(2,5-Dichloro-phenyl)-1-morpholin-2-yl-1-phenyl-ethanol hydrochloride

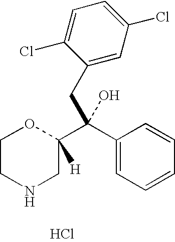

The procedure for the synthesis of example 21Bb, 1-(4-Benzyl-morpholin-2-yl)-2-(2,6-dichloro-phenyl)-1-phenyl-ethanol, was followed making non-critical variations to the title compound. ¹H NMR (300 MHz, CD₃OD D4) δ: 2.49-2.61 (d, 1H), 2.88-3.11(m, 2H), 3.12-3.24 (m, 1H), 3.24-3.35 (m, 1H), 3.41-3.53 (d, 1H), 3.82-3.96 (m, 1H), 4.04-4.25 (m, 2H), 6.90-7.00 (m, 1H), 7.02-7.29 (m, 7H). LCMS (12 minutes method) [M+H]⁺=352(Rt 4.86 min. major peak

### EXAMPLE 23B

### Preparation of 2-(2,5-Difluoro-phenyl)-1-morpholin-2-yl-1-phenyl-ethanol hydrochloride

### a) 1-(4-Benzyl-morpholin-2-yl)-2-(2,5-difluoro -phenyl)-1-phenyl-ethanol

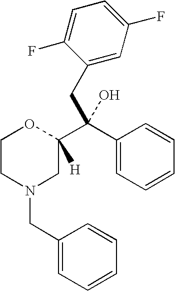

The procedure for the synthesis of example 18Ba, 1-(4-Benzyl-morpholin-2-yl)-2-(2-chloro-6-fluoro -phenyl)-1-phenyl-ethanol, using 2,5-difluorobenzyl magnesium bromide as starting material (available from Rieke Metals) was followed making non-critical variations, to yield the title compound. This material was used in step b) without further purification. LCMS (6 minutes method) [M+H]⁺=410@Rt 3.11 min. major peak.

### b) 2-(2,5-Difluoro-phenyl)-1-morpholin-2-yl-1-phenyl-ethanol hydrochloride

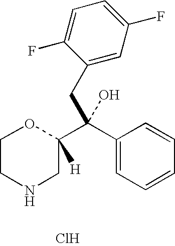

The procedure for the synthesis of example 18Bb, 2-(2-Fluoro-6-chloro-phenyl)-1-morpholin-2-yl-1-phenyl-ethanol hydrochloride, was followed making non-critical variations, to yield the title compound. H NMR (300 MHz, CD₃OD D4) δ: 2.48-2.59 (d, 1H), 2.87-3.09 (m, 2H), 3.11-3.17 (m, 1H), 3.26-3.38 (m, 2H), 3.81-3.95 (t, 1H), 4.02-4.11 (d, 1H), 4.13-4.25 (d, 1H), 6.62-6.77 (m, 3H), 7.08-7.28 (m, 5H). LCMS (12 minutes method) [M+H]⁺=320@Rt 4.20 min. single major peak.

### EXAMPLE 24B

### Preparation of 2-(2-Fluoro-5-phenyl-phenyl)-1-morpholin-2-yl-1-phenyl-ethanol hydrochloride

### a) 1-(4-Benzyl-morpholin-2-yl)-2-(-2-biphenyl-5-flouro-phenyl)-1-phenyl-ethanol

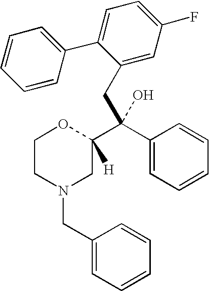

The procedure for the synthesis of example 18Ba, 1-(4-Benzyl-morpholin-2-yl)-2-(2-chloro-6-fluoro -phenyl)-1-phenyl-ethanol, using 2-phenyl-5-fluorobenzyl magnesium bromide as starting material was followed making non-critical variations, to yield the title compound. This material was used in step b) without further purification. LCMS (6 minutes method) [M+H]⁺=468@Rt 3.62 min. major peak.

### b) 2-(2-Fluoro-5-phenyl-phenyl)-1-morpholin-2-yl-1-phenyl-ethanol hydrochloride

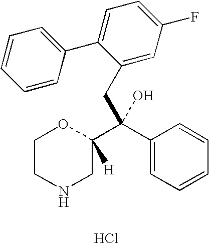

The procedure for the synthesis of example 18Bb, 2-(2-Fluoro-6-chloro-phenyl)-1-morpholin-2-yl-1-phenyl-ethanol hydrochloride, was followed making non-critical variations to the title compound. ¹H NMR (300 MHz, CD₃OD D4) δ: 2.35-2.48 (d, 1H), 2.77-2.91 (t, 1H), 2.91-3.04 (m, 1H), 3.04-3.16 (m, 1H), 3.22-3.28 (m, 1H), 3.30-3.42 (m, 1H), 3.66-3.87 (m, 2H), 4.01-4.14 (d, 1H), 6.70-6.89 (m, 5H), 6.98-7.11 (m, 4H), 7.14-7.25 (m, 4H). LCMS (12 minutes method) [M+H]⁺=378@Rt 5.22 min. major peak.

Solid Phase Synthesis of Compounds of Formulae (IB)

Compounds of the invention wherein Ar₁ is substituted with an aromatic group (i.e., pyridyl, thiophenyl, and optionally substituted phenyl) can be prepared by solid phase synthesis using the route shown below (the black dot represents polystyrene resin).

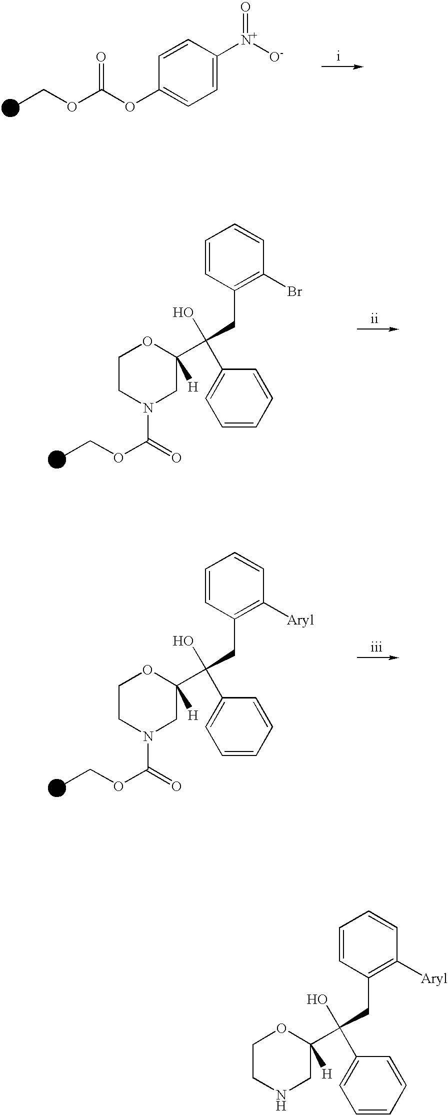

The sequence is preferably performed on a polystyrene resin, without characterization of the resin-bound intermediates.
i) Aliquots (52 mg, 0.05 mmoles) of p-nitrophenyl carbonate resin (Novabiochem) were dispensed into 4.5 ml MiniBlock reaction tubes (Mettler-Toledo). To each resin was added DMF (0.5 ml) followed by a 0.2M solution of 2-(2-bromo-phenyl)-1-morpholin-2-yl-1-phenyl-ethanol in DMF (0.5 ml, 0.1 mmoles). The tubes were sealed and agitated by orbital shaking for 24 hrs. The resins were then filtered and washed with DMF (3×1.0 ml), a solution of diisopropylethylamine (0.25 ml) in DMF (1.0 ml) and finally DMF (4×1.0 ml).
ii) To each resin was added a 2M solution of an optionally substituted aryl boronic acid in DMF (0.5 ml, 1.0 mmoles), a 0.5M solution of triphenylphosphine in DMF (0.2 ml, 0.1 mmoles), a 0.25M solution of Pd(II) acetate in DMF (0.2 ml, 0.05 mmoles) and a 1.25M solution of caesium carbonate in water (0.1 ml, 0.125 mmoles). The tubes were sealed, agitated by orbital shaking and heated at 80° for 20 hrs. The reactions were then cooled to ambient temperature and the resins washed with DMF (2×1.0 ml), MeOH (3×1.0 ml) and DCM (4×1.0 ml).
iii) To each resin was added a TFA/H₂O mixture (95:5 v/v, 1 ml). The tubes were sealed and agitated by orbital shaking for 6 hrs. The reactions were filtered and washed with DCM (2×2 ml). Appropriate filtrates and washings were combined and volatile components removed by vacuum evaporation. Each residue was dissolved in MeOH (1 ml) and the solutions applied to MeOH-washed SCX-2 cartridges (0.5 g/3.0 ml) (Jones Chromatography). After draining under gravity the cartridges were washed with MeOH (2.5 ml) and the products then eluted using a 2M solution of ammonia in MeOH (2.5 ml). Removal of volatile components by vacuum evaporation gave the desired products which were purified by preparative LCMS.

By this means were prepared:

### EXAMPLE 25B

### 2-(4′-methyl-biphenyl-2-yl)-1-morpholin-2-yl-1-phenyl-ethanol, RT (6 min gradient) 3.11 min, [M+H]⁺ 374.2

### EXAMPLE 26B

### 2-(4′-chloro-biphenyl-2-yl)-1-morpholin-2-yl-l -phenyl-ethanol, RT (6 min gradient) 3.36 min, [M+H]⁺ 394.2

### EXAMPLE 27B

### 2-(4′-methoxy-biphenyl-2-yl)-1-morpholin-2-yl-1-phenyl-ethanol, RT (6 min gradient) 3.37 min, [M+H]⁺ 390.2

### EXAMPLE 28B

### 2-(3′-fluoro-biphenyl-2-yl)-1-morpholin-2-yl-1-phenyl-ethanol, RT (6 min gradient) 3.39 min, [M+H]⁺ 378.4

### EXAMPLE 29B

### 2-(3′-chloro-biphenyl-2-yl)-1-morpholin-2-yl-1-phenyl-ethanol, RT (6 min gradient) 3.53 min, [M+H]⁺ 394.4

### EXAMPLE 30B

### 2-(3′-methoxy-biphenyl-2-yl)-1-morpholin-2-yl-1-phenyl-ethanol, RT (6 min gradient) 3.31 min, [M+H]⁺ 390.4

### EXAMPLE 31B

### 2-(3′-methyl-biphenyl-2-yl)-1-morpholin-2-yl-1-phenyl-ethanol, RT (6 min gradient) 3.45 min, [M+H]⁺ 374.4

### EXAMPLE 32B

### 2-(3′,5′-dichloro-biphenyl-2-yl)-1-morpholin-2-yl-1-phenyl-ethanol, RT (6 min gradient) 3.71 min, [M+H]⁺ 428.3

### EXAMPLE 33B

### 2-(2′,4′-dimethyl-biphenyl-2-yl)-1-morpholin-2-yl-1-phenyl-ethanol, RT (6 min gradient) 3.59 min, [M+H]⁺ 388.4

### EXAMPLE 34B

### 2-(2′,4′-dimethoxy-biphenyl-2-yl)-1-morpholin-2-yl-1-phenyl-ethanol, RT (6 min gradient) 3.33 min, [M+H]⁺ 420.4

### EXAMPLE 35B

### 1-morpholin-2-yl-1-phenyl-2-(2-pyridin-3-yl-phenyl)-ethanol, RT (6 min gradient) 2.17 min, [M+H]⁺ 361.4

### EXAMPLE 36B

### 1-morpholin-2-yl-1-phenyl-2-(2-thiophen-3-yl-phenyl)-ethanol, 3.25 min, [M+H]⁺ 366.4

### EXAMPLE 37B

### 2-(3′,4′-dichloro-biphenyl-2-yl)-1-morpholin-2-yl-1-phenyl-ethanol, RT (6 min gradient) 3.56 min, [M+H]⁺ 428.1

The following examples illustrate compounds of of Formulae (IC) above and methods for their preparation.

### General Synthetic Procedures for the Preparation of Examples IC-17C

The numbers included in the following Sections refer to the compounds illustrated in Schemes 2C to 6C herein.

General Procedure 1C: Preparation of Racemic N-Substituted Aryl Thiols

To a solution of 5Ca,5Cb (0.02 g, 0.52 mmol) and the requisite aryl thiol (1.1 eq) in anhydrous dimethylformamide (1 ml) at room temperature under nitrogen was added cesium carbonate (1.1 eq, 0.19 g, 0.57 mmol). The reaction mixture was heated to 95° C. for 2 hours. The reaction mixture was allowed to cool to room temperature, diluted with ethyl acetate, then washed sequentially with water, brine, dried over magnesium sulphate and finally concentrated in vacuo.

General Procedure 2Ca: Deprotection of N-Substituted Aryl Thiols

To a solution of the requisite N-benzyl aryl thiol in anhydrous dichloromethane (5 ml) was added solid supported Hünig's base (Argonaut, 3.56 mmol/g, 2 eq) and a-chloroethyl chloroformate (3 to 1 0 eq) at room temperature under nitrogen. The reaction mixture was heated to 40° C. and followed by LCMS analysis. After completion the reaction mixture was filtered, and the resin washed with dichloromethane. The combined organic phases were concentrated in vacuo. Methanol (HPLC grade, 25 ml) was added and the solution heated to 60° C. for 1.5 to 4 hours. After complete consumption of starting material the methanol solution was evaporated to give a solid which was further purified as detailed for individual compounds.

General Procedure 2Cb: Deprotection of N-Substituted Aryl Thiols

To a solution of the requisite N-benzyl aryl thiol (1 eq) in ethyl acetate at room temperature was added phenylchloroformate (3 eq). The mixture was warmed under reflux for 2 hours. The mixture was then cooled to room temperature and 30% NaOH with water was added over 1 hour. The biphasic system was stirred for 1.5 hours at room temperature and the organic layer was separated. The organic layer was washed with water, dried over MgSO₄, filtered and rinsed with ethyl acetate.

To the mixture of carbamate and benzylchloride in ethyl acetate was added 5.6M dimethylamine in ethanol. The solution was warmed under reflux (70-72° C.) for 2 hours. After cooling at room temperature, water and 12N HCl were added and the mixture was stirred for 10 minutes. The layers were separated and the organic phase was washed twice with water. Then the organic layer was concentrated (T=50° C.) until crystallization. MeOH was added and approx. 40% of solvent was then removed under reduce pressure, this operation was repeated. The heterogeneous mixture was stirred for 0.5 hours at room temperature and filtered. The precipitate was washed twice with MeOH and dried under reduce pressure at 40° C. to yield the carbamate.

To a biphasic mixture of 30% NaOH and isopropanol warmed to 65° C., was added the carbamate. The heterogeneous mixture was warmed under reflux for 4 hours and then cooled to room temperature and post-agitated overnight. The organic layer was concentrated under reduce pressure and the yellow solid obtained was added to a mixture of AcOEt and 1N NaOH. After separation of the layers, the organic one was washed with 1N NaOH. The aqueous layers were combined and extracted with AcOEt. The combined organic layers were dried over MgSO₄, filtered and concentrated under reduce pressure to dryness to obtain the free amine.

General Procedure 3C: Conversion of Amines Into Hydrochloride Salts

To a solution of the requisite amine in dry diethyl ether (1 ml) was added hydrochloric acid (500 μl of a 1M solution in diethyl ether). A white precipitate immediately formed. The suspension was then sonicated for 5 minutes. Ether was blown off with a stream of nitrogen and the samples were dried under high vacuum for several hours to give the hydrochloride salts in near quantitative yield as white solids.

General Procedure 4C: Aldoladdition With Substituted Benzaldehydes

Preparation of 38Ca,38Cb: 39Ca,39Cb: 40Ca,40Cb

N-Benzylmorpholinone (1.0 eq) and the requisite aldehyde (1.1 eq) were dissolved in anhydrous tetrahydrofuran (25 ml) under nitrogen and the reaction cooled to −78° C. Then, lithium diisopropylamide (1.1 eq of a 2M solution in heptane/tetrahydrofuran/ethylbenzene) was added over approximately 20 minutes, whilst maintaining the reaction temperature below −78° C. The resulting yellow solution was stirred at −78° C. for 1 hour and then allowed to warm to room temperature. The reaction was quenched with saturated ammonium chloride solution (25 ml) and extracted into ethyl acetate. The combined organic layers were dried with magnesium sulphate, filtered and concentrated in vacuo, to give a yellow oil which was purified by column chromatography on silica gel (eluent: ethyl acetate/hexane 70/100 [v/v]).

General Procedure 5C: Reduction of Substituted Aldol Adducts

Preparation of 41Ca,41Cb: 42Ca,42Cb; 43Ca,43Cb

To a solution of the requisite amide 38Ca,38Cb, 39Ca,39Cb or 40Ca,40Cb (1.1 mmol) in anhydrous tetrahydrofuran under nitrogen at room temperature was slowly added borane (4 eq of a 1M solution in tetrahydrofuran). The solution was stirred at 60° C. for 2 hours. The reaction was cooled to room temperature; dry methanol (excess) was slowly added, followed by aqueous hydrochloric acid solution (1M, excess). The reaction mixture was heated to 60° C. for 1 hour and quenched with aqueous potassium carbonate solution (1M, excess) and extracted with diethyl ether. The combined organic layers were washed with brine, dried with magnesium sulphate, filtered and concentrated in vacuo yielding a yellow oil which was purified by column chromatography on silica gel (eluent: ethyl acetate/hexane 10/100 [v/v]).

### Preparation of Intermediates for the Synthesis of Examples 1C-17C

### 4-Benzylmorpholin-3-one (2C)

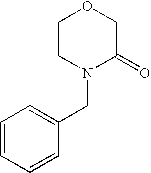

A solution of N-benzyl-N-(2-hydroxyethyl) chloroacetamide (627.7 g, 2.76 mol) in tert-butanol (0.9 l) was stirred under nitrogen while warming to 25-30° C. Potassium tert-butoxide (2.897 l of a 1M solution in tert-butanol, 2.90 mol, 1.05 eq) was added over 2 hours. The reaction mixture was then stirred at room temperature for 90 minutes. Ice-cold water (6 l) was added and the resultant cloudy solution extracted with ethyl acetate. The combined organic layers were washed with brine, dried over magnesium sulphate and evaporated in vacuo to give a light brown oil (441 g, 84%), which was used in the next stage without further purification; MW 191.23; C₁₁H₁₃NO₂; ¹H NMR (CDCl₃): 7.29-7.40 (5H, m), 4.67 (2H, s), 4.28 (2H, s), 3.87 (2H, t, 5 Hz), 3.31 (2H, t, 5 Hz); LCMS: (12 min method) m/z 192 [M+H]+@Rt 1.00 min.

### (2S)-(4-Benzyl-morpholin-2-yl)-phenyl-methanone (3Ca) and (2R)-(4-Benzyl-morpholin-2-yl)-phenyl-methanone (3Cb)

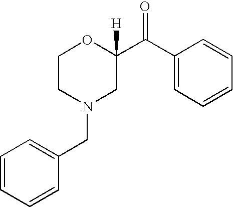

Described above under the “Synthesis of Intermediates” section for compounds of Formula (IB).

### (S)-Phenyl[(2S)-4-(phenylmethyl)morpholin-2-yl]methanol (4Ca)

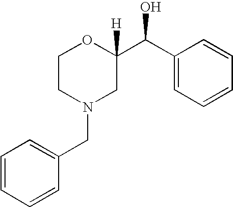

To a stirred solution of [(−)-B-chlorodisopinocampheylborane] (45 g, 140 mmol) in dry tetrahydrofuran (300 ml) under nitrogen was added 3Ca (7.97 g, 28.4 mmol) in one portion. The reaction mixture was stirred at room temperature for 18 hours. The mixture was evaporated in vacuo and extracted from 2M aqueous sodium hydroxide solution into ethyl acetate. The combined organic extracts were washed with brine, dried, filtered and evaporated. The crude product was taken up in chloroform/methanol (1:1 [v/v]) and absorbed onto 150 g SCX-2 ion exchange resin. After elution of borane residues with methanol the product was eluted with 2M ammonia in methanol. Removal of solvent in vacuo yielded the product as yellow oil. This was further purified by flash chromatography (eluent: ethyl acetate/isobexane 80/20 [v/v]). After removal of solvents, the product crystallised on standing (6.73 g, 84%); MW 283.37; C₁₈H₂₁NO₂; ¹H NMR (CDCl₃): 7.32-7.45 (10H, m), 4.67 (1H, d, 7 Hz), 4.03 (1H, dt, 11 Hz and 2 Hz), 3.86-3.73 (2H, m), 3.64 (1H, d, 13 Hz), 3.39 (1H, d, 13 Hz), 3.30 (1H, br, s), 2.68 (1H, d, 12 Hz), 2.56 (1H, d, 10 Hz), 2.28-2.15 (2H, m); LCMS: m/z 284 [M+H]+@Rt 0.95 min.

### (2S)-2-[(R)-bromo(phenyl)methyl]-4-(phenylmethyl)morpholine (5Ca)

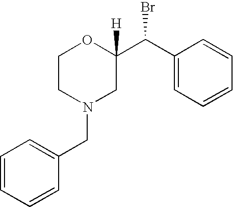

To a solution of 4Ca (4.71 g, 16.6 mmol) in anhydrous chloroform (200 ml) under nitrogen was added triphenylphosphine dibromide (14.04 g, 33.26 mmol). The reaction mixture was heated at 60° C. overnight. The mixture was allowed to cool to room temperature then washed with saturated aqueous sodium carbonate solution, dried over sodium sulphate and concentrated in vacuo. The resulting residue was purified by flash chromatography on silica (eluent: ethyl acetate/isohexane gradient 10/90 to 30/70 [v/v]) to give 5Ca as a white solid (4.63 g, 81%); MW 346.27; C₁₈H₂₀BrNO; ¹H NMR (CDCl₃): 7.14-7.39 (10H, m), 4.83 (1H, d, 7 Hz), 4.01 (1H, br, t, 8 Hz), 3.73 (1H, br, d, 11 Hz), 3.60-3.48 (2H, m), 3.39 (1H, d, 12 Hz), 3.20 (1H, d, 11 Hz), 2.50 (1H, d, 10 Hz), 2.07 (2H, t, 10 Hz); LCMS: (6 min method) m/z 346 [M]+@Rt 2.51 min.

### (2S)-2-[(S)-Hydroxy(phenyl)methyl]-4-(phenylmethyl)morpholin-3-one (6Ca) and

### (2S)-2-1(R)-Hydroxy(phenyl)methyl]-4-(phenylmethyl)morpholin-3-one (6Cb) and

### (2R)-2-[(S)-Hydroxy(phenyl)methyl]-4-(phenylmethyl)morpholin-3-one (6Cc) and

### (2R)-2-[(R)-Hydroxy(phenyl)methyl]-4-(phenylmethyl)morpholin-3-one (6Cd)

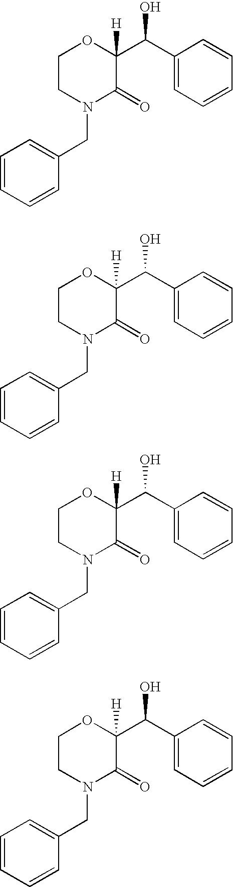

To a stirred solution of 2C (5.02 g, 26 mmol) in anhydrous tetrahydrofuran (25 ml) under nitrogen at −78° C. was added lithium diisopropylamide (1.5 eq, 39 mmol, 19.5 ml of a 2M solution in heptane/tetrahydrofuran/ethylbenzene) over approximately 20 minutes, whilst maintaining the reaction temperature below −75° C. The resulting brown solution was stirred for a further 30 minutes at −78° C., before being added over approximately 30 minutes to a solution of benzaldehyde (1.2 eq, 3.29 g, 31 mmol) in anhydrous tetrahydrofuran (15 ml) under nitrogen at −78° C., whilst again maintaining the reaction temperature below −75° C. The resulting yellow solution was stirred at −78° C. for 1 hour, before being allowed to warm to room temperature slowly over 1 hour. The reaction mixture was cautiously quenched by addition of saturated ammonium chloride solution (50 ml) and the tetrahydrofuran was evaporated in vacuo. The resulting cloudy aqueous solution was extracted with dichloromethane, and the organic extracts were combined, washed with brine, dried over sodium sulphate and the dichloromethane evaporated in vacuo to give a thick brown oil (9.2 g), which partially crystallised on standing. After purification by flash column chromatography (eluent: ethyl acetate/dichloromethane 10/90 to 20/80 gradient [v/v]) 6Ca,6Cb was obtained as light red crystals (2.46 g, 32%); MW 297.36; C₁₈H₁₉NO₃; ¹H NMR (CDCl₃): 7.36-7.41 (2H, m), 7.16-7.31 (6H, m), 6.86-6.91 (2H, m), 5.14 (1H, d, J 3 Hz), 4.71 (1H ,d, 14 Hz), 4.48 (1H, d, J 3 Hz), 4.25 (1H, d, 14 Hz), 4.20 (1H, br, s), 3.89 (1H, ddd, 12 Hz, 3 Hz, 2 Hz), 3.67 (1H, dt, 11 Hz, 3 Hz), 3.16 (1H, dt, 12 Hz and 4 Hz), 2.86 (1H, br, d, 12 Hz); LCMS: m/z 298 [M+H]+@Rt 1.24 min. 6Cc, 6Cd was isolated as a brown solid (1.42 g) contaminated with 2C. Trituration with ethyl acetate afforded pure 6Cc,6Cd as a white solid (0.484 g, 6%); MW 297.36; C₁₈H₁₉NO₃; ¹H NMR (CDCl₃): 7.55-7.61 (2H, m), 7.36-7.50 (6H, m), 7.25-7.31 (2H, m), 5.21 (1H, d, 2 Hz), 5.09 (1H, d, J 7 Hz and 2 Hz), 4.73 (2H, s), 4.37 (1H, d, J 8 Hz), 4.01 (1H, ddd, 12 Hz, 3 Hz, 2 Hz), 3.77 (1H, dt, 11 Hz, 4 Hz), 3.50 (1H, dt, 12 Hz, 4 Hz), 3.16 (1H, br, d, 12 Hz); LCMS: m/z 298 [M+H]+@Rt 1.24 min.

### (S)-Phenyl[(2S)-4-(phenylmethyl)morpholin-2-yl]methanol (4Ca) and

### (R)-Phenyl[(2R)-4-(phenylmethyl)morpholin-2-yl]methanol (4Cb)

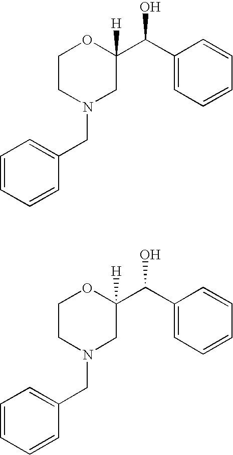

To a solution of 6Ca,6Cb (0.033 g, 1.1 mmol) in anhydrous THF (5 ml) under nitrogen at room temperature was slowly added borane (4 eq, 4.4 ml of a 1 M solution in tetrahydrofuran, 4.4 mmol). The solution was stirred at 60° C. for 2 hours. After cooling down to room temperature, dry methanol (2 ml) was slowly added to quench excess borane reagent. After addition of aqueous hydrochloric acid solution (2 ml of a 1M solution) the reaction mixture was heated to 60° C. for 1 hour. The organic solvents were evaporated in vacuo and the concentrated solution was poured onto aqueous potassium carbonate solution (10 ml of a 1M solution) and extracted with diethyl ether (2×20 ml). The combined organic layers were washed with brine, water, dried over magnesium sulphate and concentrated in vacuo. Purification by flash column chromatography (eluent: hexane/ethyl acetate/triethylamine 90/9/1 [v/v/v]) gave a viscous oil (0.19 g, 60%); MW 283.37; C₁₈H₂₁NO₂; ¹H NMR (CDCl3): 7.45-7.32 (10H, m), 4.67 (1H, d, 7 Hz), 4.03 (1H, dt, 11 Hz, 2.7 Hz), 3.86-3.73 (2H, m), 3.64 (1H, d, 13 Hz), 3.39 (1H, d, 13 Hz), 3.30 (1H, br, s), 2.68 (1H, d, 13 Hz), 2.56 (1H, d, 11 Hz), 2.28-2.15 (2H, m); LCMS: m/z 284 [M+H]+@Rt 0.95 min.

### (R)-[(2S)4-Benzylmorpholinyl](phenyl)methanol (4Cc) and

### (S)-[(2R)-4-Benzylmorpholinyl](phenyl)methanol (4Cd)

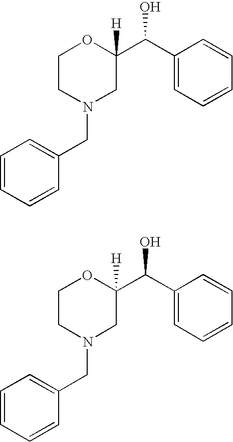

Using the procedure described for the preparation of 4Ca,4Cb starting from 6Cc,6Cd (0.14 g, 0.45 mmol) 4Cc,4Cd was obtained as a viscous oil (0.098 g, 68%); MW 283.37; C₁₈H₂₁NO₂; ¹H NMR (CDCl₃): 7.17-7.28 (10H, m), 4.80 (1H, d, 4 Hz), 3.88 (1H, dt, 11 Hz, 3 Hz), 3.72 (1H, m), 3.61-3.68 (1H, m), 3.50 (1H, d, 13 Hz), 3.25 (1H, d, 13 Hz), 2.52 (2H, br, t, 12 Hz), 2.17 (1H, t, 11 Hz), 2.08 (1H, td, 11 Hz, 3 Hz); LCMS: m/z 284 [M+H]+@Rt 0.98 min.

### (2S)-2-[(R)-Bromo(phenyl)methyl]-4-(phenylmethyl)morpholine (5Ca) and

### (2R)-2-[(S)-Bromo(phenyl)methyl]-4-(phenylmethyl)morpholine (5Cb)

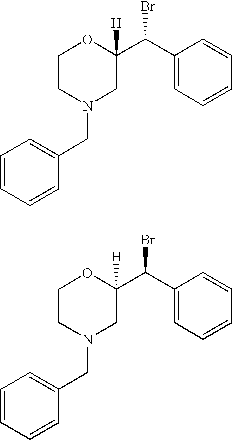

To a solution of 4Ca,4Cb (10.27 g, 36.29 mmol) in anhydrous dichloromethane (150 ml) under nitrogen at room temperature was added freshly recrystallised triphenylphosphine (13.32 g, 50.80 mmol, 1.4 eq) followed by carbon tetrabromide (16.85 g, 50.8 mmol, 1.4 eq) as a solution in anhydrous dichloromethane (50 ml). After 15 minutes the reaction mixture was diluted with dichloromethane (100 ml) and washed with saturated aqueous solution of sodium hydrogencarbonate, brine, dried over magnesium sulphate and concentrated in vacuo to give an orange oil (42.0 g). To the orange oil was added diethyl ether (200 ml) and the resulting suspension was sonicated for 30 minutes. The solvent was decanted and the process repeated with a further portion of diethyl ether. The combined organic extracts were concentrated in vacuo to yield an orange solid (22.0 g) which was purified by flash column chromatography (eluent: ethyl acetate/hexane/triethylamine 10/89.5/0.5 [v/v/v]) 5Ca,5Cb was otained as a white solid (7.20 g, 57%). Alternative Work-up: The reaction mixture was poured onto a silica (160 g) filtration pad which was washed with dichloromethane (14×250 ml). After removal of solvents in vacuo and purification by flash column chromatography (eluent: ethyl acetate/hexane/triethylamine gradient 5/94.5/0.5 to 10/89.5/0.5 [v/v/v]) to give a white solid (6.05 g, 48%); MW 346.27; C₁₈H₂₀BrNO; ¹H NMR (CDCl₃): 7.14-7.39 (10H, m), 4.83 (1H, d, 7 Hz), 4.01 (1H, br, t, 8 Hz), 3.73 (1H, br, d, 11 Hz), 3.48-3.60 (2H, m), 3.39 (1H, d, 12 Hz), 3.20 (1H, d, 11 Hz), 2.50 (1H, d, 10 Hz), 2.07 (2H, t, 11 Hz); LCMS: m/z 348/346 [M+H]+@Rt 1.20 min.

### 4-[(1R)-1-Phenylethyl]morpholine-(2S)-carbonitrile (47Ca) and

### 4-[(1R)-1-Phenylethyl]morpholine-(2R)-carbonitrile (47Cb)

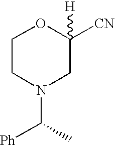

To (R)-(−)-2-hydroxyethyl-a-phenethylamine (1.65 g, 10.0 mmol) in diethyl ether (10 ml) was added at room temperature 2-chloroacrylonitrile (0.80 ml, 10.0 mmol) with stirring. The mixture was stirred at room temperature for 4.5 days when additional 2-chloroacrylonitrile (0.8 ml, 10.0 mmol) was added. After stirring another 3.5 days, the reaction mixture was concentrated in vacuo to give an oil. The oil was dissolved in dry tetrahydrofuran (30 ml), cooled under nitrogen to 0° C. and potassium tert-butoxide (1.23 g, 11.0 mmol) added. The solution was stirred at 0° C. for 2 hours then at reflux for 1.5 hours, cooled, diluted with diethyl ether and washed with aqueous saturated sodium bicarbonate. The organic phase was extracted with 2N hydrochloric acid and the aqueous made basic by addition of solid sodium bicarbonate and extracted with diethyl ether. The organic phase was dried over magnesium sulphate, filtered and evaporated to a brown oil. The crude product was purified by flash chromatography (eluent: ethyl acetate/hexane gradient 100% ethyl acetate to 50/50 [v/v]) to give 47Ca,47Cb as a colourless oil (0.58 g, 27%%); MW 216.29; C₁₃H₁₆N₂O; ¹H NMR (CDCl₃) 7.25-7.38 (5H, m), 4.6 (1H, dd), 4.54 (1H, dd), 3.91-4.06 (2H, m), 3.66-3.82 (2H, m), 3.39-3.49 (2H, m), 2.30-2. 89 (4H, m), 1.39 (3H, d). m/z [M+H]⁺ 217.

### Phenyl{(2S)-4-[(1R)-1-phenylethyl]morpholin-2-yl}methanone (48Ca) and

### Phenyl{(2R)-4-[(1R)-1-phenylethyl]morpholin-2-yl}methanone (48Cb)

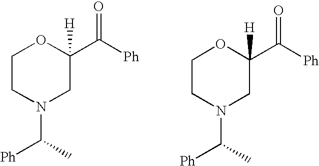

To a stirred solution of 47Ca,47Cb (0.57 g, 2.64 mmol) in dry tetrahydrofurane (10 ml) at 0° C. under nitrogen was added a solution of phenylmagnesium chloride in tetrahydrofurane (2.0 M, 2.67 ml) dropwise over 2 minutes. The pale yellow solution was stirred at 0° C. for 30 minutes and then allowed to warm to room temperature. After 2 hours the mixture was cooled, quenched with 2M hydrochloric acid and was stirred vigorously for 1 hour at room temperature. After addition of water and extraction with ethyl acetate, the combined organic layers were washed with brine, dried over magnesium sulphate, filtered and evaporated to give an oil (0.63 g). After purification by column chromatography (eluent: ethyl acetate/hexane gradient 0/100 to 20/80 [v/v]) 48Ca was obtained as an oil (0.15 g, 19%%); MW 295.38; C₁₉H₂₁NO₂; ¹H NMR (CDCl₃) 8.00 (2H, d), 7.60 (1H, t), 7.50 (2H, t), 7.20-7.35 (5H, m), 4.96 (1H, d), 3.93-4.00 (1H, m), 3.70-3.80 (1H, m), 3.41 (1H, q), 3.25 (1H, br, d), 2.59 (1H, br, d), 2.13-2. 36 (2H, m), 1.38 (3H, d). m/z [M+H]⁺ 296 followed by 48Cb as an oil (0.27 g, 35%%) ¹H NMR (CDCl₃) 7.90 (2H, d), 7.54 (1H, t), 7.45 (2H, t), 7.20-7.38 (5H, m), 4.85 (1H, d), 4.05-4.12 (1H, m), 3.80-3.92 (1H, m), 3.43 (1H, q), 2.86-3.00 (2H, m), 2.29-2.40 (1H, m), 2.21 (1H, t), 1.38 (3H, d). m/z [M+H]⁺ 296.

### (R)-Phenyl{(2S)-4-[(1R)-1-phenylethyl]morpholin-2-yl}methanol (50C)

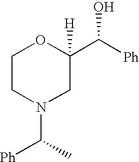

To a stirred solution of 48Ca (0.08 g, 0.26 mmol) and triphenylsilane (0.34 g, 1.31 mmol) in dichloromethane (4 ml) cooled to 0° C. was added boron trifluoride etherate (0.09 g, 0.66 mmol) followed by trifluoroacetic acid (0.36 ml, 63 mmol). The reaction mixture was allowed to warm to room temperature and diluted after three hours with dichloromethane (20 ml) and neutralised with aqueous sodium bicarbonate. The organic phase was dried over magnesium sulphate, filtered and evaporated to give the required product. This was purified as its hydrochloric acid salt crystallising from isopropanol and diethyl ether (0.05 g, 69%%); MW 297.4; C₁₉H₂₃NO₂; ¹H NMR (CDCl₃) on free base 7.08-7.29 (10H, m), 4.78 (1H, d), 3.90-4.00 (1H, m), 3.57-3.68 (2H, m), 3.33 (1H, q), 2.53-2.64 (1H, m), 2.37-2.47 (1H, m), 2.09-2.26 (2H, m), 1.29 (3H, d). m/z [M+H]⁺ 298.

### (R)-Phenyl{(2S)-4-[(1R)-1-phenylethyl]morpholin-2-yl}methyl methanesulphonate (51C)

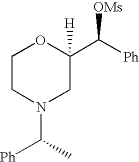

To a solution of 50C (0.05 g, 0.17 mmol) in dichloromethane (1 ml) at room temperature was added polymer supported Hünig's base ((Argonaut, 3.56 mmol/g, 0.089 g, 0.32 mmol, 1.9 eq) and methanesulphonyl chloride (0.02 g, 0.19 mmol). The mixture was stirred under nitrogen for 6 hours then filtered and concentrated in vacuo. The crude product was purified by flash column chromatography (eluent: ethyl acetate/heptane 33/67 [v/v]) to give 51C as a colourless oil (0.035 g, 55%%); MW 375.49; C₂₀H₂₅NO₄S ¹H NMR (CDCl₃) 7.20-7.35 (10H, m), 5.46 (1H, d), 3.79-3.88 (2H, m), 3.59 (1H,td), 3.4 (1H, q), 2.68-2.78 (2H, m), 2.68 (3H, s), 2.03-2.24 (2H, m), 1.34 (3H, d). m/z [M+H]⁺ 376.

### (2S)-4-[(1R)-1-Phenylethyl]-2-((S)-phenyl{[2-(trifluoromethyl)phenyl]thio}methyl)morpholine (52C)

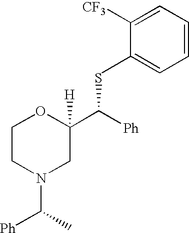

A mixture of 51C (0.035 g, 0.093 mmol), potassium carbonate (0.026 g, 0.19 mmol) and 2-trifluoromethylbenzenethiol (0.084 g, 0.47 mmol) in dry, degassed dimethylformamide (0.5 ml) was stirred under nitrogen at room temperature for 3 days. The reaction mixture was diluted with water and extracted with diethyl ether. The extracts was washed with water and brine, dried over magnesium sulphate, filtered and evaporated to give a colourless oil (0.03 g, 71%). Purification by flash column chromatography (eluent: ethyl acetate/heptane 20/80 [v/v]) gave 52C as a colourless oil (0.03 g, 71%); MW 457.56; C₂₆H₂₆F₃NOS ¹H NMR (CDCl₃) 7.53 (1H, d), 7.10-7.28 (13H, m), 4.39 (1H, d), 3.85-4.04 (2H, m), 3.8 (1H, td), 3.35 (1H, q), 2.70 (1H, d), 2.40 (1H, d), 2.30 (1H, td), 2.10-2.20 (1H, m), 1.29 (3H, d). m/z [M+H]⁺ 458.

### EXAMPLE 1C

### (2S)-2-((S)-Phenyl{[2-(trifluoromethyl)phenyl]thio}methyl)morpholine (9C)

### (S)-Phenyl[(2S)-4-(phenylmethyl)morpholin-2-yl]methyl 2-trifluoromethyl)phenyl sulfide (8C)

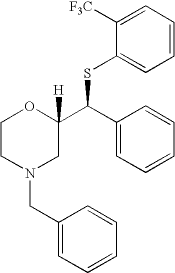

Compound 8C was obtained from 5Ca (4.00 g, 11.55 mmol), 2-trifluoromethyl thiophenol (2.47 g, 13.86 mmol, 1.2 eq) and caesium carbonate (4.95 g, 15.24 mmol, 1.1 eq) in dimethylformamide (60 ml) as a brown oil following a modification of General Procedure 1C in which the reaction was carried out over 1 hour (6.04 g). The oil was purified by flash column chromatography (eluent: hexane/ethyl acetate gradient 100 to 90/10 [v/v]) to give a yellow oil (4.83 g, 94%); MW 443.54; C₂₅H₂₄F₃NOS; ¹H NMR (CDCl₃): 7.60 (1H, dd, 7 Hz, 1 Hz), 7.17-7.39 (13H, m), 4.50 (1H, d, 7 Hz), 3.97-4.12 (2H, m), 3.73 (1H, dt, 10 Hz, 2 Hz), 3.59 (1H, d, 13 Hz), 3.37 (1H, d, 13 Hz), 2.57-2.68 (2H, m); 2.18-2.38 (2H, m); LCMS (2.5 minute method): m/z 445 [M+H]+@Rt 1.50 min.

### (2S)-2-((S)-Phenyl{[2-(trifluoromethyl)phenyl]thio}methyl)morpholine (9C)

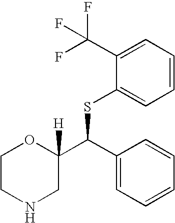

Compound 9C (Example 1C) was obtained from 8C (5.25 g, 11.84 mmol), solid supported Hünig's base (Argonaut, 3.56 mmol/g, 6.64 g, 23.67 mmol, 2 eq) and α-chloroethyl chloroformate (3.83 ml, 35.51 mmol, 3 eq) in anhydrous dichloromethane (75 ml) following General Procedure 2Ca. After evaporation of solvents a light brown solid (5.60 g) was obtained which was recrystallised from iso-propanol. The solid was suspended in ethyl acetate and washed with an aqueous solution of sodium hydroxide (50 ml of a 1M solution). The organic layer was washed with brine, dried over magnesium sulphate and concentrated in vacuo to yield the free amine as a colourless oil (3.10 g, 74%); MW 353.41; C₁₈H₁₈F₃NOS; ¹H NMR (CDCl₃): 7.46 (1H, d, 8 Hz), 7.24 (1H, d, 7 Hz), 7.05-7.2 (7H, m), 4.28 (1H, d, 8 Hz), 3.92 (1H, d, 11 Hz), 3.80 (1H, q, 7 Hz), 3.58 (1H, dt, 2 Hz and 11 Hz), 2.69-2.87 (2H, m), 2.59 (2H, d, 6 Hz), 2.13-1.90 (1H, br s); LCMS (10 minute method): m/z 354 [M+H]+@Rt 5.26 min. The hydrochloride salt of 9 was obtained following General Procedure 3C.

An alternative method for the preparation of compound 9C (Example 1C), according to Scheme 6C, is as follows:

To a suspension of polymer supported Hünig's base (0.11 g, 0.40 mmol) and 52C (0.03 g, 0.066 mmol) in dry dichloromethane (1 ml) was added α-chloroethyl chloroformate (0.09 g, 0.066 mmol) at room temperature under nitrogen. The mixture was stirred at room temperature over the weekend then filtered and concentrated in vacuo. This was taken up in methanol, heated at 70° C. for 2 hours, cooled, and purified by SCX chromatography (eluent: ammonia/methanol 1/1 [v/v]) to give 9C as a colourless oil (0.01 g, 43%). The spectroscopic data for 9C obtained by the route outlined here was identical to the data for 9C obtained as described above.

### EXAMPLE 2C

### (2S)-2-((S)-Phenyl}[2-(thiomethyl)phenyl]thiol}methyl)morpholine (11C)

### (2S)-2-[(S)-{[2-(methylthio)phenyl]thio}(phenyl)methyl]-4-(phenylmethyl)morpholine (10C)

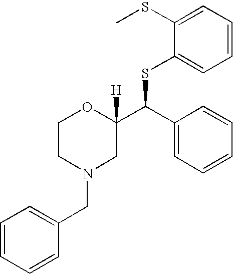

Compound 10C was obtained from 5Ca (4.0 g, 11.55 mmol), 2-methylsulphenyl-thiophenol (2.17 g, 13.86 mmol, 1.2 eq) and caesium carbonate (4.42 g, 13.63 mmol, 1.18 eq) in dimethylformamide (35 ml) following a modification of General Procedure 1C in which the mixture was heated at 50° C. for 1.5 hours, allowed to cool to room temperature, taken up in methanol and treated with SCX-2 (100 g). The SCX-2 was washed with methanol. 10C was obtained as a white solid (4.92 g) after SCX chromatography (eluent: ammonia/methanol 1/1 [v/v]) and removal of solvents in vacuo. Purification by flash column chromatography (eluent: ethyl acetate/isohexane gradient 10/90 to 30/70 [v/v]) gave 10C as a white solid (4.04 g, 83%); MW 421.63; C₂₇H₂₇NOS₂; ¹H NMR (CDCl₃): 7.03-7.15 (6H, m), 6.93-6.99 (2H, m), 6.74 (1H, td, 7 Hz, 1 Hz), 4.31 (1H, d, 8 Hz), 3.95 (1H, br, d, 12 Hz), 3.83 (1H, td, 8 Hz, 3.8 Hz), 3.59 (1H, td, 11 Hz and 3 Hz), 2.82 (1H, td, 12 Hz and Hz), 2.61-2.75 (3H, m), 2.35 (3H, s), 1.73 (1H, br, s); LCMS (6 minute method): m/z 422 [M+H]+@Rt 3.36 min.

### (2S)-2-((S)-Phenyl{[2-(trifluoromethyl)phenyl]thio}methyl)morpholine (11C)

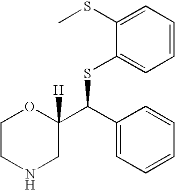

Compound 11C (Example 2C) was obtained from 10C (4.02 g, 9.53 mmol), solid supported Hünig's base (Argonaut, 3.56 mmol/g, 5.02 g, 17.87 mmol, 2 eq) and α-chloroethyl chloroformate (3.09 ml, 28.6 mmol, 3 eq) in anhydrous dichloromethane (75 ml) following General Procedure 2Ca. The mixture was heated at 40° C. for 1.5 hours then left to stir at room temperature overnight. The reaction mixture was filtered and concentrated in vacuo to give a pale orange liquid. This was taken up in methanol (70 ml) and heated at 40° C. for 2 hours. A white solid crashed out of the solution which was taken up in methanol and purified by SCX chromatography (eluent: ammonia/methanol 1/1 [v/v]). After evaporation in vacuo 11C was obtained as a pale yellow oil (3.13 g, 99%); MW 331.50; C₁₈H₂₁NOS₂; ¹H NMR (CDCl₃): 7.03-7.15 (6H, m), 6.93-6.99 (2H, m), 6.74 (1H, td, 7 Hz, 2 Hz), 4.31 (1H, d, 8 Hz), 3.95 (1H, br, d, 12 Hz), 3.83 (1H, td, 8 Hz, 4 Hz), 3.59 (1H, td, 11 Hz, 3 Hz), 2.82 (1H, td, 12 Hz, 3 Hz), 2.61-2.75 (3H, m), 2.35 (3H, s), 1.73 (1H, br, s). Compound 11C was converted into its hydrochloride salt following a modification of General Procedure 3C in which the pale yellow oil was taken up in isopropanol (˜200 ml) and filtered. Addition of hydrogen chloride (19 ml of a 1M solution in diethyl ether, 19 mmol) gave a white precipitate to which further diethyl ether (˜50 ml) was added. The solid was isolated by filtration and washed with diethyl ether to give the hydrochloride salt of 11C as a white solid (3.03 g, 78%); MW 367.96; C₁₈H₂₂ClNOS₂; ¹H NMR (CDCl₃): 9.94 (2H, br, s), 7.06-7.18 (6H, m), 6.94-7.03 (2H, m), 6.78 (1H, t, 7 Hz), 4.24-4.32 (1H, m), 4.20 (1H, d, 6 Hz), 3.89-4.06 (2H, m), 3.18 (2H, br, t, 12 Hz), 2.99 (2H, br, s), 2.37 (3H, s); LCMS (10 minute method): m/z 332 [M−HCl]+@Rt 5.07 min.

### EXAMPLE 3C

### (2S)-2-[(S)-{[2-(1-methylethyl)phenyl]thio}(phenyl)methylmorpholine (13C)

### (2S)-2-[(S)-{[2-(1-methylethyl)phenyl]thio}(phenyl)methyl]-4-(phenylmethyl)morpholine (12C)

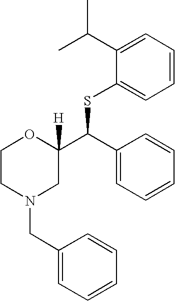

Compound 12C was obtained from 5Ca (4.04 g, 11.66 mmol), 2-isopropylsulphenyl-thiophenol (2.35 ml, 14 mmol, 1.2 eq) and caesium carbonate (4.56 g, 14 mmol, 1.2 eq) in dimethylformamide (35 ml) following a modification of General Procedure 1C in which the mixture was heated at 90° C. for 20 minutes, allowed to cool to room temperature, taken up in ethyl acetate (50 ml), washed with water and brine, dried over sodium sulphate, filtered and reduced in vacuo to give a yellow oil which was purified by SCX chromatography (eluent: ammonia/methanol 1/1 [v/v]). Removal of solvents in vacuo gave 12C as a white solid (4.45, 91%); MW 417.62; C₂₇H₃₁NOS; ¹H NMR (CDCl₃): 7.14-7.26 (7H, m), 7.03-7.1 (6H, m), 6.86-6.92 (1H, m), 4.10 (1H, d, 8 Hz), 3.88-3.94 (2H, m), 3.62 (1H, td, 11 Hz, 2 Hz), 3.37-3.47 (2H, m), 3.22 (1H, d, 13 Hz), 2.50 (2H, d, 11 Hz), 2.12-2.29 (2H, m), 1.05 (3H, d, 7 Hz), 0.92 (3H, d, 7 Hz); LCMS (6 minute method): m/z 418 [M+H]+@Rt 3.72 min.

### (2S)-2-[(S)-{[2-(1-methylethyl)phenyl]thio}(phenyl)methyl]morpholine (13C)

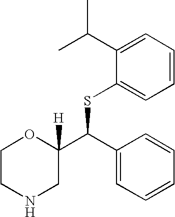

Compound 13C (Example 3C) was obtained from 12C (4.44 g, 10.65 mmol), solid supported Hünig's base (Argonaut, 3.56 mmol/g, 6.05 g, 21.54 mmol, 2 eq) and α-chloroethyl chloroformate (3.30 ml, 32.0 mmol, 3 eq) in anhydrous dichloromethane (50 ml) following General Procedure 2Ca. The mixture was heated at 40° C. for 1.5 hours then left to stir at room temperature overnight. The reaction mixture was filtered and concentrated in vacuo to give a pale yellow liquid. This was taken up in methanol (50 ml) and heated at 60° C. for 1.5 hours. The reaction mixture was allowed to cool to room temperature and purified by SCX chromatography (eluent: ammonia/methanol 1/1 [v/v]) to give 13C as a pale yellow oil; MW 327.49; C₂₀H₂₅NOS; ¹H NMR (CDCl₃): 7.22 (1H, d, 8 Hz), 7.03-7.13 (7H, m), 6.87-6.92 (1H, m), 4.04 (1H, d, 8 Hz), 3.94-3.99 (1H, m), 3.79 (1H, td, 9 Hz, 3 Hz), 3.61 (1H, td, 11 Hz, 3 Hz), 3.41 (1H, sept., 7 Hz), 2.82 (1H, td, 12 Hz and 3 Hz), 2.72 (1H, br, d, 12 Hz), 2.52-2.63 (2H, m), 1.70 (1H, br, s), 1.05 (3H, d, 7 Hz), 0.91 (3H, d, 7 Hz). Compound 13C was converted into its hydrochloride salt following a modification of General Procedure 3C in which the pale yellow oil was taken up in ether (50 ml), and filtered. Addition of hydrogen chloride in dry diethyl ether (19 ml of a 1M solution in diethyl ether) gave a white precipitate to which further diethyl ether (50 ml) was added. The reaction mixture was concentrated and the residue washed with diethyl ether to give a white solid (2.76 g, 69% overall yield from 5Ca); MW 363.95; C₂₀H₂₅NOS.HCl; ¹H NMR (CDCl₃): 9.91 (2H, br, s), 7.05-7.22 (7H, m), 6.91-6.96 (2H, m), 4.23-4.31 (1H, m), 4.08-3.90 (3H, m), 3.31-3.41 (1H, m), 3.04-3.21 (2H, br, m), 2.91-2.99 (2H, br, m), 1.06 (3H, d, 7 Hz), 0.93 (3H, d, 7 Hz); LCMS (10 minute method): m/z 327 [M−HCl]+@Rt 5.7 min.

### EXAMPLE 4C

### (2S)-2-[(S)-([1,1′-Biphenyl]-2-ylthio)(phenyl)methyl]morpholine (15C)

### (2S)-2-[(S)-([1,1′-Biphenyl]-2-ylthio)(phenyl)methyl]-4-(phenylmethyl)morpholine (14C)

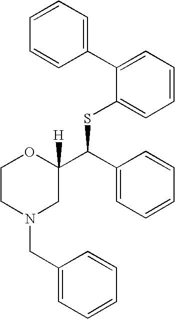

Compound 14C was obtained from 5Ca (2.16 g, 6.24 mmol), 2-phenylsulphenyl-thiophenol (2.35 ml, 14 mmol, 1.2 eq) and caesium carbonate (2.43 g, 7.5 mmol, 1.2 eq) in dimethylformamide (50 ml) following a modification of General Procedure 1C in which the mixture was heated at 90° C. for 20 minutes, allowed to cool to room temperature, taken up in ethyl acetate (50 ml), washed with water and brine, dried over sodium sulphate, filtered and reduced in vacuo to give a yellow oil. Purification by SCX-chromatography (eluent: ammonia/methanol 1/1 [v/v]) followed by evaporation in vacuo gave 14C as a white solid (0.59 g, 90%); MW 451.64; C₃₀H₂₉NOS; ¹H NMR (CDCl₃): 6.93-7.34 (19H, m), 3.92 (1H, br, d, 6 Hz), 3.63-3.76 (2H, m), 3.45 (1H, t, 10 Hz), 3.33 (1H, d, 13 Hz), 3.17 (1H, d, 12 Hz), 2.39 (1H, d, 12 Hz), 2.20 (1H, d, 11 Hz), 1.97-2.07 (1H, m), 1.82-1.92 (1H, m); LCMS (6 minute method): m/z 452 [M+H]+@Rt 3.69 min.

### (2S)-2-[(S)-([1,1′-Biphenyl]-2-ylthio)(phenyl)methyl]morpholine (15C)

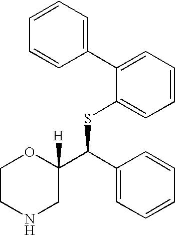

Compound 15C (Example 4C) was obtained from 14C (2.95 g, 6.54 mmol), solid supported Hünig's base (Argonaut, 3.56 mmol/g, 13.06 g, 21.54 mmol, 2 eq) and α-chloroethyl chloroformate (2.0 ml, 19.6 mmol, 3 eq) in anhydrous dichloromethane (50 ml) following General Procedure 2Ca. The reaction mixture was concentrated in vacuo to give a pale yellow liquid. This was taken up in methanol (70 ml) and heated at 40° C. for 2 hours. A white solid crashed out of the solution which was taken up in methanol and purified by SCX-chromatography (eluent: ammonia/methanol 1/1 [v/v]). After removal of solvents in vacuo 15C was obtained as a pale yellow oil; MW 361.51; C₂₃H₂₃NOS; ¹H NMR (CDCl₃): 7.0-7.45 (14H, m), 3.95 (1H, d, 8 Hz), 3.65-3.85 (2H, m), 3.35 (1H, d, 12 Hz), 3.2 (1H, d, 12 Hz), 2.45 (1H, d, 10 Hz), 2.20 (1H, d, 10 Hz), 2.0-2.15 (1H, m), 1.8-2.0 (1H, m); LCMS (12 minute method): m/z 363 [M+H]+@Rt 3.00 min. 15C was converted into its hydrochloride salt following a modification of General Procedure 3C in which the pale yellow oil was taken up in isopropanol (˜200 ml), and filtered. Addition of hydrogen chloride (19 ml of a 1M solution in diethyl ether) gave a white precipitate to which further diethyl ether (˜50 ml) was added. The solid was isolated by filtration and washed with diethyl ether to give the hydrochloride salt of 15C as a white solid (1.95 g, 75% overall yield from 5Ca); MW 397.97; C₂₃H₂₃NOS.HCl; ¹H NMR (CDCl₃): 9.80 (2H, br, s), 7.38-7.03 (12H, m), 6.90-6.96 (2H, m), 3.85-4.00 (2H, m), 3.72-3.82 (1H, m), 3.66 (1H, d, 5 Hz), 2.98-3.10 (1H, m), 2.81 (1H, br, s), 2.62 (2H, br, s); LCMS (12 minute method): m/z 362 [M+H]+@Rt 2.99 min.

### EXAMPLE 5C

### (2S)-2-[(S)-[(2-Fluorophenyl)thio](phenyl)methyl]morpholine (17C)

### (2S)-2-[(S)-[(2-Fluorophenyl)thio](phenyl)methyl]-4-phenylmethyl)morpholine (16Ca) and

### (2R)-2-[(R)-[(2-Fluorophenyl)thio](phenyl)methyl]-4-phenylmethyl)morpholine (16Cb)

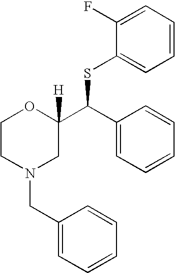

Compounds 16Ca,16Cb were obtained from 5Ca,5Cb (0.114 g, 0.33 mmol), 2-fluorothiophenol (0.045 g, 0.36 mmol, 1.2 eq) and caesium carbonate (0.12 g, 0.36 mmol, 1.2 eq) in dimethylformamide (50 ml) following General Procedure 1C as a pale yellow oil (0.14 g, 65%); MW 393.53; C₂₄H₂₄FNOS; ¹H NMR (CDCl₃): 7.12-7.36 (12H, m), 6.87-6.99 (2H, m), 4.48 (1H, d, 8 Hz), 4.00-4.11 (2H, m), 3.77 (1H, td, 11 Hz, 2 Hz), 3.60 (1H, d, 13 Hz), 3.37 (1H, d, 13 Hz); 2.63 (2H, t, 10 Hz), 2.16-2.31 (2H, m); LCMS (2.5 minute method): m/z 394 [M+H]⁺@Rₜ 1.41 min.

### (2S)-2-[(S)-[(2-Fluorophenyl)thio](phenyl)methyl]morpholine (17C)

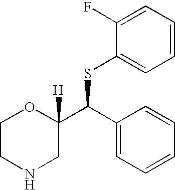

Compound 17C (Example 5C) was obtained from 16Ca,16Cb (0.72 g, 0.18 mmol), solid supported Hünig's base (Argonaut, 3.56 mmol/g, 2.0 g, 0.56 mmol, 3 eq) and α-chloroethyl chloroformate (0.62 ml, 0.56 mmol, 3 eq) in anhydrous dichloromethane (5 ml) following General Procedure 2Ca as a viscous yellow oil (0.046 g, 82%) from which 17C was obtained as a single isomer after separation by chiral HPLC (0.016 g); Chiral LC (AD): 10.83 min. LC purity=91% (UV254 nm)/98% (ELS); LCMS (10 minute method): m/z 304 [M+H]+@Rt 5.82 min; HPLC purity=84% (UV215 nm)/98% (ELS); MW 303.41; C₁₇H₁₈FNOS; ¹H NMR (CDCl₃): 7.13-7.00 (7H, m), 6.87-6.76 (2H, m), 4.29 (1H, d, 9 Hz), 3.98-3.93, (1H, m), 3.78 (1H, td, 9 Hz and 4 Hz), 3.60 (1H, td, 11 Hz and 3 Hz), 2.82 (1H, td, 12 Hz, 3 Hz), 2.76-2.70 (1H, m), 2.57-2.53, (2H, m), NH signal not observed; LCMS (10 minute method): m/z 304 [M+H]+@Rt 5.84 min; HPLC purity=100%% (ELS). Compound 17C was converted into its hydrochloride salt following General Procedure 3C.

### EXAMPLE 6C

### (2S)-2-[(S)-[(2-Ethylphenyl)thio](phenyl)methyl]morpholine (19C)

### (2S)-2-[(S)-[(2-Ethylphenyl)thio](phenyl)methyl]-4-(phenylmethyl)morpholine (18Ca) and

### (2R)-2-[(R)-[(2-Ethylphenyl)thio](phenyl)methyl]-4-(phenylmethyl)morpholine (18Cb)

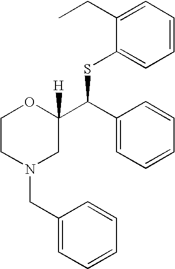

Compounds 18Ca,18Cb were obtained from 5Ca,5Cb (0.2 g, 0.58 mmol), 2-ethyl-thiophenol (0.16 g, 1.16 mmol, 2 eq) and caesium carbonate (0.23 g, 0.7 mmol, 1.2 eq) in dimethylformamide (5 ml) following modification of General Procedure 1C in which the reaction mixture was heated to 95° C. for 2 hours. After purification by flash column chromatography (eluent: ethyl acetate/hexane 9/1 [v/v]) 18Ca,18Cb was obtained as a white solid (0.15 g, 65%%); MW 403.59; C₂₆H₂₉NOS; ¹H NMR (CDCl₃): 6.96-7.40 (14H, m), 4.22 (1H, d, 7 Hz), 3.96-4.01 (2H, m), 3.72 (1H, td, 11 Hz and 2 Hz), 3.52 (1H, d, 13 Hz), 3.32 (1H, d, 13 Hz), 2.68 (2H, q, 8 Hz), 2.59 (2H, br d, 12 Hz), 2.06-2.21 (2H, m), 1.12 (3H, t, 7 Hz); LCMS (2.5 minute method) m/z 404 [M+H]+@Rt 1.49 min.

### (2S)-2-[(S)-[(2-Ethylphenyl)thio](phenyl)methyl]morpholine (19C)

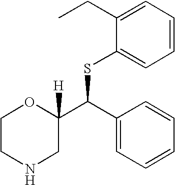

Compound 19C (Example 6C) was obtained from 18Ca,18Cb (0.18 g, 0.52 mmol), solid supported Hünig's base (Argonaut, 3.56 mmol/g, 3.7 g, 1.04 mmol, 2 eq) and α-chloroethyl chloroformate (0.34 ml, 3.12 mmol, 3 eq) in anhydrous dichloromethane (5 ml) following General Procedure 2Ca as a viscous yellow oil (0.21 g, 86%) from which 19C was obtained after separation by chiral HPLC on chiral OD semi-preparative column; chiral LC (OD): 15.95 min. LC purity=100% (UV254 nm)/100% (ELS); MW 313.47; C₁₉H₂₃NOS; ¹H NMR (CDCl₃): 7.17 (1H, d, 8 Hz), 7.12-7.05 (5H, m), 7.01 (2H, d, 4 Hz), 6.87-6.93 (1H, m), 4.07 (1H, d, 8 Hz), 3.92-3.97 (1H, m), 3.74-3.80 (1H, m), 3.59 (1H, td, 11 Hz, 3 Hz), 2.80 (1H, td, 12 Hz and 3 Hz), 2.71 (1H, br, d, 12 Hz), 2.63-2.54 (4H, m), 1.64 (1H, br, s), 1.04 (3H, t, 8 Hz); LCMS (10 minute method): m/z 314 [M+H]+@Rt 5.92 min. 19C was converted into its hydrochloride salt following General Procedure 3C; MW 349.93; C₁₉H₂₃NOS.HCl; ¹H NMR (CDCl₃): 10.10 (2H, br, s), 7.13-7.28 (8H, m), 7.02-7.08 (1H, m), 4.36 (1H, br, s), 4.01-4.17 (3H, br, m), 3.16-3.31 (2H, br, m), 2.92-3.09 (2H, br, m), 2.71 (2H, q, 8 Hz), 1.15 (3H, t, 7 Hz).

### EXAMPLE 7C

### (2S)-2-[(S)-{[2-(Methyloxy)phenyl]thio}(phenyl)methyl]morpholine (21C)

### (2S)-2-[(S)-{[2-(Methyloxy)phenyl]thio}(phenyl)methyl]-4-(phenylmethyl)morpholine (20Ca) and

### (2R)-2-[(R)-{[2-(Methyloxy)phenyl]thio}(phenyl)methyl]-4-(phenylmethyl)morpholine (20Cb)

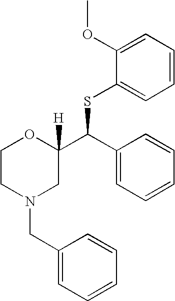

Compounds 20Ca,20Cb were obtained from 5Ca,5Cb (0.18 g, 0.52 mmol), 2-methoxy thiophenol (0.074 ml, 0.57 mmol, 1.2 eq) and caesium carbonate (0.17 g, 0.52 mmol, 1.2 eq) in dimethylformamide (5 ml) following modification of General Procedure 1C in which the reaction was heated at 95° C. for 2.5 hours. After purification by flash column chromatography (eluent: ethyl acetate/hexane gradient 15/85 to 25/75 [v/v]) 20Ca,20Cb was obtained as a viscous yellow oil (0.17 g, 83%); MW 405.56; C₂₅H₂₇NO₂S; ¹H NMR (CDCl₃): 7.01-7.26 (12H, m), 6.58-6.63 (2H, m), 4.39 (1H, d, 7 Hz), 3.86-3.91 (2H, m), 3.71 (3H, s), 3.56-3.62 (1H, m), 3.42 (1H, d, 11 Hz); 3.21 (1H, d, 11 Hz), 2.46-2.52 (2H, m), 2.01-2.11 (2H, m); LCMS (10 minute method): m/z 406 [M+H]⁺@R_{T} 6.09 min.

### (2S)-2-[(S)-{[2-(Methyloxy)phenyl]thio}(phenyl)methyl]morpholine (21C)

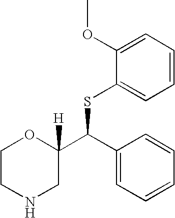

Compound 21C (Example 7C) was obtained from 20Ca,20Cb (0.1 g, 0.25 mmol), solid supported Hünig's base (Argonaut, 3.56 mmol/g, 1.78 g, 0.5 mmol, 2 eq) and α-chloroethyl chloroformate (0.16 ml, 1.5 mmol, 3 eq) in anhydrous dichloromethane (5 ml) following General Procedure 2Ca as a viscous yellow oil (0.06 g, 77%) from which 21C was obtained after separation by chiral HPLC on a Chiralcel OJ semi-preparative column. Chiral LC: 11.45 min. LC purity=100%; MW 315.44; C₁₈H₂₁NO₂S; ¹H NMR (CDCl₃): 7.14-7.34 (7H, m), 6.74-6.84 (2H, m), 4.50 (1H, d, 8 Hz), 4.10 (1H, d, 11 Hz), 3.85-4.00 (4H, m), 3.74 (1H, dt, 1 Hz, 11 Hz), 2.82-3.02 (2H, m), 2.66-3.02 (3H, m); LCMS (10 minute method): m/z 316 [M+H]⁺@Rₜ 4.87 min. 21C was converted its hydrochloride salt following General Procedure 3C.

### EXAMPLE 8C

### (2S)-2-[(S)-({2-[(1-Methylethyl)oxy]phenyl}thio)(phenyl)methyl]morpholine (23C)

### (2S)-2-[(S)-({2-[(1-Methylethyl)oxy]phenyl}thio)(phenyl)methyl]-4-(phenylmethyl)morpholine (22Ca) and

### (2R)-2-[(R)-({2-[(1-Methylethyl)oxy]phenyl}thio)(phenyl)methyl]-4-(phenylmethyl)morpholine (22Cb)

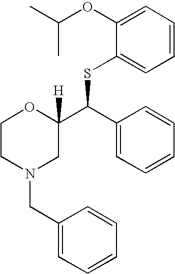

Compounds 22Ca,22Cb were obtained from 5Ca,5Cb (0.57 g, 1.7 mmol), 2-isopropoxy-thiophenol (0.94 g, 5.61 mmol) and caesium carbonate (2.18 g, 6.72 mmol, 1.2 eq) in dimethylformamide (15 ml) following modification of General Procedure 1C in which the reaction mixture was heated to 95° C. for 3 hours. After purification by SCX chromatography (eluent: ammonia/methanol 1/1 [v/v]) 22Ca,22Cb was obtained as a dark yellow oil (0.56 g, 76%%); MW 433.62; C₂₇H₃₁NO₂S; ¹H NMR (CDCl₃): 7.01-7.24 (7H, m), 6.94-7.09 (5H, m), 6.64 (1H, d, 8 Hz), 6.56 (1H, td, 8 Hz, 1 Hz), 4.42-4.51 (2H, m), 3.83-3.92 (2H, m), 3.56 (1H, td, 11 Hz and 3 Hz), 3.42 (1H, d, 13 Hz), 3.24 (1H, d, 13 Hz), 2.52 (1H, d, 11 Hz), 2.46 (1H, d, 11 Hz), 2.05-2.17 (2H, m), 1.29 (3H, d, 6 Hz), 1.27 (3H, d, 6 Hz); LCMS (2.5 minute method): m/z 434 [M+H]⁺@R_{T} 1.44 min.

### (2S)-2-[(S)-({2-[(1-Methylethyl)oxy]phenyl}thio)(phenyl)methyl]morpholine (23C)

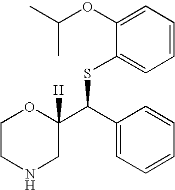

Compound 23C (Example 8C) was obtained from 22Ca,22Cb (0.56 g, 1.3 mmol), solid supported Hünig's base (Argonaut, 3.56 mmol/g, 0.73 g, 2.6 mmol, 2 eq) and a-chloroethyl chloroformate (0.16 ml, 1.5 mmol, 3 eq) in anhydrous dichloromethane (5 ml) following General Procedure 2Ca as a viscous yellow oil (0.41 g, 93%) after separation using chiral HPLC on a OD semi-preparative column. Chiral LC (OD): 12.51 min. LC purity=100% (UV254 nm)/100% (ELS); MW 343.49; C₂₀H₂₅NO₂S; ¹H NMR (CDCl₃): 7.13-7.20 (1H, m), 6.96-7.12 (6H, m), 6.67 (1H, d, 8 Hz), 6.59 (1H, td, 7 Hz, 1 Hz), 4.48 (1H, sept., 6 Hz), 4.38 (1H, d, 7 Hz), 3.90-3.95 (1H, m), 3.73 (1H, td, 8 Hz, 4 Hz), 3.54 (1H, td, 11 Hz and 3 Hz), 2.79 (1H, td, 12 Hz and 3 Hz), 2.62-2.72 (3H, m), 1.55 (1H, br, s), 1.32 (3H, d, 6 Hz), 1.29 (3H, d, 6 Hz); LCMS (10 minute method): m/z 344 [M+H]+@Rt 6.19 min; HPLC purity=92% (UV215nm). 23C was converted into its hydrochloride salt following General Procedure 3C; MW 379.95; C₂₀H₂₅NO₂S.HCl; ¹H NMR (CDCl₃): 9.81-10.04 (2H, br, m), 7.03-7.25 (7H, m), 6.71 (1H, d, 8 Hz), 6.63 (1H, t, 7 Hz), 4.51 (1H, sept., 6 Hz), 4.31 (1H, d, 6 Hz), 4.15-4.23 (1H, m), 3.83-4.03 (2H, m), 3.05-3.18 (2H, m), 2.80-3.03 (2H, m), 1.31 (3H, d, 6 Hz), 1.29 (3H, d, 6 Hz).

### EXAMPLE 9C

### 2-{[(S)-(2S)-Morpholin-2-yl(phenyl)methyl]thio}phenyl trifluoromethyl ether (25C)

### (2S)-4-(Phenylmethyl)-2-[(S)-phenyl({2-[(trifluoromethyl)oxy]phenyl}thio)methyl]morpholine (24Ca) and

### (2S)-4-(Phenylmethyl)-2-[(S)-phenyl({2-[(trifluoromethyl)oxy]phenyl}thio)methyl]morpholine (24Cb)

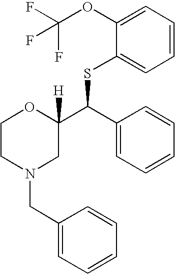

Compounds 24Ca,24Cb were obtained from 5Ca,5Cb (0.01 1 g, 0.33 mmol), 2-trifluoromethoxythiophenol (1.2 eq, 0.077 g, 0.39 mmol) and caesium carbonate (0.15 g, 0.47 mmol, 1.2 eq) in dimethylformamide (15 ml) following modification of General Procedure 1C in which the reaction was heated at 95° C. for 1.5 hours. The reaction mixture was allowed to cool to room temperature, diluted with ethyl acetate (20 ml), washed sequentially with water and brine, dried over sodium sulphate and finally concentrated in vacuo to give a pale yellow oil (0.14 g, 92%); MW 459.53; C₂₅H₂₄F₃NO₂S; ¹H NMR (CDCl₃): 7.13-7.41 (13H, m), 7.08-7.13 (1H, m), 4.51 (1H, d, 8 Hz), 3.99-4.07 (2H, m), 3.73 (1H, td, 9 Hz, 2.5 Hz), 3.57 (1H, d, 13 Hz), 3.37 (1H, d, 13 Hz); 2.57-2.66 (2H, m), 2.20-2.31 (2H, m); LCMS (10 minute method): m/z 460 [M+H]⁺@Rₜ 6.69 min.

### 2-{[(S)-(2S)-Morpholin-2-yl(phenyl)methyl]thio}phenyl trifluoromethyl ether (25C)

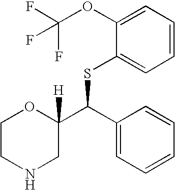

Compound 25C (Example 9C) was obtained from 24Ca,24Cb (0.06 g, 0.13 mmol), solid supported Hünig's base (Argonaut, 3.56 mmol/g, 0.073 g, 0.026 mmol, 2 eq) and α-chloroethyl chloroformate (0.04 ml, 0.39mmol, 3 eq) in anhydrous dichloromethane (5 ml) following General Procedure 2Ca as a viscous yellow oil (0.021 g, 44%) from which 25C was obtained after separation using chiral HPLC on a OD semi-preparative column. Chiral LC (OJ): 12.60 min. LC purity=98% (UV_{254 nm})/100% (ELS); MW 369.41; C₁₈H₁₈F₃NO₂S; ¹H NMR (CDCl₃): 7.02-7.21 (8H, m), 6.91-6.96 (1H, m), 4.28 (1H, d, 8 Hz), 3.93 (1H, br, d 11 Hz), 3.75-3.81 (1H, m), 3.60 (1H, td, 11 Hz and 3 Hz), 2.71-2.86 (2H, m), 2.61 (2H, d, 6 Hz), 1.90 (1H br, s); LCMS (10 minute method): m/z 370 [M+H]⁺@Rₜ 5.86 min.

### EXAMPLE 10C: (2S)-2-[(S)-[(2-Methylphenyl)thio](phenyl)methyl]morpholine (27C)

### (2S)-2-[(S)-[(2-Methylphenyl)thio](phenyl)methyl]-4-(phenylmethyl)morpholine (26Ca) and

### (2R)-2-[(R)-[(2-Methylphenyl)thio(phenyl)methyl]-4-(phenylmethyl)morpholine (26Cb)

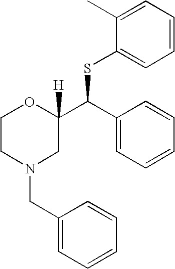

Compounds 26Ca,26Cb were obtained from 5Ca,5Cb (0.1 g, 0.29 mmol), 2-methyl thiophenol (0.04 ml, 0.31 mmol) and caesium carbonate (0.125 g, 0.37 mmol, 1.2 eq) in dimethylformamide (15 ml) following General Procedure 1C as a colourless oil (0.13 g, 85%); MW 389.56; C₂₅H₂₇NOS; ¹H NMR (CDCl₃): 6.84-7.24 (14H, m), 4.14 (1H, d, 8 Hz), 3.85-3.95 (2H, m), 3.60 (1H, dt, 10 Hz, 3 Hz), 3.42 (1H, d, 13 Hz); 3.21 (1H, d, 13 Hz), 2.46-2.54 (2H, m), 2.18 (3H, s), 1.97-2.13 (2H, m); LCMS (2.5 minute method): m/z 390 [M+H]⁺@R_{T} 1.49 min.

### (2S)-2-[(S)-[(2-Methylphenyl)thio](phenyl)methyl]morpholine (27C)

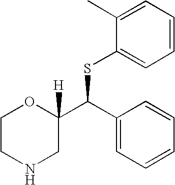

Compound 27C (Example 10C) was obtained from 26Ca,26Cb (0.04 g, 0.12 mmol), solid supported Hünig's base (Argonaut, 3.56 mmol/g, 0.89 g, 0.24 mmol, 2 eq) and α-chloroethyl chloroformate (0.04 ml, 0.36mmol, 3 eq) in anhydrous dichloromethane (5 ml) following General Procedure 2Ca as a viscous yellow oil (0.03 g, 75%) from which 27C was obtained after chiral separation. Chiral LC (OJ): 15.84 min. LC purity=98.57% (UV_{254 nm}); MW 299.44; C₁₈H₂₁NOS; ¹H NMR (CDCl₃): 6.86-7.21 (9H, m), 4.08 (1H, d, 7 Hz), 3.75 (1H, br s), 3.58 (1H, br s), 2.34-3.1 (4H, m), 2.20 (3H, s); 1.41-2.04 (2H, m); LCMS (10 minute method): m/z 300 [M+H]⁺@R_{T} 5.08 min. 27C was converted into its hydrochloride salt following General Procedure 3C.

### EXAMPLE 11C

### (2S)-2-{(S)-Phenyl[(2-propylphenyl)thio]methyl}morpholine (29C)

### (S)-Phenyl[(2S)-4-(phenylmethyl)morpholin-2-yl]methyl-2-propylphenyl sulfide (28Ca) and

### (R)-Phenyl[(2R)-4-(phenylmethyl)morpholin-2-yl]methyl-2-propylphenyl sulfide (28Cb)

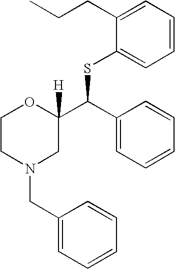

Compounds 28Ca,28Cb were obtained from 5Ca (0.53 g, 1.50 mmol), 2-n-propyl thiophenol (0.025 g, 1.65 mmol) and caesium carbonate (0.59 g, 1.8 mmol, 1.2 eq) in dimethylformamide (5 ml) following a modification of General Procedure 1C in which the reaction was heated at 95° C. for 3 hours. After purification by SCX column chromatography (eluent: ammonia/methanol 1/1 [v/v]) 28Ca,28Cb was obtained as a dark yellow oil (0.56 g, 90%%); MW 417.62; C₂₇H₃₁NOS; ¹H NMR (CDCl₃): 7.23-7.12 (6H, m), 7.06-7.11 (5H, m), 6.97-6.99 (2H, m), 6.87-6.92 (1H, m), 4.13 (1H, d, 8 Hz), 3.86-3.94 (2H, m), 3.61 (1H, td, 11 Hz, 2 Hz), 3.44 (1H, d, 13 Hz), 3.23 (1H, d, 13 Hz), 2.46-2.59 (4H, m), 2.01-2.14 (2H, m), 1.34-1.52 (2H, m), 0.83 (3H, t, 7 Hz); LCMS (2.5 minute method): m/z 418 [M+H]⁺@Rₜ 1.55 min.

### (2S)-2-{(S)-Phenyl[(2-propylphenyl)thio]methyl}morpholine (29C)

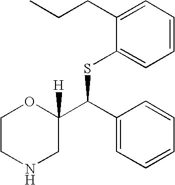

Compound 29C (Example 11C) was obtained from 28Ca,28Cb (0.56 g, 1.35 mmol), solid supported Hünig's base (Argonaut, 3.56 mmol/g, 0.75 g, 2.7 mmol, 2 eq) and α-chloroethyl chloroformate (0.44 ml, 4.05 mmol, 3 eq) in anhydrous dichlorometbane (5 ml) following General Procedure 2Ca as a viscous yellow oil (0.41 g, 93%); MW 327.49; C₂₀H₂₅NOS; ¹H NMR (CDCl₃): 7.17 (1H, br, d, 7 Hz), 7.07-7.12 (5H, m), 6.96-7.00 (2H, m), 6.88-6.93 (1H, m), 4.07 (1H, d, 8 Hz), 3.93-3.98 (1H, m), 3.74-3.80 (1H, m), 3.60 (1H, td, 11 Hz, 3 Hz), 2.81 (1H, td, 12 Hz and 3 Hz), 2.72 (1H, br, d, 12 Hz), 2.48-2.62 (4H, m), 1.36-1.59 (3H, m), 0.83 (3H, t, 7 Hz); LCMS (2.5 minute method): m/z 328 [M+H]⁺@Rₜ 1.40 min (single major peak).

### EXAMPLE 12C

### Methyl 2-{[(S)-(2S)-morpholin-2-yl(phenyl)methyl]thio}benzoate (31C)

### Methyl-2-({(S)-phenyl[(2S)-4-(phenylmethyl)morpholin-2-yl]methyl}thio)benzoate (30Ca) and

### Methyl-2-({(R)-phenyl[(2R)-4-(phenylmethyl)morpholin-2-yl]methyl}thio)benzoate (30Cb)

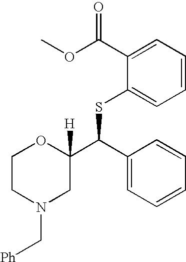

Compounds 30Ca,30Cb were obtained from 5Ca,5Cb (0.5 g, 1.45 mmol), methyl thiosalicylate (0.49 g, 2.89 mmol) and potassium carbonate (0.21 g, 1.52 mmol) in dry tetrahydrofurane (5 ml) following modification of General Procedure 1C in which the solvents were degassed and purged with nitrogen before the addition of methyl thiosalicylate. The reaction mixture was stirred at room temperature for 18 hours after which time the reaction mixture was poured onto water and extracted twice with diethyl ether. The organic layers were washed with water, dried and evaporated in vacuo. After purification by SCX column chromatography (eluent: ammonia/methanol 1/1 [v/v]) 30Ca,30Cb was obtained as a colourless solid (0.18 g, 29%%); MW 433.57; C₂₆H₂₇NO₃S; ¹H NMR (CDCl₃): 8.65-8.85 (1H, m), 6.95-7.45 (13H, m), 4.45 (1H, d, 8 Hz), 3.85-4.05 (1H, m), 3.8 (3H, s), 3.65 (1H, dt, 1 Hz and 7 Hz), 3.55 (1H, d, 11 Hz), 3.25 (1H, d, 11 Hz), 2.5-2.6 (2H, m); 2.0-2.15 (2H, m); FIA: m/z 462 [M+H]⁺.

### Methyl 2-{[(S)-(2S)-morpholin-2-yl(phenyl)methyl]thio}benzoate (31C)

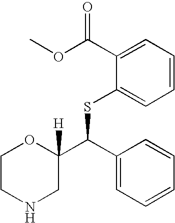

Compound 31C (Example 12C) was obtained from 30Ca,30Cb (0.2 g, 0.46 mmol), solid supported Hünig's base (Argonaut, 3.56 mmol/g, 0.08 g, 2.77 miol, 6 eq) and α-chloroethyl chloroformate (0.5 ml, 4.62 mmol, 10 eq) in anhydrous dichloromethane (5 ml) following General Procedure 2Ca as a white solid (0.16 g, 91%) from which 31C was obtained after separation using chiral HPLC on chiral OJ semi-preparative column. Chiral LC (OJ): 12.32 min. LC purity=100% (UV_{254 nm}); MW 343.45. 31 was converted into its hydrochloride salt following General Procedure 3C; ¹H NMR (d₆-DMSO): 9.30-9.5 (1H, m), 7.75-7.80 (1H, m), 7.1-7.55 (8H, m), 4.82 (1H, d, 8 Hz), 3.95-4.15 (2H, m), 3.65.3.9 (3H, m), 3.55 (3H, s), 2.80-3.25 (2H, m).

### EXAMPLE 13C

### (2S)-2-((S)-(3-Fluorophenyl){[2-(trifluoromethyl)phenyl]thio}methyl)morpholine (33C)

### (2S)-2-((S)-(3-Fluorophenyl){[2-(trifluoromethyl)phenyl]thio}methyl)-4-(phenylmethyl)morpholine (32Ca) and

### (2R)-2-((R)-(3-Fluorophenyl){[2-(trifluoromethyl)phenyl]thio}methyl)-4-(phenylmethyl)morpholine (32Cb)

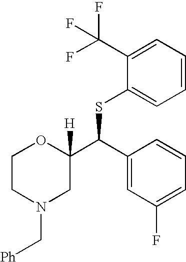

Compounds 32Ca,32Cb were obtained as outlined in Scheme 5C from 38Ca,38Cb (0.33 g, 0.91 mmol) following General Procedure 4C as a white solid after column chromatography (0.28 g, 67%); MW 461.53; C₂₅H₂₃F₄NOS; ¹H NMR (CDCl₃) 6.75-7.65 (1H, m), 6.85-7.33 (12H, m), 4.45 (2H, d, 8 Hz), 3.6-3.75 (2H, m), 3.45 (1H, d 12 Hz), 3.3 (1H, d 12 Hz), 2.45-2.7 (2H, br, m), ), 2.1-2.3 (2H, br, m); FIA: m/z 462 [M+H]⁺.

### (2S)-2-((S)-(3-Fluorophenyl){[2-(trifluoromethyl)phenyl]thio}methyl)morpholine (33C)

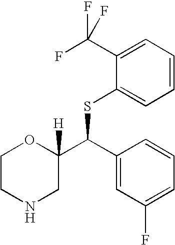

Compound 33C (Example 13C) was obtained from 32Ca,32Cb (0.28 g, 0.615 mmol), solid supported Hünig's base (Argonaut, 3.56 mmol/g, 0.19 g, 0.68 mmol, 1.1 eq) and α-chloroethyl chloroformate (0.07 ml, 0.68 mmol, 1.1 eq) in anhydrous dichloromethane (5 ml) following General Procedure 2Ca as a colourless oil (0.22 g, 95%) from which 33C was obtained after chiral chromatography on a Chiralcel OJ semi-preparative column. Chiral LC (OJ): 13.33 min. LC purity=98.37% (UV_{254 nm}); MW 371.4; C₁₈H₁₇F₄NOS. LCMS (12 minute method): m/z 372 [M+H]+@ Rt 5.2 min. 33C was converted into its hydrochloride salt following General Procedure 3C; MW 407.86; C₁₈H₁₇F₄NOS.HCl; ¹H NMR (CDCl₃) 9.8-10.2 (1H, br), 7.4-7.6 (1H, m), (6.85-7.45 (8H, m), 4.05-4.45 (4H, br, m), 2.90-3.41 (4H, br, m).

### EXAMPLE 14C

### (2S)-2-((S)-(4-Chlorophenyl){[2-(trifluoromethyl)phenyl]thio}methyl)morpholine (35C)

### (2S)-2-((S)-(4-Chlorophenyl){[2-(trifluoromethyl)phenyl]thio}methyl)-4-(phenylmethyl)morpholine (34Ca) and

### (2R)-2-((R)-(4-Chlorophenyl){[2-(trifluoromethyl)phenyl]thio}methyl)-4-(phenylmethyl)morpholine (34Cb)

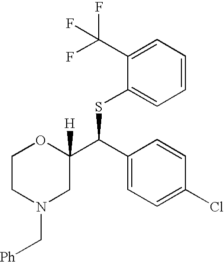

Compounds 34Ca,34Cb were obtained as outlined in Scheme 5C from 39Ca,39Cb (0.4 g, 1.06 mmol, 1.1 eq), cesium carbonate (0.33 g, 1.0 mmol, 1.1 eq), and 2-trifluoromethyl benzene thiol (0.19 g, 1.06 mmol, 1.1 eq) following a modification of General Procedure 1C in which the reaction was stirred at room temperature for 1.5 hours as a white solid after column chromatography (eluent: gradient hexane/ethyl acetate 10/90 to 25/75[v/v]) (0.409 g, 80%); MW 477.98; C₂₅H₂₃F₃ClNOS; ¹H NMR (CDCl₃) 7.1-7.65 (13H, m), 4.45 (1H, d, 8 Hz), 3.85-4.0 (2H, m), 3.55 (1H, m), 3.3 (1H, d 12 Hz), 3.3 (1H, d 12 Hz), 2.45-2.65 (2H, br),), 2.1-2.3 (2H, br, m); FIA: m/z 478 [M+H]⁺.

### (2S)-2-((S)-(4-Chlorophenyl){[2-(trifluoromethyl)phenyl]thio}methyl)morpholine (35C)

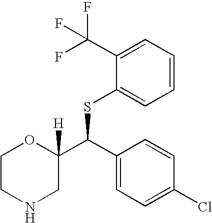

Compound 35C (Example 14C) was obtained from 34Ca,34Cb (0.41 g, 0.86 mmol), solid supported Hünig's base (Argonaut, 3.56 mmol/g, 0.27 g, 0.94 mmol, 1.1 eq) and α-chloroethyl chloroformate (0.10 ml, 0.94 mmol, 1.1 eq) in anhydrous dichloromethane (5 ml) following General Procedure 2Ca as a colourless oil (0.28 g, 84% yield) from which 35C was obtained after separation using chiral HPLC on a ChiralPak-AD OJ semi-preparative column; MW 387.85; C₁₈H₁₇ClF₃NOS; LCMS (12 minute method): m/z 372 [M+H]+@Rt 5.2 min. 35C was converted into its hydrochloride salt following General Procedure 3C; MW 423.96; C₁₈H₁₇CIF₃NOS.HCl; ¹H NMR (CDCl₃): 9.8-10.2 (1H, br), 7.4-7.6 (1H, m), 7.07-7.35 (7H, m), 3.8-4.45 (4H, br, m), 2.85-3.45 (4H, br, m).

### EXAMPLE 15C

### (2S)-2-((S)-(2-Fluorophenyl){[2-(methyloxy)phenyl]thio}methyl)morpholine (37C)

### (2S)-2-((S)-(2-Fluorophenyl){[2-(methyloxy)phenyl]thio}methyl)-4-(phenylmethyl)morpholine (36Ca) and

### (2R)-2-((R)-(2-Fluorophenyl){[2-(methyloxy)phenyl]thio}methyl)-4-(phenylmethyl)morpholine (36Cb)

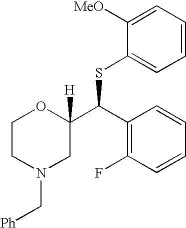

Compounds 36Ca,36Cb were obtained from 7Ca,7Cb (0.45 g, 1.17 mmol), cesium carbonate (0.42 g, 1.29 mmol, 1.1 eq), and 2-methoxy-thiophenol (0.82 g, 5.87 mmol) following a modification of General Procedure 1C in which the reaction mixture was heated to 95° C. for 2 hours and then stirred at room temperature for 18 hours. After purification by flash column chromatography (eluent: heptane/ethyl acetate 80/20 [v/v]) 36Ca,36Cb was obtained as a colourless oil (0.36 g, 72%%); MW 423.55; C₂₅H₂₆FNOS; ¹H NMR (CDCl₃): 6.65-7.5 (13H, m), 4.9 (1H, d, 7 Hz), 3.9-4.05 (2H, m), 3.8 (3H, s), 3.6 (1H, dt, 8 Hz and 1 Hz), 3.45 (1H, d, 13 Hz), 3.15 (1H, d, 13 Hz), 2.60 (2H, t, 8 Hz), 2.05-2.2 (2H, m); FIA: m/z 424 [M+H]⁺.

### (2S)-2-((S)-(2-Fluorophenyl){[2-(methyloxy)phenyl]thio}methyl)morpholine (37C)

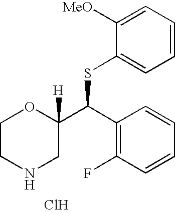

Compound 37C (Example 15C) was obtained from 36Ca,36Cb (0.43 g, 1.02 mmol), solid supported Hünig's base (Argonaut, 3.56 mmol/g, 0.37 g, 1.12 mmol, 1.1 eq) and α-chloroethyl chloroformate (1.08 ml, 10.12 mmol, 10 eq) in anhydrous dichloromethane (5 ml) following General Procedure 2Ca as a colourless oil (0.34 g, 99%) after separation by chiral HPLC on a ChiralPak-AD semi-preparative column. Chiral LC: 12.86 min. LC purity=99.1 (UV_{254 nm}); MW 369.89; C₁₈H₂₀FNOS; FIA: m/z 334 [M+H]⁺. 37C was converted into its hydrochloride salt following General Procedure 3C; MW 333.43; C₁₈H₂₀FNOS; ¹H NMR (CDCl₃): 7.2-7.3 (1H, m), 6.85-7.2 (8H, m), 4.85 (1H, d, 8 Hz), 3.95-4.15 (2H, m), 3.85.3.9 (3H, m), 3.7 (1H, dt, 1 Hz and 7 Hz), 2.6-3.0 (4H, m).

### EXAMPLE 16C

### 2-12-Methyl-1-(2-trifluoromethyl-phenylsulfanyl)-propyl]-morpholine (56C)

### 4-Benzyl-2-(1-hydroxy-2-methyl-propyl)-morpholin-3-one (53C)

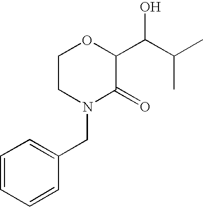

To a stirred solution of 2C (5.05 g, 26.4 mmol) in tetrahydrofuran (25 ml) at −78° C. under nitrogen was added lithium diisopropylamide (14.5 ml of a 2M solution, 29.0 mmol) dropwise over 40 minutes. The reaction mixture was stirred at the same temperature over 30 minutes after which time a solution of isobutyraldehyde (2.63 ml, 29.0 mmol) in tetrahydrofuran (15 ml) was added dropwise over 30 minutes. After one hour, the reaction mixture was allowed to warm to room temperature and quenched by addition of saturated ammonium chloride solution. Extraction with dichloromethane and drying over magnesium sulphate gave 53C as a mixture of diastereomers. Upon concentration in vacuo one diastereomer precipitated as a white solid (53Ca: 0.99 g). The remaining mother liquors were purified by column chromatography (30% ethyl acetate in hexane [v/v]) to give 53C (2.06 g). MW 263.34; C₁₅H₂₁NO₃; LCMS (6 min method): m/z 286 [M+Na]⁺; RT=2.748.

### 1-(4-Benzyl-morpholin-2-yl)-2-methyl-propan-1-ol (54C)

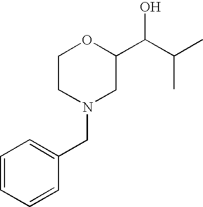

To a stirred solution of 53C (1.97 g, 7.47 mmol) in tetrahydrofuran (50 ml) at room temperature under nitrogen was added borane-tetrahydrofuran complex (30 ml of a 1M solution, ca 4 eq.). The reaction was heated to 60° C. and followed by TLC-analysis. When all starting material had been consumed a few drops of methanol were added followed by a similar amount of 1N hydrochloric acid and heating was continued for another hour. Organic solvents were removed in vacuo and the remaining solution was poured onto 1M potassium carbonate solution (30 ml), extracted with diethyl ether. The organic layers were dried over magnesium sulphate and purified by column chromatography (gradient from 15% ethyl acetate in hexane [v/v]) gave 54C (1.8 g, 97%). MW 249.36; C₁₅H₂₃NO₂; LCMS (6 min method): m/z 250 [M+H]⁺; RT=0.838.

### 4-Benzyl-2-[2-methyl-1-(2-trifluoromethyl-phenylsulfanyl)-propyl]-morpholine (55C)

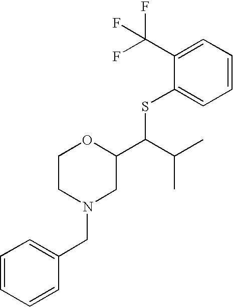

Compound 55C was obtained from 54C in a two-step procedure. To a stirred solution of 54C (1.8 g, 7.2 mmol) in dichloromethane (50 ml) at room temperature was added solid solid supported Hünig's base (Argonaut, 3.56 mmol/g, 6.2 g, 22 mmol, 3 eq) followed methanesulphonyl chloride (1.12 ml, 14 mmol). After stirring for one hour, the reaction mixture was filtered and the filtrates washed with brine and dried over magnesium sulphate to give the intermediate mesylate as a yellow oil (2.93 g of isolated crude product). The crude product was taken up in dry dimethylformamide (50 ml), 2-trifluoromethyl benzenethiol (2.1 ml, 14 mmol) and solid supported Hünig's base (Argonaut, 3.56 mmol/g, 0.55 g, 1.95 mmol) were added and the mixture heated to 70° C. and stirred for 72 hours. The reaction was quenched by addition of water (50 ml) and sodium hydroxide solution (70 ml of a 2N solution). The aqueous layer was extracted with diethyl ether (3×50 ml), washed with brine and dried over magnesium sulphate. Purification by ion-exchange chromatography followed by preparative HPLC gave 55C. MW 409.52; C₂₂H₂₆F₃NOS; LCMS (6 min method): m/z 410 [M+H]⁺; RT=3.398.

### 2-[2-Methyl-1-(2-trifluoromethyl-phenylsulfanyl)-propyl]-morpholine (56C)

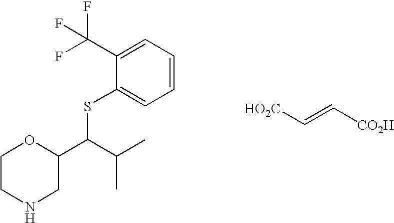

Compound 56C (Example 16C) was obtained from 55C (0.8 g, 1.95 mmol), solid supported Hünig's base (Argonaut, 3.56 mmol/g, 1.65 g, 5.85 mmol, 3 eq) and α-chloroethyl chlorofonrate (0.4 ml, 3.9 mmol, 2 eq) in anhydrous dichloromethane (20 ml) following General Procedure 2Ca as a colourless oil (0.5 g, 85% yield). Chiral HPLC on a ChiralCel-OD(3671) column using 50% heptane in ethanol [v/v] gave 2 fractions (Rt=8.793 min and 10.443 min). Conversion into fumarate salt 56C was carried out by dissolving in diethyl ether and addition of small amount of methanol. Data for 56C derived from fraction with Rt=8.793 min: MW 435.46; C₁₉H₂₄F₃NO₅S; ¹H NMR (d₃-MeOD): 6.2-6.3 (2H, m), 6.1-6.2 (1H, m), 5.2 (1H, s), 2.6-2.7 (2H, m), 2.2-2.4 (1H, m), 1.6-1.9 (4H, m), 1.6-1.7 (1H, m), −0.4-−0.5 (6H, m).

### EXAMPLE 17C

### 2-[2-Methyl-1-(2-trifluoromethyl-phenoxy)-propyl]-morpholine (58C)

### 4-Benzyl-2-[2-methyl-1-(2-trifluoromethyl-phenoxy)-propyl]-morpholine (57C)

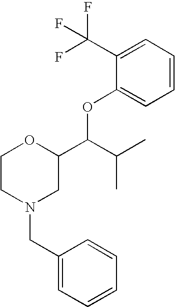

To a solution of 53Ca (0.146 g, 0.585 mmol) in dry dimethylformamide (2 ml) under nitrogen and ice-cooling was added sodium hydride (26 mg of a 60% dispersion in oil, 0.644 mmol) portionwise. The reaction was allowed to warm to room temperature for 30 minutes before addition of 2-fluoro-benzotriflouride (0.07 ml, 0.66 mmol). After stirring for 12 hours, another 0.5 equivalents of reagents were added and the reaction mixture heated to 40° C. for 30 minutes and then to 60° C. for another 2 hours. The crude reaction mixture was purified by ion-exchange column chromatography followed by preparative HPLC to give 57C (0.208 g, 92% yield) MW 393.45; C₂₂H₂₆F₃NO₂; LCMS (6 min method): m/z 394 [M+H]⁺; RT=3.150.

### 2-[2-Methyl-1-(2-trifluoromethyl-phenoxy)-propyl]-morpholine (58C)

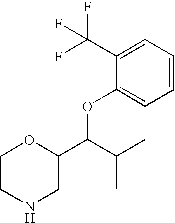

Compound 58C (Example 17C) was obtained from 57C (0.21 g, 0.53 mmol), solid supported Hünig's base (Argonaut, 3.56 mmol/g, 0.45 g, 1.5 mmol, 3 eq) and α-chloroethyl chlorofornate (0.11 ml, 1.06 mmol, 2 eq) in anhydrous dichloromethane (10 ml) following General Procedure 2C as a colourless oil (0.147 g, 92% yield) MW 303.33; C₁₅H₂₀F₃NO₂; ¹H NMR (CDCl₃): 7.5-7.6 (1H, m), 7.2-7.4 (1H, m), 7.0-7.1 (1H, m), 6.8-6.95 (1H, m), 4.15-4.25 (1H, m), 3.6-3.9 (2H, m), 3.4-3.6 (1H, m), 2.6-2.9 (4H, m), 2.15 (1H, br, s)1.8-2.1 (1H, m), 1.1-1.2 (6H, m); LCMS (12 min method): m/z 304 [M+H]⁺; RT=4.862.

The following examples illustrate compounds of of Formulae (ID) above and methods for their preparation.

### Scheme 1D—Preparation of Intermediates

### 1-Phenyl-3,4-dihydro-1H-quinolin-2-one (2Da)

A stirred mixture of 3,4-Dihydro-1H-quinolin-2-one (1Da) (1.47 g. 10 mmol), K₂CO₃ (2.9 g, 21 mmol), trans-cyclohexane-1,2-diamine (240 μL, 2 mmol) and bromobenzene (3.16 mL, 30 mmol) in 1,4-dioxane (10 mL) was heated under a nitrogen atmosphere at 125° C. for 5 min to deoxygenate the reaction mixture. Copper (I) iodide (380 mg, 2 mmol) was added in one portion and the reaction mixture was refluxed overnight at 125° C. After cooling to rt, the reaction mixture was poured into ethyl acetate (100 mL) and extracted with water. The organic layer was separated, dried over MgSO₄ and concentrated. Treatment of the residue with ether (100 mL) and cooling (ice bath) gave the product as a white solid after filtration (1.77 g, 79%).

### 6-Fluoro-1-p-tolyl-3,4-dihydro-1H-quinolin-2-one (2Db)

This was prepared using the method described for (2Da) using 6-Fluoro-3,4-dihydro-1H-quinolin-2-one (1Db) (617 mg, 3.7 mmol) and 4-bromotoluene (1.91 g, 11 mmol) to give the crude product, which was purified using automated chromatography (silica) (0 to 60% ethyl acetatecyclohexane gradient) to provide the product as a light brown solid (880 mg, 92%).

### 3-Methyl-1-phenyl-3,4-dihydro-1H-quinolin-2-one (3Da)

To a soln of (2Da) (892 mg, 4 mmol) in anhydrous THF (40 mL) at −78° C. under nitrogen was added LiHMDS (4.4 mL, 1M soln in hexanes, 4.4 mmol) dropwise over 10 min. The reaction mixture was left at −78° C. for 30 min and then a solution of methyl iodide (298 μL, 4.8 mmol) in THF (1 mL) was added dropwise. The reaction mixture was warmed slowly to rt, quenched with water (2 mL) and extracted with ethyl acetate (100 mL). The organic layer was separated, dried over MgSO₄ and concentrated. The residue was purified by column chromatograpy (silica, gradient 100% hexane to ethyl acetatehexane 3:10) giving the product as an oil (667 mg, 70%).

### 3-Ethyl-1-phenyl-3,4-dihydro-1H-quinolin-2-one (3Db)

This was prepared in a similar manner to (3Da) on a 1.5 mmol scale using 1-iodoethane (125 μL, 1.1 eq.) as the alkylating agent. The crude product (378 mg) was used directly in the next step.

### 3-(3-Chloro-propyl)-1-phenyl-3,4-dihydro-1H-quinolin-2-one (4Da)

To a soln of (2Da) (892 mg, 4 mmol) in anhydrous THF (40 mL) at −78° C. under nitrogen was added LiHMDS (4.4 mL, 1M soln in hexanes, 4.4 mmol) dropwise over 10 min. The reaction mixture was left at −78° C. for 30 min and then a solution of 1-bromo-3-chloropropane (405 μL, 4.4 mmol) in THF (1 mL) was added dropwise. The reaction mixture was warmed slowly to rt, quenched with water (2 mL) and extracted with ethyl acetate (100 mL). The organic layer was separated, dried over MgSO₄ and concentrated. The crude product (1.2 g) was used directly in the next step.

### 3-(3-Chloro-propyl)-6-fluoro-1-p-tolyl-3,4-dihydro-1H-quinolin-2-one (4Db)

This was prepared from (2Db) (300 mg, 1.17 mmol) using the method described for (4Da) using 1-bromo-3-chloropropane (140 μL, 1.4 mmol) as the alkylating agent. The crude product (399 mg) was used directly in the next step.

### 3-(2-Chloro-ethyl)-1-phenyl-3,4-dihydro-1H-quinolin-2-one (4Dc)

This was prepared from (2Da) (892 mg, 4.0 mmol) using the method described for (4Da) using 1-bromo-2-chloroethane (365 μL, 4.4 mmol) as the alkylating agent. The crude product (1 g) was used directly in the next step.

### 3-(3-Chloro-propyl)-3-methyl-1-phenyl-3,4-dihydro-1H-quinolin-2-one (5Da)

This was prepared from (3Da) (462 mg, 1.95 mmol) using the method described for (4Da) using 1-bromo-3-chloropropane (270 μL, 2.7 mmol) as the alkylating agent. The crude product (650 mg) was used directly in the next step.

### 3-(3-Chloro-propyl)-3-ethyl-1-phenyl-3,4-dihydro-1H-quinolin-2-one (5Db)

This was prepared from (3Db) (378 mg, 1.5 mmol) using the method described for (4Da) using 1-bromo-3-chloropropane (179 μL, 1.8 mmol) as the alkylating agent. The crude product (528 mg) was used directly in the next step.

### Scheme 1D—Examples

### EXAMPLE 1D

### 3-(3-Methylamino-propyl)-1-phenyl-3,4-dihydro-1H-quinolin-2-one (6Da)

A soln of (4Da) (1.2 g, 4 mmol), potassium iodide (200 mg, 1.2 mmol) and aqueous 40% methylamine (12 mL) in ethanol (30 mL) was refluxed at 100° C. under nitrogen for 3 h. The reaction mixture was cooled, poured into water and extracted with ethyl acetate (100 mL). The organic layer was separated, dried over MgSO₄ and concentrated. The product was purified by preparative LCMS to give 500 mg of the racemate. The racemate was separated into its individual enantiomers using chiral HPLC. ¹H NMR (300 MHz, CDCl₃) (racemate & isomer) δ 1.5-1.73 (m, 4H), 1.88-1.97 (m, 1H), 2.43 (s, 3H), 2.62 (t, J=6.69 Hz, 2H), 2.70-2.79 (m, 1H), 2.84-2.92 (m, 1H), 3.15 (dd, J=15.45, 5.28 Hz, 1H), 6.33 (d, J=7.73 Hz, 1H), 6.95-7.06 (m, 2H), 7.19-7.22 (m, 3H), 7.38-7.43 (m, 1H), 7.47-7.52 (m, 2H). LCMS (12 minute method) [M+H]⁺=295@Rt 4.0 min (100%).

### EXAMPLE 2D

### 6-Fluoro-3-(3-methylamino-propyl)-1-p-tolyl-3,4-dihydro-1H-quinolin-2-one (6Db)

This was prepared in an identical manner to (6Da) using crude (4Db) (399 mg) to give the crude product, which was purified by preparative LCMS to give the product (35 mg). ¹ H NMR (300 MHz, CDCl₃) (racemate) δ 1.40-1.70 (m, 3H), 1.75-1.90 (m, 4H), 2.34 (s, 3H), 2.36 (s, 3H), 2.50-2.83 (m, 2H), 3.01-3.08 (m, 1H), 6.21-6.26 (m, 1H), 6.62-6.68 (m, 1H), 6.82-6.86 (m, 1H), 6.99 (d, J=8.1 Hz, 2H), 7.22 (d, J=8.1 Hz, 2H). LCMS (12 minute method) [M+H]⁺=327@ Rt 4.8 min (100%).

### EXAMPLE 3D

### 3-(2-Methylamino-ethyl)-1-phenyl-3,4-dihydro-1H-quinolin-2-one (6Dc)

This was prepared in an identical manner to (6Da) using crude (4Dc) (1g) to give the racemate (80 mg). The racemate was separated into its individual enantiomers using chiral HPLC. ¹H NMR (300 MHz, CDCl₃) (racemate & isomer) δ ppm 1.64-1.76 (m, 1H), 1.79 (br, 1H), 2.03-2.18 (m, 1H), 2.44 (s, 3H), 2.71-2.82 (m, 2H), 2.82-2.94 (m, 2H), 3.09-3.21 (m, 1H), 6.33 (dd, J=7.91, 1.32 Hz, 1H), 6.94-7.07 (m, 2H), 7.18-7.24 (m, 3H), 7.37-7.44 (m, 1H), 7.47-7.54 (m, 2H). LCMS (12 minute method) [M+H]⁺=281@Rt 3.82 min (100%).

### EXAMPLE 4D

### 3-Methyl-3-(3-methylamino-propyl)-1-phenyl-3,4-dihydro-1H-quinolin-2-one (7Da)

This was prepared in an identical manner to (6Da) using crude (5Da) (650 mg) to give the crude product (198 mg), which was purified by preparative LCMS. The purified racemate was then separated into its individual enantiomers using chiral HPLC. ¹H NMR (300 MHz, CDCl₃) (isomer) δ ppm 1.27 (s, 3H), 1.43 (br, 1H), 1.53-1.66 (m, 4H), 2.39 (s, 3H), 2.54 (t, J=6.12 Hz, 2H), 2.91 (d, J=15.64 Hz, 1H), 2.98 (d, J=15.64 Hz, 1H), 6.28 (dd, J=7.91, 1.32 Hz, 1H), 6.97 (td, J=7.21, 1.41 Hz, 1H), 7.03 (td, J=7.68, 1.98 Hz, 1H), 7.14-7.22 (m, 3H), 7.36-7.44 (m, 1H), 7.46-7.53 (m, 2H). LCMS (12 minute method) [M+H]⁺=309@Rt 4.21 min (100%).

### EXAMPLE 5D

### 3-Ethyl-3-(3-methylamino-propyl)-1-phenyl-3,4-dihydro-1H-quinolin-2-one (7Db)

This was prepared in an identical manner to (6Da) using crude (5Db) (528 mg) to give the crude product (105 mg), which was purified by preparative LCMS. The purified racemate was then separated into its individual enantiomers using chiral HPLC. ¹H NMR (300 MHz, CDCl₃) (racemate) δ 0.93 (t, J=7.53 Hz, 3H), 1.56-1.75 (m, 6H), 1.91 (bs, 1H), 2.41 (s, 3H), 2.55-2.60 (m, 2H), 2.91 (d, J=15.82, 1H), 3.02 (d, J=15.82, 1H), 6.25-6.28 (m, 1H), 6.94-7.05 (m, 2H), 7.16-7.19 (m, 3H), 7.38-7.43 (m, 1H), 7.4-7.52 (m, 2H). ¹H NMR (300 MHz, MeOD-d4) (isomer D-tartrate salt) δ 0.85 (t, J=7.53 Hz, 3H), 1.45-1.75 (m, 6H), 2.57 (s, 2H), 2.83-2.89 (m, 2H), 3.01-3.06 (d, J=16.01, 1H), 4.32 (s, 2H), 6.11-6.14 (m, 1H), 6.89-6.97 (m, 2H), 7.09 (d, J=7.16 Hz, 2H), 7.15-7.18 (m, 1H), 7.37 (t, J=7.35 Hz, 1H), 7.46 (t, J=7.35 Hz, 2H). LCMS (12 minute method) [M+H]⁺=323@Rt 4.9 min (98%).

### Scheme 2D—Preparation of Intermediates

### 1-p-Tolyl-3,4-dihydro-1H-quinolin-2-one (2Dc)

A stirred mixture of 3,4-Dihydro-1H-quinolin-2-one (1Da) (4.41 g. 30 mmol), K₂CO₃ (8.7 g, 63 mmol), trans-cyclohexane-1,2-diamine (720 μL, 2 mmol) and 4-bromotoluene (15.4 g, 90 mmol) in 1,4-dioxane (30 mL) was heated under a nitrogen atmosphere at 125° C. for 5 min to deoxygenate the reaction mixture. Copper (I) iodide (1.14 g, 2 mmol) was added in one portion and the reaction mixture was refluxed overnight at 125° C. After cooling to rt, the reaction mixture was filtered through celite, poured into ethyl acetate (100 mL) and extracted with water. The organic layer was separated, dried over MgSO₄ and concentrated. Treatment of the residue with ether (200 mL) and cooling (ice bath) gave the product as a white solid after filtration (6.2 g, 87%).

### 1-Phenyl-3-propyl-3,4-dihydro-1H-quinolin-2-one (3Dc)

This was prepared from (2Da) (669 mg, 3 mmol) and 1-iodopropane (352 μl, 1.2 eq.) as the alkylating agent. The crude product (780 mg) was used directly in the next step.

### 3-Ethyl-1-p-tolyl-3,4-dihydro-1H-quinolin-2-one (3Dd)

This was prepared from (2Dc) (711 mg, 3 mmol) and 1-iodoethane (265 μl, 1.2 eq.) as the alkylating agent. The crude product (800 mg) was used directly in the next step.

### 3-Propyl-1-p-tolyl-3,4-dihydro-1H-quinolin-2-one (3De)

This was prepared from (2Dc) (711 mg, 3 mmol) and 1-iodopropane (352 μl, 1.2 eq.) as the alkylating agent. The crude product (840 mg) was used directly in the next step.

### 3-Butyl-1-p-tolyl-3,4-dihydro-1H-quinolin-2-one (3Df)

This was prepared from (2Dc) (711 mg, 3 mmol) and 1-iodobutane (354 μl, 1.1 eq.) as the alkylating agent. The crude product (790 mg) was used directly in the next step.

### 3-Isopropyl-1-p-tolyl-3,4-dihydro-1H-quinolin-2-one (3Dg)

This was prepared from (2Dc) (711 mg, 3 mmol) and 2-iodopropane (330 μl, 1.1 eq.) as the alkylating agent. The crude product (806 mg) was used directly in the next step.

### 3-Allyl-3-ethyl-1-p-tolyl-3,4-dihydro-1H-quinolin-2-one (11Db)

To a soln of (3Dd) (800 mg, 2.7 mmol) in anhydrous THF (30 mL) at −78° C. under nitrogen was added LiHMDS (3 mL, 1M soln in hexanes, 3 mmol) dropwise over 10 min. The reaction mixture was left at −78° C. for 30 min and then a solution of allyl bromide (280 μL, 3.2 mmol) in THF (1 mL) was added dropwise. The reaction mixture was warmed slowly to rt, quenched with water (2 mL) and extracted with ethyl acetate (100 mL). The organic layer was separated, dried over MgSO₄ and concentrated. The crude product (920 mg) was used directly in the next step.

### 3-Ethyl-3-(3-hydroxypropyl)-1-p-tolyl-3,4-dihydro-1H-quinolin-2-one (12Db)

To a soln of (11Db) (732 mg, 2.4 mmol) in anhydrous THF (25 mL) at 0° C. under nitrogen was added 9-BBN (12 mL, 0.5M soln in THF, 6 mmol, 2.5 eq.) dropwise over 10 min. The reaction mixture was warmed to rt and left to stir overnight. The resultant yellow soln was cooled to 0° C. and then quenched carefully with ethanol (3 mL), followed by aq. NaOH (1.8 mL, 3N soln). Finally, aq. H₂O₂ (1.8 mL, 37% soln) was added dropwise maintaining the internal reaction mixture temp between 5 and 10° C. The reaction mixture was warmed to rt and then refluxed for 90 min. The reaction mixture was cooled to rt, poured into ethyl acetate and water and extracted. The organic layer was separated, dried over MgSO₄ and concentrated. The crude product was purified using automated chromatography (silica) (0 to 60% ethyl acetatecyclohexane gradient) to provide (12Db) as a clear oil (540 mg, 70%).

### Scheme 2D—Examples

### EXAMPLE 6D

### 3-Ethyl-3-(3-methylamino-propyl)-1-p-tolyl-3,4-dihydro-1H-quinolin-2-one (13Db)

To a soln of (12Db) (540 mg, 1.67 mmol) and triethylamine (350 μL, 2.5 mmol) in anhydrous THF (20 mL) at 0° C. under nitrogen was added dropwise a soln of methanesulfonyl chloride (142 μL, 1.8 mmol) in THF (1 mL). The reaction mixture was warmed to rt and stirred for 3 h. The reaction mixture was poured into ethyl acetate and water and extracted. The organic layer was separated, dried over MgSO₄ and concentrated. The crude mesylate (670 mg, 100%) was dissolved in ethanol (10 mL) and aqueous 40% methylamine (5 mL) and heated at 65° C. under nitrogen for 2 h. The reaction mixture was cooled, poured into water and extracted with ethyl acetate (100 mL). The organic layer was separated, dried over MgSO₄ and concentrated. The product was purified by SCX-2 to give 384 mg of the racemate. The racemate was separated into its individual enantiomers using chiral HPLC. Each enantiomer was dissolved in CH₂Cl₂ (2 mL) and treated with 1 equivalent of D-tartaric acid dissolved in a minimum volume of warm methanol. The resultant soln was concentrated and the solid was dried under vacuo to provide the D-tartrate salt of the amine. ¹H NMR (300 MHz, CDCl₃) (racemate) δ 0.92 (t, J=7.44 Hz, 3H), 1.49-1.75 (m, 6H), 1.81 (br, 1H), 2.40 (s, 6H), 2.57 (t, J=6.59 Hz, 2H), 2.89 (d, J=15.82 Hz, 1H), 3.00 (d, J=15.82 Hz, 1H), 6.29 (d, J=7.91 Hz, 1H), 6.92-7.08 (m, 4H), 7.16 (d, J=7.16 Hz, 1H), 7.29 (d, J=7.91 Hz, 2H). ¹H NMR (300 MHz, MeOD-d4) (isomer D-tartrate salt) δ 0.93 (t, J=7.44 Hz, 3H), 1.54-1.84 (m, 6H), 2.42 (s, 3H), 2.66 (s, 3H), 2.91-3.00 (m, 3H), 3.11 (d, J=15.83 Hz, 1H), 4.41 (s, 2H), 6.22-6.27 (m, 1H), 6.80-7.07 (m, 4H), 7.21-7.27 (m, 1H), 7.36 (d, J=7.91 Hz, 2H). LCMS (12 minute method) [M+H]⁺=337@Rt 5.21 min (100%).

### EXAMPLE 7D

### 3-(3-Methylamino-propyl)-1-phenyl-3-propyl-3,4-dihydro-1H-quinolin-2-one (13Da)

This was prepared from (3Dc) (780 mg, 2.9 mmol) using the same synthetic sequence described above (3Dd to 13Db) to give 233 mg of the racemate. The racemate was separated into its individual enantiomers using chiral HPLC and each enantiomer was converted into its D-tartrate salt as described for (13Db). ¹H NMR (300 MHz, CDCl₃) (racemate) δ 0.88 (t, J=7.16 Hz, 3H), 1.26-1.48 (m, 2H), 1.50-1.78 (m, 7H), 2.40 (s, 3H), 2.56 (t, J=6.59 Hz, 2H), 2.92 (d, J=15.83 Hz, 1H), 3.01 (d, J=15.83 Hz, 1H), 6.25-6.28 (m, 1H), 6.94-7.05 (m, 2H), 7.16-7.19 (m, 3H), 7.37-7.42 (m, 1H), 7.47-7.52 (m, 2H). ¹H NMR (300 MHz, MeOD-d4) (isomer D-tartrate salt) δ 0.77-0.82 (t, J=7.06 Hz, 3H), 1.24-1.35 (m, 2H), 1.44-1.51 (m, 2H), 1.69 (bs, 3H), 2.56 (s, 3H), 2.84-2.89 (m, 3H), 3.01-3.06 (d, J=15.83 Hz, 1H), 3.20-3.22 (q, J=1.55 Hz, 2H), 4.30 (s, 2H), 6.11-6.14 (dd, J=7.72, 2.26 Hz, 1H), 6.89-6.97 (m, 2H), 7.07-7.10 (m, 2H), 7.14-7.17 (m, 1H), 7.34-7.39 (t, J=7.35 Hz, 1H), 7.43-7.48 (t, J=7.35 Hz, 2H). LCMS (12 minute method) [M+H]⁺=337@Rt 5.2 min (100%).

### EXAMPLE 8D

### 3-(3-Methylamino-propyl)-3-propyl-1-D-tolyl-3,4-dihydro-1H-quinolin-2-one (13Dc)

This was prepared from (3De) (840 mg, 2.6 mmol) using the same synthetic sequence described above (3Dd to 13Db) to give 393 mg of the racemate. The racemate was separated into its individual enantiomers using chiral HPLC and each enantiomer was converted into its D-tartrate salt as described for (13Db). ¹H NMR (300 MHz, CDCl₃) (racemate) δ 0.88 (t, J=7.16 Hz, 3H), 1.20-1.75 (m, 11H), 2.39 (s, 3H), 2.40 (s, 3H), 2.90 (d, J=15.64 Hz, 1H), 2.99 (d, J=15.64 Hz, 1H), 6.29 (d, J=7.72 Hz, 1H), 6.93-7.07 (m, 4H), 7.14-7.16 (m, 1H), 7.25-7.31 (m, 2H). ¹H NMR (300 MHz, MeOD-d4) (isomer D-tartrate salt) δ 0.91 (t, J=7.06 Hz, 3H), 1.28-1.85 (m, 8H), 2.44 (s, 3H), 2.68 (s, 3H), 2.94-2.99 (m, 3H), 3.14 (d, J=15.82 Hz, 1H), 4.41 (s, 2H), 6.25-6.28 (m, 1H), 7.02-7.07 (m, 4H), 7.25-7.28 (m, 1H), 7.38 (d, J=7.91 Hz, 2H). LCMS (12 minute method) [M+H]⁺=351@Rt 5.6 min (100%).

### EXAMPLE 9D

### 3-Butyl-3-(3-methylamino-propyl)-1-p-tolyl-3,4-dihydro-1H-quinolin-2-one (13Dd)

This was prepared from (3Df) (790 mg, 2.7 mmol) using the same synthetic sequence described above (3Dd to 13Db) to give 334 mg of the racemate. The racemate was separated into its individual enantiomers using chiral HPLC and each enantiomer was converted into its D-tartrate salt as described for (13Db). ¹H NMR (300 MHz, CDCl₃) (racemate) δ 0.87 (t, J=6.97 Hz, 3H), 1.20-1.40 (m, 4H), 1.55-1.74 (m, 6H), 2.40 (s, 3H), 2.40 (s, 3H), 2.55 (t, J=6.78 Hz, 3H), 2.91 (d, J=15.63 Hz, 1H), 2.99 (d, J=15.63 Hz, 1H), 6.28-6.31 (m, 1H), 6.93-7.00 (m, 2H), 7.02-7.06 (m, 2H), 7.14-7.16 (m, 1H), 7.29 (d, J=8.07 Hz, 2H). ¹H NMR (300 MHz, MeOD-d4) (isomer D-tartrate salt) δ 0.90 (t, J=6.97 Hz, 3H), 1.20-1.85 (m, 10H), 2.44 (s, 3H), 2.68 (s, 3H), 2.94-2.99 (m, 3H), 3.14 (d, J=15.82 Hz, 1H), 4.42 (s, 2H), 6.25-6.28 (m, 1H), 7.00-7.07 (m, 4H), 7.25-7.28 (m, 1H), 7.38 (d, J=7.91 Hz, 2H). LCMS (12 minute method) [M+H]⁺=365@Rt 5.9 min (100%).

### EXAMPLE 10D

### 3-Isopropyl-3-(3-methylamino-propyl)-1-p-tolyl-3,4-dihydro-1H-quinolin-2-one (13De)

This was prepared from (3Dg) (806 mg, 2.89 mmol) using the same synthetic sequence described above (3Dd to 13Db) to give 307 mg of the racemate. ¹H NMR (300 MHz, CDCl₃) (racemate) δ ppm 0.92 (dd, J=8.95, 6.88 Hz, 6H), 1.39-1.88 (m, 5H), 2.12-2.23 (m, 1H), 2.39 (s, 3H), 2.40 (s, 3H), 2.56 (t, J=6.78 Hz, 2H), 2.94 (d, J=15.92 Hz, 1H), 3.00 (d, J=15.92 Hz, 1H), 6.28 (dd, J=7.82, 1.04 Hz, 1H), 6.92-7.06 (m, 4H), 7.16 (dd, J=6.97, 1.13 Hz, 1H), 7.29 (d, J3 7.91 Hz, 2H). LCMS (12 minute method) [M+H]⁺=351@Rt 5.55 min (100%).

### EXAMPLE 11D

### 6-Chloro-3-ethyl-3-(3-methylamino-propyl)-1-p-tolyl-3,4-dihydro-1H-quinolin-2-one (13Df)

This was prepared from (1Dc) using the same synthetic sequence described above to give 205 mg of the racemate. The racemate was separated into its individual enantiomers using chiral HPLC and each enantiomer was converted into its D-tartrate salt as described for (13Db). ¹H NMR (300 MHz, CDCl₃) (racemate) δ ppm 0.91 (t, J=7.44 Hz, 3H), 1.50-1.75 (m, 6H), 2.15 (br, 1H), 2.40 (s, 3H), 2.41 (s, 3H), 2.55-2.64 (m, 2H), 2.85 (d, J=16.01 Hz, 1H), 2.97 (d, J=16.01 Hz, 1H), 6.23 (d, J=8.85 Hz, 1H), 6.97 (dd, J=8.67, 2.45 Hz, 1H), 7.02 (d, J=8.29 Hz, 2H), 7.14 (d, J=2.26 Hz, 1H), 7.29 (d, J=8.10 Hz, 2H). ¹H NMR (300 MHz, MeOD-d4) (isomer, D-tartrate salt) δ ppm 0.84 (t, J=7.35 Hz, 3H), 1.40-1.75 (m, 6H), 2.32 (s, 3H), 2.57 (s, 3H), 2.80-2.92 (m, 3H), 3.01 (d, J=16.20 Hz, 1H), 4.31 (s, 2H), 6.13 (d, J=8.67 Hz, 1H), 6.92-6.98 (m, 3H), 7.19 (d, J=2.26 Hz, 1H), 7.26 (d, J=7.91 Hz, 2H). LCMS (12 minute method) [M+H]⁺=371/373 @Rt 5.75 min (100%).

### EXAMPLE 12D

### 6-Chloro-1-(4-chloro-phenyl)-3-ethyl-3-(3-methylamino-propyl)-3,4-dihydro-1H-quinolin-2-one (13Dg)

This was prepared from (1Dc) using the same synthetic sequence described above to give 222 mg of the racemate, which was purified by preparative LCMS. ¹H NMR (300 MHz, CDCl₃) (racemate) δ ppm 0.84 (t, J=7.44 Hz, 3H), 1.40-1.70 (m, 6H), 2.35 (br, 4H), 2.49-2.56 (m, 2H), 2.80 (d, J=16.01 Hz, 1H), 2.90 (d, J=16.01 Hz, 1H), 6.14 (d, J=8.67 Hz, 1H), 6.93 (dd, J=8.67, 2.26 Hz, 1H), 7.04 (ddd, J=9.04, 2.83, 2.45 Hz, 2H), 7.09 (d, J=2.26 Hz, 1H), 7.36-7.43 (m, 2H). LCMS (12 minute method) [M+H]⁺=391/393 @Rt 5.67 min (92%).

Scheme 3D—Preparation of Intermediates

### 1-(4-Methoxy-benzyl)-3,4-dihydro-1H-quinolin-2-one (14D)

A 5 litre flange-neck flask equipped with an air stirrer and paddle, thermometer, nitrogen bubbler and pressure equalising dropping funnel was charged with sodium hydride (25.5 g, 60% oil dispersion, 0.637 mol) and 40-60 pet. ether (100 ml). The mixture was stirred briefly and then allowed to settle under nitrogen. After decanting the supernatant liquid, the vessel was charged with dimethylformamide (2 litres). The well stirred suspension was cooled to 7-8° C. using an external ice-bath. Then a soln of 3,4-dihydro-1H-quinolin-2-one (1a) (73.6 g, 0.5 mole) in anhydrous dimethylformamide (500 ml) was added dropwise over 25 min. The mixture was stirred at 7-8° C. for 30 min. then 4-methoxybenzyl chloride (102 g, 0.65 mole, 1.3 eq.) was added over 10 min. The reaction mixture was left to stir for 2 h. at <10° C. then allowed to warm-up to room temperature and stirred overnight. The stirred reaction mixture was quenched with ice/water (2.5 litres) and cooled to 15° C. using an external ice-bath. The white solid was isolated by filtration and washed with water. After drying in vacuo at 40° C. overnight the product was obtained (113.4 g, 85%).

### 1-(4-Methoxy-benzyl)-3-methyl-3,4-dihydro-1H-quinolin-2-one (15D)

To a soln of (14) (20 g, 75 mmol) in anhydrous THF (400 mL) at −78° C. under nitrogen was added LiHMDS (78.6 mL, 1M soln in hexanes, 78.6 mmol) dropwise over 10 min. The reaction mixture was left at −78° C. for 30 min and then a solution of methyl iodide (5.13 mL, 83 mmol) in THF (5 mL) was added dropwise. The reaction mixture was warmed slowly to rt, quenched with water (50 mL) and extracted with ethyl acetate (400 mL). The organic layer was separated, dried over MgSO₄ and concentrated to give the product as a yellow solid (21 g, 100%) that was used directly in the next step.

### 3-Allyl-1-(4-methoxy-benzyl)-3-methyl-3,4-dihydro-1H-quinolin-2-one (16Db)

To a soln of (15D) (20.5 g, 73 mmol) in anhydrous THF (400 mL) at −78° C. under nitrogen was added LiHMDS (80 mL, 1M soln in hexanes, 80 mmol) dropwise over 10 min. The reaction mixture was left at −78° C. for 30 min and then a solution of allyl bromide (7.6 mL, 87 mmol) in THF (5 mL) was added dropwise. The reaction mixture was warmed slowly to rt, quenched with water (100 mL) and extracted with ethyl acetate (400 mL). The organic layer was separated, dried over MgSO₄ and concentrated to give the product as an orange oil (23.9 g, 100%) that was used directly in the next step.

### 3-(3-Hydroxy-propyl)-1-(4-methoxy-benzyl)-3-methyl-3,4,4a,8a-tetrahydro-1H-quinolin-2-one (17Db)

To a soln of (16Db) (23.9 g, 74 mmol) in anhydrous THF (400 mL) at 0° C. under nitrogen was added 9-BBN (370 mL, 0.5M soln in THF, 185 mmol, 2.5 eq.) dropwise over 10 min. The reaction mixture was warmed to rt and left to stir overnight. The resultant yellow soln was cooled to 0° C. and then quenched carefully with ethanol (95 mL), followed by aq. NaOH (60 mL, 3N soln). Finally, aq. H₂O₂ (60 mL, 37% soln) was added dropwise maintaining the internal reaction mixture temp between 5 and 10° C. The reaction mixture was warmed to rt and then refluxed for 90 min. The reaction mixture was cooled to rt, poured into ethyl acetate and water and extracted. The organic layer was separated, dried over MgSO₄ and concentrated. The crude product was purified using automated chromatography (silica) (0 to 80% ethyl acetatecyclohexane gradient) to provide the product as a clear oil (21.3 g, 84%).

### 1-(4-Methoxy-benzyl)-3-methyl-3-(3-methylamino-propyl)-3,4,4a,8a-tetrahydro-1H-quinolin-2-one (18Db)

To a soln of (17Db) (18 g, 53 mmol) and triethylamine (11.1 mL, 79 mmol) in anhydrous THF (450 mL) at 0° C. under nitrogen was added dropwise a soln of methanesulfonyl chloride (4.52 mL, 58 mmol) in THF (50 mL). The reaction mixture was warmed to rt and stirred for 3 h. The reaction mixture was poured into ethyl acetate and water and extracted. The organic layer was separated, dried over MgSO₄ and concentrated. The crude mesylate (22 g, 99%) was dissolved in ethanol (500 mL) and aqueous 40% methylamine (200 mL) and heated at 65° C. under nitrogen for 2 h. The reaction mixture was cooled, concentrated and then extracted with ethyl acetate (300 mL). The organic layer was washed with water, brine, dried over MgSO₄ and concentrated to give the crude product (17.8 g, 96%).

### Methyl-[3-(3-methyl-2-oxo-1,2,3,4,4a,8a-hexahydro-quinolin-3-yl)-propyl]-carbamic acid tert-butyl ester (19Db)

A mixture of (18Db) (17.8 g, 50.5 mmol) and anisole (5.5 mL, 50.5 mmol) in trifluoroacetic acid (250 mL) was heated at 65° C. under nitrogen for 2 h. The reaction mixture was concentrated under vacuo and the residue was dissolved in methanol (10 mL). The methanol soln was applied to an SCX-2 column (300 g, pre-washed with methanol) and the column washed with methanol (approx 1 litre) until the soln became colourless. The product was eluted with 2N NH₃ in methanol (500 mL) and the basic soln was concentrated to provide 3-Methyl-3-(3-methylamino-propyl)-3,4-dihydro-1H-quinolin-2-one (9 g, 77%). To a soln of this amine (8.6 g, 37 mmol) in anhydrous THF (350 mL) at 0° C. was added a soln of di-tert-butyl dicarbonate (8.34 g, 97%, 50.5 mmol) in THF (20 mL) dropwise. The reaction mixture was warmed to rt and stirred for 3 h. The reaction mixture was poured into ethyl acetate (400 mL) and water (200 mL) and extracted. The organic layer was separated, dried over MgSO₄ and concentrated to give the product as a yellow solid (12.26 g, 100%). This material was used without further purification.

### Methyl-[3-(2-oxo-1,2,3,4-tetrahydro-quinolin-3-yl)-propyl]-carbamic acid tert-butyl ester (19Da)

This was prepared from (14D) using the same synthetic sequence described above.

### [3-(6-Chloro-1,2,3,4-tetrahydro-quinolin-3-yl)-propyl]-methyl-carbamic acid tert-butyl ester (20Da)

To a soln of (19Da) (2.75 g, 8.6 mmol) in anhydrous DMF (25 mL) at 0° C. was added dropwise a soln of N-chlorosuccinimide (1.17 g, 8.7 mmol) in anhydrous DMF (3 mL). The reaction mixture was warmed to rt, stirred overnight and then poured into ethyl acetate (100 mL) and water (50 mL) and extracted. The organic layer was separated, dried over MgSO₄ and concentrated to provide the product as a yellow oil 3 g, 98%) that was used without further purification.

### Scheme 3D

### EXAMPLES

### EXAMPLE 13D

### 3-(3-Methylamino-propyl)-1-p-tolyl-3,4-dihydro-1H-quinolin-2-one (21Da)

A stirred mixture of (19Da) (100 mg. 0.31 mmol), K₂CO₃ (92 mg, 0.66 mmol), trans-cyclohexane-1,2-diamine (8 μL, 0.06 mmol) and 4-bromotoluene (162 mg, 0.94 mmol) in 1,4-dioxane (0.5 mL) was heated under a nitrogen atmosphere at 125° C. for 5 min to deoxygenate the reaction mixture. Copper (I) iodide (12 mg, 0.06 mmol) was added in one portion and the reaction mixture was refluxed overnight at 125° C. After cooling to rt, the reaction mixture was poured into ethyl acetate (100 mL) and extracted with water. The organic layer was separated, dried over MgSO₄ and concentrated. The crude product was purified using automated chromatography (silica) (0 to 80% ethyl acetate\cyclohexane gradient) to provide the Boc protected product (70 mg, 54%). To a soln of this material (70 mg, 0.17 mol) in DCM (2 mL), was added trifluoroacetic acid (197 μL, 2.55 mmol, 15 eq.). The reaction mixture was left to stir at room temperature for 90 min, concentrated under vacuo poured into ethyl acetate (50 mL) and aq. NaHCO₃ (20 mL) and extracted. The organic layer was separated, dried over MgSO₄, concentrated and the crude product was purified by SCX-2 to provide the racemate (40 mg, 75%). The racemate was separated into its individual enantiomers using chiral HPLC. ¹H NMR (300 MHz, CDCl₃) (racemate) δ 1.49-1.77 (m, 3H), 1.86-1.96 (m, 1H), 2.34 (bs, 1H), 2.40 (s, 3H), 2.43 (s, 3H), 2.61-2.66 (t, J=6.88 Hz, 2H), 2.68-2.78 (m, 1H), 2.83-2.90 (m, 1H), 3.09-3.17 (m, 1H), 6.36 (dd, J=7.7 Hz, 1.0 Hz, 1H), 6.94-7.03 (m, 2H), 7.08 (d, J=8.2 Hz, 2H), 7.13-7.17 (m, 1H), 7.29 (d, J=8.1 Hz, 2H); ¹H NMR (300 MHz, MeOD-d4) (isomer, D-tartrate salt) δ 1.64 (bs, 1H), 1.89 (bs, 3H), 2.41(s, 3H), 2.70 (s, 3H), 2.75-2.87 (m, 1H), 2.91-3.06 (m, 3H), 3.20 (dd, J=5.9, 15.26 Hz, 1H), 4.45 (s, 2H), 6.32-6.35 (m, 1H), 7.00-7.12 (m, 4H), 7.28-7.30 (m, 1H), 7.37 (d, J=8.1 Hz, 2H). LCMS (12 minute method) [M+H]⁺=309 @ Rt 4.7 min (100%).

### EXAMPLE 14D

### 6-Chloro-3-(3-methylamino-propyl)-1-p-tolyl-3,4-dihydro-1H-quinolin-2-one (21Dn)

This was prepared from (20Da) (132 mg, 0.29 mmol) using the same methods described for (21Da) to provide the racemate (86 mg). ¹H NMR (300 MHz, CDCl₃) (racemate & isomer) δ 1.50-1.57 (m, 1H), 1.62-1.90 (m, 3H), 2.34 (s, 3H), 2.41 (s, 3H), 2.63-2.82 (m, 5H), 3.00-3.07 (m, 1H), 6.22 (d, J=8.6 Hz, 1H), 6.92 (dd, J=2.45, 8.66 Hz, 1H 6.99 (d, J=8.1 Hz, 2H), 7.11 (d, J=2.25 Hz, 1H), 7.23 (d, J=8.1 Hz, 2H). LCMS (12 minute method) [M+H]⁺=343/345 @ Rt 5.2 min (96%).

### EXAMPLE 15D

### 1-(3-Fluorophenyl)-3-(3-methylamino-propyl)-3,4-dihydro-1H-quinolin-2-one (21Db)

This was prepared from (19Da) (200 mg, 0.63 mmol) using the same two-step procedure described for (21Da) to provide the racemate (83 mg). ¹H NMR (300 MHz, CDCl₃) (racemate) δ 1.60-1.70 (m, 1H), 1.92 (br, 3H), 2.64 (bs, 3H), 2.72-2.74 (m, 1H), 2.86-3.09 (m, 4H), 6.35 (dd, J=7.72, 1.510 Hz, 1H), 6.94-7.23 (m, 6H), 7.43-7.51 (m, 1H). LCMS (12 minute method) [M+H]⁺=313 @ Rt 4.4 min (100%).

### EXAMPLE 16D

### 1-(4-Chlorophenyl)-3-(3-methylamino-propyl)-3,4-dihydro-1H-quinolin-2-one (21Dc)

This was prepared from (19Da) (122 mg, 0.38 mmol) using the same two-step procedure described for (21Da) to provide the crude product, which was purified by SCX-2 to give the racemate (70 mg). ¹H NMR (300 MHz, CDCl₃) (racemate) δ 1.49-1.73 (m, 3H), 1.89 (m, 2H), 2.43 (s, 3H), 2.62 (t, J=6.79, 7.15 Hz, 2H), 2.68-2.78 (m, 1H), 2.83-2.93 (m, 1H), 3.14 (dd, J=15.43, 5.37 Hz, 1H), 6.34 (dd, J=7.73, 1.14 Hz, 1H), 6.96-7.09 (m, 2H), 7.14-7.21 (m, 3H), 7.45-7.48 (m, 2H). LCMS (12 minute method) [M+H]⁺=329/331 @ Rt 5.1 min (90%).

### EXAMPLE 17D

### 1-(3,4-Dichlorophenyl)-3-(3-methylamino-propyl)-3,4-dihydro-1H-quinolin-2-one (21Dd)

This was prepared from (19Da) (150 mg, 0.47 mmol) using the same two-step procedure described for (21Da) to provide the crude product, which was purified by SCX-2 to give the racemate (111 mg). ¹H NMR (300 MHz, CDCl₃) (racemate) δ 1.49-1.75 (m, 3H), 1.83 (bs, 1H), 1.85-1.97 (m, 1H), 2.43 (s, 3H), 2.63 (t, J=13.56, 6.59 Hz, 2H), 2.68-2.77 (m, 1H), 2.83-2.94 (m, 1H), 3.13 (dd, J=15.45, 5.28 Hz, 1H), 6.36 (dd, J=7.73, 0.93 Hz, 1H), 6.99-7.11 (m, 3H), 7.20-7.21 (m, 1H), 7.35 (d, J=2.26 Hz, 1H), 7.57 (d, J=8.48 Hz, 1H). LCMS (12 minute method) [M+H]⁺=363/365 @Rt 5.4 min (92%).

### EXAMPLE 18D

### 1-(3-Chlorophenyl)-3-(3-methylamino-propyl)-3,4-dihydro-1H-quinolin-2-one (21De)

This was prepared from (19Da) (200 mg, 0.63 mmol) using the same two-step procedure described for (21Da) to provide the crude product, which was purified by SCX-2 to give the racemate (138 mg). ¹H NMR (300 MHz, CDCl₃) (racemate) δ 1.50-1.77 (m, 3H), 1.89-1.96 (m, 2H), 2.44 (s, 3H), 2.64 (t, J=6.89 Hz, 2H), 2.69-2.78 (m, 1H), 2.84-2.93 (m, 1H,), 3.10-3.17 (m, 1H), 6.33-6.36 (m, 1H), 6.97-7.10 (m, 2H), 7.11-7.15 (m, 1H), 7.21-7.24 (m, 2H), 7.37-7.47 (m, 2H). LCMS (12 minute method) [M+H]⁺=329/331 @ Rt 5.01 min (90%).

### EXAMPLE 19D

### 1-(4-Fluorophenyl)-3-(3-methylamino-propyl)-3,4-dihydro-1H-quinolin-2-one (21Df)

This was prepared from (19Da) (200 mg, 0.63 mmol) using the same two-step procedure described for (21Da) to provide the crude product, which was purified by SCX-2 to give the racemate (48 mg). ¹H NMR (300 MHz, CDCl₃) (racemate) δ 1.26-1.28 (m, 1H), 1.92 (m, 2H), 2.63 (bs, 1H), 2.72 (m, 1H), 2.85-3.08 (m, 2H), 3.48-3.51 (m, 5H), 6.32-6.34 (d, J=7.91 Hz, 1H), 7.01-7.70 (m, 2H), 7.16-7.19 (d, J=7.16 Hz, 5H), 9.46 (bs, 1H). LCMS (12 minute method) [M+H]⁺=313 @ Rt 4.5 min (100%).

### EXAMPLE 20D

### 1-(4-Ethylphenyl)-3-(3-methylamino-propyl)-3,4-dihydro-1H-quinolin-2-one (21Dg)

This was prepared from (19Da) (148 mg, 0.46 mmol) using the same two-step procedure described for (21Da) to provide the racemate (61 mg). ¹H NMR (300 MHz, CDCl₃) (racemate) δ 1.25-1.30 (m, 1H), 1.52-1.67(m, 1H), 1.69-1.80 (m, 2H), 1.87-1.98 (m, 1H), 2.46 (s, 3H), 2.67-2.92 (m, 9H), 3.11-3.16 (m, 1H), 6.34-6.37 (m, 1H), 6.94-7.06 (m, 2H), 7.09-7.11 (d, J=8.1 Hz, 2H), 7.17-7.20 (d, J=7.35 Hz, 1H), 7.30-7.33 (d, J=8.28 Hz, 2H). LCMS (12 minute method) [M+H]⁺=323 @ Rt 5.4 min (98%).

### EXAMPLE 21D

### 3-Methyl-3-(3-methylamino-propyl)-1-p-tolyl-3,4-dihydro-1H-quinolin-2-one (21Dh)

This was prepared from (19Db) (806 mg, 2.89 mmol) using the same methods described for (21Da) to provide the racemate. The racemate was separated into its individual enantiomers using chiral HPLC. ¹H NMR (300 MHz, CDCl₃) (racemate & isomer) δ 1.24 (s, 3H), 1.60-1.65 (m, 4H), 2.40 (s, 3H), 2.43 (s, 3H), 2.60-2.65 (m, 2H), 2.87 (d, J=15.73 Hz, 1H), 2.98 (d, J=15.73 Hz, 1H), 3.46 (br, 1H), 6.30 (dd, J=7.91, 1.13 Hz, 1H), 6.90-7.05 (m, 2H), 7.05 (d, J=8.29 Hz, 2H), 7.10-7.20 (m, 1H), 7.29 (d, J=7.91 Hz, 2H). LCMS (12 minute method) [M+H]⁺=323 @Rt 5.06 min (100%).

### EXAMPLE 22D

### 1-(4-Chlorophenyl)-3-methyl-3-(3-methylamino-propyl)-3,4-dihydro-1H-quinolin-2-one (21Di)

This was prepared from (19Db) (100 mg, 0.30 mmol) using the same methods described for (21Da) to provide the racemate (97 mg). 1H NMR (300 MHz, CDCl₃) (racemate) δ ppm 1.25 (s, 3H), 1.55-1.65 (m, 4H), 2.41 (s, 3H), 2.58 (m, 2H), 2.89 (d, J=15.82 Hz, 1H), 2.98 (d, J=15.82 Hz, 1H), 3.12 (br, 1H), 6.29 (dd, J=7.91, 0.94 Hz, 1H), 6.95-7.10 (m, 2H), 7.14 (d, J=8.67 Hz, 2H), 7.15 (m, 1H), 7.45 (d, J=8.67 Hz, 2H). LCMS (12 minute method) [M+H]⁺=343/345 @Rt 5.09 min (100%).

### EXAMPLE 23D

### 1-(3,4-Difluorophenyl)-3-methyl-3-(3-methylamino-propyl)-3,4-dihydro-1H-quinolin-2-one (21Dj)

This was prepared from (19Db) (100 mg, 0.30 mmol) using the same two-step procedure described for (21Da) to provide the crude product, which was purified by SCX-2 to give the racemate (100 mg). ¹H NMR (300 MHz, CDCl₃) (racemate) δ ppm 1.25 (s, 3H), 1.55-1.65 (m, 4H), 2.41 (s, 3H), 2.50-2.60 (m, 2H), 2.89 (d, J=15.45 Hz, 1H), 2.90 (s, 1H), 2.98 (d, J=15.45 Hz, 1H), 6.30 (dd, J=7.91, 1.13 Hz, 1H), 6.90-7.10 (m, 4H), 7.18 (dd, J=7.16, 1.32 Hz, 1H), 7.22-7.35 (m, 1H). LCMS (12 minute method) [M+H]⁺=345 @Rt 4.85 min (97%).

### EXAMPLE 24D

### 3-Methyl-3-(3-methylamino-propyl)-1-m-tolyl-3,4-dihydro-1H-quinolin-2-one (21Dk)

This was prepared from (19Db) (100 mg, 0.30 mmol) using the same two-step procedure described for (21Da) to provide the crude product, which was purified by SCX-2 to give the racemate (90 mg). ¹H NMR (300 MHz, CDCl₃) (racemate) δ ppm 1.26 (s, 3H), 1.50-1.70 (m, 4H), 1.75 (s, 1H), 2.38 (s, 3H), 2.39 (s, 3H), 2.50-2.60 (m, 2H), 2.89 (d, J=15.64 Hz, 1H), 2.98 (d, J=15.64 Hz, 1H), 6.30 (dd, J=7.82, 1.04 Hz, 1H), 6.90-7.07 (m, 4H), 7.18 (dd, J=13.66, 7.63 Hz, 2H), 7.37 (t, J=7.63 Hz, 1H). LCMS (12 minute method) [M+H]⁺=323 @Rt 5.09 min (98%).

### EXAMPLE 25D

### 1-(3,5-Difluorophenyl)-3-methyl-3-(3-methylamino-propyl)-3,4-dihydro-1H-quinolin-2-one (21Dl)

This was prepared from (19Db) (100 mg, 0.30 mmol) using the same two-step procedure described for (21Da) to provide the crude product, which was purified by SCX-2 to give the racemate (95 mg). ¹H NMR (300 MHz, CDCl₃) (racemate) δ ppm 1.26 (s, 3H), 1.50-1.65 (m, 4H), 2.40 (s, 3H), 2.50-2.60 (m, 2H), 2.82 (br, 1H), 2.89 (d, J=15.82 Hz, 1H), 2.97 (d, J=15.82 Hz, 1H), 6.34 (dd, J=8.01, 1.04 Hz, 1H), 6.74-6.83 (m, 2H), 6.83-6.92 (m, 1H), 6.97-7.13 (m, 2H), 7.19 (dd, J=7.06, 1.22 Hz, 1H). LCMS (12 minute method) [M+H]⁺=345 @ Rt 4.87 min, (97%).

### EXAMPLE 26D

### 6-Chloro-3-(3-methylamino-propyl)-1-phenyl-3,4-dihydro-1H-quinolin-2-one (21Dm)

This was prepared from (20Da) (285 mg, 0.8 mmol) using the same two-step procedure described for (21Da) to provide the crude product, which was purified by preparative LCMS to give the racemate (62 mg). ¹H NMR (300 MHz, CDCl₃) (racemate) δ 1.49-1.76 (m, 3H), 1.86-1.95 (m, 1H), 2.33 (bs, 1H), 2.44 (s, 3H), 2.61-2.95 (m, 4H), 3.09-3.16 (m, 1H), 6.24-6.27 (d, J=8.67 Hz, 1H), 6.99 (dd, J=8.67, 2.26 Hz, 1H), 7.17-7.19 (m, 3H), 7.39-7.44 (m, 1H), 7.47-7.52 (m, 2H). LCMS (12 minute method) [M+H]⁺=329/331 @ Rt 5.04 min (93%).

### EXAMPLE 27D

### 6-Chloro-1-(4-chlorophenyl)-3-(3-methylamino-propyl)-3,4-dihydro-1H-quinolin-2-one (21Do)

This was prepared from (20Da) (160 mg, 0.45 mmol) using the same two-step procedure described for (21Da) to provide the crude product, which was purified by preparative LCMS to give the racemate (52 mg). ¹H NMR (300 MHz, CDCl₃) (racemate) δ 1.57-1.67 (m, 1H), 1.73-1.75 (m, 2H), 1.87-1.9 (m, 1H), 2.47 (s, 2H), 2.64 (s, 1H), 2.68-2.73 (m, 2H), 2.81-2.89 (m, 1H), 3.07-3.13 (m, 3H), 6.27 (d, J=8.48 Hz, 1H), 7.02 (d, J=8.48 Hz, 1H), 7.14 (d, J=8.29 Hz, 2H), 7.19 (s, 1H), 7.47 (d, J=8.29 Hz, 2H). LCMS (12 minute method) [M+H]⁺=363/365 @ Rt 5.4 min (72%).

### EXAMPLE 28D

### 6-Chloro-3-methyl-3-(3-methylamino-propyl)-1-p-tolyl-3,4-dihydro-1H-quinolin-2-one (21Dp)

This was prepared from (20Db) (490 mg, 1.34 mmol) using the same methods described for (21Da) to provide the racemate (470 mg). The racemate was separated into its individual enantiomers using chiral HPLC. ¹H NMR (300 MHz, CDCl₃) (racemate) δ 1.25 (s, 3H), 1.50-1.65 (m, 4H), 2.39 (s, 3H), 2.40 (s, 3H), 2.50-2.60 (m, 3H), 2.86 (d, J=16.01 Hz, 1H), 2.94 (d, J=16.01 Hz, 1H), 6.24 (d, J=8.67 Hz, 1H), 6.97 (dd, J=8.76, 2.35 Hz, 1), 7.03 (d, J=8.10 Hz, 2H), 7.14 (d, J=2.26 Hz, 1H), 7.29 (d, J=7.91 Hz, 2H); ¹H NMR (300 MHz, MeOD-d4) (isomer hemi-D-tartrate salt) δ 1.15 (s, 3H), 1.50-1.75 (m, 4H), 2.32 (s, 3H), 2.51 (s, 3H), 2.78 (br, 2H), 2.84 (d, J=16.20 Hz, 1H), 2.98 (m, 1H), 3.15-3.25 (m, 2H), 4.22 (s, 1H), 6.14 (d, J=8.85 Hz, 1H), 6.90-6.70 (m, 3H), 7.19 (d, J=2.26 Hz, 1H), 7.25 (d, J=7.91 Hz, 2H). LCMS (12 minute method) [M+H]⁺=357/359 @Rt 5.43 min (100%).

### EXAMPLE 29D

### 6-Chloro-1-(4-chlorophenyl)-3-methyl-3-(3-methylamino-propyl)-3,4-dihydro-1H-quinolin-2-one (21Dq)

This was prepared from (20Db) (490 mg, 1.34 mmol) using the same methods described for (21 Da) to provide the racemate (425 mg). ¹H NMR (300 MHz, CDCl₃) (racemate) δ ppm 1.25 (s, 3H), 1.50-1.65 (m, 4H), 2.39 (s, 3H), 2.40 (br, 1H), 2.50-2.60 (m, 2H), 2.87 (d, J=16.20 Hz, 1H), 2.95 (d, J=16.20 Hz, 1H), 6.23 (d, J=8.85 Hz, 1H), 7.00 (dd, J=8.57, 2.35 Hz, 1H), 7.05-7.20 (m, 3H), 7.40-7.50 (m, 2H). LCMS (12 minute method) [M+H]⁺=377/379 (Rt 5.26 min (94%).

### EXAMPLE 30D

### 3-Methyl-3-(3-methylamino-propyl)-1-thiophen-2-yl-3,4-dihydro-1H-quinolin-2-one (22Da)

This was prepared from (19Db) (200 mg, 0.60 mmol) using the same two-step procedure described for (21Da) to provide the crude product, which was purified by SCX-2 to give the racemate (125 mg). ¹H NMR (300 MHz, CDCl₃) (racemate) δ ppm 1.25 (s, 3H), 1.50-1.65 (m, 4H), 2.39 (s, 3H), 2.50-2.60 (br, 2H), 2.88 (d, J=16.20 Hz, 1H), 2.97 (d, J=16.20 Hz, 1H), 3.17 (br, 1H), 6.58 (dd, J=8.01, 0.85 Hz, 1H), 6.89 (dd, J=3.58, 1.32 Hz, 1H), 6.95-7.15 (m, 3H), 7.16 (d, J=7.16 Hz, 1H), 7.32 (dd, J=5.65, 1.32 Hz, 1H). LCMS (12 minute method) [M+H]⁺=315 @Rt 4.35 min (98%).

### EXAMPLE 31D

### 3-Methyl-3-(3-methylamino-propyl)-1-thiophen-3-yl-3,4-dihydro-1H-quinolin-2-one (22Db)

This was prepared from (19Db) (200 mg, 0.60 mmol) using the same two-step procedure described for (21Da) to provide the crude product, which was purified by SCX-2-2 to give the racemate (128 mg). ¹H NMR (300 MHz, CDCl₃) δ 1.24 (s, 3H), 1.50-1.65 (m, 4H), 2.40 (s, 3H), 2.50-2.60 (m, 2H), 2.87 (d, J=15.82 Hz, 1H), 2.96 (d, J=15.82 Hz, 1H), 3.07 (br, 1H), 6.45 (dd, J=8.10, 0.94 Hz, 1H), 6.92 (dd, J=5.09, 1.32 Hz, 1H), 6.98 (td, J=7.35, 1.13 Hz, 1H), 7.07 (td, J=7.77, 1.60 Hz, 1H), 7.16 (d, J=7.35 Hz, 1H), 7.22 (dd, J=3.20, 1.32 Hz, 1H), 7.41 (dd, J=5.09, 3.20 Hz, 1H). LCMS (12 minute method) [M+H]⁺=315 @Rt 4.29 min (100%).

Scheme 4D—Preparation of Intermediates

### {3-[1-(4-Methoxy-benzyl)-3-methyl-2-oxo-6-phenyl-1,2,3,4-tetrahydro-quinolin-3-yl]-propyl}-methyl-carbamic acid tert-butyl ester (23D)

Step (i)

Sodium hydride (340 mg, 60% dispersion in mineral oil, 8.55 mmol, 1.3 eq.) was added portionwise to a soln of (20Dc) (2.7 g. 6.57 mmol) in DMF (40 mL) at 0° C. The reaction mixture was left for 30 min at this temperature and then 4-methoxybenzyl chloride (1.16 mL, 8.55 mmol, 1.3 eq.) in DMF (1 mL) was added dropwise over 10 min. The reaction mixture was warmed to rt slowly and after 1 h was poured into ethyl acetate (200 mL) and extracted with water (3×50 mL). The organic layer was separated, dried over MgSO₄ and concentrated under vacuo. The crude product was purified using automated chromatography (silica) (0 to 80% ethyl acetatecyclohexane gradient) to provide the 4-methoxybenzyl protected 6-bromo precursor (2.2 g, 63%).

Step (ii)

The product from Step (i) (100 mg, 0.23 mmol), phenylboronic acid (85 mg, 0.70 mmol, 3 eq.), K₂CO₃ (138 mg, 1 mmol, 4.3 eq.) and Pd(PPh₃)₄ (11 mg, 0.009 mmol, 0.04 eq.) were suspended in ethanol (1 mL) and water (0.6 mL). The reaction mixture was heated at 80° C. overnight, cooled to rt and filtered through celite. The filtrate was poured into ethyl acetate (100 mL) and water (50 mL) and extracted. The organic layer was separated, dried over MgSO₄ and concentrated to provide the product (23D) (120 mg, 98%) that was used without further purification.

### Methyl-[3-(3-methyl-2-oxo-6-phenyl-1,2,3,4-tetrahydro-quinolin-3-yl)-propyl]-carbamic acid tert-butyl ester

Step (iii) & (iv)

A mixture of (23D) (120 mg, 0.23 mmol) and anisole (25 μL, 0.23 mmol) in trifluoroacetic acid (2.3 mL) was heated at 65° C. under nitrogen for 4 h. The reaction mixture was concentrated under vacuo and the residue was dissolved in methanol (2 mL). The methanol soln was applied to an SCX-2 column (5 g) and the column washed with methanol (50 mL). The product was eluted with 2N Et₃N in methanol (50 mL) and the basic soln was concentrated to provide 3-Methyl-3-(3-methylamino-propyl)-6-phenyl-3,4-dihydro-1H-quinolin-2-one (72 mg, 100%). To a soln of this amine (72 mg, 0.23 mmol) in anhydrous THF (2 mL) at 0° C. was added di-tert-butyl dicarbonate (53 mg, 97%, 0.24 mmol) in one portion. The reaction mixture was warmed to rt and stirred for 3 h. The reaction mixture was poured into ethyl acetate (25 mL) and water (10 mL) and extracted. The organic layer was separated, dried over MgSO₄ and concentrated to give the Boc protected precursor (95 mg, 100%). This material was used without further purification.

### Scheme 4D

### EXAMPLES

### EXAMPLE 32D

### 3-Methyl-3-(3-methylamino-propyl)-6-phenyl-1-p-tolyl-3,4-dihydro-1H-quinolin-2-one (24D)

This was prepared from the above Boc protected precursor (95 mg, 0.23 mmol) using the same two-step procedure described above (19Da to 21Da) to provide the crude product, which was purified by SCX-2 to give the racemate (53 mg). ¹H NMR (300 MHz, CDCl₃) (racemate) δ 1.29 (s, 3H), 1.50-1.70 (m, 4H), 2.42 (s, 6H), 2.55-2.65 (m, 2H), 2.94 (d, J=15.64 Hz, 1H), 3.04 (d, J=15.64 Hz, 1H), 3.18 (br, 1H), 6.38 (d, J=8.29 Hz, 1H), 7.09 (d, J=8.10 Hz, 2H), 7.29 (m, 4H), 7.41 (m, 3H), 7.54 (m, 2H). LCMS (12 minute method) [M+H]⁺=399 @Rt 6.06 min (100%).

The following examples illustrate compounds of of Formulae (IE) above and methods for their preparation.

Preparation of Intermediates

### 1,1-Dimethylethyl (3S)-3-aminopyrrolidine-1-carboxylate

### a) 1,1-Dimethylethyl (3R)-3-hydroxypyrrolidine-1-carboxylate

Solid ditert-butyldicarbonate (38.8 g, 178 mmol) was added in portions over 15 minutes to a stirred solution of (3R)-pyrrolidin-3-ol hydrochloride (20 g, 162 mmol), triethylamine (24.8 mL, 178 mmol) and 4-(dimethylamino)-pyridine (20 mg) in dry dichloromethane (300 mL). After stirring for 2 hours at room temperature, the mixture was washed with aqueous citric acid, then brine. The organic extracts were dried (MgSO₄), filtered and evaporated in vacuo to give an oil. This was purified by flash chromatography on silica, eluting with ethyl acetate/cyclohexane (20:80 to 60:40), to give the title compound as a solid.

### b) 1,1-Dimethylethyl (3R)-3-[(methylsulfonyl)oxy]-pyrrolidine-1-carboxylate

Methanesulfonyl chloride (5.26 mL, 68 mmol) was added dropwise over 5 minutes to a stirred solution of 1,1-dimethylethyl (3R)-3-hydroxypyrrolidine-1-carboxylate (10.6 g, 56.7 mmol) and triethylamine (11.8 mL, 85 mmol) in dichloromethane (250 mL) at −10° C. After stirring for 1 hour at 0° C., the reaction was quenched by addition of water. The organic phase was washed with brine, dried (MgSO₄), filtered and evaporated in vacuo to give an oil. This was purified by flash chromatography on silica, eluting with ethyl acetate/cyclohexane (25:75 to 50:50), to give the title compound as an oil.

### c) 1,1-Dimethylethyl (3S)-3-azidopyrrolidine-1-carboxylate

Sodium azide (4.4 g, 67.4 mmol) was added to a solution of 1,1-dimethylethyl (3R)-3-[(methylsulfonyl)oxy]-pyrrolidine-1-carboxylate (14.3 g, 54 mmol) in dry dimethylformamide (75 mL) and the resultant suspension heated at 65° C. for 8 hours. After cooling to room temperature, the reaction mixture was diluted with water and extracted into diethyl ether. The organic phase was washed two further times with water, then brine. The organic extracts were dried (MgSO₄), filtered and evaporated in vacuo to give an oil. This was purified by flash chromatography on silica, eluting with diethyl ether/cyclohexane (20:80 to 40:60), to give the title compound as an oil.

### d) 1,1-Dimethylethyl (3S)-3-aminopyrrolidine-1-carboxylate

A mixture of 1,1-dimethylethyl (3S)-3-azidopyrrolidine-1-carboxylate (9.0 g, 2.97 mmol) and 5% palladium-on-carbon (0.70 g) in methanol (150 mL) was hydrogenated in a Parr apparatus at 65 p.s.i. for 4 hours. The catalyst was removed by filtration through Celite and the solvent evaporated in vacuo to give an oil. The resultant title compound was used in subsequent reactions without further purification.

1,1-Dimethylethyl (3R)-3-aminopyrrolidine-1-carboxylate was similarly prepared as described above, from (3S)-pyrrolidin-3-ol.

### 1,1-Dimethylethyl (3S)-3-[(1-methylethyl)amino]-pyrrolidine-1-carboxylate

A mixture of 1,1-dimethylethyl (3S)-3-aminopyrrolidine-1-carboxylate (3.0 g) and 5% palladium-on-carbon (0.35 g) in methanol (75 mL) and acetone (15 mL) was hydrogenated in a Parr apparatus at 65 p.s.i. for 3 hours. The catalyst was removed by filtration through Celite and the solvent evaporated in vacuo to give an oil. The resultant title compound was used in subsequent reactions without further purification.

¹H NMR (300 MHz, CDCl₃) δ_{H}: 1.11-1.19 (m, 6H), 1.45 (s, 9H), 1.55-1.75 (m, 1H) 2.01-2.15 (m, 1H), 2.80-2.92 (m, 1H), 2.93-3.05 (m, 1H), 3.25-3.70 (m, 4H).

The following secondary amines were similarly prepared by reductive alkylation of 1,1-dimethylethyl (3S)-3-aminopyrrolidine-1-carboxylate with the appropriate aldehyde or ketone:
1,1-Dimethylethyl (3S)-3-(cyclopentylamino)pyrrolidine-1-carboxylate
1,1-Dimethylethyl (3S)-3-[(cyclohexylmethyl)amino]-pyrrolidine-1-carboxylate

### 1,1-Dimethylethyl (3S)-3-({[2-(trifluoromethyl)phenyl]-methyl}amino)pyrrolidine-1-carboxylate

Method A

### a)(3S)-N-{(E)-[2-(Trifluoromethyl)phenyl]methylidene}-pyrrolidin-3-amine

3(S)-Pyrrolidin-3-amine (0.45 g, 5.2 mmol) and trifluoromethylbenzaldehyde (0.87 g, 5.0 mmol), a crystal of 4-toluenesulphonic acid and toluene were refluxed with stirring for one day, using a Dean and Stark apparatus. The solution was evaporated in vacuo to give the title compound as a brown oil (M+H=243).

### b) 1,1-Dimethylethyl (3S)-3-({(E)-[2-(trifluoromethyl)-phenyl]methylidene}amino)pyrrolidine-1-carboxylate

(3S)-N-{(E)-[2-(Trifluoromethyl)phenyl]methylidene}-pyrrolidin-3-amine (1.21 g, 5 mmol) was dissolved in dichloromethane (50 mL), and di-tert-butyl dicarbonate (1.1 g, 5.05 mmol) followed by DMAP (60 mg, 0.5 mmol) was added. After stirring under nitrogen for 4 hours, the solution was evaporated in vacuo to give the title compound as a brown oil (M+H=343).

### c) 1,1-Dimethylethyl (3S)-3-({[2-(trifluoromethyl)-phenyl]methyl}amino)pyrrolidine-1-carboxylate

1,1-Dimethylethyl (3S)-3-({(E)-[2-(trifluoromethyl)-phenyl]methylidene}amino)pyrrolidine-1-carboxylate (1.71 g, 5 mmol) was hydrogenated in the presence of 5% palladium on carbon (250 mg) at 65 psi in ethanol (60 mL). After 3.5 hours, the catalyst was filtered off and the filtrate evaporated in vacuo to give an oil. The oil was purified by automated flash chromatography over silica, eluting with 10% ethyl acetate in cyclohexane (10:90 to 50:50), to give the title compound as a colourless oil (1.0 g, 58%; M+H=345).

Method B

### a) (3S)-N-{[2-(Trifluoromethyl)phenyl]methyl}pyrrolidin-3-amine

A mixture of 3(S)-pyrrolidin-3-amine (4 g, 46.5 mmol), 2-trifluoromethylbenzaldehyde (9.1 g, 46.5 mmol), 5% palladium on carbon (0.4 g) and ethanol (150 mL) was hydrogenated at 60 psi for 3 hours using a Parr hydrogenator. The catalyst was filtered off and the filtrate evaporated in vacuo to give the title compound as an oil. MS: [M+H]=245.

### b) 1,1-Dimethylethyl (3S)-3-({[2-(trifluoromethyl)-phenyl]methyl}amino)pyrrolidine-1-carboxylate

(3S)-N-{[2-(Trifluoromethyl)phenyl]methyl}pyrrolidin-3-amine (12 g, 49.2 mmol) was dissolved in dichloromethane (120 mL), then di-tert-butyl dicarbonate (10.7 g, 49.2 mmol) and DMAP (40 mg, 0.33 mmol) were added. After stirring under nitrogen for 1 day, the solution was evaporated in vacuo to give an oil. The oil was purified by automated flash chromatography over silica, eluting with ethyl acetate in cyclohexane (0:100 to 40:60), to give the title compound as a colourless oil.

MS: [M+H]=345.

### 1,1-Dimethylethyl (3S)-3-({[4-fluoro-2-(trifluoromethyl)-phenyl]methyl}amino)pyrrolidine-1-carboxylate

1,1-Dimethylethyl (3S)-3-aminopiperidine-1-carboxylate (5 g) and 4-fluoro-2-(trifluoromethyl)benzaldehyde (5.15 g, 26.8 mmol)were allowed to stir in methanol for 16 h at room temperature. Sodium borohydride (1.62 g, 26.8 mmol) was then added portionwise. The resulting solution was further stirred for 2 h at room temperature. The solvent was evaporated in vacuo, water was added, and the solution extracted with dichloromethane. The organic extracts were absorbed onto a methanol washed cationic ion exchange resin (Isolute™ SCX-2). The basic components were recovered from the column by elution with 7N ammonia in methanol. The resultant solution was concentrated in vacuo to yield the desired compound as an oil. This was further purified by column chromatography on silica gel, eluting with ethyl acetate/iso-hexane (0:100 to 40:60). The title compound was used in subsequent reactions without further purification.

¹H NMR (300 MHz, CDCl₃) δ_{H}: 7.37-7.28 (m, 2H), 7.24-7.20 (m, 1H), 3.80 (s, 2H), 3.52-3.48 (m, 2H), 3.32 (m, 3H), 3.12 (m, 1H), 2.08-2.0 (m, 1H), 1.75 (m, 1H), 1.45 (s, 9H).

The following secondary amines were similarly prepared by reductive alkylation of 1,1-dimethylethyl (3S)-3-aminopiperidine-1-carboxylate with the appropriate benzaldehyde:
1,1-Dimethylethyl (3S)-3-{[(3,5-dichloro-phenyl)methyl]-amino}pyrrolidine-1-carboxylate.
1,1-Dimethylethyl (3S)-3-{[(5-fluoro-2-(trifluoromethyl)-phenyl)methyl]amino}pyrrolidine-1-carboxylate.
1,1-Dimethylethyl (3S)-3-{[(2-chloro-4-fluoro-phenyl)-methyl]amino}pyrrolidine-1-carboxylate.

### EXAMPLE 1E

### (3S)-N-(1-Methylethyl)-N-{[3,5-dichlorophenyl]-methyl}pyrrolidin-3-amine D-tartrate

### a) 1,1-Dimethylethyl (3S)-3-((1-methylethyl)-{[3,5-dichlorophenyl]methyl}amino)-pyrrolidine-1-carboxylate

To a solution of 1,1-dimethylethyl (3S)-3-[(1-methylethyl)amino]-pyrrolidine-1-carboxylate (1 g, 4.4 mmol) and 3,5-dichlorobenzaldehyde (1.53 g, 8.77 mmol) in trimethylorthoformate (10 mL) at room temperature under a nitrogen atmosphere was added portionwise sodium triacetoxyborohydride (1.3 g, 6.1 mmol). The reaction was stirred at room temperature for 72 hours, then evaporated to dryness in vacuo. The residue was taken up in aqueous saturated sodium hydrogen carbonate/dichloromethane mixture. The aqueous layer was further extracted with dichloromethane (3X), and the combined organic layers dried (MgSO₄) and evaporated to dryness in vacuo. The resulting residue was dissolved in methanol and filtered through a cationic ion exchange resin (Isolute™ SCX-2). The basic components were recovered from the column by elution with 2N ammonia in methanol. This solution was concentrated in vacuo to yield the desired compound as a yellow oil that was used in the next step without further purification. ¹H NMR (300 MHz, CDCl₃) δ_{H}: 0.95-1.04 (m, 6H), 1.45 (s, 9H), 1.56-1.77 (m, 1H), 1.8-1.94 (m, 1H), 2.9-3.09 (m, 2H), 3.11-3.25 (m, 1H), 3.32-3.56 (m, 3H), 3.59 (s, 2H), 7.15-7.27 (m, 3H). MS: [M+H]=387/389/391.

### b)(3S)-N-(1-Methylethyl)-N-{[3,5-dichlorophenyl)methyl}-pyrrolidin-3-amine D-tartrate

1,1-Dimethylethyl (3S)-3-((1-methylethyl)-{[3,5-dichlorophenyl]methyl}amino)pyrrolidine-1-carboxylate (1.36 g, 3.51 mmol) was dissolved in a mixture of dichloromethane and trifluoroacetic acid (10 mL, 2:1) and stirred at room temperature for 30 minutes. The reaction solution was concentrated in vacuo and redissolved in MeOH. This solution was filtered through a cationic ion exchange resin (Isolute™ SCX-2). The basic components were isolated by elution with 2N ammonia in methanol and further purified by UV guided prep-LC. The desired compound was isolated from the acidic prep-LC mobile phase via a cationic ion exchange resin as described above. After evaporation in vacuo the residue was dissolved in hot cyclohexane (5 mL) and to this was added an equimolar amount of D-tartaric acid (450 mg), dissolved in a minimal amount of hot isopropanol. The solution was evaporated in vacuo to yield the title compound as a solid. ¹H NMR (300 MHz, d6-DMSO) δ_{H}: 0.95-0.99 (m, 6H), 1.58-1.71 (m, 1H), 1.91-2.00 (m, 1H), 2.76-2.91 (m, 2H), 2.97-3.07 (m, 1H), 3.18-3.25 (m, 2H), 3.55-3.67 (m, 4H), 3.95 (s, 2H), 7.37-7.38 (m, 2H), 7.43-7.45 (m, 1H). MS: [M+H]=287/289/291.

The following Examples were similarly prepared as described above for Example 1E, by reductive alkylation of 1,1-dimethylethyl (3S)-3-[(1-methylethyl)amino]-pyrrolidine-1-carboxylate with the appropriate substituted benzaldehyde:

### EXAMPLE 2E

### (3S)-N-(1-Methylethyl)-N-}[2-(methylthio)phenyl[methyl}-pyrrolidin-3-amine fumarate

¹H NMR (300 MHz, CD₃OD) δ_{H}: 0.99 (s, 6H), 2.06 (m, 1H), 2.37 (s, 3H), 3.01-2.85 (m, 1H), 3.18-3.06 (m, 1H), 3.46-3.19 (m, 4H), 3.67 (dd, 2H), 6.60 (s, 2H), 7.10-7.02 (m, 1H), 7.20-7.11 (m, 2H), 7.40 (dd, 1H); MS: [M+H]=265.

The following Examples were similarly prepared as described above for Example 1E, by reductive alkylation of 1,1-dimethylethyl (3S)-3-[(cyclohexylmethyl)amino]-pyrrolidine-1-carboxylate with the appropriate substituted benzaldehyde:

### EXAMPLE 3E

### (3S)-N-(Cyclohexylmethyl)-N-{[2-(methylthio)phenyl]-methyl}pyrrolidin-3-amine fumarate

¹H NMR (300 MHz, CD₃OD) δ_{H}: 0.86-0.69 (s, 3H), 1.22-1.12 (m, 3H), 1.41-1.29 (m, ¹H), 1.84-1.67 (m, 5H), 2.16-1.95 (m, 2H), 2.34 (d, 2H), 2.38 (s, 3H), 3.23-3.05 (m, 1H), 3.44-3.28 (m, 4H), 3.78-3.55 (m, 2H), 6.70 (s, 2H), 7.16 (s, 2H), 7.35-7.32 (m, 1H); MS: [M+H]=319.

### EXAMPLE 4E

### (3S)-N-(Cyclohexylmethyl)-N-[(2-fluorophenyl)methyl]-pyrrolidin-3-amine fumarate

¹H NMR (300 MHz, CD₃OD) δ_{H}: 0.83-0.75 (s, 6H), 1.24-1.17 (m, 3H), 1.48-1.42 (m, 1H), 1.85-1.68 (m, 5H), 2.03-1.92 (m, 1H), 2.17-2.10 (m, 1H), 2.35 (d, 2H), 3.25-3.05 (m, 1H), 3.44-3.32 (m, 4H), 3.81-3.62 (m, 2H), 6.71 (s, 2H), 7.20-7.05 (m, 2H), 7.33-7.27 (m, 1H), 7.47-7.42 (m, 1H); MS: [M+H]=291.

### EXAMPLE 5E

### (3S)-N-[(2-Chlorophenyl)methyl]-N-(cyclohexylmethyl)-pyrrolidin-3-amine fumarate

¹H NMR (300 MHz, CD₃OD) δ_{H}: 0.89-0.77 (m, 2H), 1.24-1.13 (m, 3H), 1.36 (d, 6H), 1.49-1.42 (m, 1H), 1.83-1.68 (m, 5H), 2.15-1.93 (m, 2H), 2.35 (d, 2H), 3.20-3.06 (m, 1H), 3.33-3.23 (m, 4H), 3.75-3.42 (m, 2H), 4.69-4.61 (m, 1H), 6.70 (s, 2H), 6.98-6.88 (m, 2H), 7.35 (d, 1H), 7.50-7.19 (m, 1H); MS: [M+H]=307.

### EXAMPLE 6E

### (3S)-N-(Cyclohexylmethyl)-N-({2-[1-(methylethyl)oxy]-phenyl}methyl)pyrrolidin-3-amine fumarate

¹H NMR (300 MHz, CD₃OD) δ_{H}: 0.89-0.77 (m, 2H), 1.24-1.13 (m, 3H), 1.36-1.34 (dd, 6H), 1.49-1.42 (m, 1H), 1.83-1.68 (m, 5H), 1.93 (m, 2H, m), 2.35 (d, 2H), 3.20-3.06 3.20-3.06 (m, 1H), 3.33-3.23 (m, 4H), 3.75-3.42 (m, 2H), 4.69-4.61 (m, 1H), 6.70 (s, 2H), 6.98-6.88 (m, 2H), 7.35 (d, 1H), 7.50-7.19 (m, 1H); MS: [M+H]=331.

### EXAMPLE 7E

### (3S)-N-{[5-Fluoro-2-(trifluoromethyl)phenyl]methyl}-N-(tetrahydro-2H-pyran-4-yl)pyrrolidin-3-amine D-tartrate

### a) 1,1-Dimethylethyl (3S)-3-[(tetrahydro-2H-pyran-4-yl)amino]pyrrolidine-1-carboxylate

Neat tetrahydro-4H-pyran-4-one (18.7 g, 100 mmol) and 1,1-dimethylethyl (3S)-3-aminopyrrolidine-1-carboxylate (26.1 g, 140.1 mmol) were stirred together for 20 minutes prior to addition of anhydrous dichloroethane (140 mL). The solution was then cooled to 0° C. under nitrogen and stirred as sodium triacetoxyborohydride (59.2 g, 281 mmol) was added portionwise. The reaction was allowed to warm to room temperature and stirred for 5 days, after which the reaction solution was carefully poured onto ice-cold aqueous sodium hydrogen carbonate solution. The phases were separated and the aqueous phase washed with dichloromethane. The combined organic phases were dried (MgSO₄) and concentrated in vacuo. The crude product was purified by automated flash chromatography on silica, eluting with methanol in ethyl acetate (0:100 to 30:70), to provide the title compound as an off-white solid. ¹H NMR (300 MHz, d6-DMSO) δ_{H}: 1.13-1.29 (m, 2H), 1.39 (s, 9H), 1.55-1.65 (m, 1H), 1.68-1.81 (m, 2H), 1.87-2.00 (m, 1H), 2.64 (sep, 1H), 2.91 (sex, 1H), 3.10-3.45 (m, 6H), 3.81 (dt, 2H). MS: [M+H]=271, [M+H−tBu]=215.

### b) (3S)-N-{[5-Fluoro-2-(trifluoromethyl)phenyl]methyl)-N-(tetrahydro-2H-pyran-4-yl)pyrrolidin-3-amine D-tartrate

To a stirred solution of 1,1-dimethylethyl (3S)-3-[(tetrahydro-2H-pyran-4-yl)amino]pyrrolidine-1-carboxylate (1.12 g, 4.2 mmol) and 5-fluoro-2-(trifluoromethyl)benzaldehyde (4.56 g, 23.8 mmol) in anhydrous dichloroethane (50 mL) was added portionwise sodium triacetoxyborohydride (3.86 g, 18.3 mmol). The reaction mixture was stirred at room temperature under nitrogen and the reaction progress was followed by MS. After 2 days more reagents were added: 5-fluoro-2-(trifluoromethyl)benzaldehyde (0.98 g, 5.1 mmol) and sodium triacetoxyborohydride (3.00 g, 14.2 mmol), and after a further 2 days the reaction was found to be complete. The reaction solution was carefully poured onto ice-cold saturated aqueous sodium hydrogen carbonate solution and filtered through a PTFE hydrophobic frit. The organic phase was concentrated in vacuo and the residue redissolved in methanol. The methanolic solution was filtered through a cationic ion exchange resin (Isolute™ SCX-2) and the basic components isolated by elution with 2N ammonia in methanol. After concentrating in vacuo, the residue was redissolved in dichloromethane/trifluoro-acetic acid (2:1) and allowed to stir at room temperature for 4 hours. The reaction mixture was concentrated in vacuo and redissolved in methanol. The methanolic solution was filtered through a cationic ion exchange resin (Isolute™ SCX-2) and the basic components isolated by elution with 2N ammonia in methanol. The crude product was purified by UV guided prep-LC, and the desired compound collected from the acidic prep-LC mobile phase via a cationic ion exchange resin, as described above. The basic product was dissolved in hot cyclohexane and to this was added an equimolar amount of D-tartaric acid dissolved in a minimal amount of hot isopropanol. The solution was allowed to cool overnight, and the next day the resultant solid was filtered off and dried in vacuo, to yield the title compound as a white crystalline solid. ¹H NMR (300 MHz, d6-DMSO) δ_{H}: 1.40-1.80 (m, 5H), 1.91-2.06 (m, 1H), 2.61-2.74 (m, 1H), 2.81-2.93 (dd, 1H), 2.97-3.11 (dt, 1H), 3.12-3.31 (m, 4H), 3.69-3.96 (m, 7H), 7.49-7.61 (m, 2H), 7.90-7.99 (m, 1H). MS: [M+H]=347.

The following Examples were similarly prepared from 1,1-dimethylethyl (3S)-3-[(tetrahydro-2H-pyran-4-yl)amino]pyrrolidine-1-carboxylate and the appropriate benzaldehyde, as described above for Example 7E:

### EXAMPLE 8E

### (3S)-N-{[2-(Trifluoromethyl)phenyl]methyl}-N-(tetrahydro-2H-pyran-4-yl)pyrrolidin-3-amine hemi-D-tartrate

¹H NMR (300 MHz, d6-DMSO) δ_{H}: 1.35-1.75 (m, 5H), 1.90-2.04 (m,1H), 2.63-2.75 (m, 1H), 2.76-2.86 (m, 1H), 2.94-3.03 (m, 1H), 3.10-3.25 (m, 4H), 3.67-3.90 (m, 6H), 7.43 (t, 1H), 7.66 (t, 2H), 7.92 (d, 1H); MS: [M+H]=329.

### EXAMPLE 9E

### (3S)-N-(1-Methylethyl)-N-{[2-(trifluoromethyl)-5-fluorophenyl]methyl}pyrrolidin-3-amine fumarate

### a) 1,1-Dimethylethyl (3S)-3-((1-methylethyl)-{[2-(trifluoromethyl)-5-fluorophenyl]methyl}amino)-pyrrolidine-1-carboxylate

A solution of 1,1-dimethylethyl (3S)-3-[(1-methylethyl)amino]pyrrolidine-1-carboxylate (0.34 g, 1.5 mmol) and 2-(trifluoromethyl)-5-fluorobenzyl bromide (0.58 g, 2.25 mmol) in acetonitrile (5 mL) was heated at reflux with anhydrous potassium carbonate (0.41 g, 3 mmol) for 24 hours. The reaction mixture was cooled, diluted with ethyl acetate and washed with water. The organic extracts were washed with brine, dried (MgSO₄), filtered and evaporated in vacuo to give an oil. This was purified by flash chromatography on silica, eluting with ethyl acetate/cyclohexane (0:100 to 10:90), to give the title compound as an oil.

### b) (3S)-N-(1-Methylethyl)-N-{[2-(trifluoromethyl)-5-fluorophenyl]methyl}pyrrolidin-3-amine fumarate

A solution of 1,1-dimethylethyl (3S)-3-((1-methylethyl)-{[2-(trifluoromethyl)-5-fluorophenyl]-methyl}amino)-pyrrolidine-1-carboxylate (0.26 g) in a mixture of trifluoroacetic acid (2 mL), dichloromethane (8 mL) and water (0.2 mL) was stirred at room temperature for 3 hours. The reaction mixture was evaporated in vacuo. The crude mixture was taken up in methanol and absorbed onto an SCX-2 ion exchange cartridge. After initially washing with methanol, the product was eluted with 2M methanolic ammonia and the collected fractions evaporated in vacuo. The crude product was taken up in methanol and fumaric acid (1 equiv.) in methanol added. The solvent was removed in vacuo and the resultant gum triturated with diethyl ether. The solid formed was filtered off and dried in vacuo at 50° C. to yield the title compound as an off-white microcrystalline solid. ¹H NMR (300 MHz, CD₃OD) δ_{H}: 1.09 (d, 3H), 1.10 (d, 3H), 1.87 (m, 1H), 2.15 (m, 1H), 3.01 (m, 2H), 3.23 (m, 1H), 3.38 (m, 2H), 3.81 (m, 1H), 3.91 (s, 2H), 6.70 (s, 2H), 7.15 (dt, 1H), 7.73 (m, 2H); MS: [M+H]=305.

The following Examples were similarly prepared as described for Example 9E, using the appropriate substituted benzyl bromide in step b) above:

### EXAMPLE 10E

### (3S)-N-([1,1′-Biphenyl]-2-ylmethyl)-N-(1-methylethyl)-pyrrolidin-3-amine fumarate

¹H NMR (300 MHz, CD₃OD) δ_{H}: 0.95 (d, 6H), 1.75 (m, 1H), 1.91 (m, 1H), 2.75 (dd, 1H), 2.93 (sept, 1H), 3.10 (m, 2H), 3.25 (m, 1B), 3.60 (m, 3H), 6.70 (s, 2H), 7.17 (dd, 1H), 7.25-7.48 (m, 7H), 7.67 (d, 1H); MS: [M+H]=295.

### EXAMPLE 11E

### Methyl ((3S)-pyrrolidin-3-yl{[2-(trifluoromethyl)phenyl]-methyl}amino)acetate D-tartrate

60% Sodium hydride oil dispersion (39 mg, 0.95 mmol) was added to 1,1-dimethylethyl (3S)-3-({[2-(trifluoromethyl)-phenyl]methyl}amino)pyrrolidine-1-carboxylate (250 mg, 0.73 mmol) in DMF (5 mL). After heating at 50° C. for 1 hour under nitrogen, methyl bromoacetate (123 mg, 0.73 mmol) was added. After heating overnight at 50° C. overnight, excess water was added and the product was extracted into ether. The ether was washed with water, dried (MgSO₄) and evaporated in vacuo to give an oil (460 mg). The oil was dissolved in dichloromethane (5 mL) and trifluoroacetic acid (0.5 mL) was added. After stirring for 1 day, the solution was evaporated in vacuo to give an oil. The oil was purified using preparative LCMS to give the product as the acetate salt, which was converted to the free base by absorption onto a cationic ion exchange resin (Isolute™ SCX-2) and eluting the basic fractions with 2N ammonia in methanol. The resultant oil was converted to the D-tartaric acid salt (crystallised from ethanol/diethyl ether) to give the title compound as a white solid. ¹H NMR(300 MHz, CD₃OD) δ_{H}: 1.84-196 (m, 1H), 2.06-2.14 (m, 1H), 3.06-3.37 (2×m,6H), 3.57 (s, 3H), 3.77-3.86 quin,1H), 3.91-4.06 (q, 2H), 4.29 (s, 2H), 7.32-7.36 (t, 1H), 7.49-7.54 (t, 1H), 7.56-7.59 (d, 1H), 7.76-7.89 (d, 1H); MS: [M+H]=317.

The following Examples were prepared from 1,1-dimethylethyl (3S)-3-aminopyrrolidine-1-carboxylate by initial reductive alkylation with 2-methylpropanaldehyde, followed by a second reductive alkylation with the appropriate benzaldehyde and subsequent deprotection.

### EXAMPLE 12E

### (3S)-N-{[2-(Methoxy)phenyl]methyl}-N-(2-methylpropyl)pyrrolidin-3-amine fumarate

¹H NMR (300 MHz, CD₃OD) δ_{H}: 0.82 (dd, 6H), 1.66 (sept, 1H), 1.79-1.92 (m, 1H), 1.92-2.06 (m, 1H), 2.19-2.22 (m, 2H), 2.96-3.13 (m, 2H), 3.18-3.31 (m, 2H), 3.59-3.67 (m, 2H), 3.74 (s, 3H), 6.59 (s, 2H), 6.80-6.87 (m, 2H), 7.11-7.18 (m, 1H), 7.25 (dd, 1H); MS: [M+H]=263.

The following Examples were prepared from 1,1-dimethylethyl (3S)-3-({[2-(trifluoromethyl)phenyl]-methyl}amino)pyrrolidine-1-carboxylate by reductive alkylation with the appropriate aldehyde or ketone and subsequent deprotection.

### EXAMPLE 13E

### (3S)-N-(1-Methylethyl)-N-{[2-(trifluoromethyl)-phenyl]methyl}pyrrolidin-3-amine fumarate

¹H NMR (300 MHz, CD₃OD) δ_{H}: 7.98-8.00 (d, 1H), 7.60-7.68 (d+t, 2H), 7.38-7.43(t, 1H), 6.70 (s, 2H), 3.91 (bs, 2H), 3.74-3.85 (m, 1H), 3.17-3.40 (M, 5H), 2.96-3.10 (m,3H), 2.08-2.18 (m, 1H), 1.82-1.96 (m,1H), 1.08-1.11 (dd, 6H); MS: [M+H]=287.

### EXAMPLE 14E

### (3S)-N-Ethyl-N-{[2-(trifluoromethyl)phenyl]methyl}-pyrrolidin-3-amine fumarate

¹H NMR (300 MHz, CD₃OD) δ_{H}: 8.00-8.03 (d, 1H), 7.67-7.76 (d+t, 2H), 7.47-7.52 (t, 1H), 6.77 (s, 2H), 3.89-4.03 (q, 2H), 3.65-3.75 (quin, 2H), 3.43-3.53 (m, 2H), 3.28-3.41 (m, 1H), 3.17-3.23 (m, 1H), 2.73-2.84 (q, 2H), 2.19-2.30 (m, 2H), 2.19-2.30 (m, 1H), 1.98-2.14 (m, 1H), 1.10-1.15 (t, 3H); MS: [M+H]=273.

### EXAMPLE 15E

### (3S)-N-Propyl-N-{[2-(trifluoromethyl)phenyl]methyl}-pyrrolidin-3-amine fumarate

¹H NMR (300 MHz, CD₃OD) δ_{H}: 7.92-7.94 (d, 1H), 7.60-7.69) d+t, 2H), 7.40-7.45 (t, 1H), 6.69-6.73 (s, 2H), 3.82-3.98 (q, 2H), 5.59-3.69(quin, 1H), 3.35-3.45 (m, 2H), 2.80-3.21 (m, 1H), 3.08-3.15 (m, 1H), 2.54-2.59 (q, 2H), 2.10-2.21 (m, 1H), 1.90-2.06 (m, 1H), 1.44-1.56 (quin, 2H), 0.86-0.91 (T, 3H); MS: [M+H]=287.

### EXAMPLE 16E

### (3S)-N-(Cyclohexylmethyl)-N-{[2-(trifluoromethyl)-phenyl]methyl}pyrrolidin-3-amine fumarate

¹H NMR (300 MHz, CD₃OD) δ_{H}: 77.89-7.92 (d, 1H), 7.61-7.70 (d+t, 2H), 7.41-7.49 (t, 1H), 6.70 (s, 2H), 3.81-3.95 (q, 2H), 3.56-3.67 (quin, 1H), 3.31-3.43 (m, 2H), 3.14-3.23 (m, 1H), 3.04-3.11 (m, 1H), 2.39-2.41 (d, 2H), 2.06-2.13 (m, 1H), 1.70-2.01 (m, 6H), 1.34-1.46 (m, 1H), 1.12-1.23 (m, 1H), 0.83-0.89 (m, 2H); MS: [M+H]=341.

### EXAMPLE 17E

### (3S)-N-Butyl-N-{[2-(trifluoromethyl)phenyl]methyl}-pyrrolidin-3-amine fumarate

¹H NMR (300 MHz, CD₃OD) δ_{H}: 7.91-7.94 (d, 1H), 7.60-7.69 (m, 2H), 7.40-7.45 (t, 1H), 6.70 (s, 2H), 3.82-3.96 (q, 2H), 3.59-3.69 (quin, 1H), 3.32-3.50 (m, 2H), 3.22-3.29 (m, 1H), 3.09-3.15 (q, 1H), 2.58-2.63 (t, 2H), 2.10-2.21 (m, 1H), 1.90-2.04 (m, 1H), 1.42-1.51 (m, 2H), 1.17-1.37 (m, 2H), 0.87-0.91 (t, 3H); MS: [M+H]=301.

### EXAMPLE 18E

### (3S)-N-(2-Ethylbutyl)-N-{[2-(trifluoromethyl)phenyl]-methyl}pyrrolidin-3-amine sesquifumarate

¹H NMR (300 MHz, CD₃OD) δ_{H}: 7.77-7.80 (d, 1H), 7.49-7.60 (m, 2H), 7.29-7.34 (t, 1H), 6.60 (s, 1.5H), 3.70-3.81 (q, 2H), 3.46-3.57 (quin, 1H), 3.20-3.33 (m, 2H), 2.94-3.13 (m, 2H), 2.32-2.34 (d, 2H), 1.97-2.07 (m 1H), 1.78-1.91 (m, 1H), 1.05-1.40 (m, 5H), 0.69-0.76 (m, 6H). MS: [M+H]=329.

### EXAMPLE 19E

### (3S)-N-{[2-(Trifluoromethyl)phenyl]methyl}-N-(3,3,3-trifluoropropyl)pyrrolidin-3-amine fumarate

¹H NMR (300 MHz, CD₃OD) δ_{H}: 7.76-7.78 (d, 1H), 7.50-7.60 (d+t, 2H), 7.32-7.37 (t, 1H), 6.58 (s, 2H), 3.75-3.89 (q, 2H), 3.48-3.59 (quin, 1H), 3.126-3.22 (m, 1H), 2.98-3.05 (dd, 1H), 2.75-2.80 (t, 2H), 2.18-2.34 (m, 2H), 2.02-2.13 (m, 1H), 1.80-1.93 (m, 1H); MS: [M+H]=341.

### EXAMPLE 20E

### (3S)-N-(Furan-2-ylmethyl)-N-{[2-(trifluoromethyl)phenyl]-methyl}pyrrolidin-3-amine D-tartrate

¹H NMR (300 MHz, CD₃OD) δ_{H}: 7.83-7.86 (d, 1H), 7.49-7.58 (t+s, 2H), 7.29-7.38 (m, 2H), 6.23-6.26 (m, 1H), 6.14-6.15 (m, 1H), 4.30 (s, 2H), 3.78-3.91 (q, 2H), 3.66-3.67 (m, 2H), 3.25-3.55 (m, 3H), 2.30-3.17 (m, 2H), 2.05-2.16 (m, 1H), 1.83-1.96 (m, 1H); MS: [M+H]=325.

### EXAMPLE 21E

### (3S)-N-[3-(Methylthio)propyl]-N-{[2-(trifluoromethyl)-phenyl]methyl}pyrrolidin-3-amine D-tartrate

¹H NMR (300 MHz, CD₃OD) δ_{H}: 7.90-7.92 (d,1H), 7.61-7.70 (d+t, 2H), 7.41-7.46 (t, 1H), 4.42 (s, 2H), 3.84-3.97 (q, 2H), 3.59-3.69 (quin, 1H), 3.38-3.47 (m, 2H), 3.19-3.29 (m, ¹H), 3.09-3.16 (m, 1H), 2.70-2.77 (dt, 2H), 2.48-2.52 (t, 2H), 2.08-2.21 (m, 1H), 1.89-2.08 (s+m, 4H), 1.69-1.79 (quin, 2H); MS: [M+H]=333.

### EXAMPLE 22E

### N-(Phenylmethyl)-N-[(3S)-pyrrolidin-3-yl]-N-{[2-(trifluoromethyl)phenyl]methyl}amine fumarate

¹H NMR (300 MHz, CD₃OD) δ_{H}: 7.93-7.96 (d, 1H), 7.60-7.68 (q, 2H), 7.23-7.44 (m, 6H), 6.69 (s, 2H), 3.83-3.94 (s,2H), 3.61-3.80 (m, 3H), 3.32-3.44 (m, 2H), 3.08-3.25 (m, 2H), 1.99-2.22 (m, 2H); MS: [M+H]=335.

### EXAMPLE 23E

### (3S)-N-{[2-(Methyloxy)phenyl]methyl]-N-{[2-(trifluoromethyl)phenyl]methyl}pyrrolidin-3-amine fumarate

¹H NMR (300 MHz, CD₃OD) δ_{H}: 7.85-7.87 (d, 1H), 7.61-7.64 (d, 1H), 7.52-7.58 (t, 1H), 7.21-7.40 (m, 3H), 6.81-6.97 (m, 2H), 6.69 (s, 2H), 3.61-3.97 (m, 8H), 3.16-3.44 (m, 4H), 1.20-2.21 (m, 2H); MS: [M+H]=365.

### EXAMPLE 24E

### (3S)-N,N-bis{[2-(Trifluoromethyl)phenyl]methyl}-pyrrolidin-3-amine fumarate

¹H NMR (300 MHz, CD₃OD) δ_{H}: 7.90-7.92 (d, 2H), 7.66-7.69 (d, 2H),7.59-7.64 (t, 2H), 7.40-7.45 (t, 2H), 6.69 (s, 2H), 3.91 (s, 4H), 3.62-3-74 (quin, 1H), 3.36-3.46 (m, 2H), 3.16-3.26 (m, 2H), 2.02-2.24 (m, 2H); MS: [M+H]=403.

The following examples illustrate compounds of of Formulae (IF) above and methods for their preparation.

Preparation of Intermediates

### 1,1-Dimethylethyl (3S)-3-aminopiperidine-1-carboxylate

### e) 1,1-Dimethylethyl (3R)-3-hydroxypiperidine-1-carboxylate

Solid ditert-butyldicarbonate (26.6 g, 122 mmol) was added in portions over 15 minutes to a stirred solution of (3R)-piperidin-3-ol hydrochloride (15.25 g, 111 mmol), triethylamine (30.9 mL, 222 mmol) and 4-(dimethylamino)-pyridine (50 mg) in dry dichloromethane (300 mL). After stirring for 18 hours at room temperature, the mixture was washed with aqueous citric acid, then brine. The organic extracts were dried (MgSO₄), filtered and evaporated in vacuo to give an oil. This was purified by flash chromatography on silica, eluting with ethyl acetate/cyclohexane (20:80 to 80:20), to give the title compound as a solid.

### f) 1,1-Dimethylethyl (3R)-3-[(methylsulfonyl)oxy]-piperidine-1-carboxylate

Methanesulfonyl chloride (9.56 mL, 124 mmol) was added dropwise over 10 minutes to a stirred solution of 1,1-dimethylethyl (3R)-3-hydroxypiperidine-1-carboxylate (20.7 g, 103 mmol) and triethylamine (21.5 mL, 154 mmol) in dichloromethane (300 mL) at 0° C. After stirring for 3 hour at 0° C., the reaction was quenched by addition of water. The organic phase was washed with brine, dried (MgSO₄), filtered and evaporated in vacuo to give an oil. This was purified by flash chromatography on silica, eluting with ethyl acetate/cyclohexane (20:80 to 50:50), to give the title compound as an oil.

### g) 1,1-Dimethylethyl (3S)-3-azidopiperidine-1-carboxylate

Sodium azide (7.65 g, 118 mmol) was added to a solution of 1,1-dimethylethyl (3R)-3-[(methylsulfonyl)oxy]-piperidine-1-carboxylate (21.9 g, 78.5 mmol) in dry dimethylformamide (120 mL) and the resultant suspension heated at 70° C. for 48 hours. After cooling to room temperature, the reaction mixture was diluted with water and extracted into ethyl acetate. The organic phase was washed two further times with water, then brine. The organic extracts were dried (MgSO₄), filtered and evaporated in vacuo to give an oil. This was purified by flash chromatography on silica, eluting with ethyl acetate/cyclohexane (10:90 to 50:50), to give the title compound as an oil.

### h) 1,1-Dimethylethyl (3S)-3-aminopiperidine-1-carboxylate

A mixture of 1,1-dimethylethyl (3S)-3-azidopiperidine-1-carboxylate (7.5 g) and 10% palladium-on-carbon (0.75 g) in methanol (100 mL) was hydrogenated in a Parr apparatus at 70 p.s.i. for 16 hours. The catalyst was removed by filtration through Celite and the solvent evaporated in vacuo to give an oil. The resultant title compound was used in subsequent reactions without further purification.

### 2-(Bromomethyl)-4-fluoro-1,1′-biphenyl

### a) Methyl 5-fluoro-2-{[(trifluoromethyl)sulfonyl]-oxy}benzoate

5-Fluorosalicylic acid methyl ester (28.2 g, 166 mmol) was dissolved in dry dimethylformamide (165 mL) and stirred as sodium hydride (60% in oil) (7.30 g, 1.1 eq) was added portionwise over 30 mins at 0° C. The reaction mixture was stirred for a further 30 mins at room temperature, then N-phenyl trifluoromethanesulfonimide (62.8 g, 1.05 eq) was added in portions over 30 mins, then left to stir for 3 hours. The mixture was diluted with diethyl ether and washed successively with water, then brine. The organic layers were combined, dried (MgSO₄), filtered and the solvent removed in vacuo. The resulting oil was purified by flash chromatography on silica, eluting with ethyl acetate/cyclohexane (10:90 to 40:60), to give the title compound as an oil.

### b) Methyl 4-fluoro-[1,1′-biphenyl]-2-carboxylate

Palladium acetate (635 mg, 0.05 eq), tricyclohexyl-phosphine (952 mg, 0.06 eq), potassium fluoride (10.85 g, 3.3 eq) and phenyl boronic acid (7.6 g, 1.1 eq) were taken up in dry THF (150 mL) and the reaction mixture flushed with nitrogen for 5 mins. A solution of methyl 5-fluoro-2-{[(trifluoromethyl)sulfonyl]oxy}benzoate (17.12 g, 56.7 mmol) in THF (20 mL) was added in one portion and the reaction mixture stirred at reflux under nitrogen for 5 hours. The reaction mixture was cooled to room temperature, diluted with ethyl acetate, then washed with water, dried (MgSO₄), filtered and the solvent removed in vacuo. The resulting oil was purified by flash chromatography on silica, eluting with ethyl acetate/cyclohexane (3:97 to 10:90), to give the title compound as an oil.

### c) (4-Fluoro-[1,1′-biphenyl]-2-yl)methanol

A solution of methyl 4-fluoro-[1,1′-biphenyl]-2-carboxylate (3 g, 13.1 mmol) in THF (20 mL) was added at 0° C. to a suspension of lithium aluminium hydride pellets (1 g, 26 mmol) in THF (30 mL). Upon addition the reaction mixture was heated at 60° C. under nitrogen for 2 h. The reaction was then cooled to 0° C. and the excess lithium aluminium hydride destroyed by adding water, then 1N sodium hydroxide (2 mL). The mixture was extracted into diethyl ether and the organic phase was dried (MgSO₄), filtered and the solvent removed in vacuo. The resulting oil was purified by flash chromatography on silica, eluting with ethyl acetate/heptane (2:98 to 25:75), to give the title compound as an oil.

### d) 2-(Bromomethyl)-4-fluoro-1,1′-biphenyl

Triphenylphosphine dibromide (35.5 g, 2 eq) was added in one portion to a solution of (4-fluoro-[1,1′-biphenyl]-2-yl)methanol (8.5 g, 42 mmol) in chloroform (250 mL). The reaction mixture was heated at 60° C. and left to stir overnight. The solid was filtered off and the solvent removed in vacuo. The resulting oil was purified by flash chromatography on silica, eluting with ethyl acetate/cyclohexane (0:100 to 30:70), to give the title compound as an oil.

### EXAMPLE 1F

### (3S)-N-(2-Methylpropyl)-N-{[2-(trifluoromethyl)-phenyl]methyl}piperidin-3-amine, fumarate

### a) 1,1-Dimethylethyl (3S)-3-({[2-(trifluoromethyl)-phenyl]methyl}amino)piperidine-1-carboxylate

1,1-Dimethylethyl (3S)-3-aminopiperidine-1-carboxylate (1.0 g, 5 mmol), 2-trifluoromethylbenzaldehyde (0.87 g, 5 mmol), 5% palladium on carbon (0.35 g) and ethanol (40 mL) were hydrogenated at 60 psi for 2.5 h. using a Parr hydrogenator. The catalyst was filtered off and the filtrate evaporated in vacuo. The resultant oil was purified by flash chromatography on silica, eluting with ethyl acetate/cyclohexane (0:100 to 75:25), to give the title compound as an oil.

### b) 1,1-Dimethylethyl (3S)-3-((2-methylpropyl){[2-(trifluoromethyl)phenyl]methyl}amino)piperidine-1-carboxylate

Sodium triacetoxyborohydride (0.23 g, 1.08 mmol) was added to a stirred solution of 1,1-dimethylethyl (3S)-3-({[2-(trifluoromethyl)phenyl]methyl}amino)piperidine-1-carboxylate (0.19 g, 0.53 mmol), isobutyraldehyde (0.12 g, 1.6 mmol)and 1,2-dichloroethane (5 mL). After stirring under nitrogen at room temperature for 1 day, the reaction mixture was diluted with methanol (6 mL) and absorbed onto a cationic ion exchange resin (Isolute™ SCX-2). After washing the cartridge with methanol (25mL), the basic components were isolated by elution with 2N ammonia in methanol and the eluate evaporated to give an oil.

### c) (3S)-N-(2-Methylpropyl)-N-{[2-(trifluoromethyl)-phenyl]methyl}piperidin-3-amine, fumarate

1,1-Dimethylethyl (3S)-3-((2-methylpropyl){[2-(trifluoromethyl)phenyl]methyl}amino)piperidine-1-carboxylate (0.139 mg, 0.335 mmol), trifluoroacetic acid (4 mL) and dichloromethane (10 mL) were stirred at room temperature for 1 day. The solution was evaporated in vacuo to give an oil, which was redissolved in methanol and filtered through a cationic ion exchange resin (Isolute™ SCX-2). The basic components were isolated by elution with 2N ammonia in methanol. The eluate was evaporated in vacuo and the resultant oil converted to the fumaric acid salt (crystallisation from ethanol/ether), to give the title compound as a white solid. ¹H NMR (300 MHz, CD₃OD): δ_{H} 7.77-7.74 (d, H), 7.51-7.43 (m, 2H), 7.25-7.22 (t, 1H), 4.23 (s, 2H) 3.79-3.66 (q, 2H), 3.21-3.08 (m, 4H), 2.83-2.61 (m, 3H), 2.28-2.10 (m, 2H), 1.90-1.82 (m, 2H), 1.59-1.37 (m, 3H), 0.77-72 (t, 6H); MS: (M+H)=315.

The following Examples were similarly prepared as described above for Example 1F, by reductive alkylation of 1,1-dimethylethyl (3S)-3-({[2-(trifluoromethyl)-phenyl]methyl}amino)piperidine-1-carboxylate with the appropriate aldehyde or ketone, and subsequent deprotection:

### EXAMPLE 2F

### (3S)-N-(3,3-Dimethylbutyl)-N-{[2-(trifluoromethyl)-phenyl]methyl}piperidin-3-amine, D-tartrate

¹HNMR (300 MHz, CD₃OD): δ_{H} 7.79-7.86 (d, 1H), 7.47-7.56 (m, 2H), 7.27-7.32 (t, 2H), 4.30 (s, 2H), 3.73-3.84 (t, 2H), 3.16-3.28 (m, 2H), 2.71-2.89 (m, 3H), 2.47-2.52 (t, 2H), 1.84-1.97 (m, 2H), 1.47-1.63 (m, 2H), 1.22-1.33 (m, 2H), 0.75 (s, 9H); MS: [M+H]=343.

### EXAMPLE 3F

### (3S)-N-Cyclohexyl-N-{[2-(trifluoromethyl)phenyl]-methyl}piperidin-3-amine, D-tartrate

¹HNMR (300 MHz, CD₃OD): δ_{H} 7.88-7.91 (d, 1H), 7.51-7.58 (m, 2H), 7.29-7.34 (t, 1H), 4.29 (s, 2H), 3.68-3.83 (q, 2H), 3.43-3.50 (m, 1H), 3.08-3.27 (m, 1H), 2.87-3.00 (m, 2H), 2.39-2.45 (dd, 1H), 2.22-2.29 (dd, 1H), 2.22-2.16 (m, 2H), 1.76-1.90 (m, 2H), 1.58-1.62 (m, 1H), 1.27-1.41 (m, 2H), 1.08-1.22 (m, 2H), 0.97-1.03 (1H), 0.63-0.74 (m, 4H); MS: [M+H]=341.

### EXAMPLE 4F

### (3S)-N-{[5-Fluoro-2-(trifluoromethyl)phenyl]methyl}-N-tetrahydro-2H-pyran-4-ylpiperidin-3-amine, L-tartrate

### a) 1,1-Dimethylethyl (3S)-3-(tetrahydro-2H-pyran-4-ylamino)piperidine-1-carboxylate

1,1-Dimethylethyl-(3S)-3-aminopiperidine-1-carboxylate (2 g, 11 mmol), 4H-tetrahydropyran-4-one (1.1 g, 11 mmol) and dichloroethane (40 mL) were stirred under nitrogen at room temperature for 15 min. Sodium triacetoxyborohydride (2.9 g, 14 mmol) was added in 3 lots over 30 minutes and stirred overnight. The reaction was diluted with water (50 mL) and made basic by addition of 2N NaOH solution. After stirring for 1 h, the mixture was extracted into dichloromethane, and the combined organic extracts washed with brine, dried (MgSO₄), filtered and evaporated in vacuo to give the title compound as an oil.

### b) (3S)-N-{[5-Fluoro-2-(trifluoromethyl)phenyl]methyl}-N-tetrahydro-2H-pyran-4-ylpiperidin-3-amine, L-tartrate

1,1-Dimethylethyl (3S)-3-(tetrahydro-2H-pyran-4-ylamino)piperidine-1-carboxylate was reductively alkylated with 5-fluoro-2-(trifluoromethyl)benzaldehyde, then deprotected and crystallised as its L-tartrate salt as described above for Example 1 b) and c), to give the title compound. ¹HNMR (300 MHz, CD₃OD): δ_{H} 7.74-7.75 (m, 2H), 7.05-6.98 (t, 1H), 4.50 (s, 2H), 3.99-3.85 (m, 4H), 3.43-2.58 (m, 8H), 2.02-1.42 (m, 8H); MS: [M+H]=361.

The following Examples were similarly prepared as described above for Example 4F, by reductive alkylation of 1,1-dimethylethyl (3S)-3-(tetrahydro-2H-pyran-4-ylamino)-piperidine-1-carboxylate with the appropriate benzaldehyde, and subsequent deprotection:

### EXAMPLE 5F

### (3S)-N-[(2-Chloro-5-fluorophenyl)methyl]-N-tetrahydro-2H-pyran-4-ylpiperidin-3-amine, L-tartrate

¹HNMR (300 MHz, CD₃OD): δ_{H} 7.32-7.24 (m, 2H), 6.92-6.85 (t, 1H), 4.30 (s, 2H), 3.90-3.84 (m, 4H), 3.32-3.17 (m, 4H), 3.08-2.97 (m, 1H), 2.85-2.67 (m, 3H), 1.98-1.82 (m, 2H), 1.73-1.82 (m,2H), 1.73-1.46 (m, 6H); MS: [M+H]=327/329.

### EXAMPLE 6F

### (3S)-N-([1,1′-Biphenyl]-2-ylmethyl)-N-tetrahydro-2H-pyran-4-ylpiperidin-3-amine, sesqui-L-tartrate

¹HNMR (300 MHz, CD₃OD): δ_{H} 7.51-7.48 (d, 1H), 7.35-7.17 (m, 7H), 7.08-7.05 (d, 1H), 3.30 (s, 1.5H), 3.79-3.74 (dd, 2H), 3.69 (s, 2H), 3.25-3.10 (m, 9H), 3.20-3.09 (m, 2H), 2.91-2.77 (m, 2H), 2.66-2.51 (m, 3H); MS: [M+H]=351.

### EXAMPLE 7F

### (3S)-N-[(2-Chlorophenyl)methyl]-N-tetrahydro-2H-pyran-4-ylpiperidin-3-amine, D-tartrate

¹HNMR (300 MHz, CD₃OD): δ_{H} 7.52-7.49 (d, 1H), 7.26-7.87 (m, 3H), 4.30 (s, 2H), 3.92-3.80 (m, 4H), 3.16-2.34 (m, 4H), 2.92-2.05 (m, 1H), 2.90-2.66 (m, 3H), 1.93-187 (m, 2H), 1.68-1.39 (m, 6H); MS: [M+H]=309/311.

### EXAMPLE 8F

### (3S)-N-Tetrahydro-2H-pyran-4-yl-N-{[2-(trifluoromethyl)phenyl]methyl}piperidin-3-amine, D-tartrate

¹HNMR (300 MHz, CD₃OD): δ_{H} 7.98-7.95 (d, 1H), 7.71-7.62 (q, 2H), 7.47-7.42 (t, 1H), 4.44 (s, 2H), 4.14-3.98 (m, 4H), 3.43-3.29 (m, 4H), 3.11-2.82 (m, 4H), 2.06-2.03 (m, 2H), 1.82-1.66 (m, 6H); MS: [M+H]=343.

### EXAMPLE 9F

### (3S)-N-Cyclopentyl-N-{[2-(trifluoromethyl)phenyl]-methyl}piperidin-3-amine, L-tartrate

### a) 1,1-Dimethylethyl (3S)-3-(cyclopentylamino)-piperidine-1-carboxylate

1,1-Dimethylethyl (3S)-3-aminopiperidine-1-carboxylate (2.1 g, 10.5 mmol), cyclopentanone (4.65 mL, 52.5 mmol), and 10% palladium on carbon (0.2 g) in methanol (80 mL) were hydrogenated at 60 psi overnight in a Parr hydrogenator. The catalyst was filtered off and the filtrate evaporated in vacuo. The resultant oil was purified by flash chromatography on silica, eluting with ethyl acetate/cyclohexane (15:85 to 30:70), to give the title compound as an oil.

### b) 1,1-Dimethylethyl (3S)-3-(cyclopentyl{[2-(trifluoromethyl)phenyl]methyl}amino)piperidine-1-carboxylate

1,1-Dimethylethyl (3S)-3-(cyclopentylamino)-piperidine-1-carboxylate (155 mg, 0.577 mmol), 2-(trifluoromethyl)benzyl bromide (0.105 mL, 1.2 eq) and anhydrous potassium carbonate (128 mg, 1.6 eq) in acetonitrile (3 mL) were heated at refluxed under nitrogen for 2 days. The reaction mixture was cooled to room temperature, diluted with ethyl acetate and washed with water, then brine. The organic extracts were dried (MgSO₄), filtered and evaporated in vacuo. The resulting oil was purified by flash chromatography on silica eluting with ethyl acetate/cyclohexane (0:100 to 30:70), to give the title compound as an oil.

### c) (3S)-N-Cyclopentyl-N-{[2-(trifluoromethyl)phenyl]-methyl}piperidin-3-amine, L-tartrate

1,1-Dimethylethyl (3S)-3-(cyclopentyl{[2-(trifluoromethyl)phenyl]methyl}amino)piperidine-1-carboxylate (160 mg, 0.38 mmol), trifluoroacetic acid (0.5 mL) and dichloromethane (2 mL) were stirred at room temperature overnight. The solution was evaporated in vacuo to give an oil, which was redissolved in methanol and filtered through a cationic ion exchange resin (Isolute™ SCX-2). The basic components were isolated by elution with 2N ammonia in methanol. The eluate was evaporated in vacuo and the resultant oil converted to the L-tartaric acid salt (freeze drying from acetonitrile/water 1: 1), to give the title compound as a white solid. ¹H NMR (300 MHz, CD₃OD): δ_{H} 7.89-7.86 (d, 1H), 7.54-7.46 (m, 2H), 7.30-7.25 (t, 1H), 4.34 (s, 2H), 3.90-3.78 (q, 2H), 3.30-3.18 (m, 4H), 3.05-2.87 (m, 1H), 2.81-2.59 (m, 2H), 1.95-1.79 (m, 2H), 1.68-1.30 (m, 9H); MS: [M+H]=327.

The following Examples were similarly prepared as described above for Example 9F, by reaction of 1,1-dimethylethyl (3R)-3-(cyclopentylamino)piperidine-1-carboxylate with the appropriate benzyl bromide and subsequent deprotection:

### EXAMPLE 10F

### (3S)-N-([1,1′-Biphenyl]-2-ylmethyl)-N-cyclopentyl-piperidin-3-amine, L-tartrate

¹H NMR (300 MHz, CD₃OD): δ_{H} 7.57-7.55 (d, 1H), 7.35-7.13 (m, 7H), 7.06-7.03 (d, 1H), 4.30 (s, 2H), 3.58 (s, 2H), 3.12-2.98 (m, 3H), 2.82-2.73 (m, 1H), 2.65-2.42 (m, 2H), 1.79-1.75(m, 1H), 1.69-1.65 (m, 1H), 1.53-1.19(m, 10H); MS: [M+H]=335.

### EXAMPLE 11F

### (3S)-N-Cyclopentyl-N-([5-fluoro-1,1′-biphenyl]-2-ylmethyl)-piperidin-3-amine, L-tartrate

¹H NMR (300 MHz, CD₃OD): δ_{H} 7.35-7.24 (m, 4H), 7.18-7.15 (m, 2H), 7.09-7.04 (m, 1H), 6.92-6.85 (m, 1H), 4.28 (s, 2H), 3.55 (m, 2H), 3.22-3.06 (m, 3H), 2.82-2.77 (m, 1H), 2.68-2.58 (m, 2H), 1.88-1.68 (m, 2H), 1.57-1.19 (m, 10H); MS: [M+H]=353.

### EXAMPLE 12 F

### (3S)-N-(Tetrahydrofuran-3-ylmethyl)-N-{[2-(trifluoromethyl)phenyl]methyl}piperidin-3-amine, L-tartrate

### a) 1,1-Dimethylethyl (3S)-3-[(tetrahydrofuran-3-ylmethyl)amino]piperidine-1-carboxylate

To 5% palladium on carbon (0.05 g) under nitrogen was added a solution of 1,1-dimethylethyl-(3S)-3-aminopiperidine-1-carboxylate (0.50 g, 2.5 mmol) and tetrahydrofuran-3-carboxaldehyde (50% w/w in water) (0.50 g, 2.5 mmol) in ethanol (20 mL). The reaction mixture was hydrogenated overnight at 60 psi in a Parr hydrogenator. The catalyst was removed by filtration through Celite and the solvent removed in vacuo to give 1,1-dimethylethyl (3S)-3-[(tetrahydrofuran-3-ylmethyl)amino]piperidine-1-carboxylate as a colourless, slightly cloudy oil.

### b) (3S)-N-(Tetrahydrofuran-3-ylmethyl)-N-{[2-(trifluoromethyl)phenyl]methyl}piperidin-3-amine, L-tartrate

To a solution of 1,1-dimethylethyl (3S)-3-[(tetrahydrofuran-3-ylmethyl)amino]piperidine-1-carboxylate (0.67 g, 2.36 mmol) in 1,2-dichloroethane (15 mL) was added 2-(trifluoromethyl)benzaldehyde (0.93 mL, 7.07 mmol). To this mixture was added a solution of sodium triacetoxyborohydride (1.50 g, 7.07 mmol) in dimethylformamide (3 mL) and left to stir under nitrogen, at room temperature, over the weekend. To the reaction mixture was added water (10 mL) and the solution stirred vigorously for several minutes. The chlorinated organic layer was absorbed directly onto a silica column and the product eluted with methanol/ethyl acetate (0:100 to 30:70). The resultant pale yellow oil was taken up in methanol and absorbed onto a cationic ion exchange resin (Isolute™ SCX-2). After washing the cartridge with methanol (25 mL), the basic components were isolated by elution with 2N ammonia in methanol and the eluate evaporated to give 1,1-dimethylethyl (3S)-3-{(tetrahydrofuran-3-ylmethyl){[2-(trifluoromethyl)-phenyl]methyl}amino}piperidine-1-carboxylate as a colourless oil.

To a solution of this oil (0.82 g, 1.85 mmol) in dichloromethane (10 mL) was added trifluoroacetic acid (2.06 mL, 27.8 mmol). The reaction mixture was stirred overnight at room temperature, then the solvent removed in vacuo. The resulting oil was taken up in methanol and absorbed onto a cationic ion exchange resin (Isolute™ SCX-2). After washing the cartridge with methanol (50 mL), the basic components were isolated by elution with 2N ammonia in methanol. The eluate was evaporated in vacuo to give a colourless oil. The diastereomers were separated by hplc (Chiralpak AD-H column; 98% heptane, 2% ethanol and 0.2% diethylamine). The faster eluting isomer was taken up in methanol and to this was added a solution of L-tartaric acid (0.046 g, 0.31 mmol) in methanol. Solvent was removed in vacuo and the resulting oil triturated with diethyl ether. Filtration of the resultant suspension gave the title compound as a white solid.

¹HNMR (300 MHz, CD₃OD): δ_{H} 7.75 (1H, d), 7.58-7.50 (2H, m), 7.34-7.29 (1H, m), 4.30 (3H, s), 3.83 (2H, s), 3.70-3.53 (3H, m), 3.42-3.31 (2H, m), 3.16 (1H, m) 2.90-2.67 (3H, m), 2.54-2.34 (2H, m), 2.34-2.20 (1H, m), 1.95-1.84 (3H, m), 1.63-1.45 (3H, m); MS: [M+H]=343.

The following Examples were prepared from racemic 1,1-dimethylethyl 3-aminopiperidine-1-carboxylate, as described above in Example 1F:

### EXAMPLE 13F

### N-{[2-(Methyloxy)phenyl]methyl}-N-{[2-(trifluoromethyl)phenyl]methyl}piperidin-3-amine

¹HNMR (300 MHz, CDCl₃) δ_{H} 8.04-7.95 (d, 1H), 7.57-7.54 (d, 1H), 7.48-7.44 (m, 2H), 7.28-7.11 (m, 2H), 6.93-6.88 (t, 1H), 6.83-6.80 (d, 1H), 3.94-3.86 (d, 2H), 3.20-3.18 (d, 1H), 2.94-2.90 (d, 1H), 2.68-2.55 (m, 2H), 2.49-2.40 (dt, 1H), 2.08-2.04 (d, 1H), 1.76-1.72 (d, 1H), 1.52-1.25 (m, 4H); MS: [M+H]=379.

### EXAMPLE 14F

### N-Cyclohexyl-N-{[2-(trifluoromethyl)phenyl]methyl}-piperidin-3-amine

¹HNMR (300 MHz, CDCl₃) δ_{H} 8.01-7.93 (d, 1H), 7.59-7.56 (d, 1H), 7.51-7.46 (t, 1H), 7.30-7.19 (m, 1H), 3.91 (s, 2H), 3.15-3.11 (d, 1H), 3.02-2.98 (d, 1H), 2.88-2.80 (d, 1H), 2.55-2.41 (m, 3H), 1.93-1.01 (m, 14); MS: [M+H]=341.

### EXAMPLE 15F

### N-(Phenylmethyl)-N-{[2-(trifluoromethyl)phenyl]-methyl}piperidin-3-amine

¹HNMR (300 MHz, CDCl₃) δ_{H} 7.93-7.96 (d, 1H), 7.55-7.61 (d, 1H), 7.47-7.51 (t, 1H), 7.18-7.35 (m, 6H), 3.77-3.90 (q, 2H), 3.64-3.74 (q, 2H), 3.17-3.20 (d, 1H), 2.91-2.95 (d, 1H), 2.53-2.67 (m, 2H), 2.39-2.48 (dt, 1H), 1.97-2.06 (d, 1H), 1.22-1.82 (m,3H); MS: [M+H]=349.

### EXAMPLE 16F

### (3S)-N-(2-Methylpropyl)-N-{[2-(trifluoromethyl)phenyl]-methyl}-1-azabicyclo[2.2.2]octan-3-amine, sesquifumarate

### a) (3S)-N-{[2-(Trifluoromethyl)phenyl]methyl}-1-azabicyclo[2.2.2]octan-3-amine

Sodium triacetoxyborohydride (18.7 g, 88.3 mmol) was added portionwise over 20 min. to a stirred solution of (3S)-1-azabicyclo[2.2.2]octan-3-amine dihydrochloride (5 g, 25.1 mmol) and 2-trifluoromethylbenzaldehyde (4.81 g, 27.6 mmol) in DMF (100 mL). After stirring under nitrogen for 4 days, the mixture was diluted with excess water, basified with 2N sodium hydroxide and stirred for 1 h. The product was extracted into dichloromethane and evaporated in vacuo to give an oil, which was dissolved in 2N hydrochloric acid. After washing with ether, the aqueous phase was basified with 2N sodium hydroxide and extracted with dichloromethane. The organic phase was dried (MgSO₄) and evaporated in vacuo to give an oil. ¹HNMR (300 MHz, CD₃OD) δ_{H}: 7.62-7.69 (t, 2H), 7.50-7.55 (t, 1H), 7.32-7.37 (t, 1H), 3.83-3.96 (q, 2H), 3.1-3.19 (m, 1H), 2.72-2.93 (m, 5H), 2.42-2.49 (m, 1H), 1.85-1.95 (m, 1H), 1.63-1.73 (m, 1H), 1.32-1.53); MS: [M+H]=285.

### b) (3S)-N-(2-Methylpropyl)-N-{[2-(trifluoromethyl)-phenyl]methyl)-1-azabicyclo[2.2.2]octan-3-amine, sesquifumarate

(3S)-N-{[2-(Trifluoromethyl)phenyl]methyl}-1-azabicyclo[2.2.2]octan-3-amine (0.30 g, 1.06 mmol), isobutyraldehyde (0.152 g, 2.1 mmol) and 1,2-dichloroethane (6 mL) were stirred under nitrogen at room temperature for 15 min. Sodium triacetoxyborohydride (0.492 g, 2.32 mmol) was added in two lots over 5 min. TLC after 1 day showed the reaction to be incomplete, so additional sodium triacetoxyborohydride (0.24 g, 1.15 mmol) was added and the mixture heated at 50° C. for 5 days. After cooling to room temperature, methanol was added and the mixture was stirred for 1 h. This solution was filtered through a cationic ion exchange resin (Isolute™ SCX-2) and the basic fractions isolated by elution with 2N ammonia in methanol to give, after evaporation in vacuo, an oil. The crude product was purified using preparative LCMS to give the product as an acetate salt, which was converted to the free base using cationic ion exchange resin as described above. The free base was converted to the fumarate salt, to give the title compound as a white solid from ethanol/diethyl ether. ¹HNMR (300 MHz, CD₃OD) δ_{H}: 7.88-7.91 (d, 1H), 7.51-7.58 (m, H), 7.30-7.35 (t, 1H), 6.60 (s, 3H), 3.71-3.85 (q, 2H), 3.42-4.50 (m, 1H), 2.88-3.26 (m, 6H), 2.25-2.39 (m, 1H), 2.09-2.23 (m, 3H), 1.74-1.91 (m, 2H), 1.42-1.63 (m, 2H), 0.78-0.83 (t, 6H); MS: [M+H]=341.

The following Examples were similarly prepared as described above for Example 16F, from (3S)-N-{[2-(trifluoromethyl)phenyl]methyl}-1-azabicyclo-[2.2.2]octan-3-amine and the appropriate substituted benzaldehyde:

### EXAMPLE 17F

### (3S)-N-([1,1′-Biphenyl]-2-ylmethyl)-N-(2-methylpropyl)-1-azabicyclo[2.2.2]octan-3-amine, D-tartrate

¹HNMR (300 MHz, CD₃OD) δ_{H}: 7.50-7.47 (d, 1H), 7.38-7.18 (m, 7H), 7.09-7.06 (dd, 1H), 4.29 (s, 2H), 3.58-3.54 (d, 1H), 3.43-3.39 (d,1H), 3.25-3.18 (m, 1H), 3.09-3.90 (4H), 2.68-2.63 (t,1H), 2.45-2.39 (dq, 1H), 2.16-1.98 (m, 3H), 1.83-1.74 (m, 2H), 1.65-1.61 (m, 1H), 1.45-1.42 (m, 1H), 1.31-1.22 (quin, 1H), 0.65-0.61 (t, 6H); MS: [M+H]=349.

### EXAMPLE 18F

### (3S)-N-{[4-Fluoro-2-(trifluoromethyl)phenyl]methyl}-N-(2-methylpropyl)-1-azabicyclo[2.2.2]octan-3-amine, L-tartrate

¹HNMR (300 MHz, CD₃OD) δ_{H}: 7.94-7.89 (t, 1H), 7.34-7.27 (m, 2H), 4.29 (s, 4.29), 3.81-3.66 (q, 2H), 3.51-3.44 (t,1H), 3.40-2.89 (m, 6H), 2.37-2.04 (m, 4H), 1.93-1.38 (m, 4H), 0.82-0.76 (dd, 6H); MS: [M+H]=359.

### EXAMPLE 19F

### (3S)-N-1(4-Fluoro[1,1′-biphenyl]-2-yl)methyl]-N-(2-methylpropyl)-1-azabicyclo[2.2.2]octan-3-amine, L-tartrate

¹HNMR (300 MHz, CD₃OD) δ_{H}: 7.40-7.08 (m, 7H). 6.68-6.91 (dt, 1H), 4.29 (s, 2H), 3.56-4.0 (q, 2H), 3.31-2.96 (m, 5H), 2.72-2.67 (t, 1H), 2.58-2.52 (dq, 1H), 2.18-1.30 (m, 8H), 0.70-0.68 (dd, 6H); MS: [M+H]=367.

The following examples illustrate compounds of of Formulae (IG) above and methods for their preparation.

Preparation of Intermediates

### (2S)-(4-Benzyl-morpholin-2-yl)-phenyl-methanone

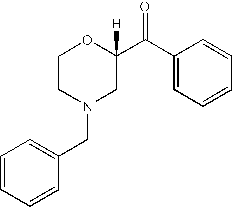

Described above in section entitled “Preparation of intermediates for the synthesis of Examples 1C-17C”.

### (S)-Phenyl[(2S)-4-(phenylmethyl)morpholin-2-yl]methanol (2)

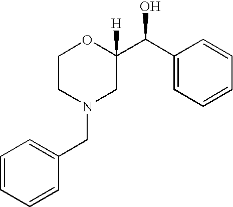

Described above in section entitled “Preparation of intermediates for the synthesis of Examples 1C-17C”.

### (2S)-2-[(R)-bromo(phenyl)methyl]-4-(phenylmethyl)morpholine (3)

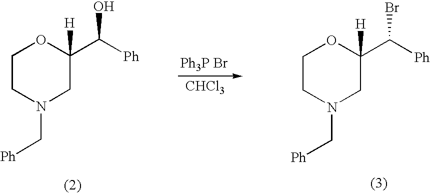

To a solution of (S)-phenyl[(2S)-4-(phenylmethyl)morpholin-2-yl]methanol (2) (4.71 g, 16.63 mmole) in chloroform (200 ml) is added the triphenylphosphine dibromide (14.04 g, 33.26 mmole). The mixture is heated at 60° C. overnight. The mixture is allowed to cool to room temperature then washed with saturated sodium carbonate solution (aqueous, ˜100 ml), dried (Na₂SO₄) and concentrated in vacuo. The resulting residue is purified by automated flash chromatography (ISCO system: 120 g column, 10-30% EtOAc in isohexane) to give (2S)-2-[(R)-bromo(phenyl)methyl]-4-(phenylmethyl)morpholine (3) as a white solid (4.63 g, 80%). LCMS 6 min gradient method, Rt=2.5 min, (M+H⁺)=346/348

### S-{(S)-phenyl[(2S)-4-(phenylmethyl)morpholin-2-yl]methyl}ethanethioate (5)

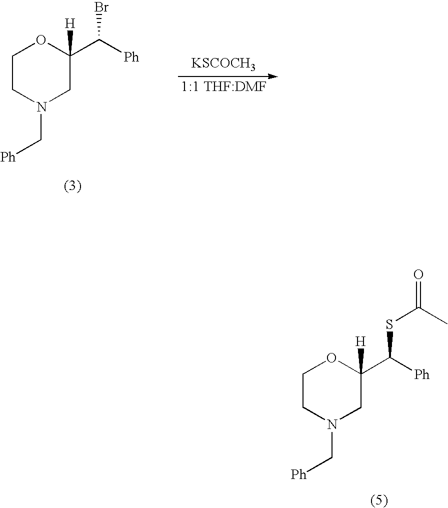

A solution of (2S)-2-[(R)-bromo(phenyl)methyl]-4-(phenylmethyl)morpholine (3) (1.76 g, 5.08 mmole) and potassium thiolacetate (1.16 g, 10.16 mmole) in 1:1 anhydrous THF:DMF (30 ml), is stirred at 40° C. under nitrogen overnight. The mixture is then taken up in acetonitrile and loaded onto an SC10-2 column (4×10 g). The SC10-2 columns are washed with further acetonitrile. The target compound is eluted with 4:1 acetonitrile: Et₃N. This is concentrated in vacuo to give an orange oil which is purified by automated flash chromatography (ISCO system: 35 g SiO₂ Redisep column, 10-30% EtOAc in isohexane over 40 minutes) to give S-{(S)-phenyl[(2S)-4-(phenylmethyl)morpholin-2-yl]methyl}ethanethioate (5) as an amber coloured crystalline solid (1.54 g, 89%). LCMS 6 min gradient method, Rt=2.5 min, (M+H⁺)=342

### (S)-phenyl[(2S)-4-(phenylmethyl)morpholin-2-yl]methanethiol (6)

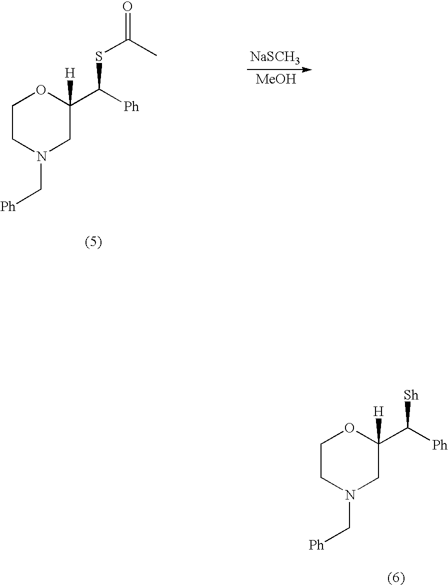

The S-{(S)-phenyl[(2S)-4-(phenylmethyl)morpholin-2-yl]methyl}ethanethioate (5) (11.02 g, 32.3 mmole) is taken up in methanol (100 ml, dry, degassed), under nitrogen. To this is added the sodium thiomethoxide (2.26 g, 32.3 mmole) in one portion (as solid). The reaction mixture is left to stir at room temperature for 2 hours. The solution is then added to an aqueous solution of HCl (0.1 M). This is extracted with DCM (3×). The extracts are dried (Na₂SO₄) and concentrated in vacuo to give (S)-phenyl[(2S)-4-(phenylmethyl)morpholin-2-yl]methanethiol (6) as a yellow solid (9.59 g, 99%). LCMS 6 min gradient method, Rt=2.7 min, (M+H⁺)=300

### EXAMPLES

### Example 1G

### (2S)-2-{(S)-phenyl[(3-phenylpyridin-2-yl)thio]methyl}morpholine hemifumarate

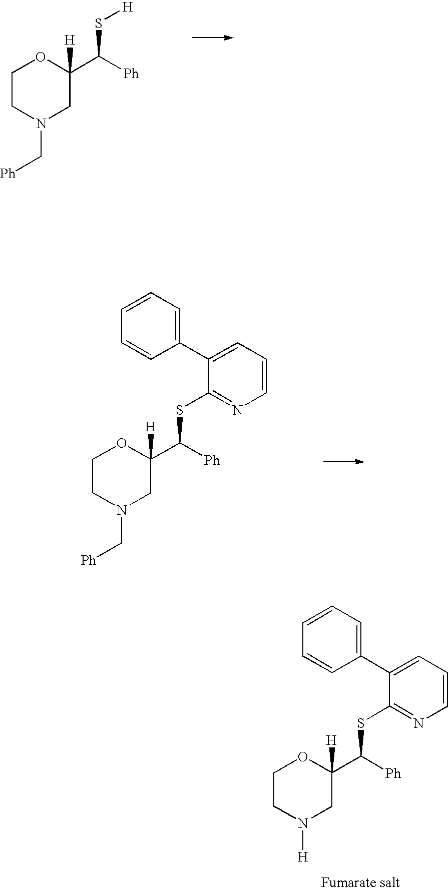

i) To palladium acetate (0.026 g, 0.12 mmole) in acetonitrile (3 ml), is added triphenylphosphine (0.122 g, 0.46 mmole), under nitrogen, at room temperature. The mixture is left to stir for 15 minutes. To this mixture is added water (distilled, 1 ml), phenylboronic acid (0.846 g, 6.94 mmole), 3-bromo-2-fluoropyridine (1.02 g, 5.78 mmole) and potassium carbonate (4.80 g, 34.70 mmole). The reaction mixture is heated at 70° C. overnight. After cooling to room temperature, the reaction mixture is loaded directly onto a 40 g Redisep SiO₂ column and components isolated by automated flash chromatography (ISCO System, 0-30% ethyl acetate in cyclohexane gradient elution over 40 minutes). This gave 2-fluoro-3-phenylpyridine as a very pale yellow oil (1.00 g, 100%). LCMS 6 min gradient method, Rt=3.7 min, (M+H⁺)=174.

ii) To a solution of (S)-phenyl[(2S)-4-(phenylmethyl)morpholin-2-yl]methanethiol (6) (1.50 g, 5.01 mmole) and 2-fluoro-3-phenylpyridine (2.44 g, 14.09 mmole) in dry, degassed DMF (10 ml) is added, under nitrogen, sodium hydride (60% dispersion in oil, 0.24 g, 6.01 mmole). The mixture is left to stir overnight at room temperature. The reaction mixture is loaded neat onto a 120 g SiO₂ Redisep column (preconditioned with cyclohexane). Automated flash chromatography (ISCO System, 0-30% ethyl acetate in cyclohexane gradient elution over 40 minutes at 40 ml/minute flow rate) yielded an orange oil (2.26 g). Chromatography is repeated using chromatography (ISCO System, 40 g column, 0-30% ethyl acetate in cyclohexane gradient elution over 40 minutes at 30 ml/minute flow rate) to give (2S)-2-{(S)-phenyl[(3-phenylpyridin-2-yl)thio]methyl}-4-(phenylmethyl)morpholine as a pale orange oil (1.65 g, 73%). LCMS 6 min gradient method, Rt=4.0 min, (M+H⁺)=453.

iii) To a suspension of polymer supported diisopropylamine (3.78 mmol/g, 0.54 g, 2.03 mmole) and (2S)-2-{(S)-phenyl[(3-phenylpyridin-2-yl)thio]methyl}-4-(phenylmethyl)morpholine (0.184 g, 0.41 mmole) in dry DCM (5 ml) is added 1-chloroethyl chloroformate (0.22 ml, 2.03 mmole) at room temperature and under nitrogen. The mixture is heated at 40° C. for 3.75 hours. The reaction mixture is filtered, concentrated in vacuo then taken up in methanol (5 ml). The solution is left to stir at room temperature overnight. After this time, the reaction mixture is loaded directly onto an SC10-2 column. The SC10-2 column is washed with methanol. The title compound is eluted with 2 N NH₃/methanol. This is concentrated in vacuo to give (2S)-2-{(S)-phenyl[(3-phenylpyridin-2-yl)thio]methyl}morpholine as white foam (0.148 g, 100%). The foam is taken up in ethyl acetate. To this is added a solution of fumaric acid (1.1 equiv, 0.052 g) in methanol. The resulting solution is filtered then concentrated in vacuo. To the resulting white solid is added methanol (1.5 ml). This is stirred for a couple of minutes, then the remaining solid collected by filtration to give the hemi-fumarate salt of (2S)-2-{(S)-phenyl[(3-phenylpyridin-2-yl)thio]methyl}morpholine as a white solid (0.127 g). LCMS 12 min gradient method, Rt=5.5 min, (M+H⁺)=363

### Example 2G

### (2S)-2-[(S)-{[3-(4-fluorophenyl)pyridin-2-yl]thio}(phenyl)methyl]morpholine fumarate

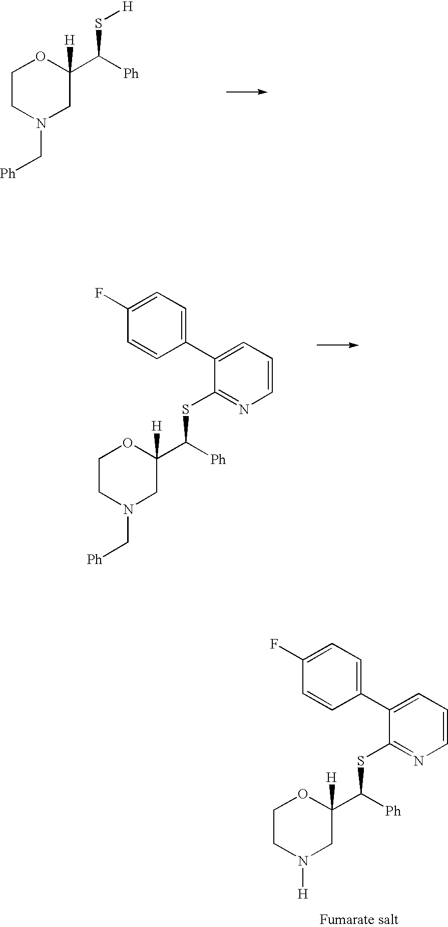

i) To bis(benzonitrile)palladium(II)dichloride (0.054 g, 0.14 mmole) and 1,4-bis(diphenylphosphine)butane (0.091 g, 0.21 mmole) is added dry toluene (6 ml), under nitrogen, and the mixture stirred for half an hour. To this is added 3-bromo-2-fluoropyridine (0.50 g, 2.83 mmole) in ethanol (1.4 ml) followed by a solution of 4-fluorophenylboronic acid (0.793 g, 5.67 mmole) in ethanol (2.4 ml). To this is added an aqueous solution of sodium carbonate (1 M, 2.83 ml, 2.83 mmole). The mixture is heated at 60° C. for 24 hours, then at 75° C. for a further 16 hours. The organic layer is loaded directly onto a 40 g Redisep SiO₂ column and components isolated by automated flash chromatography (ISCO System, 0-30% ethyl acetate in cyclohexane gradient elution over 40 minutes). This gave 3-(4-fluorophenyl)-2-fluoropyridine as a white solid (0.387 g, 71%). LCMS 6 min gradient method, Rt=3.6 min, (M+H⁺)=192

ii) To a solution of (S)-phenyl[(2S)-4-(phenylmethyl)morpholin-2-yl]methanethiol (6) (0.505 g, 1.69 mmole) and 3-(4-fluorophenyl)-2-fluoropyridine (0.387 g, 2.02 mmole) in dry, degassed DMF (3 ml) is added, under nitrogen, cesium fluoride (0.385 g, 2.54 mmole). The mixture is heated at 65° C. over the weekend. After this time, the reaction mixture is allowed to cool and loaded directly onto an SC10-2 column. The SC10-2 column is washed with methanol. The (2S)-2-[(S)-{[3-(4-fluorophenyl)pyridin-2-yl]thio}(phenyl)methyl]-4-(phenylmethyl)morpholine is eluted with 2 N NH₃/methanol. This is concentrated in vacuo to give an orange solid (0.649 g). This is purified by automated flash chromatography (ISCO System, 40 g SiO₂ Redisep column, 0-30% ethyl acetate in cyclohexane gradient elution over 40 minutes at 30 ml/minute flow rate) to give (2S)-2-[(S)-{[3-(4-fluorophenyl)pyridin-2-yl]thio}(phenyl)methyl]-4-(phenylmethyl)morpholine as a off-white foam (0.395 g, 50%). LCMS 6 min gradient method, Rt=3.3 min, (M+H⁺)=471.

iii) Deprotection of the morpholine nitrogen is carried out using the method and work up as described in Example 1G, using polymer supported diisopropylamine (3.78 mmole/g, 1.09 g, 4.14 mmole), (2S)-2-[(S)-{[3-(4-fluorophenyl)pyridin-2-yl)thio}(phenyl)methyl]-4-(phenylmethyl)morpholine (0.390 g, 0.83 mmole), dry DCM (20 ml), 1-chloroethyl chloroformate (0.45 ml, 4.14 mmole) and methanol (20 ml). This gave (2S)-2-[(S)-{[3-(4-fluorophenyl)pyridin-2-yl]thio}(phenyl)methyl]morpholine as a pale yellow oil (0.232 g, 74%). This oil is taken up in ethyl acetate. To this is added a solution of fumaric acid (1.1 equiv, 0.071 g) in methanol. The resulting solid is collected by filtration to give a white solid (0.115 g). This is recrystallised from MeOH/CHCl₃/Et₂O to give a white solid (0.061 g). LCMS 12 min gradient method, Rt=5.4 min, (M+H⁺)=381

### Example 3G

### (2S)-2-[(S)-{[3-(3-chlorophenyl)pyridin-2-yl]thio}(phenyl)methyl]morpholine fumarate

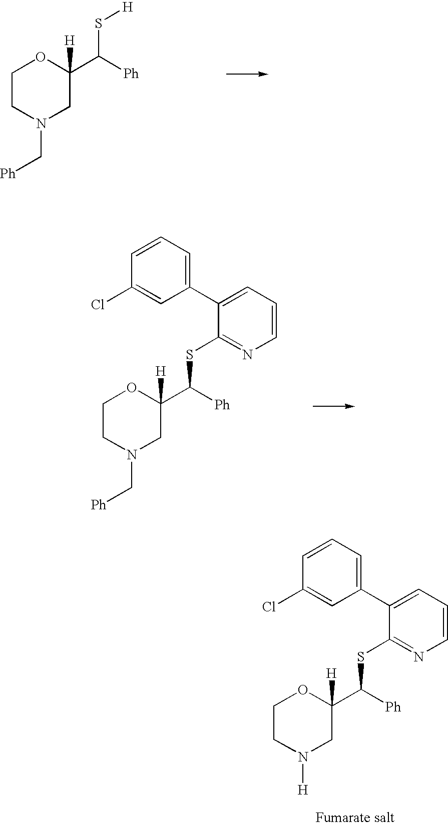

i) To bis(benzonitrile)palladium(II)dichloride (0.054 g, 0.14 mmole) and 1,4-bis(diphenylphosphine)butane (0.091 g, 0.21 mmole) is added dry toluene (6 ml), under nitrogen, and the mixture stirred for half an hour. To this is added 3-bromo-2-fluoropyridine (0.50 g, 2.83 mmole) in ethanol (1.4 ml) followed by a solution of 3-chlorophenylboronic acid (0.887 g, 5.67 mmole) in ethanol (2.4 ml). To this is added an aqueous solution of sodium carbonate (1 M, 2.83 ml, 2.83 mmole). The mixture is heated at 60° C. for 24 hours, then at 75° C. for a further 16 hours. The organic layer is loaded directly onto a 40 g Redisep SiO₂ column and components isolated by automated flash chromatography (ISCO System, 0-30% ethyl acetate in cyclohexane gradient elution over 40 minutes). This gave 3-(3-chlorophenyl)-2-fluoropyridine as an off-white solid (0.333 g, 57%). LCMS 6 min gradient method, Rt=4.0 min, (M+H⁺)=208.

ii) To a solution of (S)-phenyl[(2S)-4-(phenylmethyl)morpholin-2-yl]methanethiol (6) (0.400 g, 1.34 mmole) and 3-(3-chlorophenyl)-2-fluoropyridine (0.333 g, 1.60 mmole) in dry, degassed DMF (3 ml) is added, under nitrogen, cesium fluoride (0.305 g, 2.00 mmole). The mixture is heated at 65° C. over the weekend. After this time, the reaction mixture allowed to cool. The resulting solid is taken up in MeOH/DCM and loaded directly onto an SC10-2 column. The SC10-2 column is washed with methanol. The (2S)-2-[(S)-{[3-(3-chlorophenyl)pyridin-2-yl]thio}(phenyl)methyl]-4-(phenylmethyl)morpholine is eluted with 2 N NH₃/methanol. This is concentrated in vacuo to give a white foam (0.555 g). This is purified by automated flash chromatography (ISCO System, 0-30% ethyl acetate in cyclohexane gradient elution over 40 minutes at 40 ml/minute flow rate) to yield (2S)-2-[(S)-{[3-(3-chlorophenyl)pyridin-2-yl]thio}(phenyl)methyl]-4-(phenylmethyl)morpholine as a white foam (0.258 g, 40%). LCMS 6 min gradient method, Rt=4.2 min, (M+H⁺)=487.

iii) Deprotection of the morpholine nitrogen is carried out using the method and work up as described in Example 1G, using polymer supported diisopropylamine (3.72 mmole/g, 0.70 g, 1.80 mmole), (2S)-2-[(S)-{[3-(3-chlorophenyl)pyridin-2-yl]thio}(phenyl)methyl]-4-(phenylmethyl)morpholine (0.255 g, 0.52 mmole), dry DCM (15 ml), 1-chloroethyl chloroformate (0.29 ml, 2.62 mmole) and methanol (15 ml). This gave a colourless residue (0.211 g). This residue is taken up in ethyl acetate. To this is added a solution of fumaric acid (1.1 equiv, 0.062 g) in methanol. If the resulting solid contains impurities it may be recombined with the mother liquor and purified on a UV Guided PrepHPLC (Flex) System and treated with SC10-2 to give (2S)-2-[(S)-{[3-(3-chlorophenyl)pyridin-2-yl]thio}(phenyl)methyl]morpholine as a pale yellow oil (0.127 g, 65%). This oil is taken up in MeOH/DCM. To this is added a solution of fumaric acid (1.1 equiv, 0.0145 g) in methanol, followed by Et₂O. The resulting crystals are collected by filtration to give the fumarate salt of (2S)-2-[(S)-{[3-(3-chlorophenyl)pyridin-2-yl]thio}(phenyl)methyl]morpholine (1:1 fumarate salt) as a white solid (0.047 g). LCMS 12 min gradient method, Rt=5.7 min, (M+H⁺)=397

### Example 4G

### (2S)-2-[{[3-(2-chlorophenyl)pyridin-2-yl]thio}(phenyl)methyl]morpholine fumarate

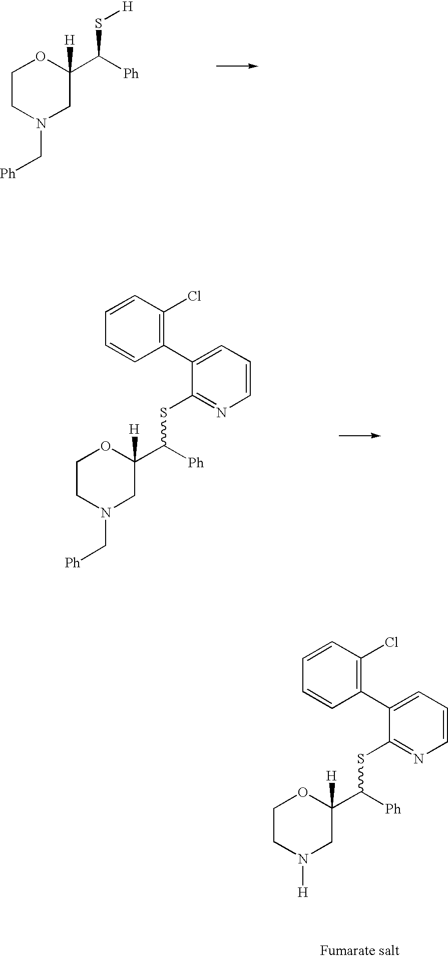

i) To palladium acetate (0.0025 g, 0.0011 mmole) in acetonitrile (3 ml), is added triphenylphosphine (0.0119 g, 0.045 mmole), under nitrogen, at room temperature. The mixture is left to stir for 15 minutes. To this mixture is added water (distilled, 1 ml), 2-chlorophenylboronic acid (0.106 g, 0.68 mmole), 3-bromo-2-fluoropyridine (0.10 g, 0.57 mmole) and potassium carbonate (0.470 g, 3.40 mmole). The reaction mixture is heated to 60° C. increasing to 75° C. over 5 hours then allowed to cool to room temperature. To the reaction mixture is added MeOH and this is loaded onto an SC10-2 column (10 g) preconditioned with MeOH. The column is washed with MeOH and the resulting solution concentrated in vacuo to give an orange oil (0.196 g). The oil is purified by automated flash chromatography (ISCO System, a 10 g Redisep SiO₂ column, 0-30% ethyl acetate in cyclohexane gradient elution over 40 minutes). This gave 2-fluoro-3-(2-chlorophenyl)pyridine as a colourless oil (0.050 g, 42%). LCMS 6 min gradient method, Rt=3.3 min, (M+H⁺)=208

ii) To a solution of (S)-phenyl[(2S)-4-(phenylmethyl)morpholin-2-yl]methanethiol (6) (0.288 g, 0.96 mmole) and 3-(2-chlorophenyl)-2-fluoropyridine (0.40 g, 1.93 mmole) in dry, degassed DMF (2 ml) is added, under nitrogen, sodium hydride (60% dispersion in oil, 0.0.046 g, 1.15 mmole). The mixture is left to stir at room temperature over the weekend. The reaction mixture is loaded directly onto an a 40 g Redisep SiO₂ column. Components are eluted using automated flash chromatography (ISCO System, 0-30% ethyl acetate in cyclohexane gradient elution over 30 minutes at 40 ml/minute flow rate) to give (2S)-2-[{[3-(2-chlorophenyl)pyridin-2-yl]thio}(phenyl)methyl]-4-(phenylmethyl)morpholine as a white solid (0.021 g, 5%). LCMS 6 min gradient method, Rt=4.3 min, (M+H⁺)=487.

iii) Deprotection of the morpholine nitrogen is carried out using the method and work up as described in Example 1G, using polymer supported diisopropylamine (3.78 mmole/g, 0.057 g, 0.216 mmole), (2S)-2-[{[3-(2-chlorophenyl)pyridin-2-yl]thio}(phenyl)methyl]-4-(phenylmethyl)morpholine (0.021 g, 0.043 mmole), dry DCM (2 ml), 1-chloroethyl chloroformate (0.024 ml, 0.216 mmole) and methanol (2 ml). This gave a colourless residue (0.017 g, 100%). This residue is taken up in ethyl acetate. To this is added a solution of fumaric acid (1 equiv, 0.005 g) in methanol. This is reduced in volume and Et₂O added. The resulting solid is collected by filtration to give the fumarate salt of (2S)-2-[{[3-(2-chlorophenyl)pyridin-2-yl]thio}(phenyl)methyl]morpholine (1:1 fumarate salt) as a pale green solid (0.012 g). LCMS 12 min gradient method, Rt=5.4 min, (M+H⁺)=397

### Example 5G

### (2S)-2-((S)-phenyl{[3-(trifluoromethyl)pyridin-2-yl]thio}methyl)morpholine

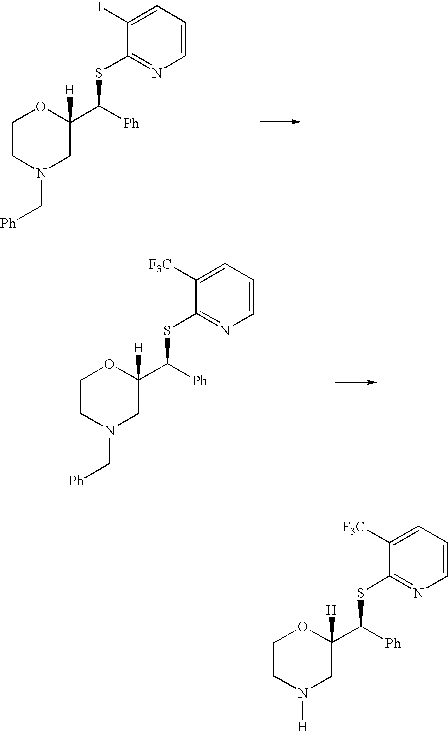

i) Potassium fluoride (0.048 g, 0.84 mmole) and copper (I) iodide (0.159 g, 0.84 mmole) are thoroughly mixed and dried under reduced pressure with a hot air gun for 20 minutes. To the resulting yellow solid, at room temperature is added (2S)-2-[(S)-[(3-iodopyridin-2-yl)thio](phenyl)methyl]-4-(phenylmethyl)morpholine (as prepared in Example 15) (0.190 g, 0.38 mmole) in anhydrous NMP (0.5 ml) followed by anhydrous DMF (0.5 ml) then (trifluoromethyl)trimethylsilane (0.11 ml, 0.76 mmole). After 3 days at room temperature, the temperature is increased to 50° C. The reaction mixture is heated at 50° C. overnight.

After cooling to room temperature, further (trifluoromethyl)trimethylsilane (0.11 ml, 0.76 mmole) is added to the reaction mixture and the mixture is left to stir overnight at room temperature. To the reaction mixture is added MeOH before loading onto an SC10-2 column (10 g) preconditioned with MeOH. The column is washed with MeOH. Basic material is eluted with 2 N NH₃/methanol. This is concentrated in vacuo to give a pale yellow solid (0.199 g). This is purified by automated flash chromatography (ISCO System, 3×4 g Redisep SiO₂ columns, in parallel, 0-20% ethyl acetate in cyclohexane gradient elution over 40 minutes) to give the (2S)-2-[(S)-[(3-iodopyridin-2-yl)thio](phenyl)methyl]-4-(phenylmethyl)morpholine as a white foam (0.108 g, 57% recovery of this starting material) and (2S)-2-((S)-phenyl {[3-(trifluoromethyl)pyridin-2-yl]thio}methyl)-4-(phenylmethyl)morpholine as a colourless oil (0.033 g, 20%). LCMS 6 min gradient method, Rt=4.2 min, (M+H⁺)=445

ii) To a suspension of polymer supported diisopropylamine (3.72 mmol/g, 0.097 g, 0.36 mmole) and (2S)-2-((S)-phenyl{[3-(trifluoromethyl)pyridin-2-yl]thio}methyl)-4-(phenylmethyl)morpholine (0.0.032 g, 0.07 mmole) in dry DCM (0.5 ml) is added 1-chloroethyl chloroformate (0.039 ml, 0.36 mmole) at room temperature and under nitrogen. The mixture is heated at 40° C. for 2 hours. The reaction mixture is filtered and concentrated in vacuo then taken up in methanol (0.5 ml). The solution left to stir at room temperature overnight. After this time, the reaction mixture is loaded directly onto an SC10-2 column. The SC10-2 column is washed with methanol. The target compound is eluted with 2 N NH₃/methanol. This is concentrated in vacuo to give a pale yellow oil (0.024 g). The pale yellow oil is purified using an automated PrepLCMS system, then liberated as the free base by treatment with SC10-2 and concentrated under vacuum to give (2S)-2-((S)-phenyl{[3-(trifluoromethyl)pyridin-2-yl]thio}methyl)morpholine as a white solid (0.005 g, 20%). LCMS 12 min gradient method, Rt=4.9 min, (M+H⁺)=354

### Example 6G

### (2S)-2-((S)-phenyl{[3-(phenylmethyl)pyridin-2-yl]thio}methyl)morpholine fumarate

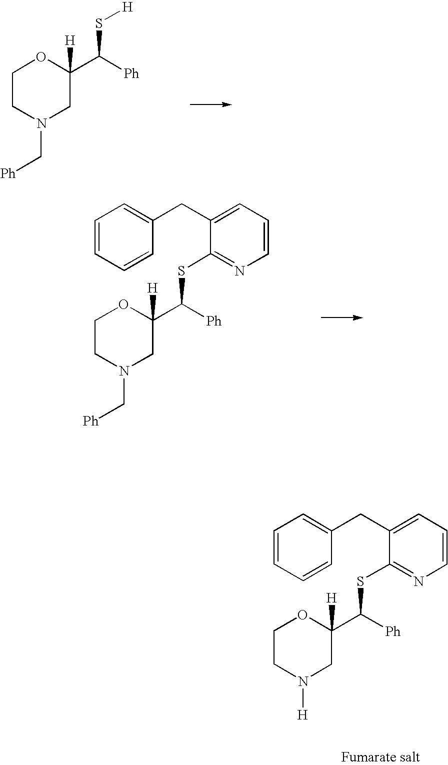

i) To a 100 ml round-bottomed flask, under nitrogen, containing dry THF (25 ml) is added n-butyllithium (1.6 M solution in hexanes, 3.99 ml, 6.39 mmole) at 0° C. followed by lithium diisopropylamide (2 M solution in THF/n-heptane, 3.19 ml, 6.39 mmole). The reaction mixture is left to stir for 1 hour at 0° C. The mixture is cooled to −70° C. then 2-fluoropyridine added. The solution is stirred at −70° C. for 4 hours. To the solution is added benzaldehyde (0.71 ml, 6.97 mmole). This is then left to stir for 1 hour at −70° C., after which time water (100 ml) is added. On warming to room temperature the solution is extracted with chloroform (2×100 ml). The combined extracts are dried (Na₂SO₄) and concentrated in vacuo to yield a yellow oil (1.58 g). Purification by automated flash chromatography (ISCO System, Redisep 10 g SiO₂ column, 0-30% ethyl acetate in cyclohexane gradient elution over 30 minutes at 20 ml/min flow rate) gave 2-fluoro-3-(phenyl-1-hydroxymethyl)pyridine as a yellow oil (0.71 g, 59%). FIA (M+H⁺)=204

ii) To 5% Pd/C (0.07 g), under nitrogen, is added a solution of 2-fluoro-3-(1-hydroxy-1-phenylmethyl)pyridine (0.71 g, 3.5 mmole) in ethanol (50 ml). This is then put on a Parr Hydrogenator at 60 psi H₂ and left over the weekend. The reaction mixture is filtered through Celite®. Removal of solvent from the resulting solution gave a pale yellow oil. This is purified by automated flash chromatography (ISCO System, 10 g SiO₂ Redisep column, 0-30% ethyl acetate in cyclohexane gradient elution over 40 minutes at 20 ml/minute flow rate) to give 2-fluoro-3-(phenylmethyl)pyridine as a colourless oil (0.18 g, 27%).

iii) To a solution of (S)-phenyl[(2S)-4-(phenylmethyl)morpholin-2-yl]methanethiol (6) (0.27 g, 0.91 mmole) and 2-fluoro-3-(1-hydroxy-1-phenylmethyl)pyridine (0.17 g, 0.91 mmole) in dry, degassed DMF (1.5 ml) is added, under nitrogen, sodium hydride (60% dispersion in oil, 0.07 g, 1.82 mmole). The mixture is left to stir overnight at room temperature. A further portion of sodium hydride (605 dispersion in oil, 0.07 g, 1.82 mmole) and DMF (1 ml) is added. After 5 hours at room temperature, the reaction mixture is taken up in MeOH and loaded onto an SC10-2 column. The SC10-2 column is washed with methanol. The (2S)-2-((S)-phenyl{[3-(phenylmethyl)pyridin-2-yl]thio}methyl)-4-(phenylmethyl)morpholine is eluted with 2 N NH₃/methanol. This is concentrated in vacuo to give a yellow residue (0.36 g). The residue is purified by automated flash chromatography (ISCO System, 35 g SiO₂ Redisep column, 0-30% ethyl acetate in cyclohexane gradient elution over 40 minutes at 40 ml/minute flow rate) which yields (2S)-2-((S)-phenyl{[3-(phenylmethyl)pyridin-2-yl]thio}methyl)-4-(phenylmethyl)morpholine as a pale yellow oil (0.10 g, 24%). LCMS 6 min gradient method, Rt=3.8 min, (M+H⁺)=467

iv) Deprotection of the morpholine nitrogen is carried out using the method and work up as described in Example 1G, using polymer supported diisopropylamine (3.78 mmole/g, 0.28 g, 1.07 mmole), of (2S)-2-((S)-phenyl{[3-(phenylmethyl)pyridin-2-yl]thio}methyl)-4-(phenylmethyl)morpholine (0.092 g, 0.20 mmole), dry DCM (5 ml), 1-chloroethyl chloroformate (0.12 ml, 1.07 mmole) and methanol (5 ml). This gives (2S)-2-((S)-phenyl {[3-(phenylmethyl)pyridin-2-yl]thio}methyl)morpholine as a colourless residue (0.076 g, 94%). This oil is taken up in ethyl acetate. To this is added a solution of fumaric acid (1.1 equiv, 0.026 g) in methanol. The resulting solution is concentrated in vacuo and the resulting oil triturated with ethyl acetate. The solid is collected by filtration to give the fumarate salt of (2S)-2-((S)-phenyl{[3-(phenylmethyl)pyridin-2-yl]thio}methyl)morpholine (1:1 fumarate salt) as a white solid (0.070 g). LCMS 12 min gradient method, Rt=5.6 min, (M+H⁺)=377

### Example 7G

### (2S)-2-((S)-phenyl{[3-(phenyloxy)pyridin-2-yl]thio}methyl)morpholine fumarate

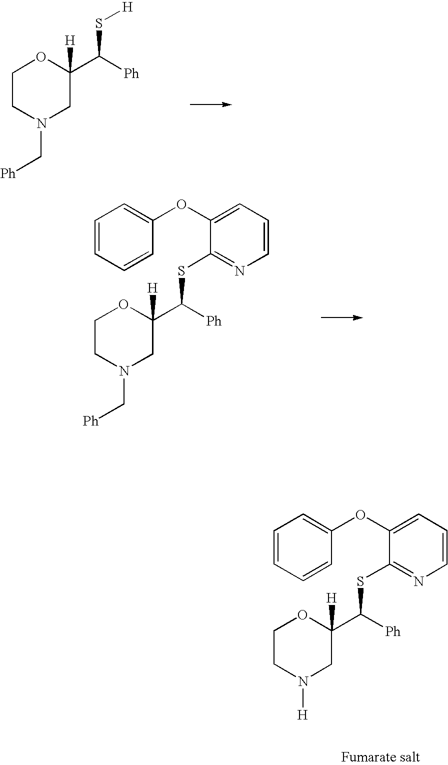

i) To a 100 ml round bottomed flask is added 2-chloro-3-pyridinol (0.50 g, 3.86 mmole), copper (II) acetate (0.70 g, 3.86 mmole), phenylboronic acid (0.94 g, 7.72 mmole) and powdered 4 Å molecular sieves. To the mixture is added DCM (39 ml) followed by triethylamine (2.69 ml, 19.30 mmole). This is stirred overnight, under nitrogen, at room temperature. The reaction mixture is poured into water (75 ml) and extracted with ethyl acetate (3×75 ml). The combined extracts are concentrated in vacuo to give a brown oil (0.65 g). Purification by automated flash chromatography (ISCO System, Redisep 35 g SiO₂ column, 0-20% ethyl acetate in cyclohexane gradient elution over 40 minutes) gives 2-chloro-3-phenoxypyridine as a colourless oil (0.32 g, 41%). LCMS 6 min gradient method, Rt=3.6min, (M+H⁺)=206

ii) To a solution of (S)-phenyl[(2S)-4-(phenylmethyl)morpholin-2-yl]methanethiol (6) (0.352 g, 1.18 mmole) and 2-chloro-3-phenoxypyridine (0.29 g, 1.41 mmole) in dry, degassed DMF (3 ml) is added, under nitrogen, cesium fluoride (0.179 g, 1.18 mmole). The mixture is left to stir for two days at 55° C. A further portion of cesium fluoride (0.063 g, 0.41 mmole) is added and the solution heated for 5 hours at 55° C. The reaction mixture is allowed to cool then loaded neat onto a 35 g SiO₂ Redisep column (preconditioned with cyclohexane). Automated flash chromatography (ISCO System, 0-40% ethyl acetate in cyclohexane gradient elution over 40 minutes at 30 ml/minute flow rate) yields a yellow oil (2.26 g). This is taken up in MeOH and loaded onto an SC10-2 column. The SC10-2 column is washed with methanol. The title compound is eluted with 2 N NH₃/methanol. This is concentrated in vacuo to give (2S)-2-{(S)-phenyl[(3-phenyloxypyridin-2-yl)thio]methyl}-4-(phenylmethyl)morpholine as a pale orange oil (0.092 g, 17%). LCMS 6 min gradient method, Rt=3.6 min, (M+H⁺)=469

iii) Deprotection of the morpholine nitrogen is carried out using the method and work up as described in Example 1G, using polymer supported diisopropylamine (3.78 mmole/g, 0.26 g, 0.98 mmole), (2S)-2-{(S)-phenyl[(3-phenyloxypyridin-2-yl)thio]methyl}-4-(phenylmethyl)morpholine (0.092 g, 0.20 mmole), dry DCM (5 ml), 1-chloroethyl chloroformate (0.11 ml, 0.98 mmole) and methanol (5 ml). This gave (2S)-2-((S)-phenyl{[3-(phenyloxy)pyridin-2-yl]thio)methyl)morpholine as a pale yellow oil (0.070 g, 95%). This oil is taken up in ethyl acetate. To this is added a solution of ftimaric acid (1.1 equiv, 0.024 g) in methanol. The resulting solution is concentrated in vacuo and the resulting oil triturated with ethyl acetate. The solid is collected by filtration to give the fumarate salt of (2S)-2-((S)-phenyl{[3-(phenyloxy)pyridin-2-yl]thio}methyl)morpholine (1:1 fumarate salt) as an off-white solid (0.094 g). LCMS 12 min gradient method, Rₜ=5.5 min, (M+H⁺)=379

### Example 8G

### (2S)-2-[(S)-[(3-chloropyridin-2-yl)thio](phenyl)methyl]morpholine fumarate

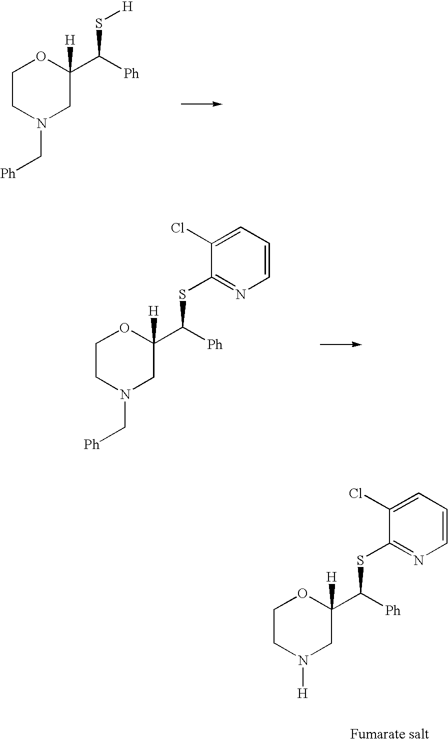

i) To a solution of (S)-phenyl[(2S)-4-(phenylmethyl)morpholin-2-yl]methanethiol (6) (0.446 g, 1.49 mmole) and 2,3-dichloropyridine (0.246 g, 1.67 mmole) in dry, degassed DMF (3 ml) is added, under nitrogen, sodium hydride (60% dispersion in oil, 0.061 g, 1.53 mmole). The mixture is left to stir overnight at room temperature. The reaction mixture is taken up in MeOH and loaded onto an SC10-2 column. The SC10-2 column is washed with methanol. The (2S)-2-[(S)-[(3-chloropyridin-2-yl)thio](phenyl)methyl]-4-(phenylmethyl)morpholine is eluted with 2 N NH₃/methanol. This is concentrated in vacuo to give (2S)-2-[(S)-[(3-chloropyridin-2-yl)thio](phenyl)methyl]-4-(phenylmethyl)morpholine as a pale yellow oil (0.61 g). LCMS 6 min gradient method, Rt=3.5 min, (M+H⁺)=411

ii) Deprotection of the morpholine nitrogen is carried out using the method and work up as described in Example 1G, using polymer supported diisopropylamine (3.78 mmole/g, 0.39 g, 1.46 mmole), (2S)-2-[(S)-[(3-chloropyridin-2-yl)thio](phenyl)methyl]-4-(phenylmethyl)morpholine (0.120 g, 0.292 mmole), dry DCM (15 ml), 1-chloroethyl chloroformate (0.16 ml, 1.46 mmole) and methanol (15 ml). This gives (2S)-2-[(S)-[(3-chloropyridin-2-yl)thio](phenyl)methyl]morpholine as a pale yellow oil (0.092 g, 98%). This oil is taken up in ethyl acetate. To this is added a solution of fumaric acid (1 equiv, 0.033 g) in methanol. The resulting solution is concentrated in vacuo to give an oil which is crystallised from IPA. The solid is collected by filtration to give the fumarate salt of (2S)-2-[(S)-[(3-chloropyridin-2-yl)thio](phenyl)methyl]morpholine (1:1 fumarate salt) as a white solid (0.111 g). LCMS 12 min gradient method, Rt=4.8 min, (M+H⁺)=321

### Example 9G

### (2S)-2-[(S)-[(3-methylpyridin-2-yl)thio](phenyl)methyl]morpholine fumarate

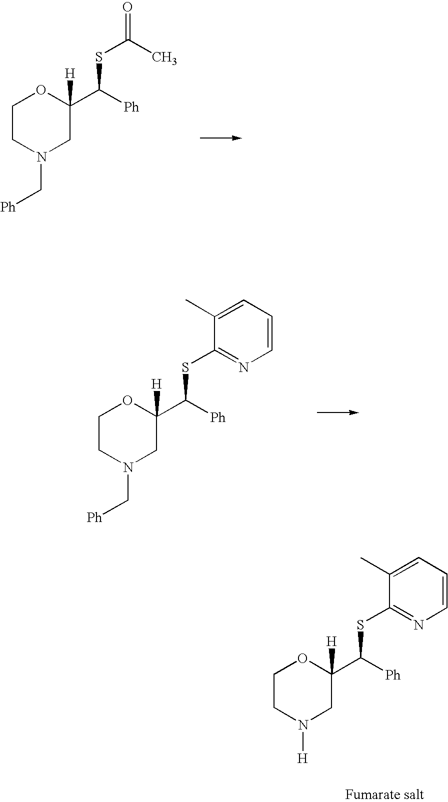

i) To a degassed solution of S-{(S)-phenyl[(2S)-4-(phenylmethyl)morpholin-2-yl]methyl}ethanethioate (5) (0.100 g, 0.293 mmole) and 2-fluoro-3-methylpyridine (0.325 g, 2.93 mmole) in DMF (1 ml) is added sodium methoxide (0.016 g, 0.293 mmole). The reaction mixture is left to stir at room temperature, under nitrogen, overnight. The reaction mixture is diluted with methanol and loaded onto an SC10-2 (5 g) column preconditioned with MeOH. The column is washed with MeOH then basic material is eluted with 2 N NH₃/methanol. This ammonia solution is concentrated in vacuo to give an orange oil (0.067 g) which is purified by automated flash chromatography (ISCO System, Redisep SiO₂ column, 0-20% ethyl acetate in cyclohexane gradient elution over 40 minutes) to give (2S)-2-[(S)-[(3-methylpyridin-2-yl)thio](phenyl)methyl]-4-(phenylmethyl)morpholine as a colourless oil (0.055 g, 44%). LCMS 6 min gradient method, Rt=2.9 min, (M+H⁺)=391

ii) To a suspension of polymer supported diisopropylamine (3.78 mmol/g, 0.167 g, 0.64 mmole) and (2S)-2-[(S)-[(3-methylpyridin-2-yl)thio](phenyl)methyl]-4-(phenylmethyl)morpholine (0.050 g, 0.13 mmole) in dry DCM (5 ml) is added 1-chloroethyl chloroformate (0.070 ml, 0.64 mmole) at room temperature and under nitrogen. The mixture is heated at 40° C. for 1.5 hours. The reaction mixture is filtered and concentrated in vacuo then taken up in methanol (5 ml). The solution is left to stir at room temperature for 2.5 hours. After this time, the reaction mixture is loaded directly onto an SC10-2 column. The SC10-2 column is washed with methanol. The free base of the title compound is eluted with 2 N NH₃/methanol. This ammonia solution is concentrated in vacuo to give (2S)-2-[(S)-[(3-methylpyridin-2-yl)thio](phenyl)methyl]morpholine as an orange oil (0.037. g, 97%). This oil is taken up in methanol. To this is added a solution of fumaric acid (1 equiv, 0.014 g) in methanol. This is stirred for a couple of minutes, then EtOAc followed by isohexane added. The resulting precipitate is collected by filtration to yield a white solid (0.048 g). This is recrystallised from ethyl acetate and isohexane to give the fumarate salt of (2S)-2-[(S)-[(3-methylpyridin-2-yl)thio](phenyl)methyl]morpholine (1:1 fumarate salt) as a white solid (0.013 g) LCMS 12 min gradient method, Rt=4.5 min, (M+H⁺)=301

### Example 10G

### (2S)-2-[(S)-{[3-(4-chlorophenyl)pyridin-2-yl]thio}(phenyl)methyl]morpholine fumarate

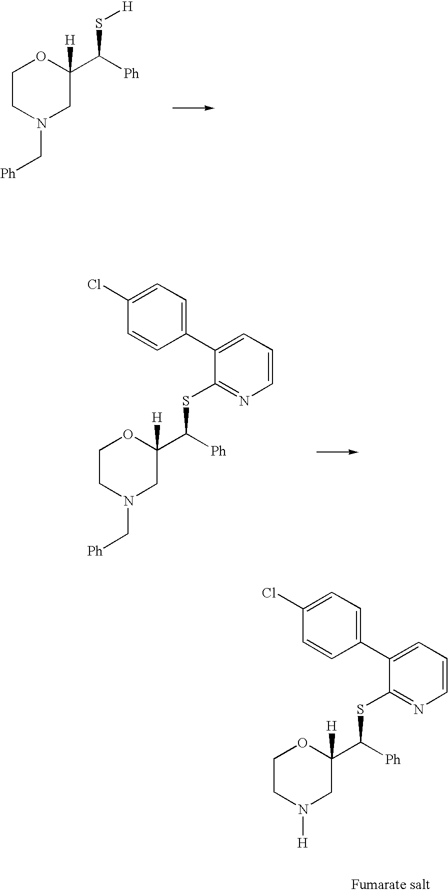

i) To bis(benzonitrile)palladium(II)dichloride (0.054 g, 0.14 mmole) and 1,4-bis(diphenylphosphine)butane (0.091 g, 0.21 mmole) is added dry toluene (6 ml), under nitrogen, and the mixture stirred for half an hour. To this is added 3-bromo-2-fluoropyridine (0.50 g, 2.83 mmole) in ethanol (1.4 ml) followed by a solution of 4-chlorophenylboronic acid (0.887 g, 5.67 mmole) in ethanol (2.4 ml). To this is added an aqueous solution of sodium carbonate (1 M, 2.83 ml, 2.83 mmole). The mixture is heated at 60° C. for 24 hours, then at 75° C. for a further 16 hours. The organic layer is loaded directly onto a 40 g Redisep SiO₂ column and components isolated by automated flash chromatography (ISCO System, 0-30% ethyl acetate in cyclohexane gradient elution over 40 minutes). This gave 3-(4-chlorophenyl)-2-fluoropyridine as a white solid (0.323 g, 55%). LCMS 6 min gradient method, Rt=4.0 min, (M+H⁺)=208

ii) To a solution of (S)-phenyl[(2S)-4-(phenylmethyl)morpholin-2-yl]methanethiol (6) (0.388 g, 1.30 mmole) and 3-(4-chlorophenyl)-2-fluoropyridine (0.323 g, 1.56 mmole) in dry, degassed DMF (3 ml) is added, under nitrogen, cesium fluoride (0.295 g, 1.94 mmole). The mixture is heated at 65° C. over the weekend. After this time, the reaction mixture is allowed to cool. The resulting solid is taken up in MeOH/DCM and loaded directly onto an SC10-2 column. The SC10-2 column is washed with methanol followed by 2 N NH₃/methanol. The ammonia solution is concentrated in vacuo to give (2S)-2-[(S)-{[3-(4-chlorophenyl)pyridin-2-yl]thio}(phenyl)methyl]-4-(phenylmethyl)morpholine as an orange foam (0.514 g). This is purified by automated flash chromatography (ISCO System, 0-30% ethyl acetate in cyclohexane gradient elution over 40 minutes at 40 ml/minute flow rate) to give (2S)-2-[(S)-{[3-(4-chlorophenyl)pyridin-2-yl]thio}(phenyl)methyl]-4-(phenylmethyl)morpholine as a white foam (0.178 g, 28%). LCMS 6 min gradient method, Rt=4.2 min, (M+H⁺)=487

iii) Deprotection of the morpholine nitrogen is carried out using the method and work up as described in Example 1G, using polymer supported diisopropylamine (3.78 mole/g, 0.48 g, 1.80 mmole), (2S)-2-[(S)-{[3-(4-chlorophenyl)pyridin-2-yl]thio}(phenyl)methyl]-4-(phenylmethyl)morpholine (0.175 g, 0.36 mmole), dry DCM (10 ml), 1-chloroethyl chloroformate (0.20 ml, 1.80 mmole) and methanol (10 ml). This gave a colourless residue (0.129 g, 90%). This residue is taken up in ethyl acetate. To this is added a solution of fumaric acid (1.1 equiv, 0.035 g) in methanol. The resulting solid is recombined with the mother liquor and purified on a UV Guided PrepHPLC (Flex) System and treated with SC10-2 to give a yellow solid. This is further purified by automated flash chromatography (ISCO System, Redisep 4 g SiO₂ column, 0-5% methanol in dichloromethane gradient elution over 40 minutes, then 10 minutes at 5% Methanol in dichloromethane with 10 ml/min flow rate) to give (2S)-2-[(S)-{[3-(4-chlorophenyl)pyridin-2-yl]thio}(phenyl)methyl]morpholine as a pale yellow oil (0.049 g, 34%). This oil is taken up in ethyl acetate. To this is added a solution of fumaric acid (1.1 equiv, 0.0145 g) in methanol. The resulting solution is concentrated in vacuo and recrystallised from MeOH and Et₂O. The solid is collected by filtration to give the fumarate salt of (2S)-2-[(S)-{[3-(4-chlorophenyl)pyridin-2-yl]thio}(phenyl)methyl]morpholine (1:1 fumarate salt) as a white solid (0.047 g). LCMS 12 min gradient method, Rt=5.7 min, (M+H⁺)=397

### Example 11G

### (2S)-2-[(S)-[(5-bromopyridin-2-yl)thio](phenyl)methyl]morpholine fumarate

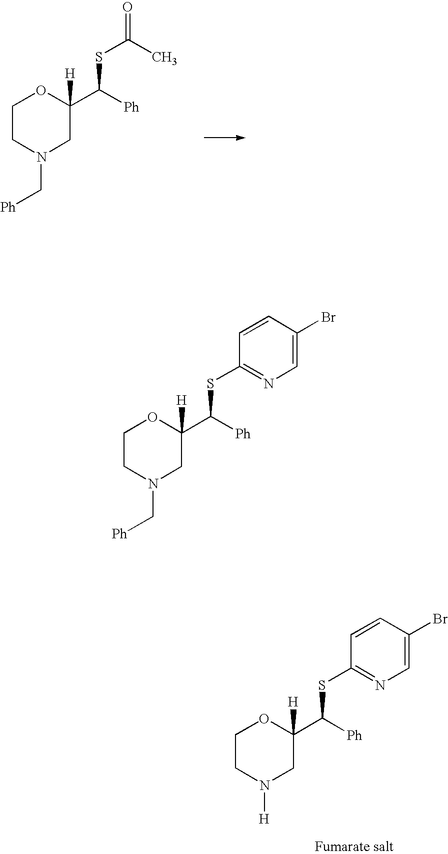

i) To a solution of S-{(S)-phenyl[(2S)-4-(phenylmethyl)morpholin-2-yl]methyl}ethanethioate (5) (0.25 g, 0.73 mmole) in dry methanol (2 ml) is added sodium methoxide (0.040 g, 0.73 mmole) under nitrogen. This is left to stir at room temperature for 1 hour. Methanol is removed in vacuo and replaced with DMF (1 ml). To this is then added the 5-bromo-2-fluoropyridine (0.11 ml, 1.02 mmole). The reaction mixture is left to stir at room temperature, under nitrogen, overnight. The reaction mixture is diluted with DCM and loaded directly onto a 35 g Redisep column. Purification by automated flash chromatography (ISCO System, Redisep 35 g SiO₂ column, 0-20% ethyl acetate in cyclohexane gradient elution over 40 minutes) gave (2S)-2-[(S)-[(5-bromopyridin-2-yl)thio](phenyl)methyl]-4-(phenylmethyl)morpholine as a colourless oil (0.186 g, 56%). LCMS 6 min gradient method, Rt=3.6 min, (M+H⁺)=455/457

ii) To a suspension of polymer supported diisopropylamine (3.78 mmol/g, 0.108 g, 20.4 mmole) and (2S)-2-[(S)-[(5-bromopyridin-2-yl)thio)(phenyl)methyl]-4-(phenylmethyl)morpholine (0.186 g, 0.408 mmole) in dry DCM (10 ml) is added 1-chloroethyl chloroformate (0.22 ml, 2.04 mmole) at room temperature and under nitrogen. The mixture is heated at 40° C. for 2.5 hours. The reaction mixture is then filtered and concentrated in vacuo then taken up in methanol (10 ml). The solution is left to stir at room temperature overnight. After this time, the reaction mixture is loaded directly onto an SC10-2 column (5 g). The SC10-2 column is washed with methanol. The target compound is eluted with 2 N NH₃/methanol. This is concentrated in vacuo to give (2S)-2-[(S)-[(5-bromopyridin-2-yl)thio](phenyl)methyl]morpholine as a colourless oil (0.108. g, 72%). This oil is taken up in ethanol. To this is added a solution of fumaric acid (1.2 equiv, 0.041 g) in ethanol. Solvent is removed in vacuo and the resulting residue triturated with EtOAc. This solid is collected by filtration to give the fumarate salt of (2S)-2-[(S)-[(5-bromopyridin-2-yl)thio](phenyl)methyl]morpholine (1:1 fumarate salt) as a white solid (0.105 g). LCMS 12 min gradient method, Rt=5.0 min, (M+H⁺)=365/367

### Example 12G

### 2-{[(S)-(2S)-morpholin-2-yl(phenyl)methyl]thio}pyridine-3-carboxamide fumarate

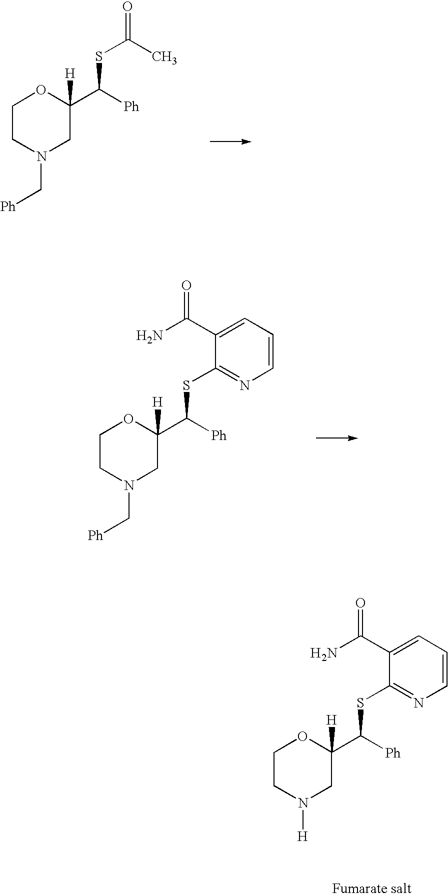

i) To a degassed solution of S-{(S)-phenyl[(2S)-4-(phenylmethyl)morpholin-2-yl]methyl}ethanethioate (5) (0.100 g, 0.293 mmole) and 2-chloronicotinamide (0.046 g, 0.293 mmole) in ethanol (3 ml) is added a solution of sodium hydroxide in water (2 M, 0.293 ml, 0.586 mmole). The resulting solution is stirred at room temperature overnight. An additional portion of 2-chloronicotinamide (0.046 g, 0.293 mmole) is added to the reaction mixture which is then heated at 40° C. overnight. The reaction mixture is diluted with methanol and loaded onto an SC10-2 column preconditioned with MeOH. The column is washed with MeOH then basic material is eluted with 2 N NH₃/methanol. This ammonia solution is concentrated in vacuo to give 2-({[(S)-phenyl[(2S)-4-(phenylmethyl)morpholin-2-yl)methyl)thio)pyridine-3-carboxamide as a pale orange residue (0.124 g, 100%). LCMS 6 min gradient method, Rt=2.1 min, (M+H⁺)=420

ii) To a suspension of polymer supported diisopropylamine (3.78 mmol/g, 0.38 g, 1.47 mmole) and 2-({[(S)-phenyl[(2S)-4-(phenylmethyl)morpholin-2-yl)methyl}thio)pyridine-3-carboxamide (0.123 g, 0.29 mmole) in dry DCM (10 ml) is added 1-chloroethyl chloroformate (0.16 ml, 1.47 mmole) at room temperature and under nitrogen. The mixture is heated at 40° C. for 2 hours. The reaction mixture is then filtered and concentrated in vacuo to give a pale yellow residue. This is taken up in methanol (10 ml) and the solution left to stir at room temperature for 3 hours. After this time, the reaction mixture is loaded directly onto an SC10-2 column. The SC10-2 column is washed with methanol then more basic compounds are eluted with 2 N NH₃/methanol. The ammonia solution is concentrated in vacuo to give 2-{[(S)-(2S)-morpholin-2-yl(phenyl)methyl]thio}pyridine-3-carboxamide as a pale yellow oil (0.097 g, 100%). The pale yellow oil is taken up in methanol. To this is added a solution of fumaric acid (1 equiv, 0.0153 g) in methanol. This is stirred for a couple of minutes, then EtOAc added. The resulting precipitate is collected by filtration to give the fumarate salt of 2-{[(S)-(2S)-morpholin-2-yl(phenyl)methyl]thio}pyridine-3-carboxamide (1:1 fumarate salt) as a white solid (0.095 g). LCMS 12 min gradient method, Rt=2.4 min, (M+H⁺)=330

### Example 13G

### 2-{[(S)-(2S)-morpholin-2-yl(phenyl)methyl]thio}pyridine-3-carbonitrile fumarate

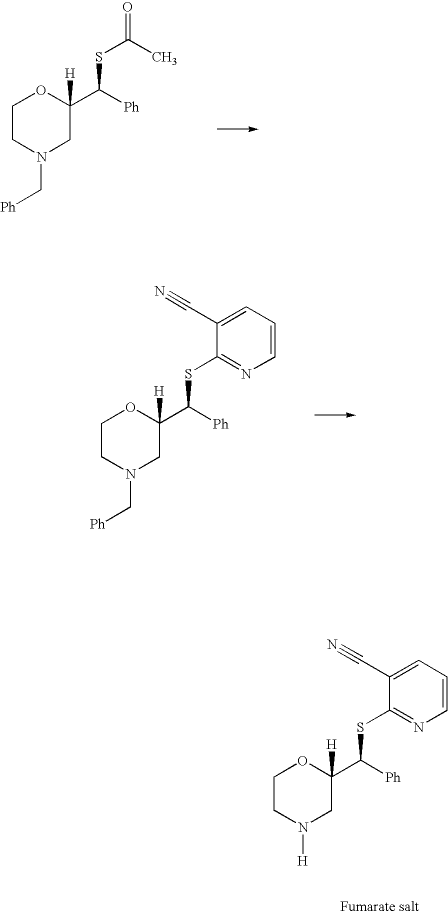

i) To a degassed solution of S-{(S)-phenyl[(2S)-4-(phenylmethyl)morpholin-2-yl]methyl}ethanethioate (5) (0.050 g, 0.147 mmole) and 2-chloro-3-cyanopyridine (0.020 g, 0.146 mmol) in ethanol (1.5 ml) is added a solution of sodium hydroxide in water (2 M, 0.146 ml, 0.293 mmole). The resulting solution is stirred at room temperature for ˜17 hours. The reaction mixture is diluted with methanol and loaded onto an SC10-2 column preconditioned with MeOH. The column is washed with MeOH then basic material is eluted with 2 N NH₃/methanol. This ammonia solution is concentrated in vacuo to give 2-({[(S)-phenyl[(2S)-4-(phenylmethyl)morpholin-2-yl]methyl}thio)pyridine-3-carbonitrile as an off white solid (0.055 g, 93%). LCMS 6 min gradient method, Rt≈2.8 min, (M+H⁺)=402

ii) To a suspension of polymer supported diisopropylamine (3.78 mmol/g, 0.181 g, 0.685 mmole) and 2-({[(S)-phenyl[(25)-4-(phenylmethyl)morpholin-2-yl]methyl}thio)pyridine-3-carbonitrile (0.055 g, 0.137 mmole) in dry DCM (5 ml) is added 1-chloroethyl chloroformate (0.075 ml, 0.685 mmole) at room temperature and under nitrogen. The mixture is heated at 40° C. for 2 hours. The reaction mixture is then filtered and concentrated in vacuo to give a pale orange liquid. This is taken up in methanol (5 ml) and the solution left to stir at room temperature overnight. After this time, the reaction mixture is loaded directly onto an SC10-2 column. The SC10-2 column is washed with methanol then more basic material is eluted with 2 N NH₃/methanol. The ammonia solution is concentrated in vacuo to give 2-{[(S)-(2S)-morpholin-2-yl(phenyl)methyl]thio}pyridine-3-carbonitrile as a pale yellow oil (0.041 g, 95%). The pale yellow oil is taken up in methanol. To this is added a solution of fumaric acid (1 equiv, 0.0153 g) in methanol. This is stirred for a couple of minutes, then EtOAc followed by cyclohexane added. The resulting precipitate is collected by filtration to give the fumarate salt of 2-{[(S)-(2S)-morpholin-2-yl(phenyl)methyl]thio}pyridine-3-carbonitrile (1:1 fumarate salt) as a white solid (0.042 g). LCMS 12 min gradient method, Rt=4.6 min, (M+H⁺)=312

### Example 14G

### (2S)-2-[phenyl(pyridin-2-ylthio)methyl]morpholine hydrochloride

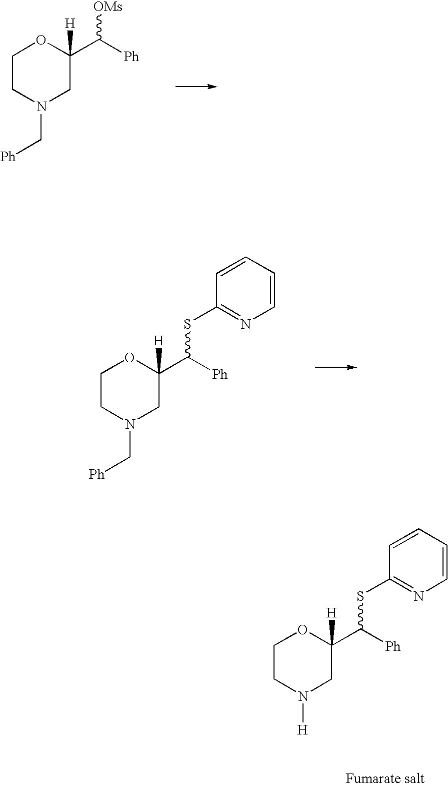

i) To a stirred solution of (R)-phenyl[(2S)-4-(phenylmethyl)morpholin-2-yl]methyl methanesulfonate (0.70 g, 1.94 mmole) and 2-mercaptopyridine (0.54 g, 4.84 mmole) in anhydrous DMF, at room temperature and under nitrogen, is added potassium carbonate (0.80 g, 5.81 mmole). The reaction is left to stir at room temperature for 6 days. The reaction mixture is diluted with methanol and loaded onto an SC10-2 column preconditioned with MeOH. The column is washed with MeOH then basic material is eluted with 2 N NH₃/methanol. This ammonia solution is concentrated in vacuo to give an orange residue (0.881 g). Purification by automated flash chromatography (ISCO System, 0-30% ethyl acetate in isohexane gradient elution over 30 minutes) gave (2S)-2-[phenyl(pyridin-2-ylthio)methyl]-4-(phenylmethyl)morpholine as a colourless oil (0.245 g, 34%). LCMS 6 min gradient method, Rt=2.7 min, (M+H⁺)=377.

ii) Deprotection of the morpholine nitrogen is carried out using the method and work up as described in Example 1G, using polymer supported diisopropylamine (3.78 mmole/g, 0.43 g, 1.64 mmole), (2S)-2-[phenyl(pyridin-2-ylthio)methyl]-4-(phenylmethyl)morpholine (0.1 03g, 0.274 mmole), dry DCM (10 ml), 1-chloroethyl chloroformate (0.15 ml, 1.37 mmole) and methanol (10 ml). This gave a pale yellow oil (0.058 g, 74%).). Purification of this residue by automated flash chromatography (ISCO System, SiO₂ Redisep column, 10% MeOH in DCM) gave a colourless oil (0.044 g, 54%). This oil is taken up in ethyl acetate. To this is added a solution of hydrochloric acid in dioxane (4 M, 0.1 ml). Concentration in vacuo gave the hydrochloride salt of (2S)-2-[phenyl(pyridin-2-ylthio)methyl] as a white solid (0.045 g). LCMS 6 min gradient method, Rt=1.8 min, (M+H⁺)=287

### Example 15G

### (2S)-2-[(S)-[(3-iodopyridin-2-yl)thio](phenyl)methyl]morpholine fumarate

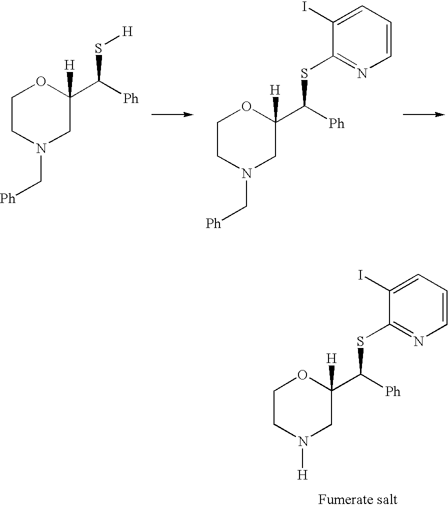

i) To (S)-phenyl[(2S)-4-(phenylmethyl)morpholin-2-yl]methanethiol (6) (0.50 g, 1.67 mmole) and 2-chloro-3-iodopyridine (0.48 g, 2.00 mmole) in degassed DMF (3 ml) is added cesium fluoride (0.38 g, 2.50 mmole) at room temperature and under nitrogen. The mixture is heated at between 55-75° C. for 3 days. The organic layer is then loaded directly onto a 35 g ISCO column (SiO₂) and columned using automated flash chromatography (0-30% EtOAc in cyclohexane over 30 minutes) to give a pale yellow crystalline solid (0.55 g). The solid is taken up in DCM:MeOH (1:1) and loaded onto an SC10-2 column (10 g) preconditioned with MeOH. The column is washed with MeOH to remove 2-chloro-3-iodopyridine, then more basic material is eluted with 2 N NH₃/methanol. The ammonia solution is concentrated in vacuo to give (2S)-2-[(S)-[(3-iodopyridin-2-yl)thio](phenyl)methyl]-4-(phenylmethyl)morpholine as a pale yellow solid (0.19 g, 23%). LCMS 6 min gradient method, Rt=3.8 min, (M+H⁺)=503

ii) To a suspension of polymer supported diisopropylamine (3.72 mmol/g, 0.285 g, 1.06 mmole) and (2S)-2-[(S)-[(3-iodopyridin-2-yl)thiol(phenyl)methyl]-4-(phenylmethyl)morpholine (0.107 g, 0.21 mmole) in dry DCM (1.5 ml) is added 1-chloroethyl chloroformate (0.116 ml, 1.06 mmole) at room temperature and under nitrogen. The mixture is heated at 40° C. for 2 hours. The reaction mixture is then filtered and concentrated in vacuo to give a pale orange liquid. This is taken up in methanol (1.5 ml) and the solution left to stir at room temperature overnight. After stirring overnight at room temperature, the reaction mixture is loaded directly onto an SC10-2 column. The SC10-2 column is washed with methanol, then more basic material is eluted with 2 N NH₃/methanol. The ammonia solution is concentrated in vacuo to give (2S)-2-[(S)-[(3-iodopyridin-2-yl)thio](phenyl)methyl]morpholine as a pale yellow oil (0.047 g, 53%). This oil is taken up in methanol and to this is added a solution of fumaric acid (1 equiv, 0.013 g) in methanol. This is stirred for a couple of minutes, then EtOAc followed by Et₂O added. The resulting precipitate is collected by filtration to give the fumarate salt of (2S)-2-[(S)-[(3-iodopyridin-2-yl)thio](phenyl)methyl]morpholine (1:1 fumarate salt) as a white solid (0.036 g). LCMS 12 min gradient method, Rt=4.9 min, (M+H⁺)=413

The pharmacological profile of the compounds of Formulae (IA), (IB), (IC), (ID), (IE), (IF) and (IG) can be demonstrated as follows. The preferred exemplified compounds above exhibit a Kᵢ value less than 500 nM at the norepinephrine transporter as determined using the scintillation proximity assay described below. Furthermore, the preferred exemplified compounds above selectively inhibit the norepinephrine transporter relative to the serotonin and dopamine transporters by a factor of at least five using the scintillation proximity assays as described below.

Generation of Stable Cell-Lines Expressing the Human Dopamine, Norepinephrine and Serotonin Transporters

Standard molecular cloning techniques are used to generate stable cell-lines expressing the human dopamine, norepinephrine, and serotonin transporters. The polymerase chain reaction (PCR) was used in order to isolate and amplify each of the three full-length cDNAs from an appropriate cDNA library. Primers for PCR were designed using the following published sequence data:

Human dopamine transporter: GenBank M95 167. Reference: Vandenbergh D J, Persico A M and Uhl G R. A human dopamine transporter cDNA predicts reduced glycosylation, displays a novel repetitive element and provides racially-dimorphic TaqI RFLPs. Molecular Brain Research (1992) Volume 15, pages 161-166.

Human norepinephrine transporter: GenBank M65105. Reference: Pacholczyk T, Blakely, R D and Amara S G. Expression cloning of a cocaine- and antidepressant-sensitive human noradrenaline transporter. Nature (1991) Volume 350, pages 350-354.

Human serotonin transporter: GenBank L05568. Reference: Ramamoorthy S, Bauman A L, Moore K R, Han H, Yang-Feng T, Chang A S, Ganapathy V and Blakely R D. Antidepressant- and cocaine-sensitivehuman serotonin transporter: Molecular cloning, expression, and chromosomal localization. Proceedings of the National Academy of Sciences of the USA (1993) Volume 90, pages 2542-2546.

The PCR products are cloned into a mammalian expression vector (e.g., pcDNA3.1 (Invitrogen)) using standard ligation techniques. The constructs are then used to stably transfect HEK293 cells using a commercially available lipofection reagent (Lipofectamine™—Invitrogen) following the manufacturer's protocol.

Scintillation Proximity Assays for Determining the Affinity of Test Lipands at the Norepinephrine Transporter

The compounds of Formulae (II) and (III) of the present invention are norepinephrine reuptake inhibitors, and possess excellent activity in, for example, a scintillation proximity assay (e.g., J. Gobel, D. L. Saussy and A. Goetz, J. Pharmacol. Toxicol. (1999) 42:237-244). Thus, ³H-nisoxetine binding to norepinephrine re-uptake sites in a cell line transfected with DNA encoding human norepinephrine transporter binding protein has been used to determine the affinity of ligands at the norepinephrine transporter.

Membrane Preparation:

Cell pastes from large scale production of HEK-293 cells expressing cloned human norepinephrine transporters were homogenized in 4 volumes 50 mM Tris-HCl containing 300 mM NaCl and 5 mM KCl, pH 7.4. The homogenate was centrifuged twice (40,000 g, 10 min, 4° C.) with pellet re-suspension in 4 volumes of Tris-HCl buffer containing the above reagents after the first spin and 8 volumes after the second spin. The suspended homogenate was centrifuged (100 g, 10 min, 4° C.) and the supernatant kept and re-centrifuged (40,000 g, 20 min, 4° C.). The pellet was resuspended in Tris-HCl buffer containing the above reagents along with 10% w/v sucrose and 0.1 mM phenylmethylsulfonyl fluoride (PMSF). The membrane preparation was stored in aliquots (1 ml) at −80° C. until required. The protein concentration of the membrane preparation was determined using a bicinchoninic acid (BCA) protein assay reagent kit (available from Pierce).

[³H]-Nisoxetine Binding Assay:

Each well of a 96 well microtitre plate was set up to contain the following:
50 μl 2 nM [N-methyl-³H]-Nisoxetine hydrochloride (70-87 Ci/mmol, from NEN Life Science Products)
75 μl Assay buffer (50 mM Tris-HCl pH 7.4 containing 300 mM NaCl and 5 mM KCl)
25 μl Test compound, assay buffer (total binding) or 10 μM Desipramine HCl (non-specific binding)
50 μl Wheatgerm agglutinin coated poly (vinyltoluene) (WGA PVT) SPA Beads (Amersham Biosciences RPNQ0001) (10 mg/ml)
50 μl Membrane (0.2 mg protein per ml)

The microtitre plates were incubated at room temperature for 10 hours prior to reading in a Trilux scintillation counter. The results were analysed using an automatic spline fitting programme (Multicalc, Packard, Milton Keynes, UK) to provide Ki values for each of the test compounds.

Serotonin Binding Assay

The ability of a test compound to compete with [³H]-citalopram for its binding sites on cloned human serotonin transporter containing membranes has been used as a measure of test compound ability to block serotonin uptake via its specific transporter (Ramamoorthy, S., Giovanetti, E., Qian, Y., Blakely, R., (1998) J. Biol. Chem. 273: 2458).

Membrane Preparation:

Membrane preparation is essentially similar to that for the norepinephrine transporter containing membranes as described above. The membrane preparation was stored in aliquots (1 ml) at −70° C. until required. The protein concentration of the membrane preparation was determined using a BCA protein assay reagent kit.

[³H]-Citalopram Binding Assay:

Each well of a 96 well microtitre plate was set up to contain the following:
50 μl 2 nM [³H]-Citalopram (60-86 Ci/mmol, Amersham Biosciences)
75 μl Assay buffer (50 mM Tris-HCl pH 7.4 containing 150 mM NaCl and 5 mM KCl)
25 μl Diluted compound, assay buffer (total binding) or 100 μM Fluoxetine (non-specific binding)
50 μl WGA PVT SPA Beads (40 mg/ml)
50 μl Membrane preparation (0.4 mg protein per ml)

The microtitre plates were incubated at room temperature for 10 hours prior to reading in a Trilux scintillation counter. The results were analysed using an automatic spline fitting programme (Multicalc, Packard, Milton Keynes, UK) to provide Ki (nM) values for each of the test compounds.

Dopamine Binding Assay

The ability of a test compound to compete with [³ H]-WIN35,428 for its binding sites on human cell membranes containing cloned human dopamine transporter has been used as a measure of the ability of such test compounds to block dopamine uptake via its specific transporter (Ramamoorthy et al 1998 supra).

Membrane Preparation:

Is essentially the same as for membranes containing cloned human serotonin transporter as described above.

[³H]-WIN35,428 Binding Assay:

Each well of a 96 well microtitre plate was set up to contain the following:
50 μl 4 nM [³H]-WIN35,428 (84-87 Cl/mmol, from NEN Life Science Products)
75 μl Assay buffer (50 mM Tris-HCl pH 7.4 containing 150 mM NaCl and 5 mM KCl)
25 μl Diluted compound, assay buffer (total binding) or 100 μM Nomifensine (non-specific binding)
50 μl WGA PVT SPA Beads (10 mg/ml)
50 μl Membrane preparation (0.2 mg protein per ml.)

The microtitre plates were incubated at room temperature for 120 minutes prior to reading in a Trilux scintillation counter. The results were analysed using an automatic spline fitting programme (Multicalc, Packard, Milton Keynes, UK) to provide Ki values for each of the test compounds.

Acid Stability

The acid stability of a compound according to the present invention was determined as a solution in buffer at 6 different pH values (HCl 0.1 N, pH 2, pH 4, pH 6, pH 7, and pH 8) at 40° C. over a time course of 72 hours. Samples were taken at the beginning of the study and after 3, 6 and 24 hours and analysed by capillary electrophoresis. The original sample used in this study contained 0.8% of the undesired epimer as internal standard. The samples taken at the different time points during the study did not show any significant change in the percentage of the undesired epimer. This assay confirms that compounds of the present invention are chemically and configurationally stable under acidic conditions.

In Vitro Determination of the Interaction of Compounds with CYP2D6 in Human Hepatic Microsomes

Cytochrome P450 2D6 (CYP2D6) is a mammalian enzyme which is commonly associated with the metabolism of around 30% of pharmaceutical compounds. Moreover, this enzyme exhibits genetic polymorphism, resulting in the presence of both normal and poor metabolizers in the population. A low involvement of CYP2D6 in the metabolism of compounds (i.e. the compound being a poor substrate of CYP2D6) is desirable in order to reduce any variability from subject to subject in the pharmacokinetics of the compound. Also, compounds with a low inhihibitor potential for CYP2D6 are desirable in order to avoid drug-drug interactions with co-administered drugs that are substrates of CYP2D6. Compounds can be tested both as substrates and as inhibitors of this enzyme by means of the following assays.

CYP2D6 Substrate Assay

Principle:

This assay determines the extent of the CYP2D6 enzyme involvement in the total oxidative metabolism of a compound in microsomes. Preferred compounds of the present invention exhibit less than 75% total metabolism via the CYP2D6 pathway.

For this in vitro assay, the extent of oxidative metabolism in human liver microsomes (HLM) is determined after a 30 minute incubation in the absence and presence of Quinidine, a specific chemical inhibitor of CYP2D6. The difference in the extent of metabolism in absence and presence of the inhibitor indicates the involvement of CYP2D6 in the metabolism of the compound.

Materials and Methods:

Human liver microsomes (mixture of 20 different donors, mixed gender) were acquired from Human Biologics (Scottsdale, Ariz., USA). Quinidine and β-NADPH (β-Nicotinamide Adenine Dinucleotide Phosphate, reduced form, tetrasodium salt) were purchased from Sigma (St Louis, Mo., USA). All the other reagents and solvents were of analytical grade. A stock solution of the new chemical entity (NCE) was prepared in a mixture of Acetonitrile/Water to reach a final concentration of acetonitrile in the incubation below 0.5%.

The microsomal incubation mixture (total volume 0.1 mL) contained the NCE (4 μM), μ-NADPH (1 mM), microsomal proteins (0.5 mg/mL), and Quinidine (0 or 2 μM) in 100 mM sodium phosphate buffer pH 7.4. The mixture was incubated for 30 minutes at 37° C. in a shaking waterbath. The reaction was terminated by the addition of acetonitrile (75 μL). The samples were vortexed and the denaturated proteins were removed by centrifugation. The amount of NCE in the supernatant was analyzed by liquid chromatography /mass spectrometry (LC/MS) after addition of an internal standard. A sample was also taken at the start of the incubation (t=0), and analysed similarly.

Analysis of the NCE was performed by liquid chromatography/mass spectrometry. Ten μL of diluted samples (20 fold dilution in the mobile phase) were injected onto a Spherisorb CN Column, 5 μM and 2.1 mm×100 mm (Waters corp. Milford, Mass., USA). The mobile phase consisting of a mixture of Solvent A/Solvent B, 30/70 (v/v) was pumped (Alliance 2795, Waters corp. Milford, Mass., USA) through the column at a flow rate of 0.2 ml/minute. Solvent A and Solvent B were a mixture of ammonium formate 5.10⁻³ M pH 4.5/methanol in the proportions 95/5 (v/v) and 10/90 (v/v), for solvent A and solvent B, respectively. The NCE and the internal standard were quantified by monitoring their molecular ion using a mass spectrometer ZMD or ZQ (Waters-Micromass corp, Machester, UK) operated in a positive electrospray ionisation.

The extent of CYP2D6 involvement (% of CYP2D6 involvement) was calculated comparing the extent of metabolism in absence and in presence of quinidine in the incubation.

$$The extent of metabolism without inhibitor ( \% ) was calculated as follows \text{:}$$

$$\frac{\begin{matrix}( NCE response in samples without inhibitor ) time 0 - \\ ( NCE response in samples without inhibitor ) time 30\end{matrix}}{( NCE response in samples without inihbitor ) time 0} \times 100$$

$$The extent of metabolism with inhibitor ( \% ) was calculated as follows \text{:}$$

$$\frac{\begin{matrix}( NCE response in samples without inhibitor ) time 0 - \\ ( NCE response in samples with inihbitor ) time 30\end{matrix}}{( NCE response in samples without inhibitor ) time 0} \times 100$$

where the NCE response is the area of the NCE divided by the area of the internal standard in the LC/MS analysis chromatogram, time0 and time30 correspond to the 0 and 30 minutes incubation time.

The % of CYP2D6 involvement was calculated as follows:

$$\frac{\begin{matrix}( \% extent of metabolism without inhibitor ) - \\ ( \% extent of metabolism with inhibitor )\end{matrix}}{\% extent of metabolism without inhibitor} \times 100$$

CYP2D6 Inhibitor Assay

Principle:

The CYP2D6 inhibitor assay evaluates the potential for a compound to inhibit CYP2D6. This is performed by the measurement of the inhibition of the bufuralol 1′-hydroxylase hydroxylase activity by the compound compared to a control. The 1′-hydroxylation of bufuralol is a metabolic reaction specific to CYP2D6. Preferred compounds of the present invention exhibit an IC₅₀ higher than 6 μM for CYP2D6 activity, the IC₅₀ being the concentration of the compound that gives 50% of inhibition of the CYP2D6 activity.

Materials and Methods:

Human liver microsomes (mixture of 20 different donors, mixed gender) were acquired from Human Biologics (Scottsdale, Ariz.). β-NADPH was purchased from Sigma (St Louis, Mo.). Bufuralol was purchased from Ultrafine (Manchester, UK). All the other reagents and solvents were of analytical grade.

Microsomal incubation mixture (total volume 0.1 mL) contained bufuralol 10 μM, β-NADPH (2 mM), microsomal proteins (0.5 mg/mL), and the new chemical entity (NCE) (0, 5, and 25 μM) in 100 mM sodium phosphate buffer pH 7.4. The mixture was incubated in a shaking waterbath at 37° C. for 5 minutes. The reaction was terminated by the addition of methanol (75 μL). The samples were vortexed and the denaturated proteins were removed by centrifugation. The supernatant was analyzed by liquid chromatography connected to a fluorescence detector. The formation of the 1′-hydroxybufuralol was monitored in control samples (0 μM NCE) and in the samples incubated in presence of the NCE. The stock solution of NCE was prepared in a mixture of Acetonitrile/Water to reach a final concentration of acetonitrile in the incubation below 1.0%.

The determination of 1′hydroxybufuralol in the samples was performed by liquid chromatograhy with fluorimetric detection as described below. Twenty five μL samples were injected onto a Chromolith Performance RP-18e column (100 mm×4.6 mm) (Merck KGAa, Darmstadt, Germany). The mobile phase, consisting of a mixture of solvent A and solvent B whose the proportions changed according the following linear gradient, was pumped through the column at a flow rate of 1 ml/min:

| Time (minutes) | Solvent A (%) | Solvent B (%) |
|---|---|---|
| 0 | 65 | 35 |
| 2.0 | 65 | 35 |
| 2.5 | 0 | 100 |
| 5.5 | 0 | 100 |
| 6.0 | 65 | 35 |

Solvent A and Solvent B consisted of a mixture of 0.02 M potassium dihydrogenophosphate buffer pH3/methanol in the proportion 90/10 (v/v) for solvent A and 10/90 (v/v) for solvent B. The run time was 7.5 minutes. Formation of 1′-hydroxybufuralol was monitored by fluorimetric detection with extinction at λ 252 nm and emission at λ 302 nm.

The IC₅₀ of the NCE for CYP2D6 was calculated by the measurement of the percent of inhibition of the formation of the 1′-hydroxybufuralol in presence of the NCE compared to control samples (no NCE) at a known concentration of the NCE.
The percent of inhibition of the formation of the 1′-hydroxybufuralol is calculated as follows:

$$\frac{\begin{matrix}( {1}^{′} \text{-} hydroxybufuralol formed without inhibitor ) - \\ ( {1}^{′} \text{-} hyfroxybufuralol formed with inhibitor )\end{matrix}}{( {1}^{′} \text{-} hydroxybufuralol area formed without inhibitor )} \times 100$$

The IC₅₀ is calculated from the percent inhibition of the formation of the 1′-hydroxybufuralol as follows (assuming competitive inhibition):

$$\frac{NCE Concentration \times ( 100 - Percent of inhibition )}{Percent of inhibition}$$

The IC₅₀ estimation is assumed valid if inhibition is between 20% and 80% (Moody G C, Griffin S J, Mather A N, McGinnity D F, Riley R J. (1999) Fully automated analysis of activities catalyzed by the major human liver cytochrome P450 (CYP) enzymes: assessment of human CYP inhibition potential. Xenobiotica 29(1): 53-75).

The invention being thus described, it is obvious that the same can be varied in many ways. Such variations are not to be regarded as a departure from the spirit and scope of the present invention, and all such modifications as would be obvious to one skilled in the art are intended to be included within the scope of the following claims.

1. A method of treating stuttering or another communication disorder, comprising administering to a patient in need of such treatment an effective amount of a norepinephrine reuptake inhibitor selected from the group consisting of
atomoxetine or a pharmaceutically acceptable salt thereof and a compound of formula (I): 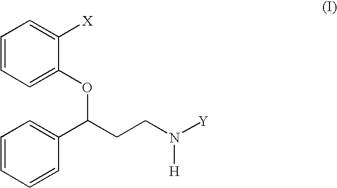
wherein X is C₁-C₄ alkylthio, and Y is C₁-C₂ alkyl, or a pharmaceutically acceptable salt thereof. 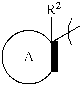
is a group of formula (a) or (b)

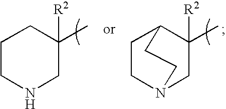

2. (canceled)

3. The method of claim 1, wherein said norepinephrine reuptake inhibitor is atomoxetine hydrochloride.
